(19) United States
(12) Patent Application Publication
(10) Pub. No.: US 2025/0340510 A1
(43) Pub. Date: Nov. 6, 2025

(54) Substituted Phenylbenzenesulfonamide Derivatives and Uses Thereof

(71) Applicant: Nine Square Therapeutics Corporation, South San Francisco, CA (US)

(72) Inventors: Mark D. Rosen, San Diego, CA (US); Qiang Cong, Palo Alto, CA (US); Chakrapani Kalyanaraman, Redwood City, CA (US); Robert Lee Dow, Groton, CT (US)

(73) Assignee: Nine Square Therapeutics Corporation, South San Francisco, CA (US)

(21) Appl. No.: 19/123,480

(22) PCT Filed: Oct. 27, 2023
(86) PCT No.: PCT/US2023/078090

§ 371 Date: Apr. 23, 2025

Related U.S. Application Data
(60) Provisional application No. 63/420,369, filed on Oct. 28, 2022.

(51) Int. Cl. C07C 311/44; A61K 31/18; A61K 31/337; A61K 31/341; A61K 31/351; A61K 31/352; A61K 31/381; A61K 31/415; A61K 31/417; A61K 31/4188; A61K 31/425; A61K 31/426; A61K 31/44; A61K 31/4402; A61K 31/4406; A61K 31/4409; A61K 31/4468; A61K 31/5375; C07D 211/58; C07D 213/42; C07D 213/61; C07D 231/12; C07D 233/64; C07D 275/02; C07D 277/28; C07D 295/125; C07D 305/06; C07D 307/22; C07D 309/06; C07D 309/14; C07D 311/96; C07D 333/20; C07D 487/04

(52) U.S. Cl. CPC C07C 311/44; A61K 31/18; A61K 31/337; A61K 31/341; A61K 31/351; A61K 31/352; A61K 31/381; A61K 31/415; A61K 31/417; A61K 31/4188; A61K 31/425; A61K 31/426; A61K 31/44; A61K 31/4402; A61K 31/4406; A61K 31/4409; A61K 31/4468; A61K 31/5375; C07D 211/58; C07D 213/42; C07D 213/61; C07D 231/12; C07D 233/64; C07D 275/02; C07D 277/28; C07D 295/125; C07D 305/06; C07D 307/22; C07D 309/06; C07D 309/14; C07D 311/96; C07D 333/20; C07D 487/04; C07C 2601/02; C07C 2601/04; C07C 2601/08; C07C 2601/14; C07C 2602/16

(57) ABSTRACT

Provided herein are substituted phenylbenzenesulfonamide compounds and compositions thereof for modulating TRPML1. In some embodiments, the compounds and compositions are provided for treatment of neurodegenerative diseases.

### CROSS REFERENCE TO RELATED APPLICATIONS

This application claims the benefit of priority of U.S. Provisional Patent Application No. 63/420,369, filed Oct. 28, 2022, which application is hereby incorporated by reference in its entirety. Throughout this application various publications, patents, and/or patent applications are referenced. The disclosures of the publications, patents and/or patent applications are hereby incorporated by reference in their entireties into this application in order to more fully describe the state of the art to which this disclosure pertains.

### FIELD

The present disclosure relates generally to compounds, compositions, and methods for their preparation and use of substituted phenylbenzenesulfonamide derivative compounds and compositions, e.g., for treating neurodegenerative diseases.

### BACKGROUND

Transient receptor potentials (TRP) are multifunctional signalling molecules involved in sensory perception and cellular physiology. The 28 members of the mammalian TRP channel superfamily are divided into six subfamilies, one of which is mucolipins (TRPML1-3). Mucolipin TRP channel subfamily 1 (TRPML1) is the main Ca²⁺-releasing channel localized at the lysosomal membrane (Zeevi et al., Biochim. Biophys. Acta 2007, 1772, 851-858). Lysosomal Ca²⁺ released through the TRPML1 channel promotes the dephosphorylation and subsequent nuclear translocation of transcription factor EB (TFEB), which increases the transcription of genes that promote autophagy and lysosomal biogenesis (Tedeschi et al., Cell 2019, 8, 1216). Due to its important regulatory function and ability to clear pathogenic molecules, TRPML1 has attracted attention as a potential target for lysosomal storage diseases, metabolic diseases, cardiovascular diseases, inflammatory disorders, immunological disorders, cancer, aging, and neurodegenerative diseases (Krogsaeter et al., Cell Calcium 2022, 103, 102553 and Park et al., Front. Cell Dev. Biol. 2022, 10, 811701). For instance, TRPML1 was shown to regulate α-synuclein exocytosis in dopaminergic neurons in a Parkinson's disease mouse model (Tsunemi et al., J. Neurosci. 2019, 39, 5760-5772). These studies identify novel small molecule agonists that activate TRPML1 and that are useful for treatment of diseases and disorders related to lysosomal and autophagy-related diseases and disorders.

### SUMMARY

The present embodiments can be understood more fully by reference to the detailed description and examples, which are intended to exemplify non-limiting embodiments.

Disclosed herein are compounds of Formula (I):

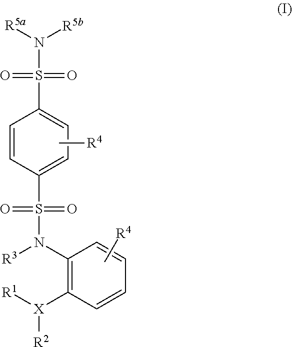

or a pharmaceutically acceptable salt thereof, wherein
X is N or H;
R¹ is chosen from H and optionally substituted alkyl;
R² is chosen from optionally substituted alkyl, optionally substituted cycloalkyl, aryl, and optionally substituted heterocyclyl;
   or, when X is H, then R¹ and R² are absent;
R³ is H, —CH₃, or optionally substituted C₂-C₆ alkyl;
or R¹ and R³ together with the atoms attached thereto form a 5- to 7-membered ring;
each R⁴ is independently H, halo, cyano, or optionally substituted alkyl; and
R^{5a} and R^{5b} are each independently H or optionally substituted alkyl,
with the proviso that R² is not pyrrolidine.

In another aspect, provided herein is a compound selected from the compounds disclosed herein or a pharmaceutically acceptable salt thereof.

In further aspect, provided herein is a pharmaceutical composition comprising a compound disclosed herein, or a pharmaceutically acceptable salt thereof, and a pharmaceutically acceptable excipient.

In another aspect, provided herein is a method of modulating Mucolipin TRP channel subfamily 1 (TRPML1) comprising contacting TRPML1 with an effective amount of a compound disclosed herein, or a pharmaceutically acceptable salt thereof, or the pharmaceutical composition disclosed herein.

In related aspect, provided herein is a method of treating a disease associated with TRPML1 comprising administering to the subject an effective amount of a compound disclosed herein, or a pharmaceutically acceptable salt thereof, or the pharmaceutical composition disclosed herein.

In another aspect, provided herein is a use of a compound disclosed herein, or a pharmaceutically acceptable salt thereof, in the manufacture of a medicament for modulating TRPML1 in a subject.

In another aspect, provided herein is a use of a compound disclosed herein, or a pharmaceutically acceptable salt thereof, in the manufacture of a medicament for treating a disease associated with TRPML1 in a subject.

In related aspect, provided herein is a pharmaceutical formulation comprising a compound disclosed herein, or a pharmaceutically acceptable salt thereof, and a pharmaceutically acceptable carrier.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group has monovalency. Examples of alkyl groups with monovalency include, but are not limited to, —CH₃, —CH₂CH₃, —CH₂CH₂CH₃, —CH₂CH₂(CH₃), —CH₂(CH₂)₂CH₃, —CH₂CH(CH₃)CH₃, —CH₂(CH₂)₃CH₃, —CH₂(CH₂)₄CH₃, —CH₂(CH₂)₅CH₃, —CH₂(CH₂)₆CH₃, and the like. In some embodiments, the alkyl group has bivalency. Examples of alkyl groups with bivalency include, but are not limited to, —CH₂—, —CH₂CH₂—, —CH₂CH₂CH₂—, —CH₂CH(CH₃)—, —CH₂(CH₂)₂CH₂—, —CH₂CH(CH₃)CH₂—, —CH₂(CH₂)₃CH₂—, —CH₂(CH₂)₄CH₂—, —CH₂(CH₂)₅CH₂—, —CH₂(CH₂)₆CH₂—, and the like. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen, hydroxy, alkoxy, cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy, oxo (═O), amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino, acylamino, sulfonylamino, oxime, hydroxylamino, hydrazine, hydrazido, hydrazono, azido, nitro, thio (—SH), alkylthio, ═S, sulfinyl, sulfonyl, aminosulfonyl, acyl, formyl, carboxy, ester, carbamate, amido, cyano, or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; aminocarbonyl; acylamino; thiocarbonyl; sulfinyl; sulfone; sulfonamide; ketone; aldehyde; ester; oxime; hydroxyl amine; N-oxide; hydrazine; hydrazide; hydrazone; azide; —B(OH)₂; or —O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1methylcyclopropyl, 2methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom. In some embodiments, heterocyclyl groups include 3 to 10 ring members, whereas other such groups have 3 to 5, 3 to 6, or 3 to 8 ring members. In some embodiments, heterocyclyl groups include one to three heteroatoms, whereas other such groups have one to two heteroatoms or one heteroatom. Heterocyclyls can also be bonded to other groups at any ring atom (i.e., at any carbon atom or heteroatom of the heterocyclic ring). A heterocyclyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4dioxaspiro[4.5]decanyl, homopiperazinyl, quinuclidyl, or tetrahydropyrimidin-2(1H)-one. Representative substituted heterocyclyl groups may be monosubstituted or substituted more than once, such as, but not limited to, pyridyl or morpholinyl groups, which are 2-, 3-, 4-, 5-, or 6 substituted, or disubstituted with various substituents such as those listed below. In some embodiments, when the heterocyclyl group described herein is said to contain only heteroatom X, it exclusively contains at least one heteroatom X, and does not contain any other heteroatoms. For example, “heterocyclyl that contains only N” describes a heterocyclyl that contains one, two, three, or more nitrogen heteroatoms, and no other heteroatoms such as oxygen or sulfur. In some embodiments, when the heterocyclyl group described herein is said to contain at least heteroatom X, it contains at least one heteroatom X and may or may not also contain additional heteroatoms of different types. For example, “heterocyclyl that contains at least N” may describe a heterocyclyl that contains one, two, three, or more nitrogen heteroatoms with zero, one, two, three, or more oxygen heteroatoms and/or one, two, three, or more sulfur heteroatoms.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 10 ring atoms, and in others from 6 to 9 or even 6 to 10 atoms in the ring portions of the groups. In some embodiments, heterocyclyl groups have one to three heteroatoms, whereas other such groups have one to two heteroatoms or one heteroatom. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1Hpyrrolo[2,3b]pyridyl), indazolyl, benzimidazolyl (e.g., 1Hbenzo[d]imidazolyl), imidazopyridyl (e.g., azabenzimidazolyl or 1Himidazo[4,5b]pyridyl), pyrazolopyridyl, triazolopyridyl, benzotriazolyl (e.g., 1Hbenzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4dihydroisoquinolin-1(2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “oxo” group is a “═O” group bonded to a carbon.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); —B(OH)₂, —O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocycloalkyl group is substituted with an alkyl group and situations where the heterocycloalkyl group is not substituted with alkyl.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan.

Embodiments of the disclosure are meant to encompass pharmaceutically acceptable salts, tautomers, isotopologues, and stereoisomers of the compounds provided herein, such as the compounds of Formulas (I), (II), (III), (IV), and (V), as well as the compounds in Table 1.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds disclosed herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (Nmethyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and ptoluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically-pure compound having one chiral center will be substantially free of the opposite enantiomer of the compound. A stereoisomerically pure compound having two chiral centers will be substantially free of other diastereomers of the compound. A typical stereoisomerically-pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically-pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KgaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

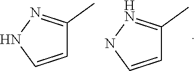

As readily understood by one skilled in the art, a wide variety of functional groups and other structures may exhibit tautomerism and all tautomers of compounds disclosed herein are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Activation” as used herein, means a method of making a biological molecule reactive, active, or effective in carrying out its function. In one embodiment, the biological molecule is a signalling molecule. In one embodiment, the biological molecule is TRPML1.

The term “agonist” as used herein refers to a molecule that can bind to and activate a receptor to produce a biological response.

“Modulation” as used herein, means a method of altering the activity of another biological molecule, wherein the activity can increase or decrease.

“Treating” or “treatment” of a disease or a disorder, which are herein used interchangeably, in a subject refers to 1) preventing at least one symptom or preventing the recurrence of at least one symptom; 2) inhibiting the disease or at least one symptom thereof or arresting its development; or 3) ameliorating or causing regression of the disease, or at least one symptom thereof. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the invention contemplate any one or more of these aspects of treatment.

The term “effective amount” in connection with a compound disclosed herein means an amount capable of treating a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an TRPML1 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

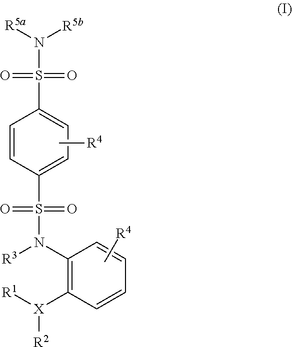

or a pharmaceutically acceptable salt thereof, wherein
X is N or H;
R¹ is chosen from H and optionally substituted alkyl;
R² is chosen from optionally substituted alkyl, optionally substituted cycloalkyl, aryl, and optionally substituted heterocyclyl;
   or, when X is H, then R¹ and R² are absent;
R³ is H, —CH₃, or optionally substituted C₂-C₆ alkyl;
   or R¹ and R³ together with the atoms attached thereto form a 5- to 7-membered ring;
each R⁴ is independently H, halo, cyano, or optionally substituted alkyl; and
R^{5a} and R^{5b} are each independently H or optionally substituted alkyl,
with the proviso that R² is not pyrrolidine.

In some embodiments, X is N or H. In some embodiments, X is N. In some embodiments, X is H.

In some embodiments, R¹ is chosen from H and optionally substituted alkyl. In some embodiments, R¹ is chosen from H and optionally substituted C₁-C₆ alkyl. In some embodiments, R¹ is chosen from H and C₁-C₆ alkyl optionally substituted with halo, oxo, or 3-6 membered heterocyclyl that contains at least O. In some embodiments, R¹ is chosen from H and C₁-C₃ alkyl optionally substituted with halo, oxo, or 4-6 membered heterocyclyl that contains at least O. In some embodiments, R¹ is chosen from H and C₁-C₃ alkyl optionally substituted with F, Cl, Br, I, oxo, or 4-6 membered heterocyclyl that contains at least O. In some embodiments, R¹ is chosen from H and C₁-C₃ alkyl optionally substituted with F, oxo, or 4-6 membered heterocyclyl that contains only O.

In some embodiments, R¹ is H.

In some embodiments, R¹ is optionally substituted alkyl. In some embodiments, R¹ is optionally substituted C₁-C₆ alkyl alkyl. In some embodiments, R¹ is C₁-C₆ alkyl optionally substituted with halo, oxo, or 3-6 membered heterocyclyl that contains at least O. In some embodiments, R¹ is C₁-C₆ alkyl optionally substituted with F, Cl, Br, I, oxo, or 4-6 membered heterocyclyl that contains at least O. In some embodiments, R¹ is C₁-C₃ alkyl optionally substituted with F, Cl, Br, I, oxo, or 4-6 membered heterocyclyl that contains at least O. In some embodiments, R¹ is C₁-C₃ alkyl optionally substituted with F, oxo, or 4-6 membered heterocyclyl that contains only O. In some embodiments, R¹ is

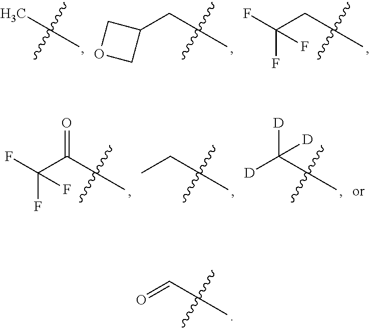

In some embodiments, R² is chosen from optionally substituted alkyl, optionally substituted, aryl, and optionally substituted heterocyclyl. In some embodiments, R² is chosen from optionally substituted C₁-C₆ alkyl, optionally substituted C₃-C₆ cycloalkyl, 6-membered aryl, optionally substituted heterocyclyl that contains at least N, and optionally substituted heterocyclyl that contains at least O. In some embodiments, R² is chosen from optionally substituted C₁-C₆ alkyl, optionally substituted C₃-C₆ cycloalkyl, 6-membered aryl, 4-6 membered heterocyclyl that contains at least N optionally substituted with C₁-C₆ alkyl, —C(O)O(C₁-C₆ alkyl), —C(O)(C₁-C₆ alkyl), or —C(O)(C₁-C₆ cycloalkyl), and 3-10 membered heterocyclyl that contains at least O optionally substituted with C₁-C₆ alkyl. In some embodiments, R² is chosen from optionally substituted C₁-C₅ alkyl, optionally substituted C₄-6 cycloalkyl, 6-membered aryl, 6-membered heterocyclyl that contains at least N optionally substituted with C₁-C₃ alkyl, —C(O)O(C₁-C₆ alkyl), —C(O)(C₁-C₆ alkyl), or —C(O)(C₁-C₆ cycloalkyl), and 3-10 membered heterocyclyl that contains at least O optionally substituted with C₁-C₃ alkyl. In some embodiments, R² is chosen from optionally substituted C₁-C₅ alkyl, optionally substituted C₄-C₆ cycloalkyl, 6-membered aryl, 6-membered heterocyclyl that contains only N optionally substituted with C₁-C₃ alkyl, —C(O)O(C₁-C₄ alkyl), —C(O)(C₁-C₄ alkyl), or —C(0)(C₁-C₄ cycloalkyl), and 4-9 membered heterocyclyl that contains at least O optionally substituted with C₁-C₃ alkyl. In some embodiments, R² is chosen from optionally substituted C₁-C₅ alkyl, optionally substituted C₄-C₆ cycloalkyl, phenyl, 6-membered heterocyclyl that contains only N optionally substituted with C₁-C₃ alkyl, —C(O)O(C₁-C₄ alkyl), —C(O)(C₁-C₄ alkyl), or —C(O)(C₁-C₄ cycloalkyl), and 4-9 membered heterocyclyl that contains only O optionally substituted with C₁-C₃ alkyl. In some embodiments, R² is chosen from optionally substituted C₁-C₅ alkyl, optionally substituted C₄-C₆ cycloalkyl, phenyl, 6-membered heterocyclyl that contains only N optionally substituted with C₁-C₃ alkyl, —C(O)O(C₁-C₄ alkyl), —C(O)(C₁-C₄ alkyl), or —C(O)(C₁-C₄ cycloalkyl), and 4-9 membered heterocyclyl that contains only O optionally substituted with —CH₃.

In some embodiments, R² is chosen from optionally substituted alkyl. In some embodiments, R² is chosen from optionally substituted C₁-C₆ alkyl. In some embodiments, R² is chosen from optionally substituted C₁-C₅ alkyl. In some embodiments, R² is

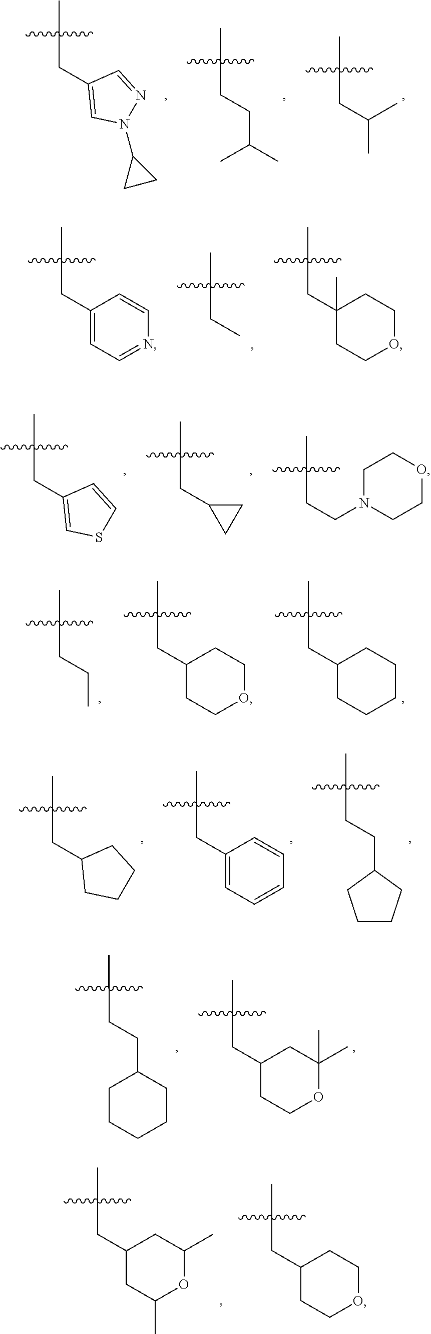

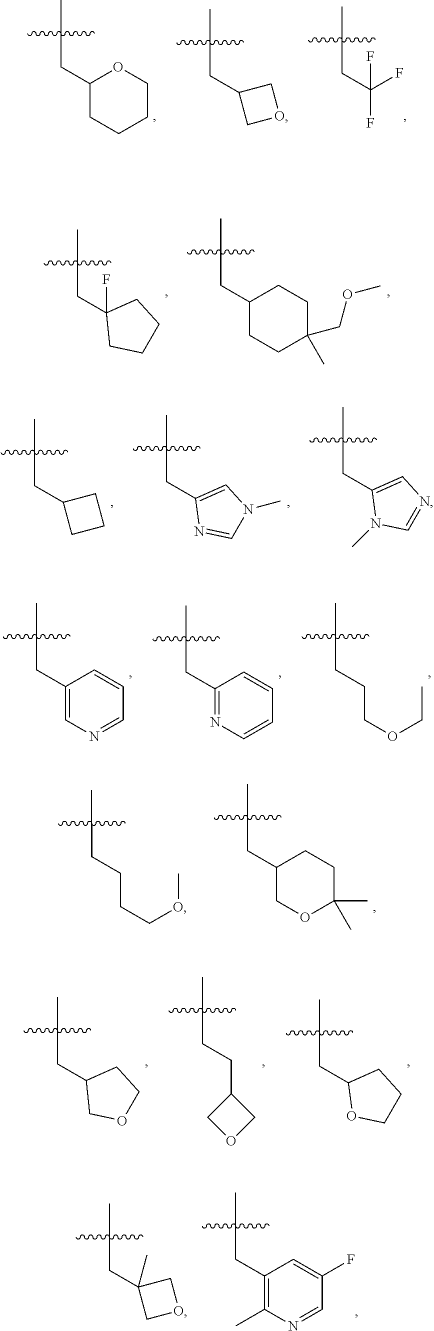

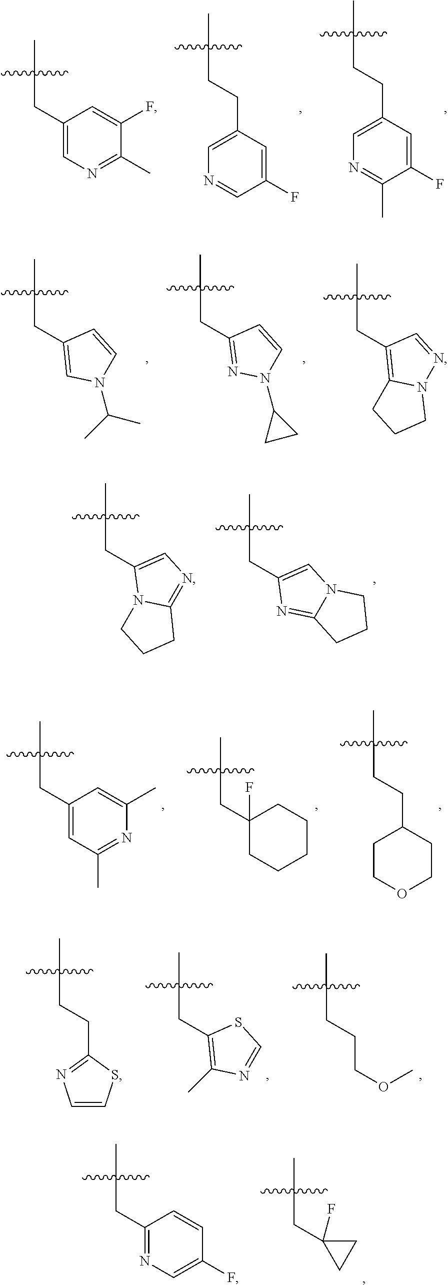

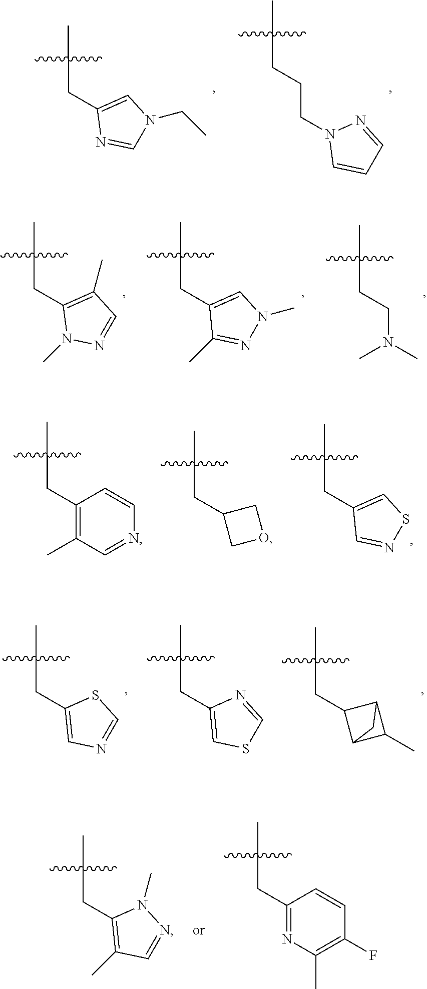

In some embodiments, R² is optionally substituted cycloalkyl. In some embodiments, R² is optionally substituted C₃₋₆ cycloalkyl. In some embodiments, R² is optionally substituted C₄-6 cycloalkyl. In some embodiments, R² is

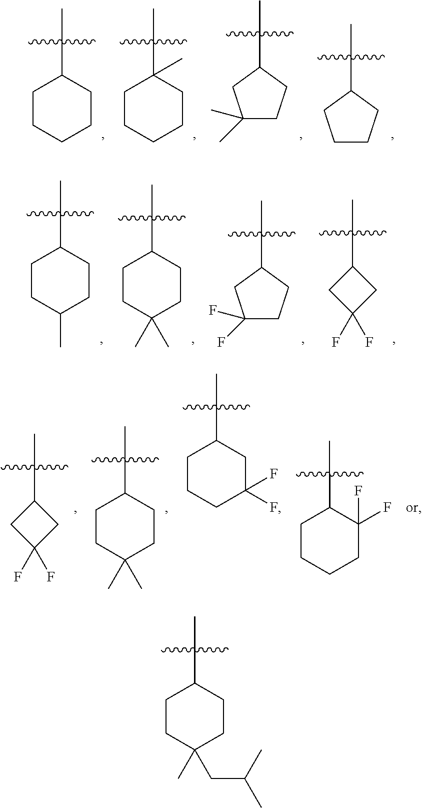

In some embodiments, R² is aryl. In some embodiments, R² is 6-membered aryl. In some embodiments, R² is phenyl.

In some embodiments, R² is optionally substituted heterocyclyl. In some embodiments, R² is optionally substituted heterocyclyl that contains at least N. In some embodiments, R² is 4-6 membered heterocyclyl that contains at least N optionally substituted with C₁-C₆ alkyl, —C(O)O(C₁-C₆ alkyl), —C(O)(C₁-C₆ alkyl), or —C(O)(C₁-C₆ cycloalkyl). In some embodiments, R² is 6-membered heterocyclyl that contains at least N optionally substituted with C₁-C₆ alkyl, —C(O)O(C₁-C₆ alkyl), —C(O)(C₁-C₆ alkyl), or —C(O)(C₁-C₆ cycloalkyl). In some embodiments, R² is 6-membered heterocyclyl that contains at least N optionally substituted with C₁-C₃ alkyl, —C(O)O(C₁-C₄ alkyl), —C(O)(C₁-C₄ alkyl), or —C(O)(C₁-C₄ cycloalkyl). In some embodiments, R² is 6-membered heterocyclyl that contains only N optionally substituted with C₁-C₃ alkyl, —C(O)O(C₁-C₄ alkyl), —C(O)(C₁-C₄ alkyl), or —C(O)(C₁-C₄ cycloalkyl). In some embodiments, R² is

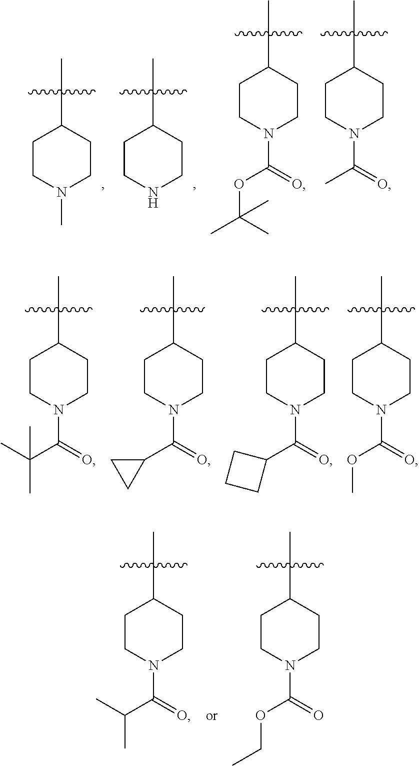

In some embodiments, R² is optionally substituted heterocyclyl that contains at least O. In some embodiments, R² is 3-10 membered heterocyclyl that contains at least O optionally substituted with C₁-C₆ alkyl. In some embodiments, R² is 3-10 membered heterocyclyl that contains at least O optionally substituted with C₁-C₃ alkyl. In some embodiments, R² is 4-9 membered heterocyclyl that contains at least O optionally substituted with C₁-C₃ alkyl. R² is 4-9 membered heterocyclyl that contains only O optionally substituted with C₁-C₃ alkyl. In some embodiments, R² is 4-9 membered heterocyclyl that contains only O optionally substituted with —CH₃. In some embodiments, R² is

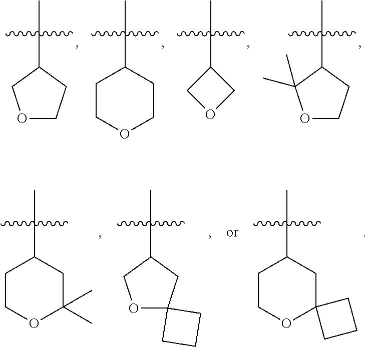

In some embodiments, R³ is H, —CH₃, or optionally substituted C₂-C₆ alkyl. In some embodiments, R³ is H, —CH₃, or optionally substituted C₂ alkyl. In some embodiments, R³ is H. In some embodiments, R³ is —CH₃. In some embodiments, R³ is optionally substituted C₂-C₆ alkyl. In some embodiments, R³ is optionally substituted C₂ alkyl.

In some embodiments, R¹ and R³ together with the atoms attached thereto form a 5-7 membered ring. In some embodiments, R¹ and R³ together with the atoms attached thereto form a 5-6 membered ring.

In some embodiments, R⁴ is H, halo, cyano, or optionally substituted alkyl. In some embodiments, R⁴ is H, halo, cyano, or optionally substituted C₁-C₆ alkyl. In some embodiments, R⁴ is H, F, Cl, Br, I, cyano, or optionally substituted C₁-C₃ alkyl. In some embodiments, R⁴ is H, F, C₁, cyano, or C₁-C₃ alkyl optionally substituted with halo. In some embodiments, R⁴ is H, F, C₁, cyano, or C₁-C₃ alkyl optionally substituted with F. In some embodiments, R⁴ is H, F, C₁, cyano, or —CH₃ optionally substituted with one or more F. In some embodiments, R⁴ is H. In some embodiments, R⁴ is halo. In some embodiments, R⁴ is F, Cl, Br, or I. In some embodiments, R⁴ is halo. In some embodiments, R⁴ is F or Cl. In some embodiments, R⁴ is cyano. In some embodiments, R⁴ is optionally substituted alkyl. In some embodiments, R⁴ is optionally substituted C₁-C₆ alkyl. In some embodiments, R⁴ is optionally substituted C₁-C₃ alkyl. In some embodiments, R⁴ is C₁-C₃ alkyl optionally substituted with halo. In some embodiments, R⁴ is C₁-C₃ alkyl optionally substituted with F. In some embodiments, R⁴ is CH₃ optionally substituted with one or more F.

In some embodiments, R^{5a} and R^{5b} are each independently H or optionally substituted alkyl. In some embodiments, R^{5a} and R^{5b} are each independently H or optionally substituted C₁-C₆ alkyl. In some embodiments, R^{5a} and R^{5b} are each independently H or optionally substituted C₁-C₃ alkyl. In some embodiments, R^{5a} and R^{5b} are each independently H or optionally substituted —CH₃. In some embodiments, R^{5a} and R^{5b} are both —CH₃.

In some embodiments, the compound of Formula (I) is a compound of Formula (II):

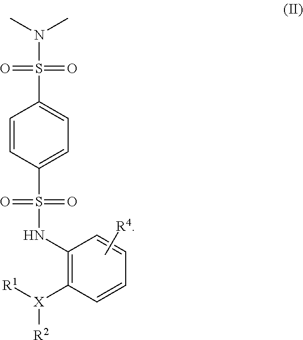

wherein R¹, R², and R⁴ are as described for Formula (I).

In some embodiments, the compound of Formula (I) is a compound of Formula (III):

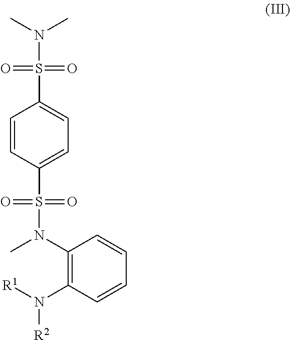

wherein R¹ and R² are as described for Formula (I).

In some embodiments, the compound of Formula (I) is a compound of Formula (IV):

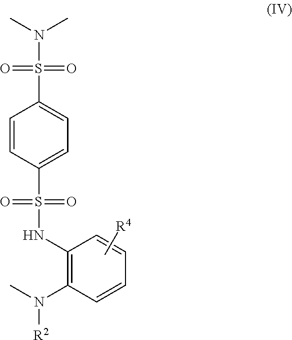

wherein R² and R⁴ are as described for Formula (I).

In some embodiments, the compound of Formula (I) is a compound of Formula (V):

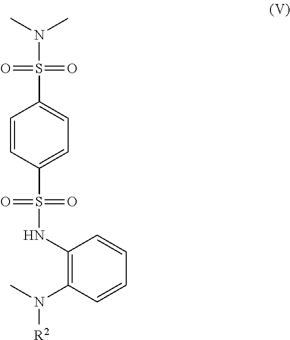

wherein R² is as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to X of Formula (I) may be combined with every description, variation, embodiment, or aspect of R¹, R², R³, and R⁴ the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (III), (IV), and (V), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, may be presented as specific stereoisomers and/or in a non-stereochemical form, it is understood that any or all stereochemical forms, including any enantiomeric or diastereomeric forms, and any tautomers or other forms of any of the compounds of the present disclosure (e.g., compounds of Formulas (I), (II), (III), (IV), and (V) as well as those in Table 1), are herein described.

TABLE 1

| Compound | | |
|---|---|---|
| No. | Structure | Name |
| 1 | 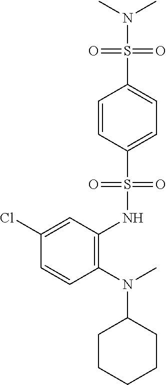 [SMILES: CN(c1ccc(Cl)cc1NS(=O)(=O)c1ccc(S(=O)(=O)N(C)C)cc1)C1CCCCC1] | N1-(5-chloro-2- (cyclohexyl(methyl)amino)phenyl)-N4,N4- dimethylbenzene-1,4-disulfonamide |
| 2 | 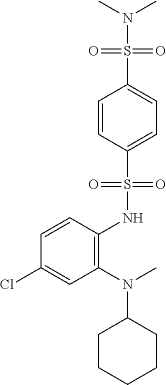 [SMILES: CN(c1cc(Cl)ccc1NS(=O)(=O)c1ccc(S(=O)(=O)N(C)C)cc1)C1CCCCC1] | N1-(4-chloro-2- (cyclohexyl(methyl)amino)phenyl)-N4,N4- dimethylbenzene-1,4-disulfonamide |
| 3 | 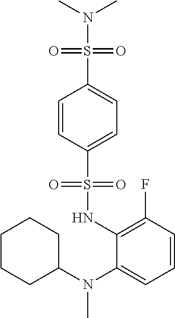 [SMILES: CN(c1cccc(F)c1NS(=O)(=O)c1ccc(S(=O)(=O)N(C)C)cc1)C1CCCCC1] | N1-(2-(cyclohexyl(methyl)amino)-6- fluorophenyl)-N4,N4-dimethylbenzene-1,4- disulfonamide |
| 4 | 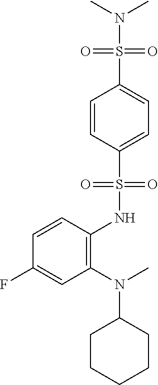 [SMILES: CN(c1cc(F)ccc1NS(=O)(=O)c1ccc(S(=O)(=O)N(C)C)cc1)C1CCCCC1] | N1-(2-(cyclohexyl(methyl)amino)-4- fluorophenyl)-N4,N4-dimethylbenzene-1,4- disulfonamide |
| 5 | 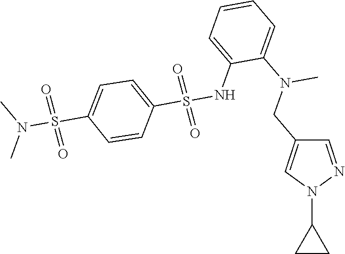 [SMILES: CN(Cc1cnn(C2CC2)c1)c1ccccc1NS(=O)(=O)c1ccc(S(=O)(=O)N(C)C)cc1] | N1-(2-(((1-cyclopropyl-1H-pyrazol-4- yl)methyl)(methyl)amino)phenyl)-N4,N4- dimethylbenzene-1,4-disulfonamide |
| 6 | 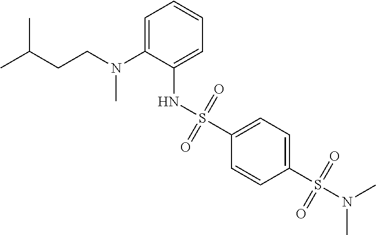 [SMILES: CC(C)CCN(C)c1ccccc1NS(=O)(=O)c1ccc(S(=O)(=O)N(C)C)cc1] | N1-(2-(isopentyl(methyl)amino)phenyl)- N4,N4-dimethylbenzene-1,4-disulfonamide |
| 7 | 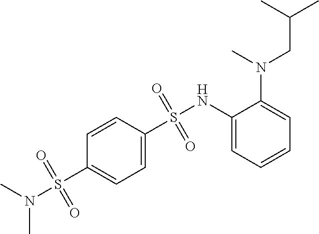 [SMILES: CC(C)CN(C)c1ccccc1NS(=O)(=O)c1ccc(S(=O)(=O)N(C)C)cc1] | N1-(2-(isobutyl(methyl)amino)phenyl)- N4,N4-dimethylbenzene-1,4-disulfonamide |
| 8 | 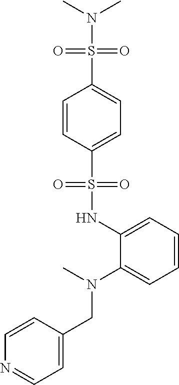 [SMILES: CN(Cc1ccncc1)c1ccccc1NS(=O)(=O)c1ccc(S(=O)(=O)N(C)C)cc1] | N1,N1-dimethyl-N4-(2-(methyl(pyridin-4- ylmethyl)amino)phenyl)benzene-1,4- disulfonamide |
| 9 | 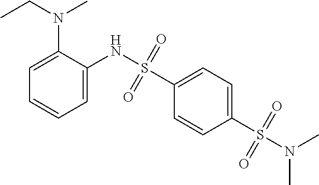 [SMILES: CCN(C)c1ccccc1NS(=O)(=O)c1ccc(S(=O)(=O)N(C)C)cc1] | N1-(2-(ethyl(methyl)amino)phenyl)- N4,N4-dimethylbenzene-1,4-disulfonamide |
| 10 | 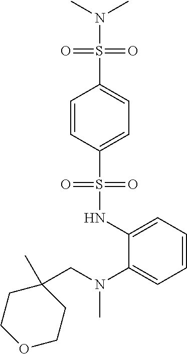 [SMILES: CN(CC1(C)CCOCC1)c1ccccc1NS(=O)(=O)c1ccc(S(=O)(=O)N(C)C)cc1] | N1,N1-dimethyl-N4-(2-(methyl((4- methyltetrahydro-2H-pyran-4- yl)methyl)amino)phenyl)benzene-1,4- disulfonamide |
| 11 | 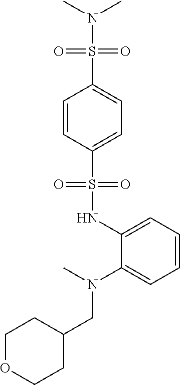 [SMILES: CN(CC1CCOCC1)c1ccccc1NS(=O)(=O)c1ccc(S(=O)(=O)N(C)C)cc1] | N1,N1-dimethyl-N4-(2- (methyl((tetrahydro-2H-pyran-4- yl)methyl)amino)phenyl)benzene-1,4- disulfonamide |
| 12 | 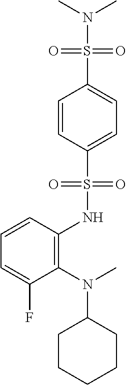 [SMILES: CN(c1c(F)cccc1NS(=O)(=O)c1ccc(S(=O)(=O)N(C)C)cc1)C1CCCCC1] | N1-(2-(cyclohexyl(methyl)amino)-3- fluorophenyl)-N4,N4-dimethylbenzene-1,4- disulfonamide |
| 13 | 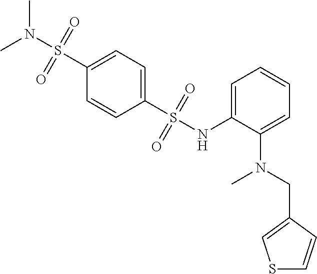 [SMILES: CN(Cc1ccsc1)c1ccccc1NS(=O)(=O)c1ccc(S(=O)(=O)N(C)C)cc1] | N1,N1-dimethyl-N4-(2-(methyl(thiophen- 3-ylmethyl)amino)phenyl)benzene-1,4- disulfonamide |
| 14 | 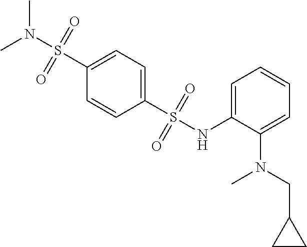 [SMILES: CN(CC1CC1)c1ccccc1NS(=O)(=O)c1ccc(S(=O)(=O)N(C)C)cc1] | N1-(2- ((cyclopropylmethyl)(methyl)amino)phenyl)- N4,N4-dimethylbenzene-1,4- disulfonamide |
| 15 | 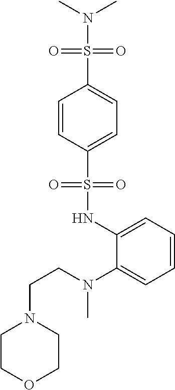 [SMILES: CN(CCN1CCOCC1)c1ccccc1NS(=O)(=O)c1ccc(S(=O)(=O)N(C)C)cc1] | N1,N1-dimethyl-N4-(2-(methyl(2- morpholinoethyl)amino)phenyl)benzene- 1,4-disulfonamide |
| 16 | 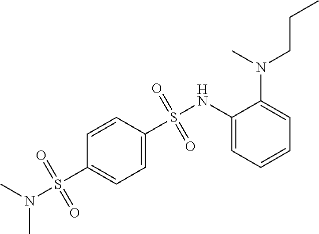 [SMILES: CCCN(C)c1ccccc1NS(=O)(=O)c1ccc(S(=O)(=O)N(C)C)cc1] | N1,N1-dimethyl-N4-(2- (methyl(propyl)amino)phenyl)benzene-1,4- disulfonamide |
| 17 | 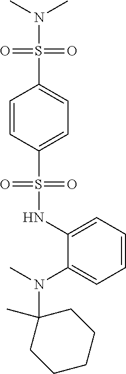 [SMILES: CN(c1ccccc1NS(=O)(=O)c1ccc(S(=O)(=O)N(C)C)cc1)C1(C)CCCCC1] | N1,N1-dimethyl-N4-(2-(methyl(1- methylcyclohexyl)amino)phenyl)benzene- 1,4-disulfonamide |
| 18 | 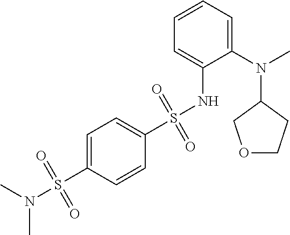 | N1,N1-dimethyl-N4-(2- (methyl(tetrahydrofuran-3- yl)amino)phenyl)benzene-1,4- disulfonamide |
| 19 | 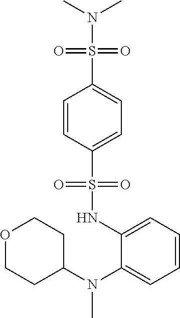 [SMILES: CN(c1ccccc1NS(=O)(=O)c1ccc(S(=O)(=O)N(C)C)cc1)C1CCOCC1] | N1,N1-dimethyl-N4-(2-(methyl(tetrahydro- 2H-pyran-4-yl)amino)phenyl)benzene-1,4- disulfonamide |
| 20 | 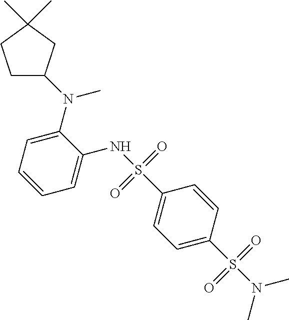 [SMILES: CN(c1ccccc1NS(=O)(=O)c1ccc(S(=O)(=O)N(C)C)cc1)C1CCC(C)(C)C1] | N1-(2-((3,3- dimethylcyclopentyl)(methyl)amino)phenyl)- N4,N4-dimethylbenzene-1,4- disulfonamide |
| 21 | 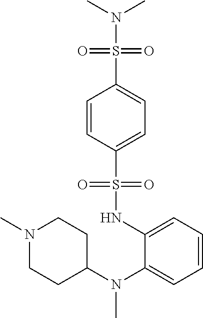 [SMILES: CN1CCC(N(C)c2ccccc2NS(=O)(=O)c2ccc(S(=O)(=O)N(C)C)cc2)CC1] | N1,N1-dimethyl-N4-(2-(methyl(1- methylpiperidin-4- yl)amino)phenyl)benzene-1,4- disulfonamide |
| 22 | 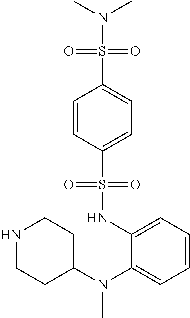 [SMILES: CN(c1ccccc1NS(=O)(=O)c1ccc(S(=O)(=O)N(C)C)cc1)C1CCNCC1] | N1,N1-dimethyl-N4-(2-(methyl(piperidin- 4-yl)amino)phenyl)benzene-1,4- disulfonamide |
| 23 | 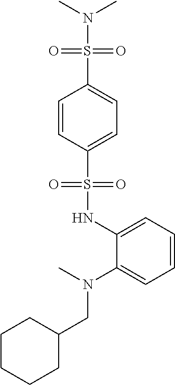 [SMILES: CN(CC1CCCCC1)c1ccccc1NS(=O)(=O)c1ccc(S(=O)(=O)N(C)C)cc1] | N1-(2- ((cyclohexylmethyl)(methyl)amino)phenyl)- N4,N4-dimethylbenzene-1,4- disulfonamide |
| 24 | 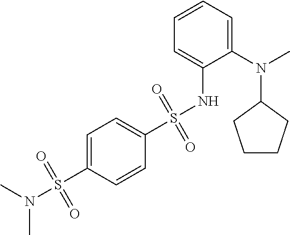 | N1-(2-(cyclopentyl(methyl)amino)phenyl)- N4,N4-dimethylbenzene-1,4-disulfonamide |
| 25 | 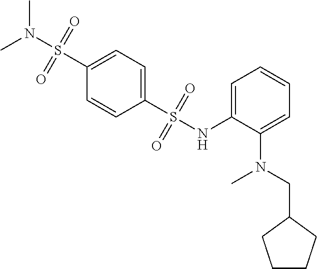 [SMILES: CN(CC1CCCC1)c1ccccc1NS(=O)(=O)c1ccc(S(=O)(=O)N(C)C)cc1] | N1-(2- ((cyclopentylmethyl)(methyl)amino)phenyl)- N4,N4-dimethylbenzene-1,4- disulfonamide |
| 26 | 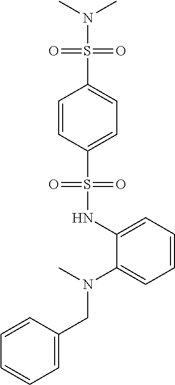 [SMILES: CN(Cc1ccccc1)c1ccccc1NS(=O)(=O)c1ccc(S(=O)(=O)N(C)C)cc1] | N1-(2-(benzyl(methyl)amino)phenyl)- N4,N4-dimethylbenzene-1,4-disulfonamide |
| 27 | 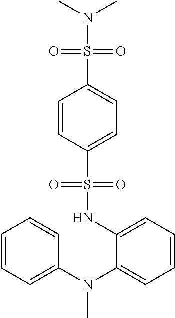 [SMILES: CN(c1ccccc1)c1ccccc1NS(=O)(=O)c1ccc(S(=O)(=O)N(C)C)cc1] | N1,N1-dimethyl-N4-(2- (methyl(phenyl)amino)phenyl)benzene-1,4- disulfonamide |
| 28 | 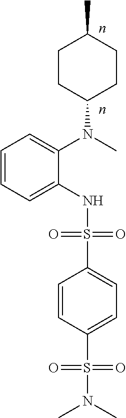 [SMILES: CN(C)S(=O)(=O)c1ccc(S(=O)(=O)Nc2ccccc2N(C)[C@H]2CC[C@H](C)CC2)cc1] | N1,N1-dimethyl-N4-(2-(methyl((1r,4r)-4- methylcyclohexyl)amino)phenyl)benzene- 1,4-disulfonamide |
| 29 | 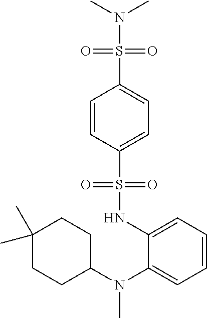 [SMILES: CN(c1ccccc1NS(=O)(=O)c1ccc(S(=O)(=O)N(C)C)cc1)C1CCC(C)(C)CC1] | N1-(2-((4,4- dimethylcyclohexyl)(methyl)amino)phenyl)- N4,N4-dimethylbenzene-1,4- disulfonamide |
| 30 | 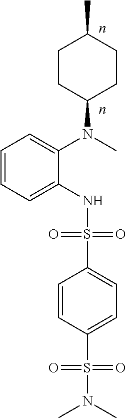 [SMILES: CN(C)S(=O)(=O)c1ccc(S(=O)(=O)Nc2ccccc2N(C)[C@H]2CC[C@@H](C)CC2)cc1] | N1,N1-dimethyl-N4-(2-(methyl((1s,4s)-4- methylcyclohexyl)amino)phenyl)benzene- 1,4-disulfonamide |
| 31 | 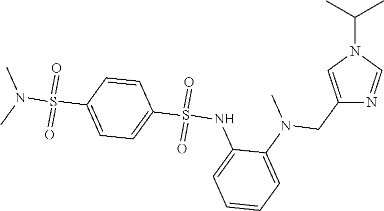 [SMILES: CC(C)n1cnc(CN(C)c2ccccc2NS(=O)(=O)c2ccc(S(=O)(=O)N(C)C)cc2)c1] | N1-(2-(((1-isopropyl-1H-imidazol-4- yl)methyl)(methyl)amino)phenyl)-N4,N4- dimethylbenzene-1,4-disulfonamide |
| 32 | 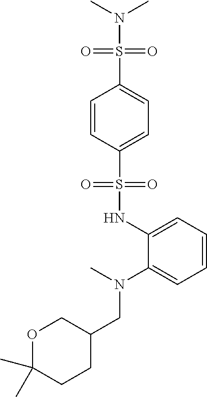 [SMILES: CN(CC1CCC(C)(C)OC1)c1ccccc1NS(=O)(=O)c1ccc(S(=O)(=O)N(C)C)cc1] | N1-(2-(((6,6-dimethyltetrahydro-2H-pyran- 3-yl)methyl)(methyl)amino)phenyl)- N4,N4-dimethylbenzene-1,4-disulfonamide |
| 33 | 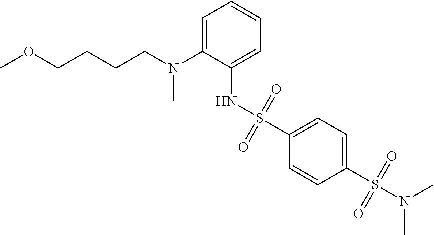 | N1-(2-((4- methoxybutyl)(methyl)amino)phenyl)- N4,N4-dimethylbenzene-1,4-disulfonamide |
| 34 | 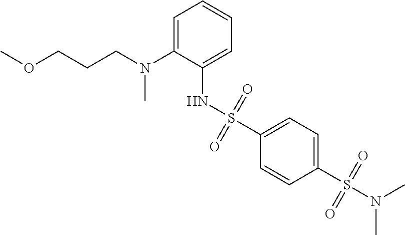 | N1-(2-((3- methoxypropyl)(methyl)amino)phenyl)- N4,N4-dimethylbenzene-1,4-disulfonamide |
| 35 | 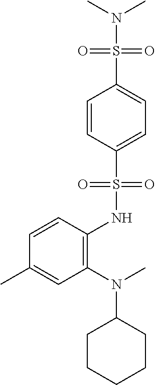 [SMILES: Cc1ccc(NS(=O)(=O)c2ccc(S(=O)(=O)N(C)C)cc2)c(N(C)C2CCCCC2)c1] | N1-(2-(cyclohexyl(methyl)amino)-4- methylphenyl)-N4,N4-dimethylbenzene- 1,4-disulfonamide |
| 36 | 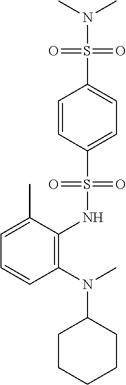 [SMILES: Cc1cccc(N(C)C2CCCCC2)c1NS(=O)(=O)c1ccc(S(=O)(=O)N(C)C)cc1] | N1-(2-(cyclohexyl(methyl)amino)-6- methylphenyl)-N4,N4-dimethylbenzene- 1,4-disulfonamide |
| 37 | 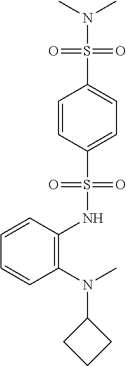 [SMILES: CN(c1ccccc1NS(=O)(=O)c1ccc(S(=O)(=O)N(C)C)cc1)C1CCC1] | N1-(2-(cyclobutyl(methyl)amino)phenyl)- N4,N4-dimethylbenzene-1,4-disulfonamide |
| 38 | 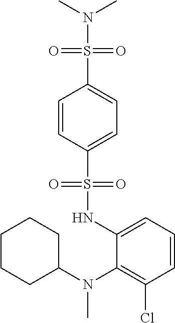 [SMILES: CN(c1c(Cl)cccc1NS(=O)(=O)c1ccc(S(=O)(=O)N(C)C)cc1)C1CCCCC1] | N1-(3-chloro-2- (cyclohexyl(methyl)amino)phenyl)-N4,N4- dimethylbenzene-1,4-disulfonamide |
| 39 | 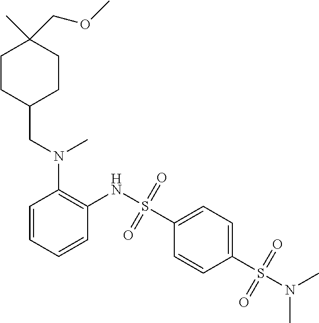 [SMILES: COCC1(C)CCC(CN(C)c2ccccc2NS(=O)(=O)c2ccc(S(=O)(=O)N(C)C)cc2)CC1] | N1-(2-(((4-(methoxymethyl)-4- methylcyclohexyl)methyl)(methyl)amino) phenyl)-N4,N4-dimethylbenzene-1,4- disulfonamide |
| 40 | 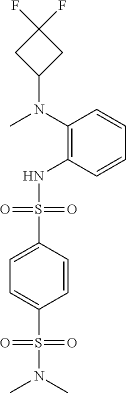 [SMILES: CN(c1ccccc1NS(=O)(=O)c1ccc(S(=O)(=O)N(C)C)cc1)C1CC(F)(F)C1] | N1-(2-((3,3- difluorocyclobutyl)(methyl)amino)phenyl)- N4,N4-dimethylbenzene-1,4-disulfonamide |
| 41 | 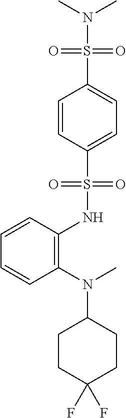 [SMILES: CN(c1ccccc1NS(=O)(=O)c1ccc(S(=O)(=O)N(C)C)cc1)C1CCC(F)(F)CC1] | N1-(2-((4,4- difluorocyclohexyl)(methyl)amino)phenyl)- N4,N4-dimethylbenzene-1,4-disulfonamide |
| 42 | 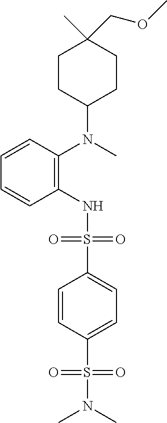 [SMILES: COCC1(C)CCC(N(C)c2ccccc2NS(=O)(=O)c2ccc(S(=O)(=O)N(C)C)cc2)CC1] | N1-(2-((4-(methoxymethyl)-4- methylcyclohexyl)(methyl)amino)phenyl)- N4,N4-dimethylbenzene-1,4-disulfonamide |
| 43 | 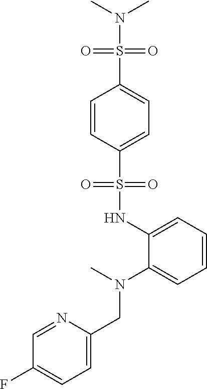 [SMILES: CN(Cc1ccc(F)cn1)c1ccccc1NS(=O)(=O)c1ccc(S(=O)(=O)N(C)C)cc1] | N1-(2-(((5-fluoropyridin-2- yl)methyl)(methyl)amino)phenyl)-N4,N4- dimethylbenzene-1,4-disulfonamide |
| 44 | 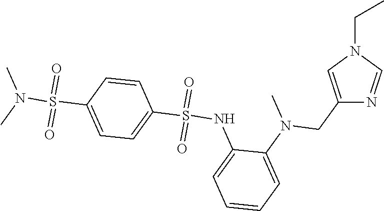 [SMILES: CCn1cnc(CN(C)c2ccccc2NS(=O)(=O)c2ccc(S(=O)(=O)N(C)C)cc2)c1] | N1-(2-(((1-ethyl-1H-imidazol-4- yl)methyl)(methyl)amino)phenyl)-N4,N4- dimethylbenzene-1,4-disulfonamide |
| 45 | 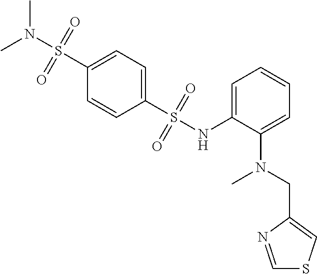 [SMILES: CN(Cc1cscn1)c1ccccc1NS(=O)(=O)c1ccc(S(=O)(=O)N(C)C)cc1] | N1,N1-dimethyl-N4-(2-(methyl(thiazol-4- ylmethyl)amino)phenyl)benzene-1,4- disulfonamide |
| 46 | 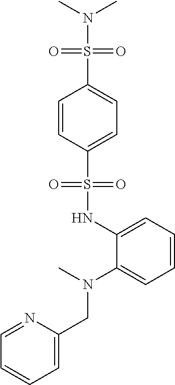 [SMILES: CN(Cc1ccccn1)c1ccccc1NS(=O)(=O)c1ccc(S(=O)(=O)N(C)C)cc1] | N1,N1-dimethyl-N4-(2-(methyl(pyridin-2- ylmethyl)amino)phenyl)benzene-1,4- disulfonamide |
| 47 | 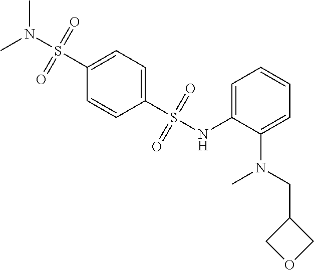 [SMILES: CN(CC1COC1)c1ccccc1NS(=O)(=O)c1ccc(S(=O)(=O)N(C)C)cc1] | N1,N1-dimethyl-N4-(2-(methyl(oxetan-3- ylmethyl)amino)phenyl)benzene-1,4- disulfonamide |
| 48 | 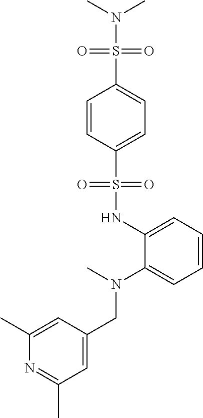 [SMILES: Cc1cc(CN(C)c2ccccc2NS(=O)(=O)c2ccc(S(=O)(=O)N(C)C)cc2)cc(C)n1] | N1-(2-(((2,6-dimethylpyridin-4- yl)methyl)(methyl)amino)phenyl)-N4,N4- dimethylbenzene-1,4-disulfonamide |
| 49 | 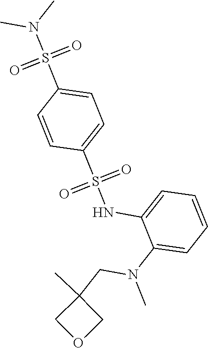 | N1,N1-dimethyl-N4-(2-(methyl((3- methyloxetan-3- y1)methyl)amino)phenyl)benzene-1,4- disulfonamide |
| 50 | 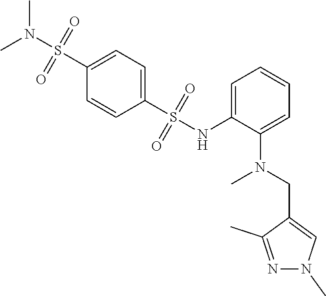 | N1-(2-(((1,3-dimethyl-1H-pyrazol-4- yl)methyl)(methyl)amino)phenyl)-N4,N4- dimethylbenzene-1,4-disulfonamide |
| 51 | 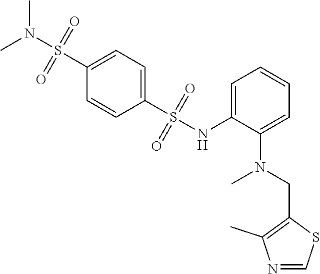 [SMILES: Cc1ncsc1CN(C)c1ccccc1NS(=O)(=O)c1ccc(S(=O)(=O)N(C)C)cc1] | N1,N1-dimethyl-N4-(2-(methyl((4- methylthiazol-5- yl)methyl)amino)phenyl)benzene-1,4- disulfonamide |
| 52 | 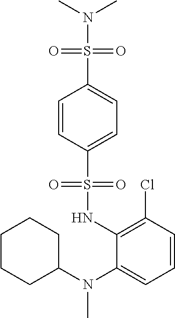 [SMILES: CN(c1cccc(Cl)c1NS(=O)(=O)c1ccc(S(=O)(=O)N(C)C)cc1)C1CCCCC1] | N1-(2-chloro-6- (cyclohexyl(methyl)amino)phenyl)-N4,N4- dimethylbenzene-1,4-disulfonamide |
| 53 | 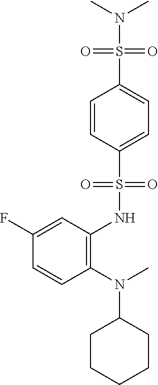 [SMILES: CN(c1ccc(F)cc1NS(=O)(=O)c1ccc(S(=O)(=O)N(C)C)cc1)C1CCCCC1] | N1-(2-(cyclohexyl(methyl)amino)-5- fluorophenyl)-N4,N4-dimethylbenzene-1,4- disulfonamide |
| 54 | 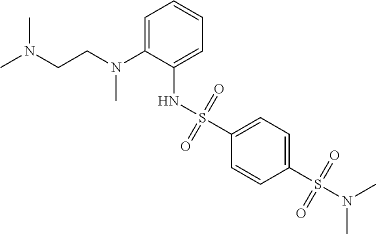 | N1-(2-((2- (dimethylamino)ethyl)(methyl)amino) phenyl)-N4,N4-dimethylbenzene-1,4- disulfonamide |
| 55 | 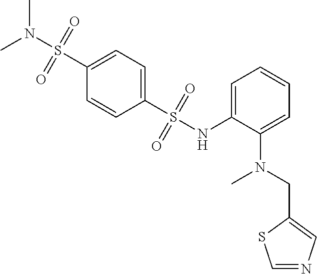 [SMILES: CN(Cc1cncs1)c1ccccc1NS(=O)(=O)c1ccc(S(=O)(=O)N(C)C)cc1] | N1,N1-dimethyl-N4-(2-(methyl(thiazol-5- ylmethyl)amino)phenyl)benzene-1,4- disulfonamide |
| 56 | 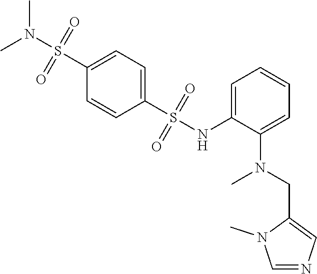 [SMILES: CN(Cc1cncn1C)c1ccccc1NS(=O)(=O)c1ccc(S(=O)(=O)N(C)C)cc1] | N1,N1-dimethyl-N4-(2-(methyl((1-methyl- 1H-imidazol-5- yl)methyl)amino)phenyl)benzene-1,4- disulfonamide |
| 57 | 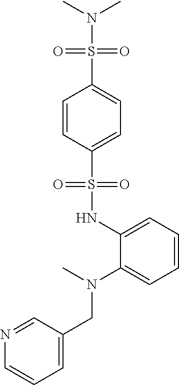 [SMILES: CN(Cc1cccnc1)c1ccccc1NS(=O)(=O)c1ccc(S(=O)(=O)N(C)C)cc1] | N1,N1-dimethyl-N4-(2-(methyl(pyridin-3- ylmethyl)amino)phenyl)benzene-1,4- disulfonamide |
| 58 | 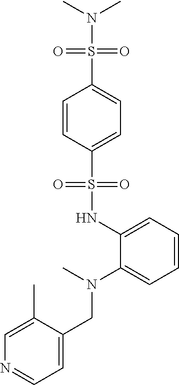 [SMILES: Cc1cnccc1CN(C)c1ccccc1NS(=O)(=O)c1ccc(S(=O)(=O)N(C)C)cc1] | N1,N1-dimethyl-N4-(2-(methyl((3- methylpyridin-4- yl)methyl)amino)phenyl)benzene-1,4- disulfonamide |
| 59 | 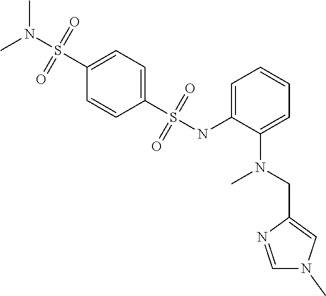 | N1,N1-dimethyl-N4-(2-(methyl((1-methyl- 1H-imidazol-4- yl)methyl)amino)phenyl)benzene-1,4- disulfonamide |
| 60 | 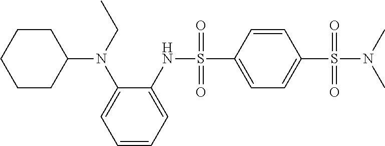 [SMILES: CCN(c1ccccc1NS(=O)(=O)c1ccc(S(=O)(=O)N(C)C)cc1)C1CCCCC1] | N1-(2-(cyclohexyl(ethyl)amino)phenyl)- N4,N4-dimethylbenzene-1,4-disulfonamide |
| 61 | 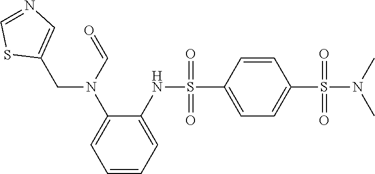 [SMILES: CN(C)S(=O)(=O)c1ccc(S(=O)(=O)Nc2ccccc2N(C=O)Cc2cncs2)cc1] | N-(2-((4-(N,N- dimethylsulfamoyl)phenyl)sulfonamido) phenyl)-N-(thiazol-5-ylmethyl)formamide |
| 62 | 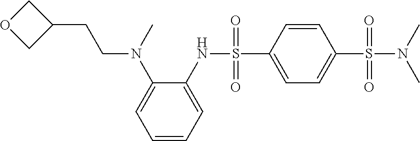 [SMILES: CN(CCC1COC1)c1ccccc1NS(=O)(=O)c1ccc(S(=O)(=O)N(C)C)cc1] | N1,N1-dimethyl-N4-(2-(methyl(2-(oxetan- 3-yl)ethyl)amino)phenyl)benzene-1,4- disulfonamide |
| 63 | 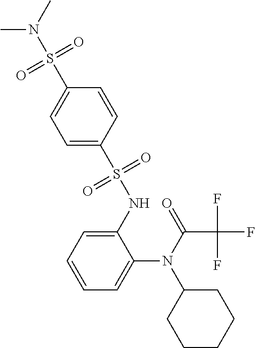 [SMILES: CN(C)S(=O)(=O)c1ccc(S(=O)(=O)Nc2ccccc2N(C(=O)C(F)(F)F)C2CCCCC2)cc1] | N-cyclohexyl-N-(2-((4-(N,N- dimethylsulfamoyl)phenyl)sulfonamido) phenyl)-2,2,2-trifluoroacetamide |
| 64 | 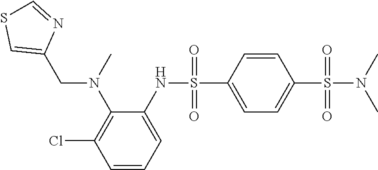 [SMILES: CN(Cc1cscn1)c1c(Cl)cccc1NS(=O)(=O)c1ccc(S(=O)(=O)N(C)C)cc1] | N1-(3-chloro-2-(methyl(thiazol-4- ylmethyl)amino)phenyl)-N4,N4- dimethylbenzene-1,4-disulfonamide |
| 65 | 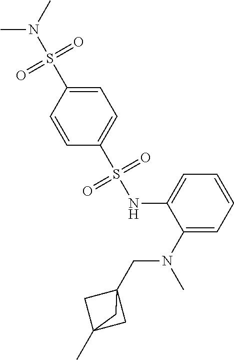 [SMILES: CN(CC12CC(C)(C1)C2)c1ccccc1NS(=O)(=O)c1ccc(S(=O)(=O)N(C)C)cc1] | N1,N1-dimethyl-N4-(2-(methyl((3- methylbicyclo[1.1.1]pentan-1- yl)methyl)amino)phenyl)benzene-1,4- disulfonamide |
| 66 | 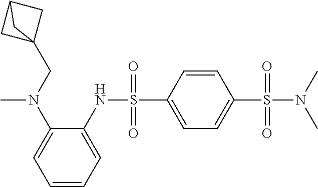 | N1-(2-((bicyclo[1.1.1]pentan-1- ylmethyl)(methyl)amino)phenyl)-N4,N4- dimethylbenzene-1,4-disulfonamide |
| 67 | 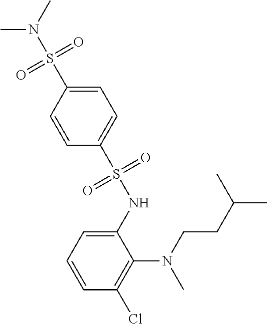 [SMILES: CC(C)CCN(C)c1c(Cl)cccc1NS(=O)(=O)c1ccc(S(=O)(=O)N(C)C)cc1] | N1-(3-chloro-2- (isopentyl(methyl)amino)phenyl)-N4,N4- dimethylbenzene-1,4-disulfonamide |
| 68 | 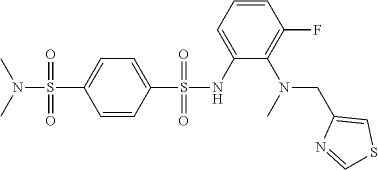 [SMILES: CN(Cc1cscn1)c1c(F)cccc1NS(=O)(=O)c1ccc(S(=O)(=O)N(C)C)cc1] | N1-(3-fluoro-2-(methyl(thiazol-4- ylmethyl)amino)phenyl)-N4,N4- dimethylbenzene-1,4-disulfonamide |
| 69 | 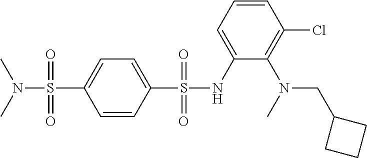 [SMILES: CN(CC1CCC1)c1c(Cl)cccc1NS(=O)(=O)c1ccc(S(=O)(=O)N(C)C)cc1] | N1-(3-chloro-2- ((cyclobutylmethyl)(methyl)amino)phenyl)- N4,N4-dimethylbenzene-1,4- disulfonamide |
| 70 | 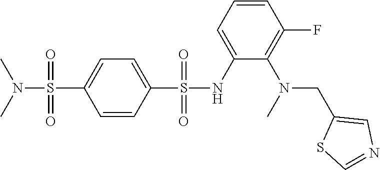 [SMILES: CN(Cc1cncs1)c1c(F)cccc1NS(=O)(=O)c1ccc(S(=O)(=O)N(C)C)cc1] | N1-(3-fluoro-2-(methyl(thiazol-5- ylmethyl)amino)phenyl)-N4,N4- dimethylbenzene-1,4-disulfonamide |
| 71 | 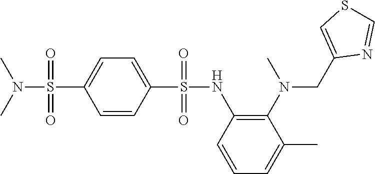 [SMILES: Cc1cccc(NS(=O)(=O)c2ccc(S(=O)(=O)N(C)C)cc2)c1N(C)Cc1cscn1] | N1,N1-dimethyl-N4-(3-methyl-2- (methyl(thiazol-4- ylmethyl)amino)phenyl)benzene-1,4- disulfonamide |
| 72 | 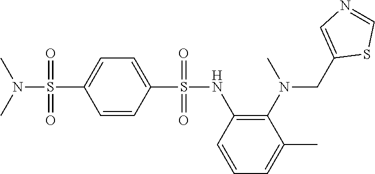 [SMILES: Cc1cccc(NS(=O)(=O)c2ccc(S(=O)(=O)N(C)C)cc2)c1N(C)Cc1cncs1] | N1,N1-dimethyl-N4-(3-methyl-2- (methyl(thiazol-5- ylmethyl)amino)phenyl)benzene-1,4- disulfonamide |
| 73 | 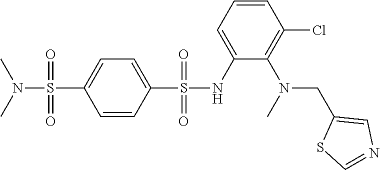 [SMILES: CN(Cc1cncs1)c1c(Cl)cccc1NS(=O)(=O)c1ccc(S(=O)(=O)N(C)C)cc1] | N1-(3-chloro-2-(methyl(thiazol-5- ylmethyl)amino)phenyl)-N4,N4- dimethylbenzene-1,4-disulfonamide |
| 74 | 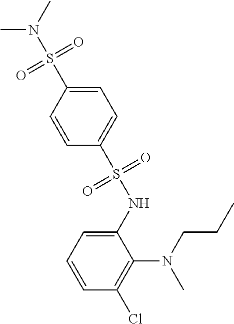 [SMILES: CCCN(C)c1c(Cl)cccc1NS(=O)(=O)c1ccc(S(=O)(=O)N(C)C)cc1] | N1-(3-chloro-2- (methyl(propyl)amino)phenyl)-N4,N4- dimethylbenzene-1,4-disulfonamide |
| 75 | 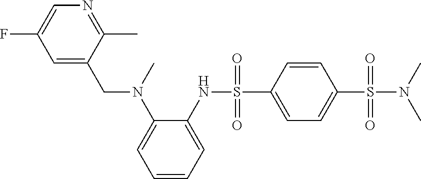 [SMILES: Cc1ncc(F)cc1CN(C)c1ccccc1NS(=O)(=O)c1ccc(S(=O)(=O)N(C)C)cc1] | N1-(2-(((5-fluoro-2-methylpyridin-3- yl)methyl)(methyl)amino)phenyl)-N4,N4- dimethylbenzene-1,4-disulfonamide |
| 76 | 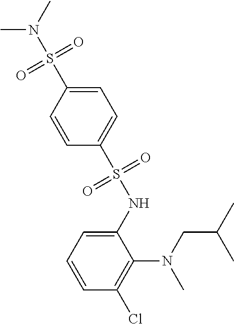 [SMILES: CC(C)CN(C)c1c(Cl)cccc1NS(=O)(=O)c1ccc(S(=O)(=O)N(C)C)cc1] | N1-(3-chloro-2- (isobutyl(methyl)amino)phenyl)-N4,N4- dimethylbenzene-1,4-disulfonamide |
| 77 | 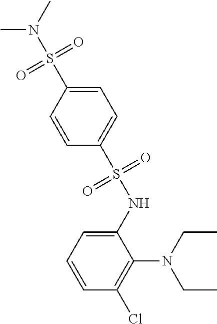 [SMILES: CCN(CC)c1c(Cl)cccc1NS(=O)(=O)c1ccc(S(=O)(=O)N(C)C)cc1] | N1-(3-chloro-2-(diethylamino)phenyl)- N4,N4-dimethylbenzene-1,4-disulfonamide |
| 78 | 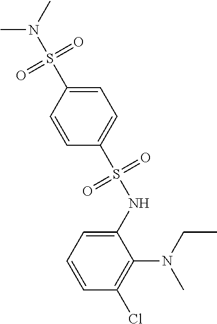 [SMILES: CCN(C)c1c(Cl)cccc1NS(=O)(=O)c1ccc(S(=O)(=O)N(C)C)cc1] | N1-(3-chloro-2- (ethyl(methyl)amino)phenyl)-N4,N4- dimethylbenzene-1,4-disulfonamide |
| 79 | 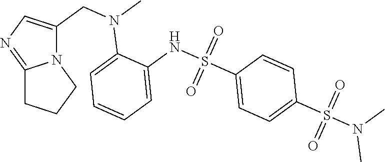 [SMILES: CN(Cc1cnc2n1CCC2)c1ccccc1NS(=O)(=O)c1ccc(S(=O)(=O)N(C)C)cc1] | N1-(2-(((6,7-dihydro-5H-pyrrolo[1,2- a]imidazol-3- yl)methyl)(methyl)amino)phenyl)-N4,N4- dimethylbenzene-1,4-disulfonamide |
| 80 | 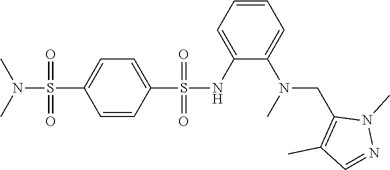 | N1-(2-(((1,4-dimethyl-1H-pyrazol-5- yl)methyl)(methyl)amino)phenyl)-N4,N4- dimethylbenzene-1,4-disulfonamide |
| 81 | 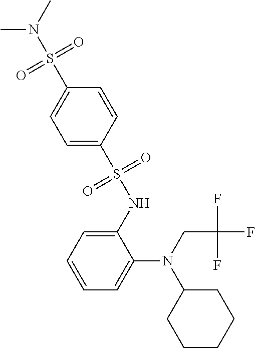 [SMILES: CN(C)S(=O)(=O)c1ccc(S(=O)(=O)Nc2ccccc2N(CC(F)(F)F)C2CCCCC2)cc1] | N1-(2-(cyclohexyl(2,2,2- trifluoroethyl)amino)phenyl)-N4,N4- dimethylbenzene-1,4-disulfonamide |
| 82 | 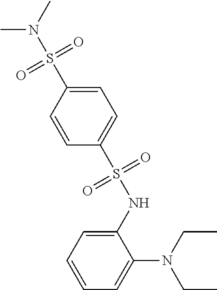 [SMILES: CCN(CC)c1ccccc1NS(=O)(=O)c1ccc(S(=O)(=O)N(C)C)cc1] | N1-(2-(diethylamino)phenyl)-N4,N4- dimethylbenzene-1,4-disulfonamide |
| 83 | 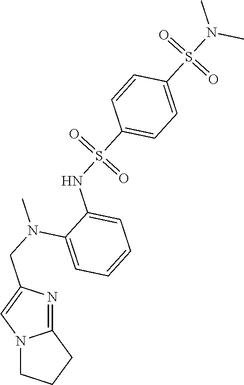 [SMILES: CN(Cc1cn2c(n1)CCC2)c1ccccc1NS(=O)(=O)c1ccc(S(=O)(=O)N(C)C)cc1] | N1-(2-(((6,7-dihydro-5H-pyrrolo[1,2- a]imidazol-2- yl)methyl)(methyl)amino)phenyl)-N4,N4- dimethylbenzene-1,4-disulfonamide |
| 84 | 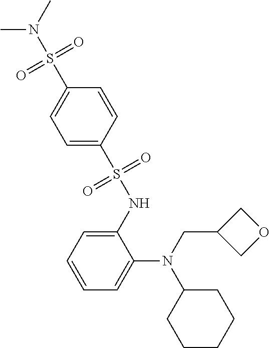 [SMILES: CN(C)S(=O)(=O)c1ccc(S(=O)(=O)Nc2ccccc2N(CC2COC2)C2CCCCC2)cc1] | N1-(2-(cyclohexyl(oxetan-3- ylmethyl)amino)phenyl)-N4,N4- dimethylbenzene-1,4-disulfonamide |
| 85 | 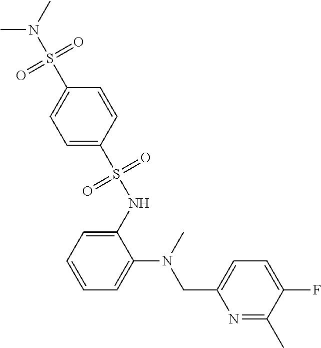 [SMILES: Cc1nc(CN(C)c2ccccc2NS(=O)(=O)c2ccc(S(=O)(=O)N(C)C)cc2)ccc1F] | N1-(2-(((5-fluoro-6-methylpyridin-2- yl)methyl)(methyl)amino)phenyl)-N4,N4- dimethylbenzene-1,4-disulfonamide |
| 86 | 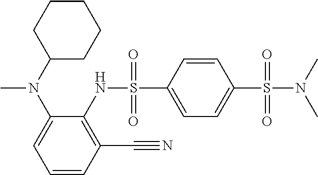 | N1-(2-cyano-6- (cyclohexyl(methyl)amino)phenyl)-N4,N4- dimethylbenzene-1,4-disulfonamide |
| 87 | 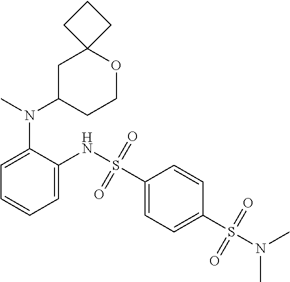 [SMILES: CN(c1ccccc1NS(=O)(=O)c1ccc(S(=O)(=O)N(C)C)cc1)C1CCOC2(CCC2)C1] | N1,N1-dimethyl-N4-(2-(methyl(5- oxaspiro[3.5]nonan-8- yl)amino)phenyl)benzene-1,4- disulfonamide |
| 88 | 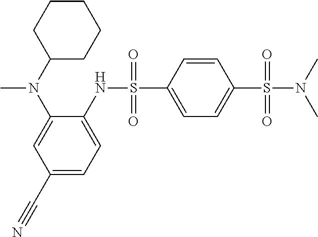 | N1-(4-cyano-2- (cyclohexyl(methyl)amino)phenyl)-N4,N4- dimethylbenzene-1,4-disulfonamide |
| 89 | 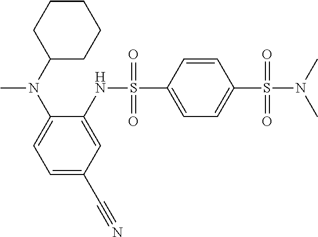 | N1-(5-cyano-2- (cyclohexyl(methyl)amino)phenyl)-N4,N4- dimethylbenzene-1,4-disulfonamide |
| 90 | 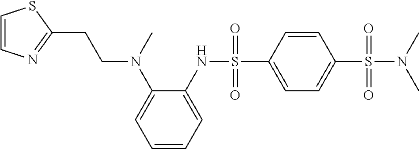 [SMILES: CN(CCc1nccs1)c1ccccc1NS(=O)(=O)c1ccc(S(=O)(=O)N(C)C)cc1] | N1,N1-dimethyl-N4-(2-(methyl(2-(thiazol- 2-yl)ethyl)amino)phenyl)benzene-1,4- disulfonamide |
| 91 | 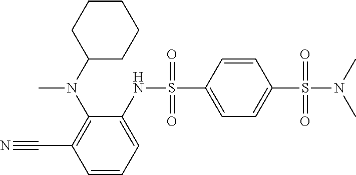 [SMILES: CN(c1c(C#N)cccc1NS(=O)(=O)c1ccc(S(=O)(=O)N(C)C)cc1)C1CCCCC1] | N1-(3-cyano-2- (cyclohexyl(methyl)amino)phenyl)-N4,N4- dimethylbenzene-1,4-disulfonamide |
| 92 | 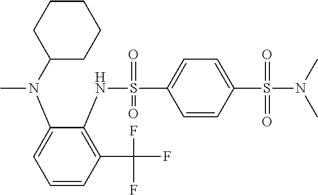 | N1-(2-(cyclohexyl(methyl)amino)-6- (trifluoromethyl)phenyl)-N4,N4- dimethylbenzene-1,4-disulfonamide |
| 93 | 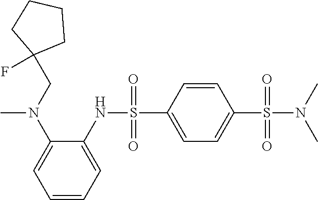 | N1-(2-(((1- fluorocyclopentyl)methyl)(methyl)amino) phenyl)-N4,N4-dimethylbenzene-1,4- disulfonamide |
| 94 | 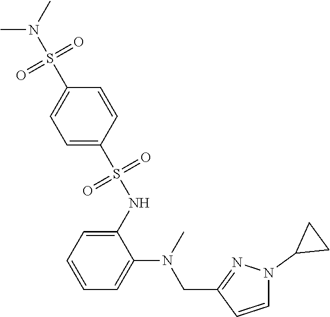 [SMILES: CN(Cc1ccn(C2CC2)n1)c1ccccc1NS(=O)(=O)c1ccc(S(=O)(=O)N(C)C)cc1] | N1-(2-(((1-cyclopropyl-1H-pyrazol-3- yl)methyl)(methyl)amino)phenyl)-N4,N4- dimethylbenzene-1,4-disulfonamide |
| 95 | 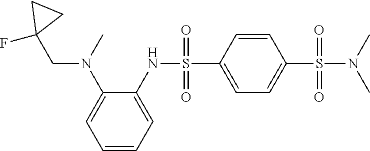 [SMILES: CN(CC1(F)CC1)c1ccccc1NS(=O)(=O)c1ccc(S(=O)(=O)N(C)C)cc1] | N1-(2-(((1- fluorocyclopropyl)methyl)(methyl)amino) phenyl)-N4,N4-dimethylbenzene-1,4- disulfonamide |
| 96 | 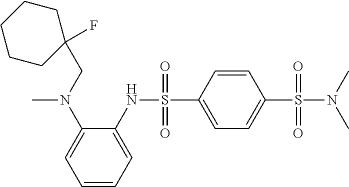 [SMILES: CN(CC1(F)CCCCC1)c1ccccc1NS(=O)(=O)c1ccc(S(=O)(=O)N(C)C)cc1] | N1-(2-(((1- fluorocyclohexyl)methyl)(methyl)amino) phenyl)-N4,N4-dimethylbenzene-1,4- disulfonamide |
| 97 | 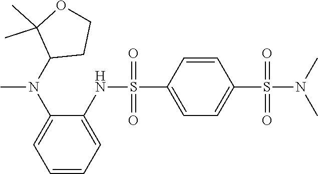 | N1-(2-((2,2-dimethyltetrahydrofuran-3- yl)(methyl)amino)phenyl)-N4,N4- dimethylbenzene-1,4-disulfonamide |
| 98 | 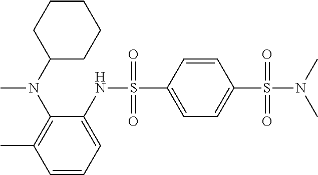 | N1-(2-(cyclohexyl(methyl)amino)-3- methylphenyl)-N4,N4-dimethylbenzene- 1,4-disulfonamide |
| 99 | 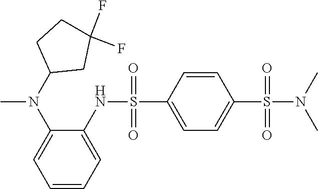 | N1-(2-((3,3- difluorocyclopentyl)(methyl)amino)phenyl)- N4,N4-dimethylbenzene-1,4- disulfonamide |
| 100 | 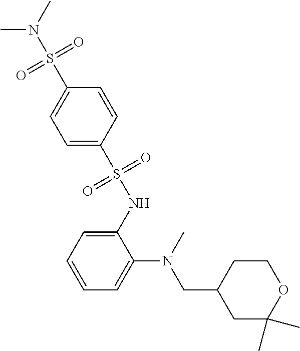 | N1-(2-(((2,2-dimethyltetrahydro-2H-pyran- 4-yl)methyl)(methyl)amino)phenyl)- N4,N4-dimethylbenzene-1,4-disulfonamide |
| 101 | 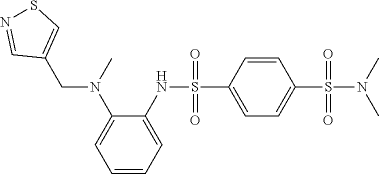 [SMILES: CN(Cc1cnsc1)c1ccccc1NS(=O)(=O)c1ccc(S(=O)(=O)N(C)C)cc1] | N1-(2-((isothiazol-4- ylmethyl)(methyl)amino)phenyl)-N4,N4- dimethylbenzene-1,4-disulfonamide |
| 102 | 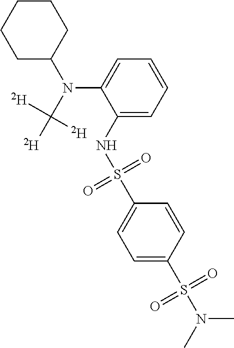 [SMILES: [2H]C([2H])([2H])N(c1ccccc1NS(=O)(=O)c1ccc(S(=O)(=O)N(C)C)cc1)C1CCCCC1] | N1-(2-(cyclohexyl(methyl- d3)amino)phenyl)-N4,N4- dimethylbenzene-1,4-disulfonamide |
| 103 | 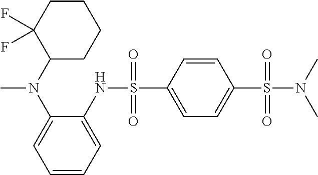 | N1-(2-((2,2- difluorocyclohexyl)(methyl)amino)phenyl)- N4,N4-dimethylbenzene-1,4-disulfonamide |
| 104 | 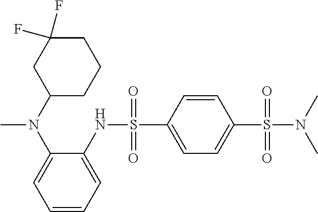 | N1-(2-((3,3- difluorocyclohexy1)(methyl)amino)phenyl)- N4,N4-dimethylbenzene-1,4-disulfonamide |
| 105 | 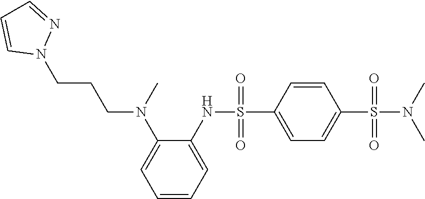 [SMILES: CN(CCCn1cccn1)c1ccccc1NS(=O)(=O)c1ccc(S(=O)(=O)N(C)C)cc1] | N1-(2-((3-(1H-pyrazol-1- yl)propyl)(methyl)amino)phenyl)-N4,N4- dimethylbenzene-1,4-disulfonamide |
| 106 | 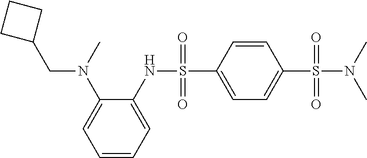 [SMILES: CN(CC1CCC1)c1ccccc1NS(=O)(=O)c1ccc(S(=O)(=O)N(C)C)cc1] | N1-(2- ((cyclobutylmethyl)(methyl)amino)phenyl)- N4,N4-dimethylbenzene-1,4- disulfonamide |
| 107 | 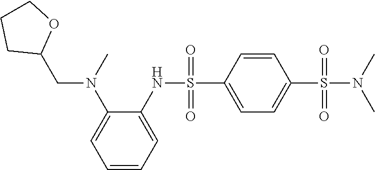 [SMILES: CN(CC1CCCO1)c1ccccc1NS(=O)(=O)c1ccc(S(=O)(=O)N(C)C)cc1] | N1,N1-dimethyl-N4-(2- (methyl((tetrahydrofuran-2- y1)methyl)amino)phenyl)benzene-1,4- disulfonamide |
| 108 | 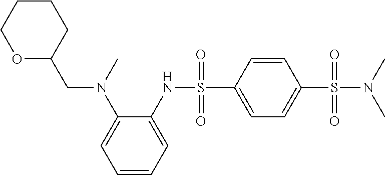 [SMILES: CN(CC1CCCCO1)c1ccccc1NS(=O)(=O)c1ccc(S(=O)(=O)N(C)C)cc1] | N1,N1-dimethyl-N4-(2- (methyl((tetrahydro-2H-pyran-2- yl)methyl)amino)phenyl)benzene-1,4- disulfonamide |
| 109 | 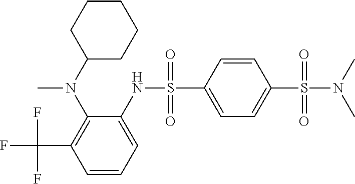 [SMILES: CN(c1c(NS(=O)(=O)c2ccc(S(=O)(=O)N(C)C)cc2)cccc1C(F)(F)F)C1CCCCC1] | N1-(2-(cyclohexyl(methyl)amino)-3- (trifluoromethyl)phenyl)-N4,N4- dimethylbenzene-1,4-disulfonamide |
| 110 | 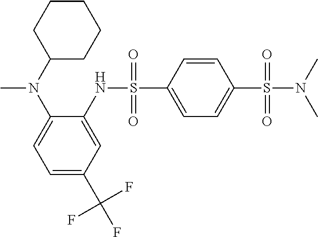 | N1-(2-(cyclohexyl(methyl)amino)-5- (trifluoromethyl)phenyl)-N4,N4- dimethylbenzene-1,4-disulfonamide |
| 111 | 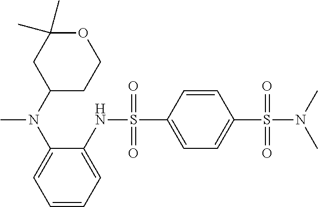 | N1-(2-((2,2-dimethyltetrahydro-2H-pyran- 4-yl)(methyl)amino)phenyl)-N4,N4- dimethylbenzene-1,4-disulfonamide |
| 112 | 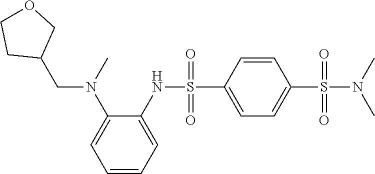 [SMILES: CN(CC1CCOC1)c1ccccc1NS(=O)(=O)c1ccc(S(=O)(=O)N(C)C)cc1] | N1,N1-dimethyl-N4-(2- (methyl((tetrahydrofuran-3- y1)methyl)amino)phenyl)benzene-1,4- disulfonamide |
| 113 | 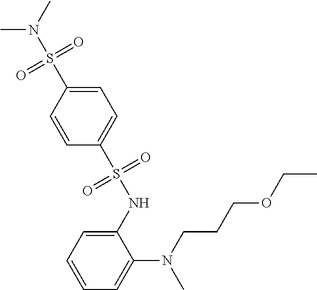 [SMILES: CCOCCCN(C)c1ccccc1NS(=O)(=O)c1ccc(S(=O)(=O)N(C)C)cc1] | N1-(2-((3- ethoxypropyl)(methyl)amino)phenyl)- N4,N4-dimethylbenzene-1,4-disulfonamide |
| 114 | 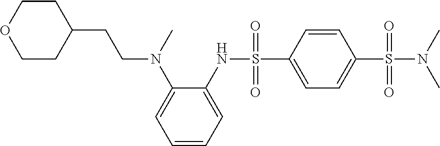 [SMILES: CN(CCC1CCOCC1)c1ccccc1NS(=O)(=O)c1ccc(S(=O)(=O)N(C)C)cc1] | N1,N1-dimethyl-N4-(2-(methyl(2- (tetrahydro-2H-pyran-4- yl)ethyl)amino)phenyl)benzene-1,4- disulfonamide |
| 115 | 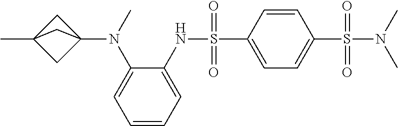 | N1,N1-dimethyl-N4-(2-(methyl(3- methylbicyclo[1.1.1 ]pentan-1- yl)amino)phenyl)benzene-1,4- disulfonamide |
| 116 | 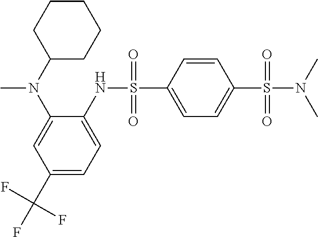 | N1-(2-(cyclohexyl(methyl)amino)-4- (trifluoromethyl)phenyl)-N4,N4- dimethylbenzene-1,4-disulfonamide |
| 117 | 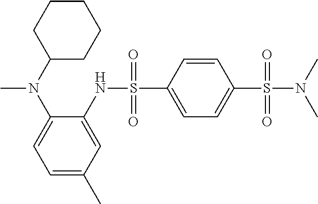 | N1-(2-(cyclohexyl(methyl)amino)-5- methylpheny1)-N4,N4-dimethylbenzene- 1,4-disulfonamide |
| 118 | 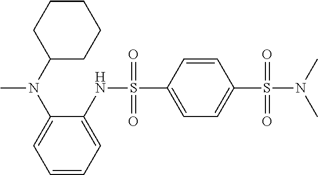 | N1-(2-(cyclohexyl(methyl)amino)phenyl)- N4,N4-dimethylbenzene-1,4-disulfonamide |

or a pharmaceutically acceptable salt thereof. In one embodiment, a tautomer, stereoisomer, and deuterated form of any of the compounds of the present disclosure (e.g., compounds of Formulas (I), (II), (III), (IV), and (V) as well as those in Table 1) is encompassed.

It is understood that in the present description, combinations of substituents and/or variables of the depicted formulae are permissible only if such contributions result in stable compounds.

Furthermore, all compounds of the present disclosure (e.g., compounds of Formulas (I), (II), (III), (IV), and (V) as well as those in Table 1) that exist in free base or acid form can be converted to their pharmaceutically acceptable salts by treatment with the appropriate inorganic or organic base or acid by methods known to one skilled in the art. Salts of the compounds of the present disclosure (e.g., compounds of Formulas (I), (II), (III), (IV), and (V) as well as those in Table 1) can be converted to their free base or acid form by standard techniques.

### Methods of Synthesis

The compounds described herein can be made using conventional organic syntheses and commercially available starting materials, and the methods provided herein. By way of example and not limitation, compounds of the present disclosure (e.g., compounds of Formulas (I), (II), (III), (IV), and (V) as well as those in Table 1) can be prepared as outlined in the examples set forth herein. It should be noted that one skilled in the art would know how to modify the procedures set forth in the illustrative examples to arrive at the desired products.

### Methods of Use

Embodiments of the present disclosure provide a method for modulating TRPML1 in a subject in need thereof, the method comprising administering to the subject an effective amount of a compound of the present disclosure (e.g., compounds of Formulas (I), (II), (III), (IV), and (V) as well as those in Table 1). Modulation (e.g., inhibition or activation) of TRPML1 can be assessed and demonstrated by a wide variety of ways known in the art. Published assays can be utilized for determining whether and to what degree TRPML1 has been modulated.

In one aspect, provided herein is a method of modulating TRPML1 comprising contacting TRPML1 with an effective amount of a compound of the present disclosure (e.g., compounds of Formulas (I), (II), (III), (IV), and (V) as well as those in Table 1). In some embodiments, a compound of the present disclosure activates TRPML1. In some embodiments, a compound of the present disclosure is an agonist of TRPML1.

In some embodiments, a compound of compounds of the present disclosure (e.g., compounds of Formulas (I), (II), (III), (IV), and (V) as well as those in Table 1) modulates the activity of TRPML1 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of the present disclosure (e.g., compounds of Formulas (I), (II), (III), (IV), and (V) as well as those in Table 1) modulates the activity of TRPML1 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for activating TRPML1 in a subject in need thereof, the method comprising administering to the subject an effective amount of a compound of the present disclosure (e.g., compounds of Formulas (I), (II), (III), (IV), and (V) as well as those in Table 1). Activation of TRPML1 can be assessed and demonstrated by a wide variety of ways known in the art. Published assays, including cell-based assays, can be utilized for determining whether and to what degree TRPML1 has been activated.

TRPML1 activation can be measured in cells over expressing TRPML1 at the plasma membrane and measuring Calcium influx into cells using a FLIPR Tetra instrument. Activation can also be measured in cells over expressing a calcium sensitive Gcamp protein tag onto wild type TRPML1. Activation can also be measured using various electrophysiological measurements, e.g. patch clamp at enlarged lysosomes overexpressing Trpml1.

In one aspect, provided herein is a method of activating TRPML1 comprising contacting TRPML1 with an effective amount of a compound of the present disclosure (e.g., compounds of Formulas (I), (II), (III), (IV), and (V) as well as those in Table 1). In some embodiments, the compound of the present disclosure partially activates TRPML1. In some embodiments, the compound of the present disclosure fully activates TRPML1.

In some embodiments, a compound of the present disclosure (e.g., compounds of Formulas (I), (II), (III), (IV), and (V) as well as those in Table 1) activates TRPML1 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of the present disclosure (e.g., compounds of Formulas (I), (II), (III), (IV), and (V) as well as those in Table 1) activates TRPML1 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating a disease associated with TRPML1 in a subject in need thereof, comprising administering to the subject an effective amount of a compound of the present disclosure (e.g., compounds of Formulas (I), (II), (III), (IV), and (V) as well as those in Table 1). In some embodiments, provided herein is a method for treating a disease associated with TRPML1 in a subject in need thereof, comprising administering to the subject an effective amount of a compound of the present disclosure (e.g., compounds of Formulas (I), (II), (III), (IV), and (V) as well as those in Table 1). In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In some embodiments, administering a compound of the present disclosure (e.g., compounds of Formulas (I), (II), (III), (IV), and (V) as well as those in Table 1) to a subject that is predisposed to a disease associated with TRPML1 prevents the subject from developing any symptoms of the disease associated with TRPML1. In some embodiments, administering a compound of the present disclosure (e.g., compounds of Formulas (I), (II), (III), (IV), and (V) as well as those in Table 1) to a subject that is does not yet display symptoms of a disease associated with TRPML1 prevents the subject from developing any symptoms of the disease associated with TRPML1. In some embodiments, administering a compound of the present disclosure (e.g., compounds of Formulas (I), (II), (III), (IV), and (V) as well as those in Table 1) to a subject in need thereof diminishes the extent of the disease associated with TRPML1 in the subject. In some embodiments, administering a compound of the present disclosure (e.g., compounds of Formulas (I), (II), (III), (IV), and (V) as well as those in Table 1) to a subject in need thereof stabilizes the disease associated with TRPML1 (prevents or delays the worsening of the disease associated with TRPML1). In some embodiments, administering a compound of Formula (I) to a subject in need thereof delays the occurrence or recurrence of the disease associated with TRPML1. In some embodiments, administering a compound of the present disclosure (e.g., compounds of Formulas (I), (II), (III), (IV), and (V) as well as those in Table 1) to a subject in need thereof slows the progression of the disease associated with TRPML1. In some embodiments, administering a compound of the present disclosure (e.g., compounds of Formulas (I), (II), (III), (IV), and (V) as well as those in Table 1) to a subject in need thereof provides a partial remission of the disease associated with TRPML1. In some embodiments, administering a compound of the present disclosure (e.g., compounds of Formulas (I), (II), (III), (IV), and (V) as well as those in Table 1) to a subject in need thereof provides a total remission of the disease associated with TRPML1. In some embodiments, administering a compound of the present disclosure (e.g., compounds of Formulas (I), (II), (III), (IV), and (V) as well as those in Table 1) to a subject in need thereof decreases the dose of one or more other medications required to treat the disease associated with TRPML1. In some embodiments, administering a compound of the present disclosure (e.g., compounds of Formulas (I), (II), (III), (IV), and (V) as well as those in Table 1) to a subject in need thereof enhances the effect of another medication used to treat the disease associated with TRPML1. In some embodiments, administering a compound of the present disclosure (e.g., compounds of Formulas (I), (II), (III), (IV), and (V) as well as those in Table 1) to a subject in need thereof delays the progression of the disease associated with TRPML1. In some embodiments, administering a compound of the present disclosure (e.g., compounds of Formulas (I), (II), (III), (IV), and (V) as well as those in Table 1) to a subject in need thereof increases the quality of life of the subject having a disease associated with TRPML1. In some embodiments, administering a compound of the present disclosure (e.g., compounds of Formulas (I), (II), (III), (IV), and (V) as well as those in Table 1) to a subject in need thereof prolongs survival of a subject having a disease associated with TRPML1. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In one aspect, provided herein is method of preventing a subject that is predisposed to a disease associated with TRPML1 from developing any symptoms of the disease associated with TRPML1, the method comprising administering a compound of compounds of the present disclosure (e.g., compounds of Formulas (I), (II), (III), (IV), and (V) as well as those in Table 1) to the subject. In some embodiments, provided herein is a method of treating a subject that does not yet display symptoms of a disease associated with TRPML1 from developing any symptoms of the disease associated with TRPML1, the method comprising administering a compound of compounds of the present disclosure (e.g., compounds of Formulas (I), (II), (III), (IV), and (V) as well as those in Table 1) to the subject. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In some aspects, provided herein is a method of diminishing the extent of a disease associated with TRPML1 in a subject, the method comprising administering a compound of the present disclosure (e.g., compounds of Formulas (I), (II), (III), (IV), and (V) as well as those in Table 1) to the subject. In some embodiments, provided herein is a method of stabilizing a disease associated with TRPML1 in a subject, the method comprising administering a compound of the present disclosure (e.g., compounds of Formulas (I), (II), (III), (IV), and (V) as well as those in Table 1) to the subject. In some embodiments, the method prevents the worsening of the disease associated with TRPML1. In some embodiments, the method delays the worsening of the disease associated with TRPML1. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In another aspect, provided herein is a method of delaying the occurrence or recurrence of a disease associated with TRPML1 in a subject, the method comprising administering a compound of the present disclosure (e.g., compounds of Formulas (I), (II), (III), (IV), and (V) as well as those in Table 1) to the subject. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In some embodiments, provided herein is a method of slowing the progression of a disease associated with TRPML1 in a subject, the method comprising administering a compound of the present disclosure (e.g., compounds of Formulas (I), (II), (III), (IV), and (V) as well as those in Table 1) to the subject. In some embodiments, the method provides a partial remission of the disease associated with TRPML1. In some embodiments, the method provides a total remission of the disease associated with TRPML1. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In further aspects, provided herein is a method of decreasing the dose of one or more other medications required to treat a disease associated with TRPML1 in a subject, the method comprising administering a compound of the present disclosure (e.g., compounds of Formulas (I), (II), (III), (IV), and (V) as well as those in Table 1) to the subject. In some embodiments, provided herein is a method of enhancing the effect of another medication used to treat a disease associated with TRPML1 in a subject, the method comprising administering a compound of the present disclosure (e.g., compounds of Formulas (I), (II), (III), (IV), and (V) as well as those in Table 1) to the subject. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

Also provided here is a method of delaying the progression of a disease associated with TRPML1 in a subject, the method comprising administering a compound of the present disclosure (e.g., compounds of Formulas (I), (II), (III), (IV), and (V) as well as those in Table 1) to the subject. In some embodiments, the method increases the quality of life of the subject having a disease associated with TRPML1. In some embodiments, the method prolongs survival of the subject having a disease associated with TRPML1. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In another aspect, provided herein is a method for treating symptoms associated with TRPML1 caused by a disease in a subject in need thereof, comprising administering to the subject an effective amount of a compound of the present disclosure (e.g., compounds of Formulas (I), (II), (III), (IV), and (V) as well as those in Table 1). In some embodiments, provided herein is a method for treating symptoms associated with TRPML1 caused by a disease in a subject in need thereof, comprising administering to the subject an effective amount of a compound of the present disclosure (e.g., compounds of Formulas (I), (II), (III), (IV), and (V) as well as those in Table 1). In some embodiments, administering a compound of the present disclosure (e.g., compounds of Formulas (I), (II), (III), (IV), and (V) as well as those in Table 1) to a subject that is predisposed to a disease which causes symptoms associated with TRPML1 prevents the subject from developing any symptoms associated with TRPML1. In some embodiments, administering a compound of the present disclosure (e.g., compounds of Formulas (I), (II), (III), (IV), and (V) as well as those in Table 1) to a subject that is does not yet display symptoms associated with TRPML1 of a disease which causes symptoms associated with TRPML1 prevents the subject from developing any symptoms associated with TRPML1. In some embodiments, administering a compound of the present disclosure (e.g., compounds of Formulas (I), (II), (III), (IV), and (V) as well as those in Table 1) to a subject in need thereof diminishes the extent of the symptoms associated with TRPML1 caused by the disease in the subject. In some embodiments, administering a compound of the present disclosure (e.g., compounds of Formulas (I), (II), (III), (IV), and (V) as well as those in Table 1) to a subject in need thereof stabilizes the symptoms associated with TRPML1 of the disease (prevents or delays the worsening of the symptoms associated with TRPML1). In some embodiments, administering a compound of the present disclosure (e.g., compounds of Formulas (I), (II), (III), (IV), and (V) as well as those in Table 1) to a subject in need thereof delays the occurrence or recurrence of the symptoms associated with TRPML1 caused by the disease. In some embodiments, administering a compound of the present disclosure (e.g., compounds of Formulas (I), (II), (III), (IV), and (V) as well as those in Table 1) to a subject in need thereof slows the progression of the symptoms associated with TRPML1 caused by the disease. In some embodiments, administering a compound of the present disclosure (e.g., compounds of Formulas (I), (II), (III), (IV), and (V) as well as those in Table 1) to a subject in need thereof provides a partial remission of the disease which causes symptoms associated with TRPML1. In some embodiments, administering a compound of the present disclosure (e.g., compounds of Formulas (I), (II), (III), (IV), and (V) as well as those in Table 1) to a subject in need thereof provides a total remission of the disease which causes symptoms associated with TRPML1. In some embodiments, administering a compound of the present disclosure (e.g., compounds of Formulas (I), (II), (III), (IV), and (V) as well as those in Table 1) to a subject in need thereof decreases the dose of one or more other medications required to treat the disease which causes symptoms associated with TRPML1. In some embodiments, administering a compound of the present disclosure (e.g., compounds of Formulas (I), (II), (III), (IV), and (V) as well as those in Table 1) to a subject in need thereof enhances the effect of another medication used to treat the symptoms associated with TRPML1 of the disease. In some embodiments, administering a compound of the present disclosure (e.g., compounds of Formulas (I), (II), (III), (IV), and (V) as well as those in Table 1) to a subject in need thereof delays the progression of the disease which causes symptoms associated with TRPML1. In some embodiments, administering a compound of the present disclosure (e.g., compounds of Formulas (I), (II), (III), (IV), and (V) as well as those in Table 1) to a subject in need thereof increases the quality of life of the subject having a disease which causes symptoms associated with TRPML1. In some embodiments, administering a compound of the present disclosure (e.g., compounds of Formulas (I), (II), (III), (IV), and (V) as well as those in Table 1) to a subject in need thereof prolongs survival of a subject having a disease which causes symptoms associated with TRPML1. In some embodiments, the disease is associated with TRPML1. In some embodiments, the disease associated with TRPML1 is a neurodegenerative disease, lysosomal storage disease, Charcot-Marie-Tooth disease, mitochondrial disease, renal disease, metabolic disease, cardiovascular disease, inflammatory disorder, immunological disorder, cancer, or aging. In some embodiments, the neurodegenerative disease is Parkinson's disease, amyotrophic lateral sclerosis (ALS), HIV-associated dementia, Huntington's disease, or Alzheimer's disease (AD).

In some embodiments, compounds of the present disclosure (e.g., compounds of Formulas (I), (II), (III), (IV), and (V) as well as those in Table 1) are useful for treating a disease selected from Parkinson's disease, amyotrophic lateral sclerosis (ALS), HIV-associated dementia, Huntington's disease, or Alzheimer's disease (AD).

### Pharmaceutical Compositions and Routes of Administration

The compounds the present disclosure (e.g., compounds of Formulas (I), (II), (III), (IV), and (V) as well as those in Table 1) can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds the present disclosure (e.g., compounds of Formulas (I), (II), (III), (IV), and (V) as well as those in Table 1) can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of the present disclosure (e.g., compounds of Formulas (I), (II), (III), (IV), and (V) as well as those in Table 1) to be administered to a subject is rather widely variable and can be subject to the judgment of a health-care practitioner.

A compound of the present disclosure (e.g., compounds of Formulas (I), (II), (III), (IV), and (V) as well as those in Table 1) can be administered orally for reasons of convenience. In one embodiment, when administered orally, a compound of the present disclosure is administered with a meal and water. In another embodiment, the compound of the present disclosure is dispersed in water or juice (e.g., apple juice or orange juice) or any other liquid and administered orally as a solution or a suspension.

The compounds the present disclosure (e.g., compounds of Formulas (I), (II), (III), (IV), and (V) as well as those in Table 1) can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend inpart upon the site of the medical condition.

In one embodiment, provided herein are capsules containing a compound of the present disclosure (e.g., compounds of Formulas (I), (II), (III), (IV), and (V) as well as those in Table 1) without an additional carrier, excipient or vehicle.

In another embodiment, provided herein are compositions comprising an effective amount of a compound of the present disclosure (e.g., compounds of Formulas (I), (II), (III), (IV), and (V) as well as those in Table 1) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of the present disclosure (e.g., compounds of Formulas (I), (II), (III), (IV), and (V) as well as those in Table 1) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of the present disclosure (e.g., compounds of Formulas (I), (II), (III), (IV), and (V) as well as those in Table 1) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of the present disclosure (e.g., compounds of Formulas (I), (II), (III), (IV), and (V) as well as those in Table 1) can be delayed or prolonged by proper formulation. For example, a slowly soluble pellet of the compound of the present disclosure (e.g., compounds of Formulas (I), (II), (III), (IV), and (V) as well as those in Table 1) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of the present disclosure (e.g., compounds of Formulas (I), (II), (III), (IV), and (V) as well as those in Table 1) in oily or emulsified vehicles that allow it to disperse slowly in the serum.

### EXAMPLES

The following Examples are presented by way of illustration, not limitation. Compounds are named using the automatic name generating tool provided in ChemDraw (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

Abbreviations

| | |
|---|---|
| ACN | acetonitrile |
| AcOH | acetic acid |
| B₂Pin₂ | bis(pinacolato)diboron |
| Boc | tert-butyloxycarbonyl |
| calc or calcd | calculated |
| DCM | dichloromethane |
| DIBAL-H | diisobutylaluminium hydride |
| DIEA or DIPEA | N,N-diisopropylethylamine |
| DMAC | N,N dimethyl acetamide |
| DMF | N,N-dimethylformamide |
| DMSO | dimethyl sulfoxide |
| EA or EtOAc | ethyl acetate |
| EtOH | ethanol |
| FA | formic acid |
| GCMS | gas chromatography-mass spectrometry |
| HPLC | high-performance liquid chromatography |
| hr(s) | hour(s) |
| IPA | isopropyl alcohol |
| LCMS | liquid chromatography mass spectrometry |
| LDA | lithium diisopropylamide |
| MeOH | methanol |
| min(s) | minute(s) |
| MTBE | methyl tert-butyl ether |
| OMs | mesylate |
| PE | petroleum ether |
| rt | room temperature |
| TCCA | trichloroisocyanuric acid |
| TEA | triethylamine |
| TFA | trifluoroacetic acid |
| THF | tetrahydrofuran |
| TLC | thin layer chromatography |

### SYNTHETIC EXAMPLES

### Analytical Methods

Unless otherwise noted, all reagents were used without further purification. ¹H NMR spectra were obtained in DMSO-d₆, CD₃OD or CDCl₃ at room temperature on a Bruker 300 MHz or an Agilent 400 MHz instrument. When more than one conformer was detected, the chemical shifts for the most abundant one is reported. Chemical shifts of ¹H NMR spectra were recorded in parts per million (ppm) on the 6 scale from an internal standard of residual solvent. Splitting patterns are designed as s, singlet; d, doublet; t, triplet; q, quartet; m, multiplet; br, broad. LC-MS and HPLC conditions were described below:

The following are general LC methods of LC/MS.

### General LC Method A

Column: Agilent Zorbax XDB C18 4.6×50 mm, 3.5 μm

Mobile phase A: 0.1% formic acid in water

Mobile phase B: MeOH

Flow rate: 1.0 mL/min, Run time: 2 min gradient (20%-90% B), then 3 min @90% B, Column Temperature: 30° C.

### General LC Method B

Column: X Select CSH C18 2.5 um; 3.0×50 mm

Mobile Phase A: 0.05% FA in Water+5% ACN

Mobile Phase B: 0.05% FA in ACN

Flow Rate: 1.2 mL/min, Column Temperature: 50° C.

Gradient Program (Time/B %): 0/2, 0.2/2, 2.2/98, 3/98, 3.2/2, 4/2

### General LC Method C

Column: X-Bridge BEH C18, (50 mm*3.0 mm, 2.5 μm)

Mobile Phase A: 2.5 mM Ammonium Bicarbonate in Water+5% ACN

Mobile Phase B: 100% ACN

Flow rate: 1.2 mL/min, Column temperature: 50° C.

Gradient Program (B %): 0.0/0, 1.4/100, 2.4/100, 2.6/0, 3.0/0

The following are general HPLC methods.

### General HPLC Method A

Column: Agilent SB-C18 4.6×150 mm, 3.5 μm

Mobile phase A: 0.02% TFA in water

Mobile phase B: MeOH

Flow rate: 1.0 mL/min, Run time: 0.5 min @10% B, 9.5 min gradient (10%-90% B), then 10 min @90% B, Temperature: 30° C.

### General HPLC Method B

Column: X-Select CSH C18 (4.6*150) mm 5 μm,

Mobile Phase A: 0.1% Formic acid in water: Acetonitrile (95:05),

Mobile phase B: Acetonitrile,

Flow Rate: 1.0. mL/minute,

Gradient program: Time(min)/B Conc.: 0.01/5, 1.0/5, 8.0/100, 12.0/100, 14.0/5, 18.0/5

### General HPLC Method C

Column: X-Bridge C18 (4.6*150) mm 5 μm,

Mobile Phase A: 5 mM Ammonium Bicarbonate in water,

Mobile Phase B: Acetonitrile, Inj Volume; 5.0 μL,

Flow Rate: 1.0 mL/minute,

Gradient program: Time(min)/B Conc.: 0.01/5, 1.0/5, 8.0/100, 12.0/100, 14.0/5, 18.0/5

### General HPLC Method D

Column: X-Select CSH C18 (4.6*150) mm 5 μm

Mobile Phase A: 0.1% TFA in water,

Mobile Phase B: Acetonitrile, Inj Volume; 5.0 μL,

Flow Rate: 1.2 mL/minute,

Gradient program: Time(min)/B Conc: 0.01/5, 1.0/5, 8.0/100, 12.0/100, 14.0/5, 18.0/5

### General HPLC Method E

Column: X-Select CSH C18 (4.6*150) mm 5u,

Mobile phase A: 0.02% TFA in water

Mobile phase B: MeOH

Flow rate: 1.0 mL/min,

Run time: 0.5 min @10% B, 9.5 min gradient (10%-90% B), then 10 min @90% B

Temperature: 30° C.

The following is the general preparative LC method.

### General Preparative LC Method

Column: Phenomenex Luna 5u 100A, 21.2×250 mm, 5 μm

Mobile phase A: Water

Mobile phase B: MeOH

Flow rate: 10 mL/min, Run time: 1 min @20% B, 30 min gradient (20%-80% B), then 10 min @90% B, Temperature: Ambient

### Preparation of Common Intermediates

Reference to a particular intermediate compound by number, such as 1 or 2, is specific to the example in which it is described. As such, multiple examples may refer to the same intermediate compound number, such as 1 or 2, but the chemical structure of the compound will be different across the different examples.

### Example I-1. Synthesis of Common Intermediate 1

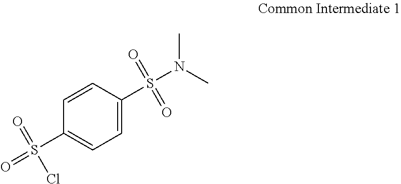 [SMILES: CN(C)S(=O)(=O)c1ccc(S(=O)(=O)Cl)cc1]

Common Intermediate 1 was made according to procedures described in WO 2017/141049 and WO 2018/005713.

### Example I-2. Synthesis of Common Intermediate 2

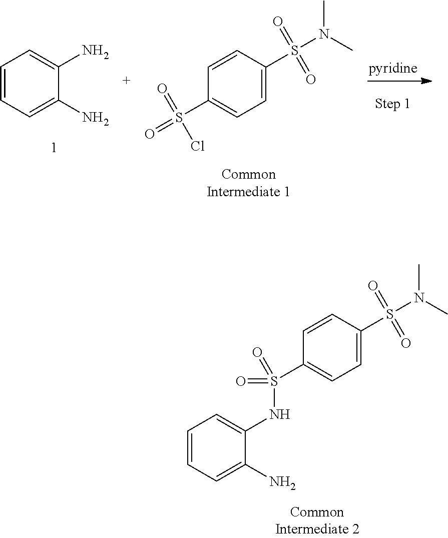 [SMILES: Nc1ccccc1N ; CN(C)S(=O)(=O)c1ccc(S(=O)(=O)Cl)cc1 ; CN(C)S(=O)(=O)c1ccc(S(=O)(=O)Nc2ccccc2N)cc1]

To a solution of benzene-1,2-diamine (620 mg, 5.74 mmol) in THE (50 mL) was added Common Intermediate 1 (1.30 g, 4.59 mmol) and pyridine (1.82 g, 22.96 mmol). The reaction mixture was stirred at 65° C. overnight. After the reaction was completed as indicated by TLC, the reaction was quenched with water (50 mL) and extracted with EtOAc (70 mL×2). The combined organic layers were washed with brine, dried over anhydrous Na₂SO₄, filtered, and concentrated under vacuum. The resulting solid was purified by silica gel chromatography (DCM=100%) to give 1.3 g of Common Intermediate 2. The yield was 63.8%.

LCMS [M+H]⁺ calcd for C₁₄H₁₈N₃O₄S₂, 356.07; found, 356.

### Example I-3. Synthesis of Common Intermediate 3

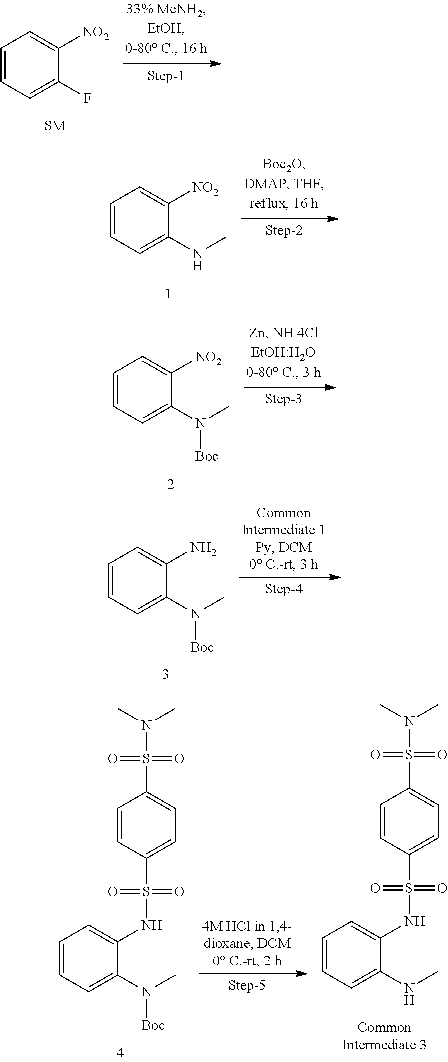 [SMILES: O=[N+]([O-])c1ccccc1F ; CNc1ccccc1[N+](=O)[O-] ; CN(C(=O)OC(C)(C)C)c1ccccc1[N+](=O)[O-] ; CN(C(=O)OC(C)(C)C)c1ccccc1N ; CN(C(=O)OC(C)(C)C)c1ccccc1NS(=O)(=O)c1ccc(S(=O)(=O)N(C)C)cc1 ; CNc1ccccc1NS(=O)(=O)c1ccc(S(=O)(=O)N(C)C)cc1]

### N-methyl-2-nitroaniline (1)

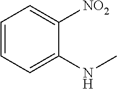

To a solution of 1-fluoro-2-nitrobenzene (SM) (5 g, 35.46 mmol) in EtOH (37.5 mL) was added 33% MeNH₂ in ethanol (8 mL, 85.2 mmol) slowly at 0° C. The resulting reaction mixture was heated at 80° C. and stirred for 16 h. After the reaction was completed as indicated by TLC, the reaction mixture was concentrated under vacuum. The crude was dissolved in EtOAc (50 mL) and washed with water (2×100 mL). The organic layers were dried over anhydrous Na₂SO₄, filtered, and concentrated under reduced pressure to give 5.1 g of the title compound. The yield was 95%.

LCMS [M+H]⁺ calcd for C₇H₉N₂O₂, 153.06; found, 153.11.

### Tert-butyl methyl(2-nitrophenyl)carbamate (2)

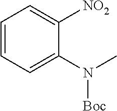 [SMILES: CN(C(=O)OC(C)(C)C)c1ccccc1[N+](=O)[O-]]

To a stirred solution of N-methyl-2-nitroaniline (1) (5.6 g, 36.84 mmol) in THE (56 mL) were added DMAP (449 mg, 3.68 mmol) and Boc₂O (25 mL, 110.52 mmol) at room temperature. The resulting reaction mixture was refluxed for 16 h. After the reaction was completed as indicated by TLC, the reaction mixture was diluted with water (50 mL) and extracted with EtOAc (2×100 mL). The combined organic layers were dried over anhydrous Na₂SO₄, filtered, and concentrated under reduced pressure. The crude product was purified by silica gel column chromatography by eluting with 10-20% EtOAc in heptane to give 4.5 g of the title compound. The yield was 48%.

¹H NMR (400 MHz, DMSO-d₆) δ 7.97 (d, J=7.8 Hz, 1H), 7.79-7.68 (m, 1H), 7.59 (d, J=7.8 Hz, 1H), 7.50 (t, J=7.6 Hz, 1H), 3.21 (s, 3H), 1.22 (s, 9H); LCMS [M-Boc+H]⁺ calcd for C₇H₉N₂O₂, 153.06; found, 153.2.

Tert-butyl (2-aminophenyl)(methyl)carbamate (3)

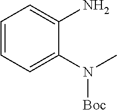 [SMILES: CN(C(=O)OC(C)(C)C)c1ccccc1N]

To a stirred solution of tert-butyl methyl(2-nitrophenyl)carbamate (2) (4.5 g, 17.85 mmol) in EtOH: H₂O (60 mL, 5:1) were added Zn dust (11.6 g, 178.5 mmol) and NH₄Cl (9.5 g, 177.6 mmol) at 0° C. The reaction mixture was heated at 80° C. and stirred for 3 h. After the reaction was completed as indicated by TLC, the reaction mixture was filtered through a Celite pad and the filtrate was concentrated under reduced pressure. The crude product was diluted with water (100 mL) and extracted with EtOAc (3×100 mL). The organic layers were dried over anhydrous Na₂SO₄, filtered, and concentrated under reduced pressure to give 3.2 g of the title compound. The yield was 80.6%.

¹H NMR (400 MHz, DMSO-d₆) δ 6.99-6.83 (m, 2H), 6.69 (d, J=7.8 Hz, 1H), 6.50 (t, J=7.1 Hz, 1H), 3.32 (s, 2H), 2.98 (s, 3H), 1.29 (br s, 9H); LCMS [M-Boc+H]⁺ calcd for C₇H₁₁N₂, 123.08; found, 123.2.

### Tert-butyl (2-((4-(N,N-dimethylsulfamoyl)phenyl)sulfonamido)phenyl)(methyl)carbamate (4)

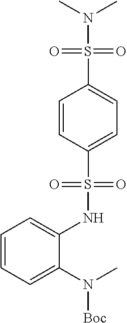 [SMILES: CN(C(=O)OC(C)(C)C)c1ccccc1NS(=O)(=O)c1ccc(S(=O)(=O)N(C)C)cc1]

To a stirred solution of tert-butyl (2-aminophenyl)(methyl)carbamate (3) (2 g, 9.01 mmol) in CH₂Cl₂ (20 mL) were added pyridine (1.5 mL, 18.6 mmol) and 4-(N,N-dimethylsulfamoyl)benzenesulfonyl chloride (Common Intermediate 1) (3 g, 10.6 mmol) at 0° C. The reaction mixture was stirred at room temperature for 3 h. After the reaction was completed as indicated by TLC, the reaction mixture was diluted with water (5 mL) and extracted with EtOAc (2×30 mL). The combined organic layers were dried over anhydrous Na₂SO₄, filtered, and concentrated under reduced pressure to give 3.2 g of the title compound. The yield was 76%.

¹H NMR (400 MHz, DMSO-d₆) δ 10.09 (br s, 1H), 8.14-7.84 (m, 4H), 7.35-7.04 (m, 4H), 2.91 (s, 3H), 2.64 (s, 6H), 1.28 (s, 9H); LCMS [M-Boc+H]⁺ calcd for C₁₅H₂₀N₃O₄S₂, 370; found, 370.3.

### N1,N1-dimethyl-N4-(2-(methylamino)phenyl)benzene-1,4-disulfonamide (Common Intermediate 3)

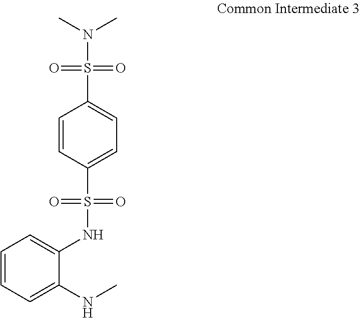

To a stirred solution of tert-butyl (2-((4-(N,N-dimethylsulfamoyl)phenyl)sulfonamido)phenyl)(methyl)carbamate (4) (1.5 g, 3.19 mmol) in CH₂Cl₂ (10 mL) was added 4M HCl in dioxane (15 mL) at 0° C. The reaction mixture was stirred at room temperature for 2 h. After the reaction was completed as indicated by TLC, the reaction mixture was concentrated under reduced pressure. The crude product was triturated with diethyl ether (2×10 mL) and dried under vacuum to give 1.1 g of the title compound. The yield was 93%.

¹H NMR (400 MHz, DMSO-d₆) δ 9.80-9.43 (br s, 1H), 7.93-7.86 (m, 4H), 7.09-7.01 (m, 1H), 6.56 (dd, J=1.5, 7.8 Hz, 1H), 6.50 (dd, J=1.1, 8.3 Hz, 1H), 6.41 (td, J=1.3, 7.5 Hz, 1H), 5.47-5.42 (br s, 1H), 2.62 (s, 6H), 2.57 (s, 3H); LCMS [M+H]⁺ calcd for C₁₅H₂₀N₃O₄S₂, 370.08; found, 370.55.

### Example 1-4. Synthesis of Common Intermediate 3

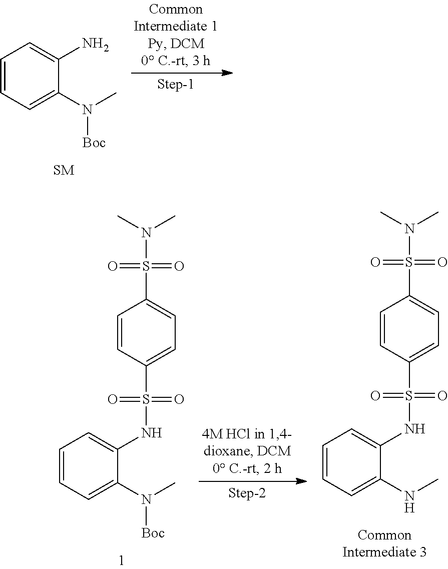 [SMILES: CN(C(=O)OC(C)(C)C)c1ccccc1N ; CN(C(=O)OC(C)(C)C)c1ccccc1NS(=O)(=O)c1ccc(S(=O)(=O)N(C)C)cc1 ; CNc1ccccc1NS(=O)(=O)c1ccc(S(=O)(=O)N(C)C)cc1]

### Tert-butyl (2-((4-(N,N-dimethylsulfamoyl)phenyl) sulfonamido)phenyl) (methyl)carbamate (1)

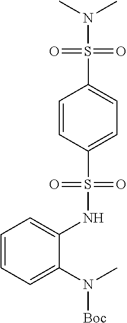 [SMILES: CN(C(=O)OC(C)(C)C)c1ccccc1NS(=O)(=O)c1ccc(S(=O)(=O)N(C)C)cc1]

A solution of SM (2 g, 9.01 mmol) in CH₂Cl₂ (20 mL) was added to pyridine (1.5 mL, 18.6 mmol) and 4-(N,N-dimethylsulfamoyl)benzenesulfonyl chloride (Common Intermediate 1) (3 g, 10.6 mmol) at 0° C. The reaction mixture was stirred at room temperature for 3 h. After the reaction was completed as indicated by TLC, the reaction mixture was diluted with water (5 mL) and extracted with EtOAc (2×30 mL). The combined organic layers were dried over anhydrous Na₂SO₄, filtered, and concentrated under reduced pressure to give 3.2 g of the title compound. The yield was 76%.

¹H NMR (400 MHz, DMSO-d₆) δ 10.09 (br s, 1H), 8.14-7.84 (m, 4H), 7.35-7.04 (m, 4H), 2.91 (s, 3H), 2.64 (s, 6H), 1.28 (s, 9H); LCMS [M-Boc+H]⁺ calcd for C₁₅H₂₀N₃O₄S₂, 370; found, 370.3

### N1,N1-dimethyl-N4-(2-(methylamino)phenyl)benzene-1,4-disulfonamide (Common Intermediate 3)

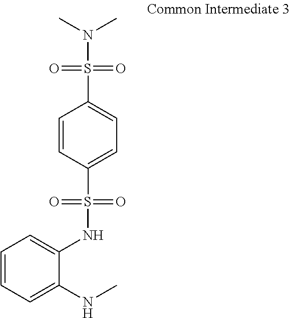 [SMILES: CNc1ccccc1NS(=O)(=O)c1ccc(S(=O)(=O)N(C)C)cc1]

A solution of intermediate 1 (1.5 g, 3.19 mmol) in CH₂Cl₂ (10 mL) was added to 4M HCl in dioxane (15 mL) at 0° C. The reaction mixture was stirred at room temperature for 2 h. After the reaction was completed as indicated by TLC, the reaction mixture was concentrated under reduced pressure. The crude product was triturated with diethyl ether (2×10 mL) and dried under vacuum to give 1.1 g of the title compound. The yield was 93%.

¹H NMR (400 MHz, DMSO-d₆) δ 9.80-9.43 (br s, 1H), 7.93-7.86 (m, 4H), 7.09-7.01 (m, 1H), 6.56 (dd, J=1.5, 7.8 Hz, 1H), 6.50 (dd, J=1.1, 8.3 Hz, 1H), 6.41 (td, J=1.3, 7.5 Hz, 1H), 5.47-5.42 (br s, 1H), 2.62 (s, 6H), 2.57 (s, 3H);

LCMS [M+H]⁺ calcd for C₁₅H₂₀N₃O₄S₂, 370; found, 370.55

### Example I-5. Synthesis of Common Intermediate 4

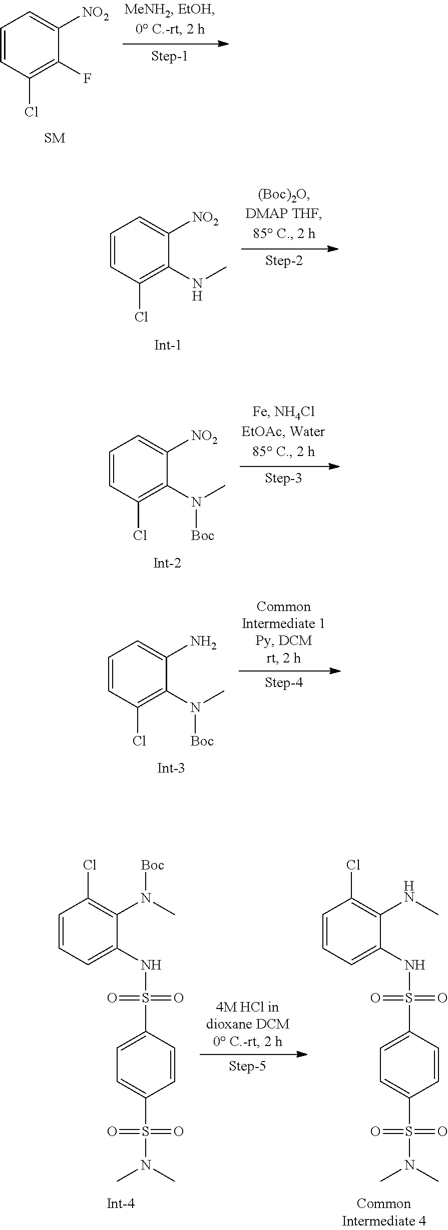 [SMILES: O=[N+]([O-])c1cccc(Cl)c1F ; CNc1c(Cl)cccc1[N+](=O)[O-] ; CN(C(=O)OC(C)(C)C)c1c(Cl)cccc1[N+](=O)[O-] ; CN(C(=O)OC(C)(C)C)c1c(N)cccc1Cl ; CN(C(=O)OC(C)(C)C)c1c(Cl)cccc1NS(=O)(=O)c1ccc(S(=O)(=O)N(C)C)cc1 ; CNc1c(Cl)cccc1NS(=O)(=O)c1ccc(S(=O)(=O)N(C)C)cc1]

### 2-Chloro-N-methyl-6-nitroaniline (1)

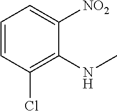

To a solution of 1-chloro-2-fluoro-3-nitrobenzene SM (5 g, 28.57 mmol) in ethanol (10 mL) was added methylamine (2.6 g, 85.71 mmol) at 0° C. The reaction mixture was allowed to reach room temperature and stirred for 2 h. After the reaction was completed as indicated by TLC, the reaction mixture was extracted with ethyl acetate and water. The combined organic layers were dried over Na₂SO₄, filtered, and concentrated under reduced pressure. The crude product was purified by medium pressure liquid chromatography by eluting with 30% EtOAc in hexane to give 4 g of the title compound. The yield was 45%.

### Tert-butyl (2-chloro-6-nitrophenyl)(methyl)carbamate (2)

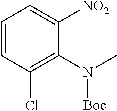 [SMILES: CN(C(=O)OC(C)(C)C)c1c(Cl)cccc1[N+](=O)[O-]]

To a solution of Int-1 (1.2 g, 7.05 mmol) in THE (10 mL) was added DMAP (0.07 g, 0.705 mmol) and Boc anhydride (5.9 g, 21.15 mmol) at 0° C. The reaction mixture was heated to 85° C. and stirred for 2 h. After the reaction was completed as indicated by TLC, the reaction mixture was extracted with ethyl acetate and water. The combined organic layers were concentrated under reduced pressure to give 800 mg crude. The crude product was used as such in the next step without further purification and analysis.

### Tert-butyl (2-amino-6-chlorophenyl)(methyl)carbamate (3)

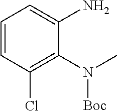 [SMILES: CN(C(=O)OC(C)(C)C)c1c(N)cccc1Cl]

To a solution of Int-2 (1 g, 3.48 mmol) in a mixture of solvents ethyl acetate and water (2 mL, 5:1) was added NH₄Cl (1.7 g, 32.01 mmol) and iron powder (1.92 g, 34.8 mmol) at room temperature. The reaction mixture was heated to 85° C. and stirred for 2 h. After the reaction was completed as indicated by TLC, the reaction mixture was extracted with ethyl acetate and water. The combined organic layers were dried over anhydrous Na₂SO₄, filtered, and concentrated under reduced pressure to give 800 mg of crude was used for next step without further purification and analysis.

### Tert-butyl (2-chloro-6-((4-(N,N-dimethylsulfamoyl)phenyl)sulfonamido)phenyl)(methyl)carbamate (4)

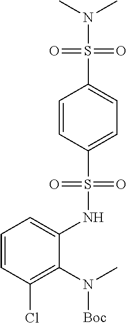 [SMILES: CN(C(=O)OC(C)(C)C)c1c(Cl)cccc1NS(=O)(=O)c1ccc(S(=O)(=O)N(C)C)cc1]

A solution of Int-3 (280 mg, 0.55 mmol) in DCM (10 mL) was added to pyridine (1.5 mL, 10.44 mmol) and 4-(N,N-dimethylsulfamoyl)benzenesulfonyl chloride (Common Intermediate 1) (1.18 g, 4.17 mmol) at room temperature for 2 h. After the reaction was completed as indicated by TLC, the reaction mixture was extracted with DCM and water. The combined organic layers were dried over anhydrous Na₂SO₄, filtered, and concentrated under reduced pressure. The crude product was purified by medium pressure liquid chromatography by eluting with 30% EtOAc in hexane to give 800 mg of the title compound. The yield was 45%.

### N1-(3-chloro-2-(methylamino)phenyl)-N4,N4-dimethylbenzene-1,4-disulfonamide (Common Intermediate 4)

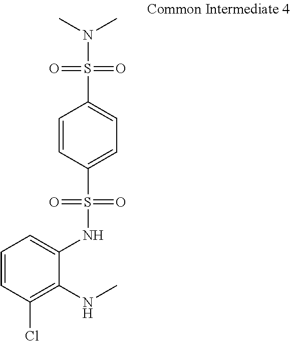 [SMILES: CNc1c(Cl)cccc1NS(=O)(=O)c1ccc(S(=O)(=O)N(C)C)cc1]

A solution of Int-4 (280 mg, 0.55 mmol) in CH₂Cl₂ (10 mL) was added to 4 M HCl in dioxane (10 mL) at 0° C. The reaction mixture was stirred at room temperature for 2 h. After the reaction was completed as indicated by TLC, the reaction mixture was washed with DCM and concentrated under reduced pressure. The crude product was triturated with pentane and dried under red to give 130 mg of the title compound. The yield was 59%.

LCMS [M+H]+ calcd for C₁₅H₁₉ClN₃O₄S₂, 404.04; found, 404.3.

### Example 1-6. Synthesis of Common Intermediate 5

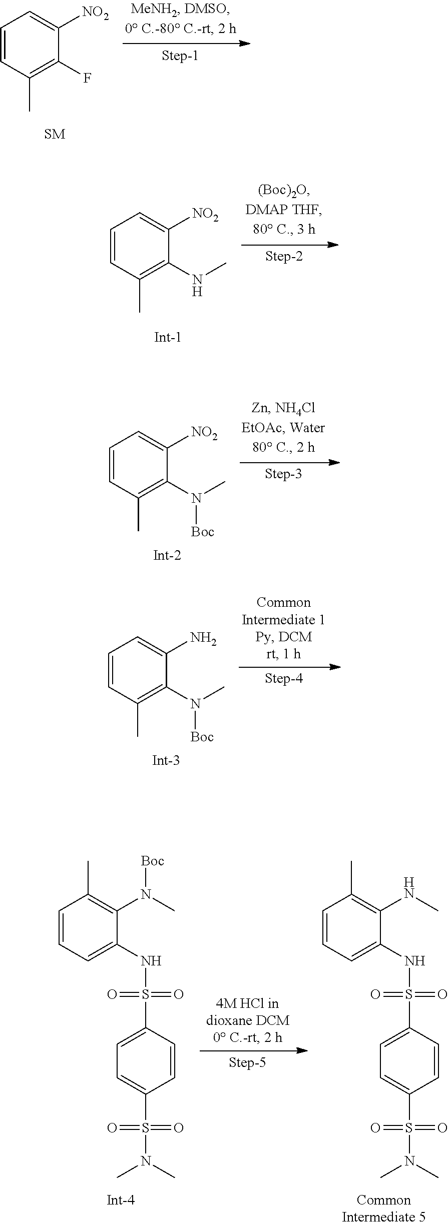 [SMILES: Cc1cccc([N+](=O)[O-])c1F ; CNc1c(C)cccc1[N+](=O)[O-] ; Cc1cccc([N+](=O)[O-])c1N(C)C(=O)OC(C)(C)C ; Cc1cccc(N)c1N(C)C(=O)OC(C)(C)C ; Cc1cccc(NS(=O)(=O)c2ccc(S(=O)(=O)N(C)C)cc2)c1N(C)C(=O)OC(C)(C)C ; CNc1c(C)cccc1NS(=O)(=O)c1ccc(S(=O)(=O)N(C)C)cc1]

### N,2-Dimethyl-6-nitroaniline (1)

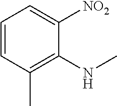

To a solution of 2-fluoro-1-methyl-3-nitrobenzene SM (2 g, 12.90 mmol) in DMSO (20 mL) was added methyl amine (1.45 mL, 15.48 mmol) at 0° C. The reaction mixture was heated to 80° C. and stirred for 2 h. After the reaction was completed as indicated by TLC, the reaction mixture was extracted with ethyl acetate and water. Combined organic layers were washed with brine, dried over Na₂SO₄, filtered, and concentrated under reduced pressure to give 1.07 g which was used as such in the next step without purification.

LCMS [M+H]⁺ calcd for C₈H₁₁N₂O₂, 167.07; found, 167.02.

### Tert-butyl methyl(2-methyl-6-nitrophenyl)carbamate (2)

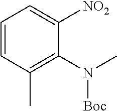 [SMILES: Cc1cccc([N+](=O)[O-])c1N(C)C(=O)OC(C)(C)C]

To a solution of intermediate 1 (2.2 g, 13.25 mmol) in THE (22 mL) was added DMAP (0.16 g, 1.32 mmol) and Boc anhydride (12.5 mL, 39.76 mmol) at 0° C. The reaction mixture was heated to 80° C. and stirred for 3 h. After the reaction was completed as indicated by TLC, the reaction mixture was extracted with ethyl acetate and water. The combined organic layers were washed with brine, dried over Na₂SO₄, and filtered, and the filtrate was concentrated under reduced pressure. The crude product was purified by medium pressure liquid chromatography, eluted with 80-90% ethyl acetate in heptane to give 0.55 g. The yield was 15%.

LCMS [M−H]+ calcd for C₁₃H₁₇N₂O₄, 265.13; found, 265.30

### Tert-butyl (2-amino-6-methylphenyl)(methyl)carbamate (3)

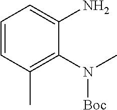 [SMILES: Cc1cccc(N)c1N(C)C(=O)OC(C)(C)C]

To a solution of intermediate 2 (0.55 g, 2.06 mmol) in a mixture of solvents ethyl acetate and water (4:1) was added NH₄Cl (1.1 g, 20.67 mmol) and zinc powder (1.3 g, 20.67 mmol) at room temperature. The reaction mixture was heated to 80° C. and stirred for 2 h. After the reaction was completed as indicated by TLC, the reaction mixture was extracted with ethyl acetate and water. The combined organic layers were dried over anhydrous Na₂SO₄, and filtered, and the filtrate was concentrated under reduced pressure to give 0.4 g of the title compound. The yield was 83%.

LCMS calcd for C₁₃H₂₀N₂O₂, 236.15; found, [M-Boc+H]+ 137.03.

### Tert-butyl (2-((4-(N,N-dimethylsulfamoyl)phenyl)sulfonamido)-6-methylphenyl)(methyl)carbamate (4)

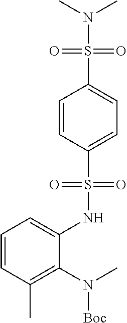 [SMILES: Cc1cccc(NS(=O)(=O)c2ccc(S(=O)(=O)N(C)C)cc2)c1N(C)C(=O)OC(C)(C)C]

A solution of compound 3 (0.4 g, 1.69 mmol) in DCM (4 mL) was added to pyridine (0.4 mL, 5.08 mmol) and 4-(N,N-dimethylsulfamoyl)benzenesulfonyl chloride (Common Intermediate 1) (0.57 g, 2.04 mmol) at 0° C. The reaction mixture was allowed to reach room temperature and stirred for 1 h. After the reaction was completed as indicated by TLC, the reaction mixture was extracted with water and ethyl acetate. The organic layers were washed with brine, dried over anhydrous Na₂SO₄, and filtered, and the filtrate was concentrated under reduced pressure. The crude product was purified by medium pressure liquid chromatography by eluting with 50-60% EtOAc in heptane to give 0.4 g of the title compound. The yield was 50%.

### N1,N1-dimethyl-N4-(3-methyl-2-(methylamino)phenyl)benzene-1,4-disulfonamide (Common Intermediate 5)

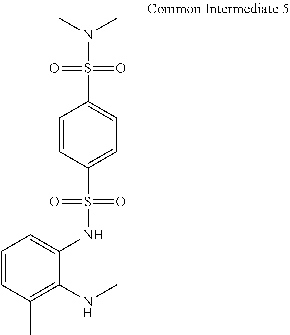 [SMILES: CNc1c(C)cccc1NS(=O)(=O)c1ccc(S(=O)(=O)N(C)C)cc1]

A solution of compound 4 (0.4 g, 0.83 mmol) in CH₂Cl₂ (4 mL) was added to 4 M HCl in dioxane (2 mL) at 0° C. The reaction mixture was stirred at room temperature for 1 h. After the reaction was completed as indicated by TLC, the reaction mixture was concentrated under reduced pressure. The crude product was triturated with pentane and dried under reduced pressure to give 0.28 g of the title compound. The yield was 90%.

LCMS [M−H]+ calcd for C₁₆H₂₂N₃O₄S₂, 384.10; found, 384.3

### Example I-7. Synthesis of Common Intermediate 6

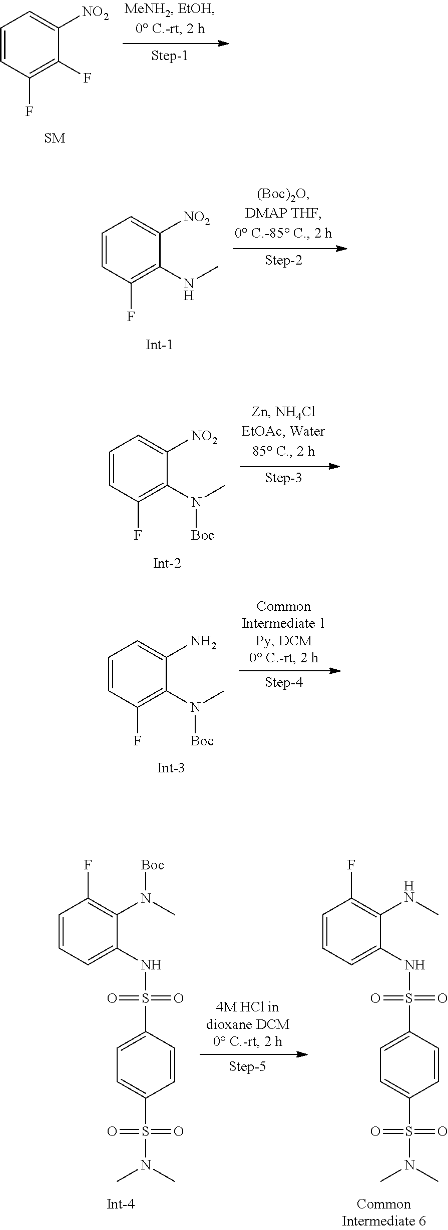 [SMILES: O=[N+]([O-])c1cccc(F)c1F ; CNc1c(F)cccc1[N+](=O)[O-] ; CN(C(=O)OC(C)(C)C)c1c(F)cccc1[N+](=O)[O-] ; CN(C(=O)OC(C)(C)C)c1c(N)cccc1F ; CN(C(=O)OC(C)(C)C)c1c(F)cccc1NS(=O)(=O)c1ccc(S(=O)(=O)N(C)C)cc1 ; CNc1c(F)cccc1NS(=O)(=O)c1ccc(S(=O)(=O)N(C)C)cc1]

### 2-Fluoro-N-methyl-6-nitroaniline (1)

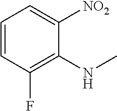

To a solution of 1,2-difluoro-3-nitrobenzene SM (1 g, 6.29 mmol) in ethanol (5 mL) was added methyl amine (0.58 g, 18.87 mmol) at 0° C. The reaction mixture was allowed to reach room temperature and stirred for 2 h. After the reaction was completed as indicated by TLC, the reaction mixture was extracted with ethyl acetate and water. The combined organic layers were dried over Na₂SO₄, filtered, and concentrated under reduced pressure. The crude product was purified by medium pressure liquid chromatography by eluting with 10% EtOAc in hexane to give 1.2 g crude, which was used as such in the next step without analysis.

### Tert-butyl (2-fluoro-6-nitrophenyl)(methyl)carbamate (2)

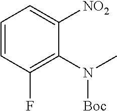 [SMILES: CN(C(=O)OC(C)(C)C)c1c(F)cccc1[N+](=O)[O-]]

To a solution of intermediate 1 (1.2 g, 7.05 mmol) in THF (10 mL) was added DMAP (0.07 g, 0.705 mmol) and Boc anhydride (5.9 g, 21.15 mmol) at 0° C. The reaction mixture was heated to 85° C. and stirred for 2 h. After the reaction was completed as indicated by TLC, the reaction mixture was extracted with ethyl acetate and water. The combined organic layers were concentrated under reduced pressure to give 800 mg crude. The crude product was used as such in the next step without further purification and analysis.

### Tert-butyl (2-amino-6-fluorophenyl)(methyl)carbamate (3)

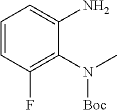 [SMILES: CN(C(=O)OC(C)(C)C)c1c(N)cccc1F]

A solution of intermediate 2 (500 mg, 1.85 mmol) in a mixture of solvents ethyl acetate and water (5 mL, 4:1) was added NH₄Cl (294 mg, 5.55 mmol) and zinc powder (1.20 g, 185 mmol) at room temperature. The reaction mixture was heated to 85° C. and stirred for 2 h. After the reaction was completed as indicated by TLC, the reaction mixture was extracted with ethyl acetate and water. The combined organic layers were dried over anhydrous Na₂SO₄, filtered, and concentrated under reduced pressure. The crude product was purified by medium pressure liquid chromatography by eluting with 30% EtOAc in hexane to give 400 mg of the title compound. The yield was 90%.

LCMS [M-Boc+H]+ calcd for C₁₂H₁₇FN₂O₂, 240.13; found, 140.9.

### Tert-butyl (2-((4-(N,N-dimethylsulfamoyl)phenyl)sulfonamido)-6-fluorophenyl)(methyl)carbamate (4)

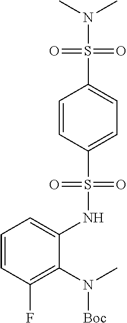 [SMILES: CN(C(=O)OC(C)(C)C)c1c(F)cccc1NS(=O)(=O)c1ccc(S(=O)(=O)N(C)C)cc1]

A solution of compound 3 (400 mg, 1.66 mmol) in DCM (5 mL) was added to pyridine (394 mg, 4.99 mmol) and 4-(N,N-dimethylsulfamoyl)benzenesulfonyl chloride (Common Intermediate 1) (567 mg, 1.99 mmol) at 0° C. The reaction mixture was allowed to reach room temperature and stirred for 2 h. After the reaction was completed as indicated by TLC, the reaction mixture was extracted with water. The combined organic layers were dried over anhydrous Na₂SO₄, filtered, and concentrated under reduced pressure. The crude product was purified by medium pressure liquid chromatography by eluting with 30% EtOAc in hexane to give 150 mg of the title compound. The yield was 18%.

### N1-(3-Fluoro-2-(methylamino)phenyl)-N4,N4-dimethylbenzene-1,4-disulfonamide (Common Intermediate 6)

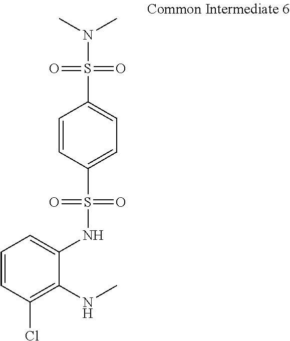 [SMILES: CNc1c(Cl)cccc1NS(=O)(=O)c1ccc(S(=O)(=O)N(C)C)cc1]

A solution of compound 4 (150 mg, 0.30 mmol) in CH₂Cl₂ (5 mL) was added to 4 M HCl in dioxane (5 mL) at 0° C. The reaction mixture was stirred at room temperature for 2 h. After the reaction was completed as indicated by TLC, the reaction mixture was washed with DCM and concentrated under reduced pressure. The crude product was triturated with pentane and dried under reduced pressure to give 110 mg of the title compound. The yield was 91%.

LCMS [M+H]⁺ calcd for C₁₅H₁₉FN₃O₄S₂, 388; found, 388.3.

### Example I-8. Synthesis of Common Intermediate 7

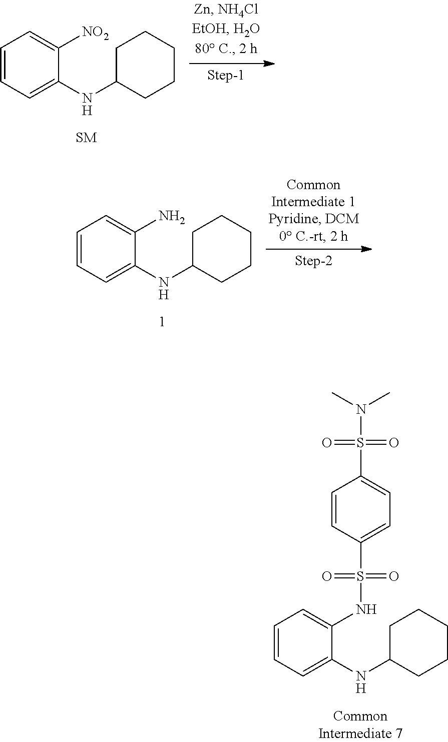 [SMILES: O=[N+]([O-])c1ccccc1NC1CCCCC1 ; Nc1ccccc1NC1CCCCC1 ; CN(C)S(=O)(=O)c1ccc(S(=O)(=O)Nc2ccccc2NC2CCCCC2)cc1]

### N1-Cyclohexylbenzene-1,2-diamine (1)

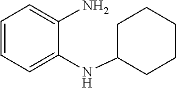 [SMILES: Nc1ccccc1NC1CCCCC1]

A solution of N-cyclohexyl-2-nitroaniline SM (1 g, 4.54 mmol) in a mixture of solvents ethanol and water (10 mL, 8:2) was added to Zn dust (1.47 g, 22.7 mmol) and NH₄C₁ (2.4 g, 45.4 mmol) at 0° C. The reaction mixture was heated to 80° C. and stirred for 2 h. After the reaction was completed as indicated by TLC, the reaction mixture was diluted with water and extracted with DCM (2×25 mL). The organic layers were dried over anhydrous Na₂SO₄, filtered, and concentrated under reduced pressure to give 700 mg of title compound. The yield was 81%.

LCMS [M+H]+ calcd for C₁₂H₁₉N₂, 191; found, 191.37.

### N1-(2-(Cyclohexylamino)phenyl)-N4,N4-dimethylbenzene-1,4-disulfonamide (Common Intermediate 7)

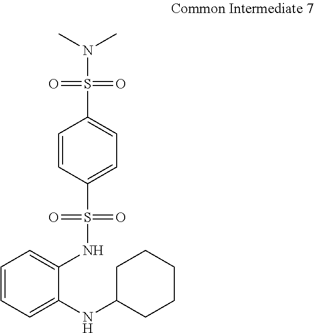 [SMILES: CN(C)S(=O)(=O)c1ccc(S(=O)(=O)Nc2ccccc2NC2CCCCC2)cc1]

A solution of intermediate 1 (600 mg, 3.16 mmol) in DCM (6 mL) was added to pyridine (0.6 mL, 7.9 mmol) and 4-(N,N-dimethylsulfamoyl)benzenesulfonyl chloride Common Intermediate 1 (983 mg, 3.47 mmol) at 0° C. The reaction mixture was allowed to reach room temperature and stirred for 2 h. After the reaction was completed as indicated by TLC, the reaction mixture was diluted with water and extracted with DCM (2×25 mL). The organic layers were dried over anhydrous Na₂SO₄, filtered, and concentrated under reduced pressure. The crude product was purified by medium pressure liquid chromatography, eluted with 20% ethyl acetate in heptane to give 1 g of title compound. The yield was 72%.

LCMS [M+H]+ calcd for C₂₀H₂₈N₃O₄S₂, 438; found, 438.4.

### Example S1. Synthesis of Compound 20

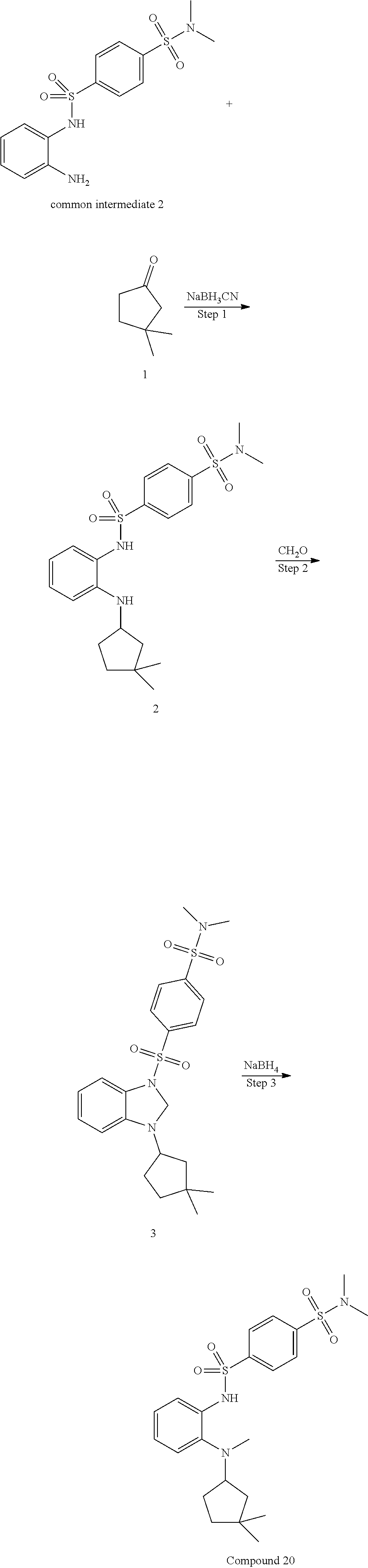

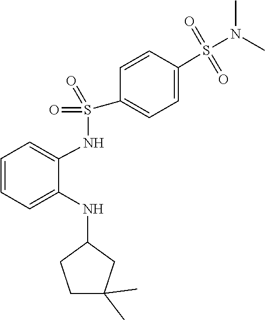 [SMILES: CN(C)S(=O)(=O)c1ccc(S(=O)(=O)Nc2ccccc2NC2CCC(C)(C)C2)cc1]

To a solution of Common Intermediate 2 (300 mg, 0.84 mmol) in MeOH (4 mL) and THF (4 mL) was added 3,3-dimethylcyclopentanone (1) (188 mg, 1.68 mmol), NaBH₃CN (211 mg, 3.36 mmol) and three drops of AcOH. The reaction mixture was stirred at 50° C. overnight. After the reaction was completed as indicated by TLC, the reaction was quenched with water (20 mL) and extracted with EtOAc (25 mL×2). The combined organic layers were washed with brine, dried over anhydrous Na₂SO₄, filtered, and concentrated under reduced pressure. The resulting solid was purified by silica gel chromatography (PE:EtOAc=10:1 to 5:1) to give 236 mg of N1-(2-((3,3-dimethylcyclopentyl)amino)phenyl)-N4,N4-dimethylbenzene-1,4-disulfonamide (2). The yield was 62.3%.

LCMS [M+H]⁺ calcd for C₂₁H₃₀N₃O₄S₂, 452.16; found, 452.

### Step 2

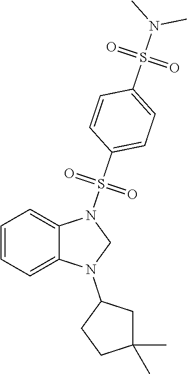 [SMILES: CN(C)S(=O)(=O)c1ccc(S(=O)(=O)N2CN(C3CCC(C)(C)C3)c3ccccc32)cc1]

To a solution of N1-(2-((3,3-dimethylcyclopentyl)amino)phenyl)-N4,N4-dimethylbenzene-1,4-disulfonamide (2) (250 mg, 0.55 mmol) in MeOH (4 mL) and DCM (4 mL) was added formaldehyde (180 mg, 2.22 mmol, 37%), NaBH₃CN (141 mg, 2.22 mmol) and two drops AcOH. The reaction mixture was stirred at ambient temperature for 6 hr. After the reaction was completed as indicated by TLC, the reaction was quenched with water (20 mL) and extracted with DCM (30 mL×2). The combined organic layers were dried over anhydrous Na₂SO₄, filtered, and concentrated under reduced pressure to give 250 mg of crude 4-((3-(3,3-dimethylcyclopentyl)-2,3-dihydro-1H-benzo[d]74midazole-1-yl) sulfonyl)-N,N-dimethylbenzenesulfonamide (3). The crude title compound was used in the next step without purification.

LCMS [M+H]⁺ calcd for C₂₂H₃₀N₃O₄S₂, 464.16; found, 464.

### Step 3

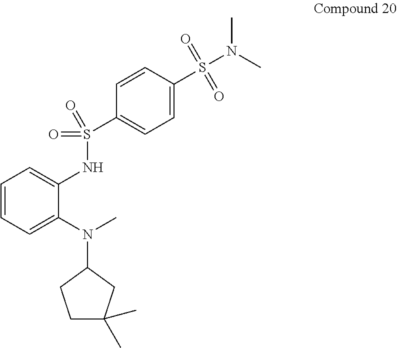 [SMILES: CN(c1ccccc1NS(=O)(=O)c1ccc(S(=O)(=O)N(C)C)cc1)C1CCC(C)(C)C1]

To a solution of 4-((3-(3,3-dimethylcyclopentyl)-2,3-dihydro-1H-benzo[d]75midazole-1-yl) sulfonyl)-N,N-dimethylbenzenesulfonamide (3) (250 mg, 0.55 mmol) in MeOH (10 mL) was added NaBH₄ (141 mg, 3.72 mmol) in four portions over 30 mins. The reaction mixture was stirred at ambient temperature for 2 hr. After the reaction was completed as indicated by TLC, the reaction was quenched with water (20 mL) and extracted with EtOAc (20 mL×2). The combined organic layers were washed with brine, dried over anhydrous Na₂SO₄, filtered, and concentrated under reduced pressure. The resulting oil was purified by silica gel chromatography to give the impure title compound. The impure title compound was further purified by preparative HPLC to give 68.3 mg of N1-(2-((3,3-dimethylcyclopentyl)(methyl)amino)phenyl)-N4,N4-dimethylbenzene-1,4-disulfonamide (Compound 20). The yield was 26.7%.

¹HNMR (300 MHz, CDCl₃) δ 8.55 (br s, 1H), 8.01 (d, J=8.4 Hz, 2H), 7.82 (d, J=8.4 Hz, 2H), 7.63 (d, J=6.0 Hz, 1H), 7.18-7.01 (m, 3H), 3.31 (m, 1H), 2.70 (s, 6H), 2.22 (s, 3H), 1.69 (m, 1H), 1.55 (m, 1H), 1.40 (m, 1H), 1.38 (m, 1H), 1.20 (m, 1H), 1.09 (m, 1H), 1.00 (s, 3H), 0.92 (s, 3H). LCMS [M+H]⁺ calcd for C₂₂H₃₂N₃O₄S₂, 466.18; found, 466.

### Example S2. Synthesis of Compound 27

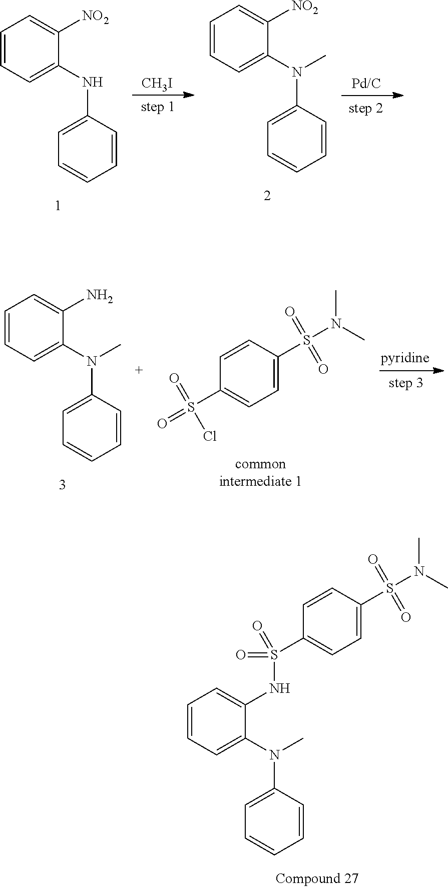

### Step 1

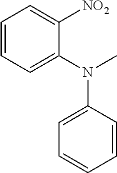 [SMILES: CN(c1ccccc1)c1ccccc1[N+](=O)[O-]]

Under nitrogen protection, to a solution of 2-nitro-N-phenylaniline (1) (300 mg, 1.40 mmol) in DMF (15 mL) was added NaH (168.04 mg, 4.20 mmol, 60%) portionwise over 2 mins. After the resulting mixture was stirred at rt for 10 mins, iodomethane (397.55 mg, 2.80 mmol) was added to the reaction mixture and the reaction was continued at rt overnight. After the reaction was completed as indicated by TLC, the reaction was quenched with water (30 mL) and extracted with EtOAc (30 mL×2). The combined organic layers were washed with brine, dried over anhydrous Na₂SO₄, filtered, and concentrated under reduced pressure. The residue was purified by silica gel chromatography (PE:EtOAc=50:1) to give 300 mg (1.31 mmol) of N-methyl-2-nitro-N-phenylaniline (2). The yield was 93.6%.

LCMS [M+H]⁺ calcd for C₁₃H₁₃N₂O₂, 229.09; found, 229.

### Step 2

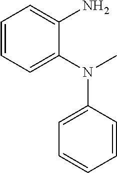

To a solution of N-methyl-2-nitro-N-phenylaniline (2) (300 mg, 1.31 mmol) in IPA (15 mL) was added Pd/C (50 mg, 10%). The suspension was evacuated and then refilled with hydrogen for three times. The reaction mixture was then stirred under hydrogen (balloon) overnight. After the reaction was completed as indicated by TLC, the suspension was filtered through a pad of Celite and the filtered cake was washed with IPA (10 mL). The combined filtrate was concentrated under reduced pressure to give 220 mg of crude N1-methyl-N1-phenylbenzene-1,2-diamine (3). The yield was 84.4%.

LCMS [M+H]⁺ calcd for C₁₃H₁₅N₂, 199; found, 199.

### Step 3

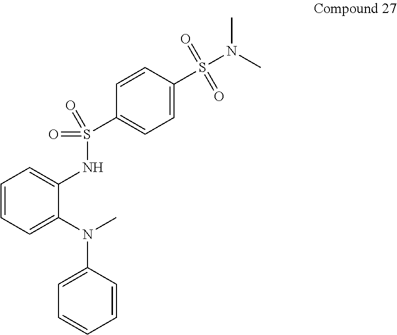 [SMILES: CN(c1ccccc1)c1ccccc1NS(=O)(=O)c1ccc(S(=O)(=O)N(C)C)cc1]

To a solution of N1-methyl-N1-phenylbenzene-1,2-diamine (3) (100 mg, 0.50 mmol) in THE (5 mL) was added the common intermediate 1 (171.74 mg, 0.60 mmol) and pyridine (119.69 mg, 1.51 mmol). The reaction mixture was stirred at 60° C. overnight. After the reaction was completed as indicated by TLC, the reaction was quenched with water (20 mL) and extracted with EtOAc (20 mL×2). The combined organic layers were washed with brine, dried over anhydrous Na₂SO₄, filtered, and concentrated under reduced pressure. The residue was purified by silica gel chromatography to give the impure title compound. The impure product was further purified by preparative HPLC to give 77.7 mg of N1,N1-dimethyl-N4-(2-(methyl(phenyl)amino)phenyl)benzene-1,4-disulfonamide (Compound 27). The yield was 34.6%.

¹HNMR (300 MHz, CDCl₃) δ 7.87-7.74 (m, 5H), 7.35-7.30 (m, 2H), 7.17-7.05 (m, 4H), 6.84 (t, J=7.2 Hz, 1H), 6.34 (d, J=7.8 Hz, 1H), 2.92 (s, 3H), 2.73 (s, 6H). LCMS [M+H]⁺ calcd for C₂₁H₂₄N₃O₄S₂, 446.11; found, 445.9.

### Example S3. Synthesis of Compound 26

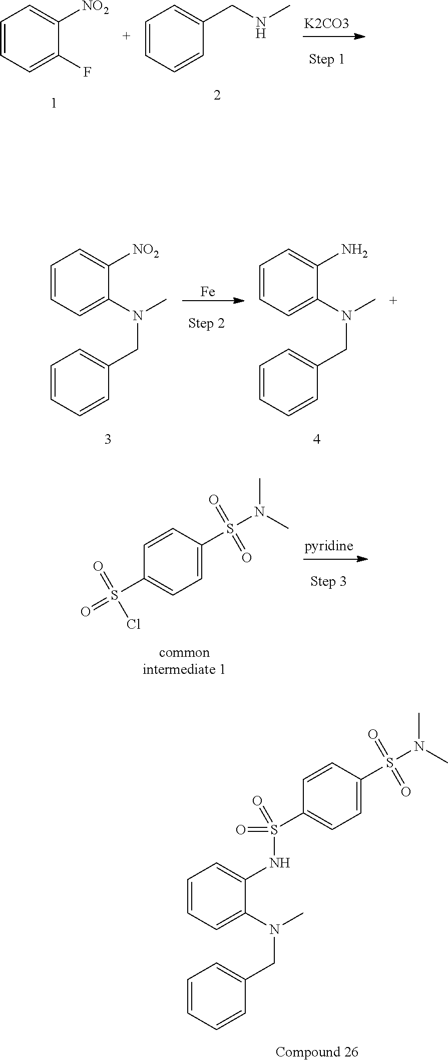

### Step 1

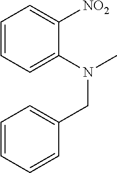 [SMILES: CN(Cc1ccccc1)c1ccccc1[N+](=O)[O-]]

To a solution of 1-fluoro-2-nitrobenzene (1) (350 mg, 2.48 mmol) in DMF (25 mL) was added N-methyl-1-phenylmethanamine (2) (902 mg, 7.44 mmol) and K₂CO₃ (1028 mg, 7.44 mmol). The reaction mixture was stirred at 100° C. for 2 hr. After the reaction was completed as indicated by TLC, the reaction was quenched with water (50 mL) and extracted with EtOAc (40 mL×2). The combined organic layers were washed with water and brine, dried over anhydrous Na₂SO₄, filtered, and concentrated under reduced pressure. The residual oil was purified by silica gel chromatography (PE:EtOAc=60:1) to give 560 mg of N-benzyl-N-methyl-2-nitroaniline (3). The yield was 93.3%.

¹HNMR (300 MHz, CDCl₃) δ 7.76 (d, J=8.1 Hz, 1H), 7.46-7.20 (m, 6H), 7.07 (d, J=8.1 Hz, 1H), 6.89 (t, J=7.8 Hz, 1H), 4.38 (s, 2H), 2.78 (s, 3H).

### Step 2

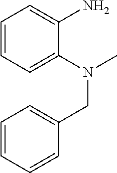 [SMILES: CN(Cc1ccccc1)c1ccccc1N]

To a solution of N-benzyl-N-methyl-2-nitroaniline (3) (400 mg, 1.65 mmol) in an aqueous saturated NH₄Cl solution (5 mL), THE (5 mL) and EtOH (5 mL) was added Fe powder (370 mg, 6.60 mmol) in one portion. The reaction mixture was stirred at 70° C. for 2 hr. After the reaction was completed as indicated by TLC, the suspension was filtered to remove the undissolved solid. The filtrate was quenched with water (30 mL) and extracted with EtOAc (40 mL×2). The combined organic layers were washed with water and brine, dried over anhydrous Na₂SO₄, filtered, and concentrated under reduced pressure. The residue was purified by silica gel chromatography (PE:EtOAc=10:1 to 20:1) to give 170 mg of N1-benzyl-N1-methylbenzene-1,2-diamine (4). The yield was 48.6%.

LCMS [M+H]⁺ calcd for C₁₄H₁₇N₂, 213.13; found, 213.

### Step 3

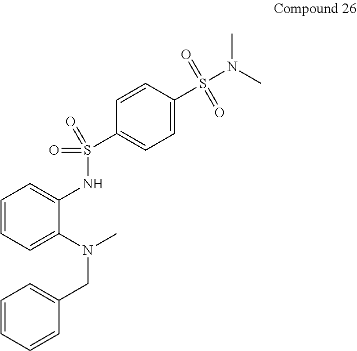 [SMILES: CN(Cc1ccccc1)c1ccccc1NS(=O)(=O)c1ccc(S(=O)(=O)N(C)C)cc1]

Compound 26 was synthesized using N1-benzyl-N1-methylbenzene-1,2-diamine (4) and common intermediate 1 according to the procedure of Step 3 in the preparation of Compound 27.

¹HNMR (300 MHz, CDCl₃) δ 8.28 (br s, 1H), 7.90 (d, J=8.4 Hz, 2H), 7.74 (d, J=8.4 Hz, 2H), 7.55 (d, J=8.4 Hz, 1H), 7.31-7.26 (m, 3H), 7.18-7.03 (m, 5H), 3.67 (s, 2H), 2.67 (s, 6H), 2.38 (s, 3H). LCMS [M+H]⁺ calcd for C₂₂H₂₆N₃O₄S₂, 460.13; found, 459.8.

### Example S4. Synthesis of Compound 23

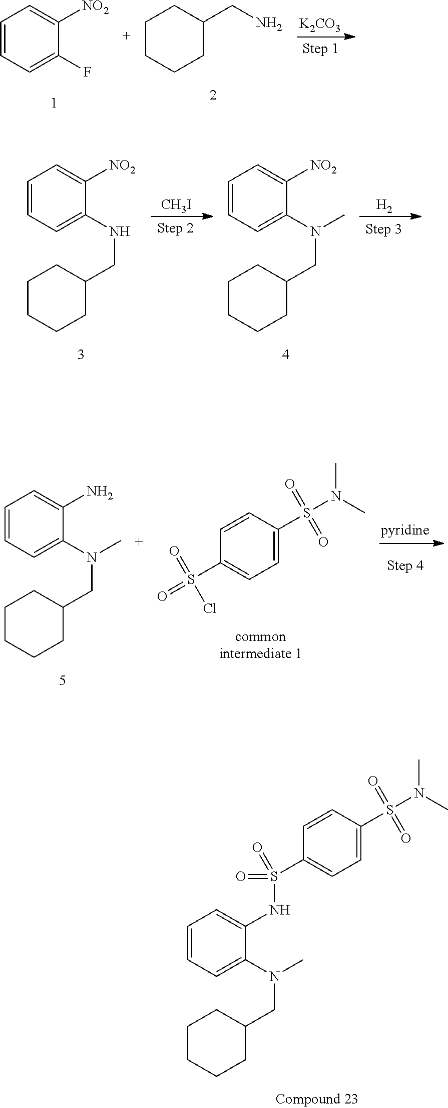

### Step 1

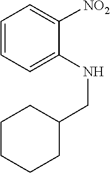 [SMILES: O=[N+]([O-])c1ccccc1NCC1CCCCC1]

N-(cyclohexylmethyl)-2-nitroaniline (3) was synthesized from starting materials (1) and (2) in the scheme above according to the procedure of Step 1 in the preparation of Compound 27.

LCMS [M+H]⁺ calcd for C₁₃H₁₉N₂O₂, 235.14; found, 235.

### Step 2

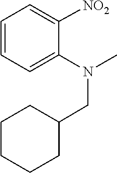 [SMILES: CN(CC1CCCCC1)c1ccccc1[N+](=O)[O-]]

N-(cyclohexylmethyl)-N-methyl-2-nitroaniline (4) was synthesized from N-(cyclohexylmethyl)-2-nitroaniline (3) according to the procedure of Step 1 in the preparation of Compound 27.

LCMS [M+H]⁺ calcd for C₁₄H₂₁N₂O₂, 249.15; found, 249.

### Step 3

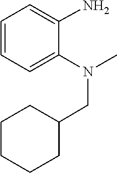 [SMILES: CN(CC1CCCCC1)c1ccccc1N]

N1-(cyclohexylmethyl)-N1-methylbenzene-1,2-diamine (5) was synthesized from N-(cyclohexylmethyl)-N-methyl-2-nitroaniline (4) according to the procedure of Step 2 in the preparation of Compound 27.

LCMS [M+H]⁺ calcd for C₁₄H₂₃N₂, 219; found, 219.

### Step 4

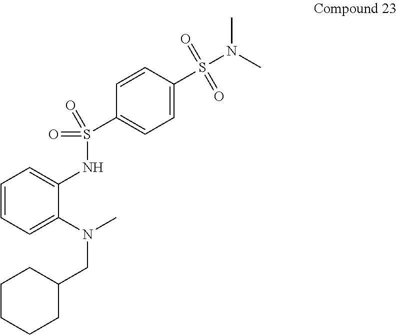 [SMILES: CN(CC1CCCCC1)c1ccccc1NS(=O)(=O)c1ccc(S(=O)(=O)N(C)C)cc1]

N1-(2-((cyclohexylmethyl)(methyl)amino)phenyl)-N4,N4-dimethylbenzene-1,4-disulfonamide (Compound 23) was synthesized from N1-(cyclohexylmethyl)-N1-methylbenzene-1,2-diamine (5) and common intermediate 1 according to the procedure of Step 3 in the preparation of Compound 27.

¹HNMR (300 MHz, CDCl₃) δ 8.35 (br s, 1H), 8.01 (d, J=8.1 Hz, 2H), 7.87 (d, J=8.1 Hz, 2H), 7.58 (d, J=7.2 Hz, 1H), 7.12-7.01 (m, 3H), 2.70 (s, 6H), 2.47 (d, J=7.2 Hz, 2H), 2.32 (s, 3H), 1.72-1.55 (m, 5H), 1.39-1.36 (m, 1H), 1.15-1.13 (m, 3H), 0.83-0.70 (m, 2H). LCMS [M+H]⁺ calcd for C₂₂H₃₂N₃O₄S₂, 466.18; found, 466.3.

### Example S5. Synthesis of Compound 25

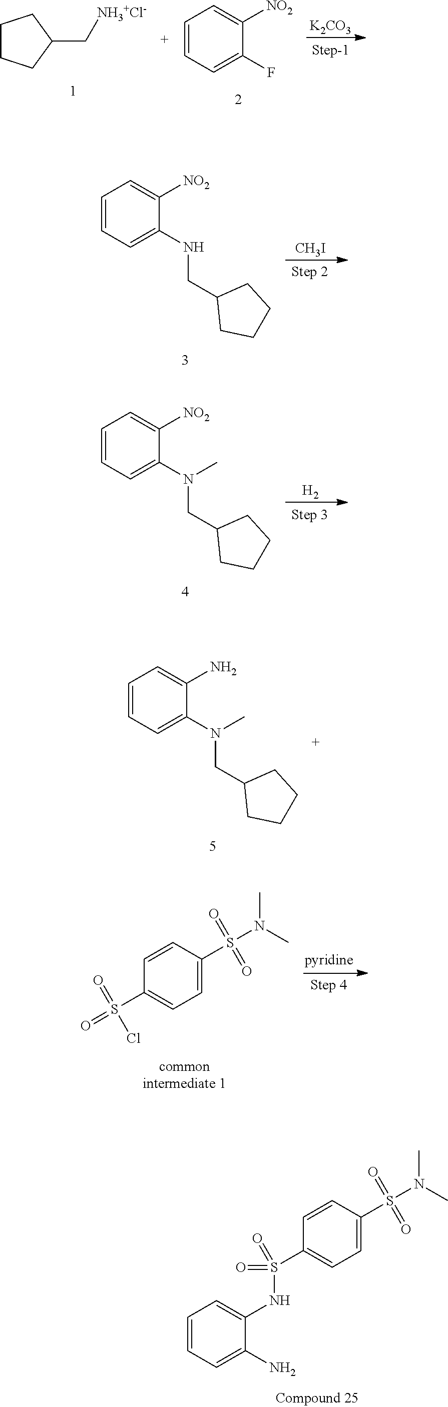

### Step 1

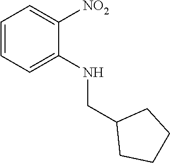 [SMILES: O=[N+]([O-])c1ccccc1NCC1CCCC1]

N-(cyclopentylmethyl)-2-nitroaniline (3) was synthesized from starting materials (1) and (2) according to the procedure of Step 1 in the preparation of Compound 26.

LCMS [M+H]⁺ calcd for C₁₂H₁₇N₂O₂, 221.12; found, 221.

### Step 2

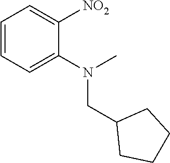 [SMILES: CN(CC1CCCC1)c1ccccc1[N+](=O)[O-]]

N-(cyclopentylmethyl)-N-methyl-2-nitroaniline (4) was synthesized from N-(cyclopentylmethyl)-2-nitroaniline (3) according to the procedure of Step 1 in the preparation of Compound 27.

LCMS [M+H]⁺ calcd for C₁₃H₁₉N₂O₂, 235.14; found, 235.

### Step 3

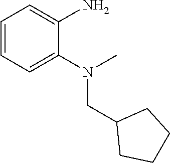

N1-(cyclopentylmethyl)-N1-methylbenzene-1,2-diamine (5) was synthesized from N-(cyclopentylmethyl)-N-methyl-2-nitroaniline (4) according to the procedure of Step 2 in the preparation of Compound 27.

LCMS [M+H]⁺ calcd for C₁₃H₂₁N₂, 205.16; found, 205.

### Step 4

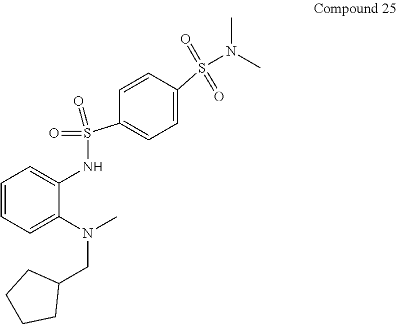 [SMILES: CN(CC1CCCC1)c1ccccc1NS(=O)(=O)c1ccc(S(=O)(=O)N(C)C)cc1]

N1-(2-((cyclopentylmethyl)(methyl)amino)phenyl)-N4,N4-dimethylbenzene-1,4-disulfonamide (Compound 25) was synthesized from N1-(cyclopentylmethyl)-N1-methylbenzene-1,2-diamine (5) and common intermediate 1 according to the procedure of Step 3 in the preparation of Compound 27.

¹HNMR (300 MHz, CDCl₃) δ 8.40 (br s, 1H), 8.02 (d, J=8.4 Hz, 2H), 7.82 (d, J=8.4 Hz, 2H), 7.61-7.58 (m, 1H), 7.14-7.01 (m, 3H), 2.71 (s, 6H), 2.63 (d, J=7.5 Hz, 2H), 2.33 (s, 3H), 1.81-1.74 (m, 1H), 1.64-1.47 (m, 6H), 1.26-1.07 (m, 2H). LCMS [M+H]⁺ calcd for C₂₁H₃₀N₃O₄S₂, 452.16; found, 451.9.

### Example S6. Synthesis of Compound 28

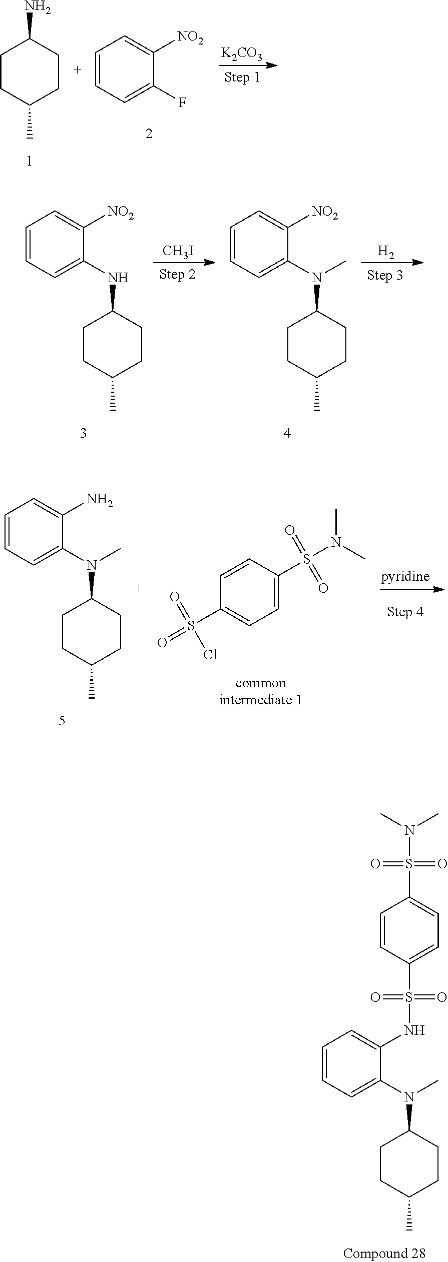

### Step 1

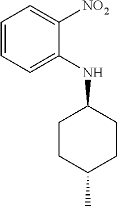 [SMILES: C[C@H]1CC[C@H](Nc2ccccc2[N+](=O)[O-])CC1]

N-((1r,4r)-4-methylcyclohexyl)-2-nitroaniline (3) was synthesized from starting materials (1) and (2) according to the procedure of Step 1 in the preparation of Compound 26.

LCMS [M+H]⁺ calcd for C₁₃H₁₉N₂O₂, 235.14; found, 235.

### Step 2

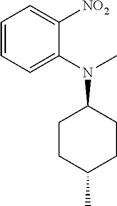 [SMILES: CN(c1ccccc1[N+](=O)[O-])[C@H]1CC[C@H](C)CC1]

N-methyl-N-((1r,4r)-4-methylcyclohexyl)-2-nitroaniline (4) was synthesized from N-((1r,4r)-4-methylcyclohexyl)-2-nitroaniline (3) according to the procedure of Step 1 in the preparation of Compound 27.

LCMS [M+H]⁺ calcd for C₁₄H₂₁N₂O₂, 249.15; found, 249.

### Step 3

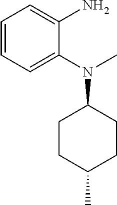 [SMILES: CN(c1ccccc1N)[C@H]1CC[C@H](C)CC1]

N1-methyl-N1-((1r,4r)-4-methylcyclohexyl)benzene-1,2-diamine (5) was synthesized from N-methyl-N-((1r,4r)-4-methylcyclohexyl)-2-nitroaniline (4) according to the procedure of Step 2 in the preparation of Compound 27.

LCMS [M+H]⁺ calcd for C₁₄H₂₃N₂, 219.18; found, 219.

### Step 4

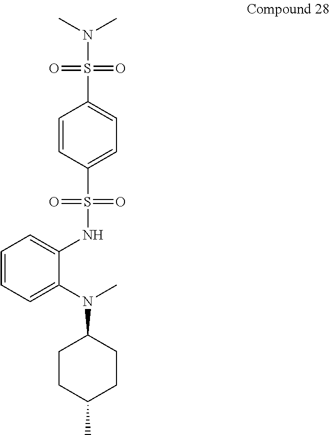 [SMILES: CN(C)S(=O)(=O)c1ccc(S(=O)(=O)Nc2ccccc2N(C)[C@H]2CC[C@H](C)CC2)cc1]

N1,N1-dimethyl-N4-(2-(methyl((1r,4r)-4-methylcyclohexyl)amino)phenyl)benzene-1,4-disulfonamide (Compound 28) was synthesized from N1-methyl-N1-((1r,4r)-4-methylcyclohexyl)benzene-1,2-diamine (5) and common intermediate 1 according to the procedure of Step 3 in the preparation of Compound 27.

¹HNMR (300 MHz, CDCl₃) δ 8.51 (br, 1H) 7.99 (d, J=8.1 Hz, 2H), 7.81 (d, J=8.4 Hz, 2H), 7.62 (d, J=7.2 Hz, 1H), 7.01-7.16 (m, 3H), 2.70 (s, 6H), 2.39-2.55 (m, 1H), 2.32 (s, 3H), 1.55-1.69 (m, 5H), 1.19-1.23 (m, 1H), 0.96-1.05 (m, 2H), 0.71-0.85 (m, 4H). LCMS [M+H]⁺ calcd for C₂₂H₃₂N₃O₄S₂, 466.18; found, 465.9.

### Example S7. Synthesis of Compound 29

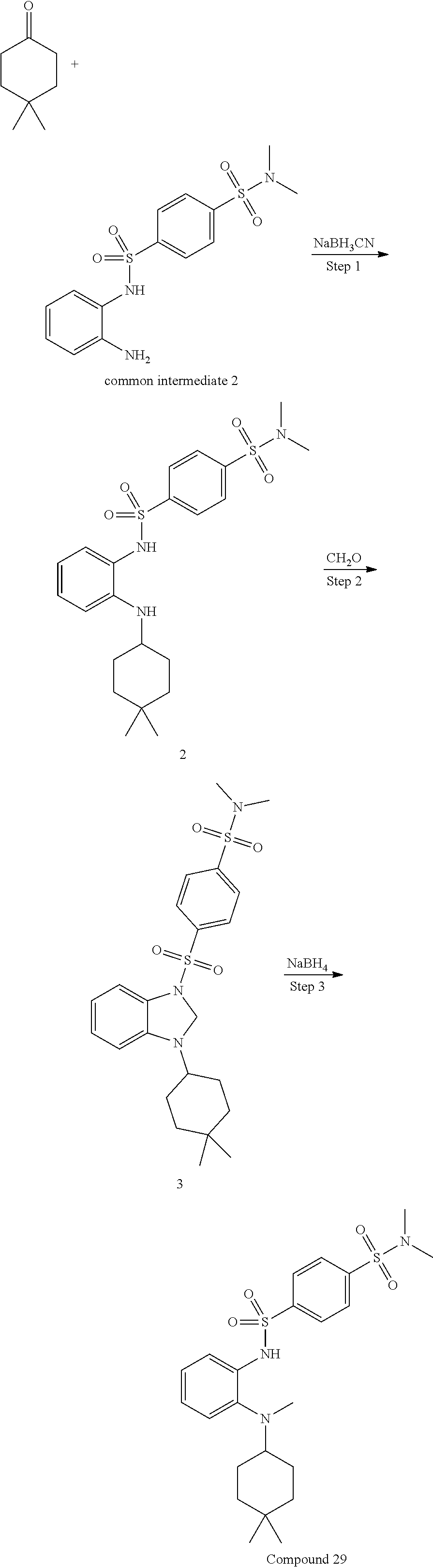

### Step 1

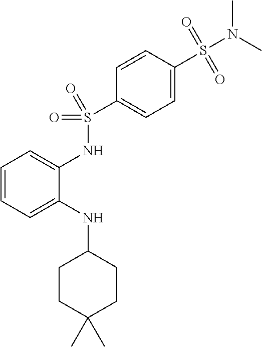 [SMILES: CN(C)S(=O)(=O)c1ccc(S(=O)(=O)Nc2ccccc2NC2CCC(C)(C)CC2)cc1]

N1-(2-((4,4-dimethylcyclohexyl)amino)phenyl)-N4,N4-dimethylbenzene-1,4-disulfonamide (2) was synthesized from starting material (1) and common intermediate 2 according to the procedure of Step 1 in the preparation of Compound 20.

LCMS [M+H]⁺ calcd for C₂₂H₃₂N₃O₄S₂, 466; found, 466.

### Step 2

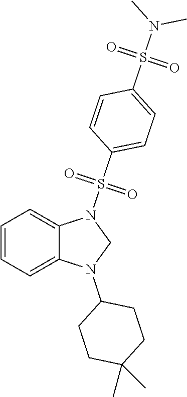 [SMILES: CN(C)S(=O)(=O)c1ccc(S(=O)(=O)N2CN(C3CCC(C)(C)CC3)c3ccccc32)cc1]

4-((3-(4,4-dimethylcyclohexyl)-2,3-dihydro-1H-benzo[d]imidazol-1-yl)sulfonyl)-N,N-dimethylbenzenesulfonamide (3) was synthesized from N1-(2-((4,4-dimethylcyclohexyl)amino)phenyl)-N4,N4-dimethylbenzene-1,4-disulfonamide (2) according to the procedure of Step 2 in the preparation of Compound 20.

LCMS [M+H]⁺ calcd for C₂₃H₃₂N₃O₄S₂, 478; found, 478.

### Step 3

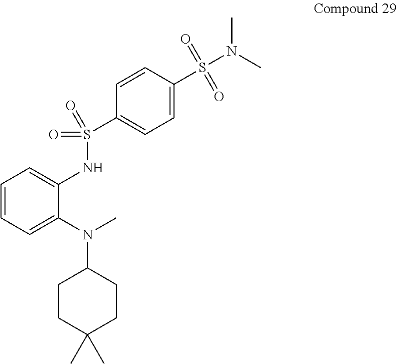 [SMILES: CN(c1ccccc1NS(=O)(=O)c1ccc(S(=O)(=O)N(C)C)cc1)C1CCC(C)(C)CC1]

N1-(2-((4,4-dimethylcyclohexyl)(methyl)amino)phenyl)-N4,N4-dimethylbenzene-1,4-disulfonamide (Compound 29) was synthesized from 4-((3-(4,4-dimethylcyclohexyl)-2,3-dihydro-1H-benzo[d]imidazol-1-yl)sulfonyl)-N,N-dimethylbenzenesulfonamide (3) according to the procedure of Step 3 in the preparation of Compound 20.

¹HNMR (300 MHz, CDCl₃) δ 8.50 (br, 1H), 8.00 (d, J=8.4 Hz, 2H), 7.81 (d, J=8.4 Hz, 2H), 7.65 (d, J=7.5 Hz, 1H), 7.19-7.01 (m, 3H), 2.70 (s, 6H), 2.38 (m, 1H), 2.39 (s, 3H), 1.51-1.40 (m, 2H), 1.39-1.22 (m, 2H), 1.20-0.98 (m, 4H), 0.86 (s, 3H), 0.84 (s, 3H). LCMS [M+H]⁺ calcd for C₂₃H₃₄N₃O₄S₂, 480.19; found, 479.9.

### Example S8. Synthesis of Compound 17

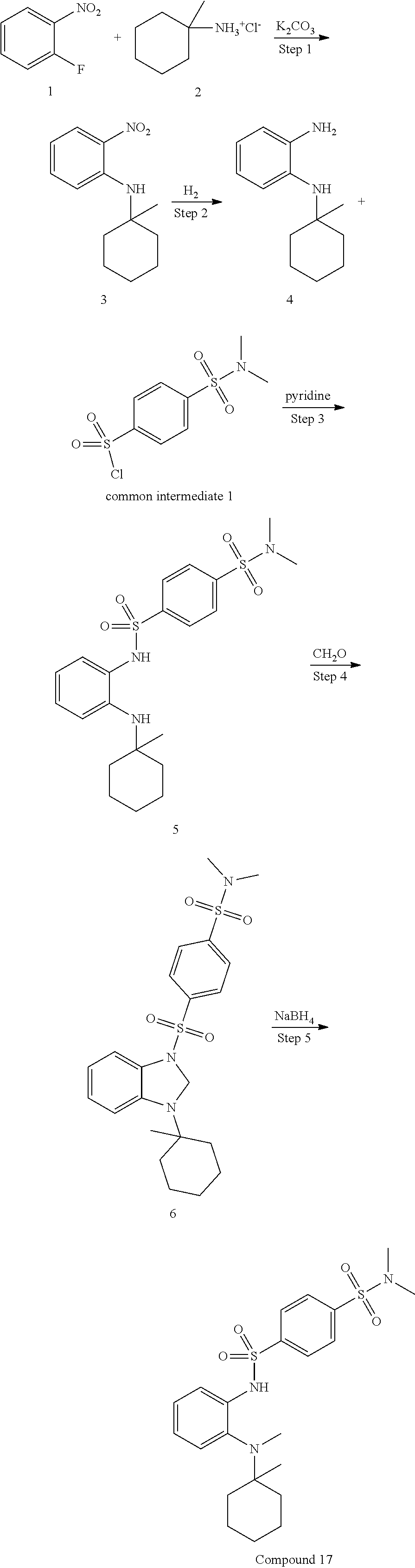

### Step 1

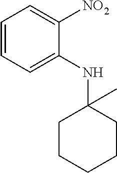 [SMILES: CC1(Nc2ccccc2[N+](=O)[O-])CCCCC1]

N-(1-methylcyclohexyl)-2-nitroaniline (3) was synthesized from starting materials (1) and (2) according to the procedure of Step 1 in the preparation of Compound 26.

LCMS [M+H]⁺ calcd for C₁₃H₁₈N₂O₂, 235.14; found, 235.

### Step 2

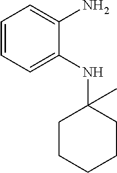

N1-(1-methylcyclohexyl)benzene-1,2-diamine (4) was synthesized from N-(1-methylcyclohexyl)-2-nitroaniline (3) according to the procedure of Step 2 in the preparation of Compound 27.

LCMS [M+H]⁺ calcd for C₁₃H₂₁N₂, 205.16; found, 205.

### Step 3

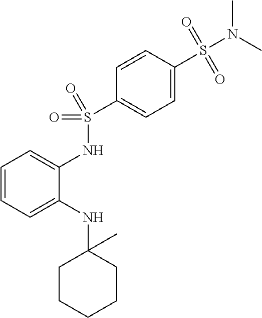 [SMILES: CN(C)S(=O)(=O)c1ccc(S(=O)(=O)Nc2ccccc2NC2(C)CCCCC2)cc1]

N1,N1-dimethyl-N4-(2-((1-methylcyclohexyl)amino)phenyl)benzene-1,4-disulfonamide (5) was synthesized from N1-(1-methylcyclohexyl)benzene-1,2-diamine (4) and common intermediate 1 according to the procedure of Step 3 in the preparation of Compound 27.

LCMS [M+H]⁺ calcd for C₂₁H₃₀N₃O₄S₂, 452.16; found, 452.

### Step 4

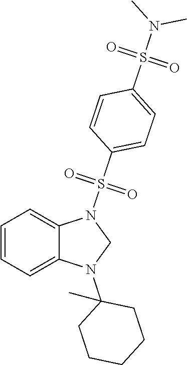 [SMILES: CN(C)S(=O)(=O)c1ccc(S(=O)(=O)N2CN(C3(C)CCCCC3)c3ccccc32)cc1]

N,N-dimethyl-4-((3-(1-methylcyclohexyl)-2,3-dihydro-1H-benzo[d]imidazol-1-yl)sulfonyl)benzenesulfonamide (6) was synthesized from N1,N1-dimethyl-N4-(2-((1-methylcyclohexyl)amino)phenyl)benzene-1,4-disulfonamide (5) according to the procedure of Step 2 in the preparation of Compound 20.

LCMS [M+H]⁺ calcd for C₂₂H₃₀N₃O₄S₂, 464.16; found, 464.

### Step 5

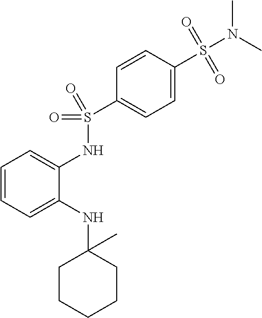 [SMILES: CN(C)S(=O)(=O)c1ccc(S(=O)(=O)Nc2ccccc2NC2(C)CCCCC2)cc1]

N1,N1-dimethyl-N4-(2-(methyl(1-methylcyclohexyl)amino)phenyl)benzene-1,4-disulfonamide (Compound 17) was synthesized from N,N-dimethyl-4-((3-(1-methylcyclohexyl)-2,3-dihydro-1H-benzo[d]imidazol-1-yl)sulfonyl)benzenesulfonamide (6) according to the procedure of Step 3 in the preparation of Compound 20.

¹HNMR (300 MHz, CD₃OD) δ 8.06 (d, J=8.4 Hz, 2H), 7.86 (d, J=8.4 Hz, 2H), 7.64 (d, J=7.8 Hz, 1H), 7.30 (d, J=7.8 Hz, 1H), 7.17 (t, J=7.8 Hz, 1H), 7.01 (t, J=7.8 Hz, 1H), 2.65 (s, 6H), 2.33 (s, 3H), 1.73-1.49 (m, 3H), 1.43-1.21 (m, 3H), 1.19-1.08 (m, 4H), 1.14 (s, 3H). LCMS [M+H]⁺ calcd for C₂₂H₃₂N₃O₄S₂, 466.18; found, 465.7.

### Example S9. Synthesis of Compound 22

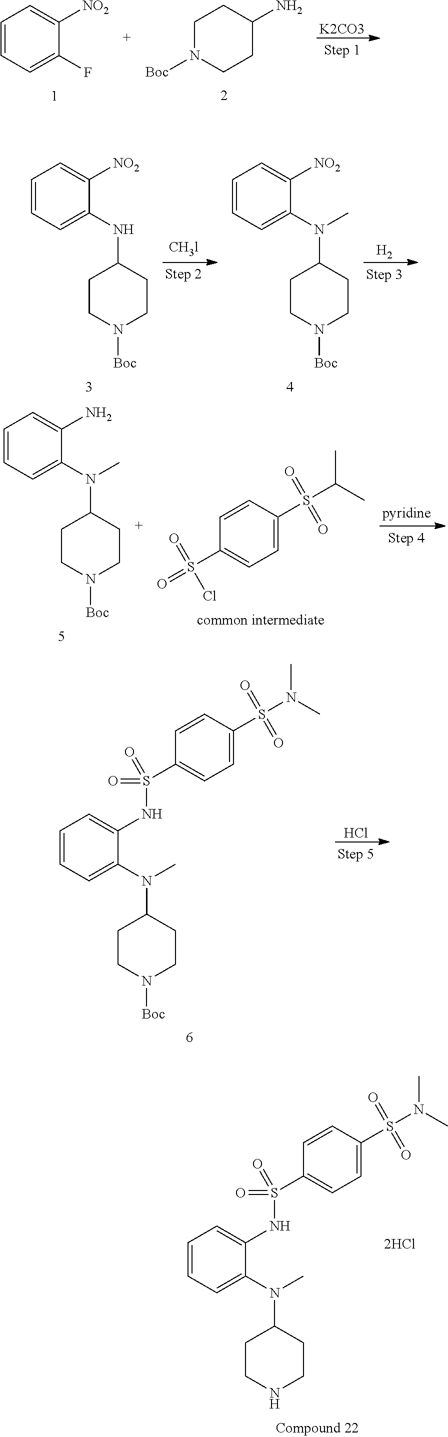

### Step 1

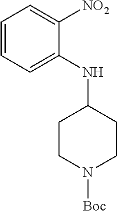 [SMILES: CC(C)(C)OC(=O)N1CCC(Nc2ccccc2[N+](=O)[O-])CC1]

Tert-butyl 4-((2-nitrophenyl)amino)piperidine-1-carboxylate (3) was synthesized from starting materials (1) and (2) according to the procedure of Step 1 in the preparation of Compound 26.

LCMS [M+Na]⁺ calcd for C₁₆H₂₃N₃O₄, 321.17; found, 344.

### Step 2

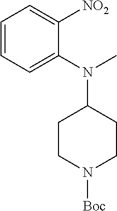 [SMILES: CN(c1ccccc1[N+](=O)[O-])C1CCN(C(=O)OC(C)(C)C)CC1]

Tert-butyl 4-(methyl(2-nitrophenyl)amino)piperidine-1-carboxylate (4) was synthesized from tert-butyl 4-((2-nitrophenyl)amino)piperidine-1-carboxylate (3) according to the procedure of Step 1 in the preparation of Compound 27.

LCMS [M+Na]+ calcd for C₁₇H₂₅N₃O₄, 335.18; found, 358.

### Step 3

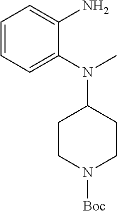 [SMILES: CN(c1ccccc1N)C1CCN(C(=O)OC(C)(C)C)CC1]

Tert-butyl 4-((2-aminophenyl)(methyl)amino)piperidine-1-carboxylate (5) was synthesized from tert-butyl 4-(methyl(2-nitrophenyl)amino)piperidine-1-carboxylate (4) according to the procedure of Step 2 in the preparation of Compound 27.

LCMS [M+H]⁺ calcd for C₁₇H₂₈N₃O₂, 306.21; found, 306.

### Step 4

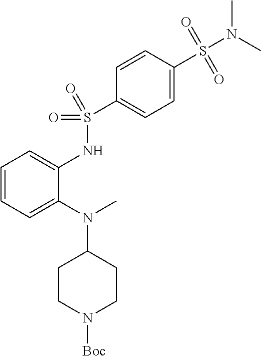 [SMILES: CN(c1ccccc1NS(=O)(=O)c1ccc(S(=O)(=O)N(C)C)cc1)C1CCN(C(=O)OC(C)(C)C)CC1]

Tert-butyl 4-((2-((4-(N,N-dimethylsulfamoyl)phenyl)sulfonamido)phenyl)(methyl)amino)piperidine-1-carboxylate (6) was synthesized from tert-butyl 4-((2-aminophenyl)(methyl)amino)piperidine-1-carboxylate (5) and common intermediate 1 according to the procedure of Step 3 in the preparation of Compound 27.

¹HNMR (300 MHz, CD₃OD) δ 8.06 (d, J=8.4 Hz, 2H), 7.90 (d, J=8.4 Hz, 2H), 7.58 (d, J=6.6 Hz, 1H), 7.20 (t, J=6.0 Hz, 1H), 7.16-7.01 (m, 2H), 4.01-3.92 (m, 2H), 2.79-2.54 (m, 3H), 2.65 (s, 6H), 2.32 (s, 3H), 1.71-1.60 (m, 2H), 1.45 (s, 9H), 1.32-1.17 (m. 2H). LCMS [M+H]⁺ calcd for C₂₅H₃₇N₄O₆S₂, 553.21; found, 552.9.

### Step 5

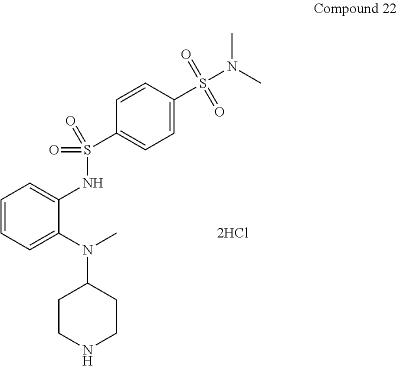 [SMILES: CN(c1ccccc1NS(=O)(=O)c1ccc(S(=O)(=O)N(C)C)cc1)C1CCNCC1]

To a solution of tert-butyl 4-((2-((4-(N,N-dimethylsulfamoyl)phenyl)sulfonamido)phenyl)(methyl)amino)piperidine-1-carboxylate (6) (900 mg, 1.63 mmol) in DCM (3 mL) was added HCl/Et₂O (6 mL, 5 mol/L). The reaction mixture was stirred at rt for 4 hr. After the reaction was completed as indicated by LC-MS, the reaction mixture was concentrated in vacuo to give 790 mg of N1,N1-dimethyl-N4-(2-(methyl(piperidin-4-yl)amino)phenyl)benzene-1,4-disulfonamide (Compound 22) as HCl salt. The yield was 99.2%.

¹HNMR (300 MHz, CD₃OD) δ 8.09 (d, J=8.4 Hz, 2H), 7.93 (d, J=8.4 Hz, 2H), 7.53 (d, J=6.6 Hz, 1H), 7.26 (d, J=6.9 Hz, 1H), 7.18-7.05 (m, 2H), 3.41-3.34 (m, 2H), 3.08-2.86 (m, 3H), 2.69 (s, 6H), 2.39 (s, 3H), 1.99-1.88 (m, 2H), 1.62-1.47 (m, 2H). LCMS [M+H]+ calcd for C₂₀H₂₉N₄O₄S₂, 453.16; found, 452.9.

### Example S10. Synthesis of Compound 21

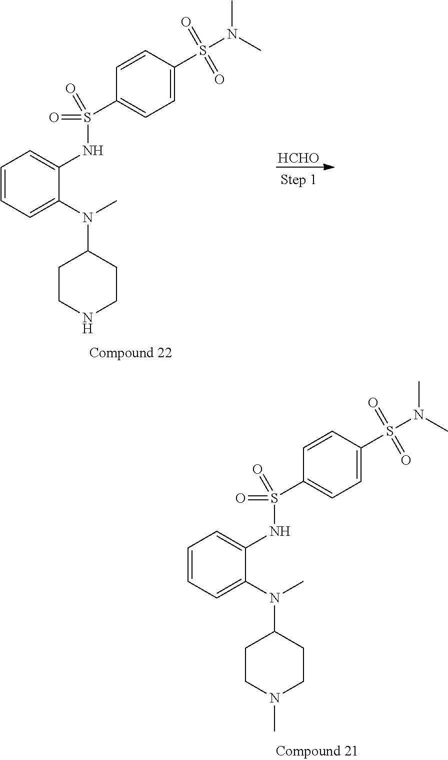 [SMILES: CN(c1ccccc1NS(=O)(=O)c1ccc(S(=O)(=O)N(C)C)cc1)C1CCNCC1 ; "HCHO" ; CN1CCC(N(C)c2ccccc2NS(=O)(=O)c2ccc(S(=O)(=O)N(C)C)cc2)CC1]

### Step 1

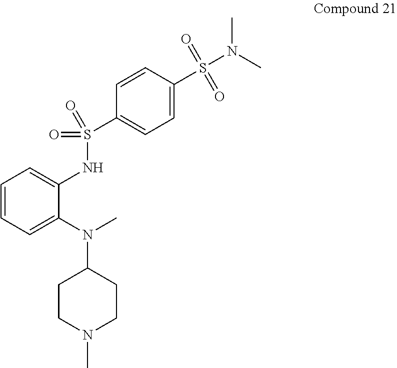 [SMILES: CN1CCC(N(C)c2ccccc2NS(=O)(=O)c2ccc(S(=O)(=O)N(C)C)cc2)CC1]

To a solution of N1,N1-dimethyl-N4-(2-(methyl(piperidin-4-yl)amino)phenyl)benzene-1,4-disulfonamide (Compound 22) (HCl salt, 220 mg, 0.46 mmol) in MeOH (3 mL) was added an aqueous formaldehyde solution (149 mg, 1.84 mmol, 37 wt %), NaBH₃CN, (115.6 mg, 1.84 mmol) and one drop of AcOH. The reaction mixture was stirred at ambient temperature overnight. After the reaction was completed as indicated by TLC, it was quenched with water (20 mL) and extracted with EtOAc (30 mL×2). The combined organic layers were washed with brine, dried over anhydrous Na₂SO₄, filtered, and concentrated in vacuo. The resulting oil was purified by silica gel chromatography to give the impure N1,N1-dimethyl-N4-(2-(methyl(1-methylpiperidin-4-yl)amino)phenyl)benzene-1,4-disulfonamide (Compound 21). The impure compound was further purified by prep-HPLC to give 22.8 mg of purified N1,N1-dimethyl-N4-(2-(methyl(1-methylpiperidin-4-yl)amino)phenyl)benzene-1,4-disulfonamide (Compound 21).

¹HNMR (300 MHz, CDCl₃) δ 8.39 (br s, 1H), 8.01 (d, J=8.4 Hz, 2H), 7.83 (d, J=8.4 Hz, 2H), 7.61 (d, J=7.8 Hz, 1H), 7.14-7.01 (m, 3H), 2.77-2.70 (m, 2H), 2.70 (s, 6H), 2.49 (m, 1H), 2.36 (s, 3H), 2.33 (s, 3H), 1.83-1.75 (m, 2H), 1.71-1.53 (m, 2H), 1.41-1.27 (m, 2H). LCMS [M+H]⁺ calcd for C₂₁H₃₁N₄O₄S₂, 467.17; found, 466.8.

### Example S11. Synthesis of Compound 19

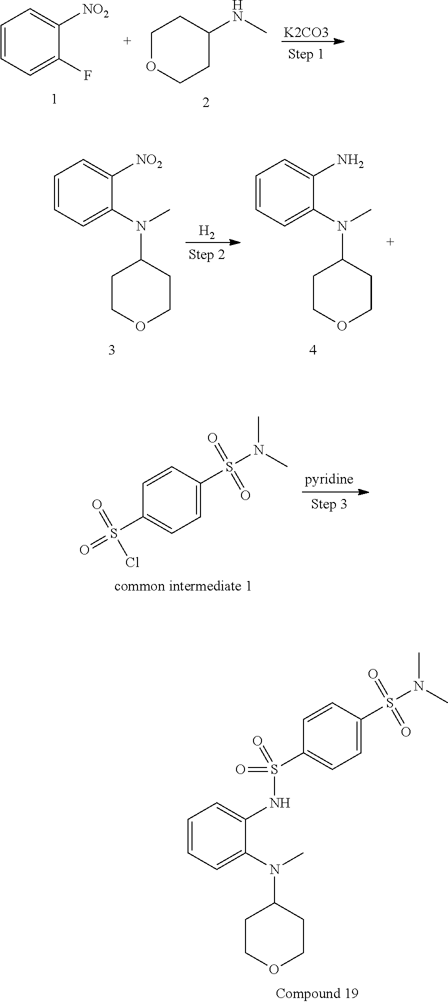

### Step 1

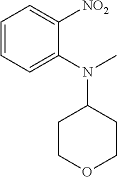 [SMILES: CN(c1ccccc1[N+](=O)[O-])C1CCOCC1]

N-methyl-N-(2-nitrophenyl)tetrahydro-2H-pyran-4-amine (3) was synthesized from starting materials (1) and (2) according to the procedure of Step 1 in the preparation of Compound 26.

LCMS [M+H]⁺ calcd for C₁₂H₁₇N₂O₃, 237.12; found, 237.

### Step 2

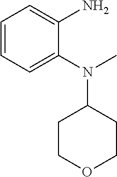

N1-methyl-N1-(tetrahydro-2H-pyran-4-yl)benzene-1,2-diamine (4) was synthesized from N-methyl-N-(2-nitrophenyl)tetrahydro-2H-pyran-4-amine (3) according to the procedure of Step 2 in the preparation of Compound 27.

LCMS [M+H]⁺ calcd for C₁₂H₁₉N₂O, 207.14; found, 207.

### Step 3

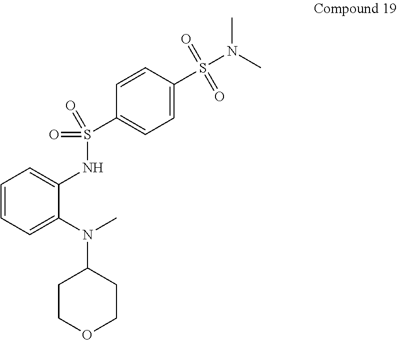 [SMILES: CN(c1ccccc1NS(=O)(=O)c1ccc(S(=O)(=O)N(C)C)cc1)C1CCOCC1]

N1,N1-dimethyl-N4-(2-(methyl(tetrahydro-2H-pyran-4-yl)amino)phenyl)benzene-1,4-disulfonamide (Compound 19) was synthesized from N1-methyl-N1-(tetrahydro-2H-pyran-4-yl)benzene-1,2-diamine (4) and common intermediate 1 according to the procedure of Step 3 in the preparation of Compound 27.

¹HNMR (400 MHz, CDCl₃) δ 8.45 (br, 1H), 8.01 (d, J=8.4 Hz, 2H), 7.83 (d, J=8.4 Hz, 2H), 7.61 (d, J=5.4 Hz, 1H), 7.16-7.14 (m, 2H), 7.07-7.03 (m, 1H), 3.91-3.87 (m, 2H), 3.26 (t, J=7.8 Hz, 2H), 2.75-2.74 (m, 1H), 2.73 (s, 6H), 2.33 (s, 3H), 1.56-1.58 (m, 2H), 1.38-1.25 (m, 2H). LCMS [M+H]⁺ calcd for C₂₀H₂₈N₃O₅S₂, 454.14; found, 454.3.

### Example S12. Synthesis of Compound 24

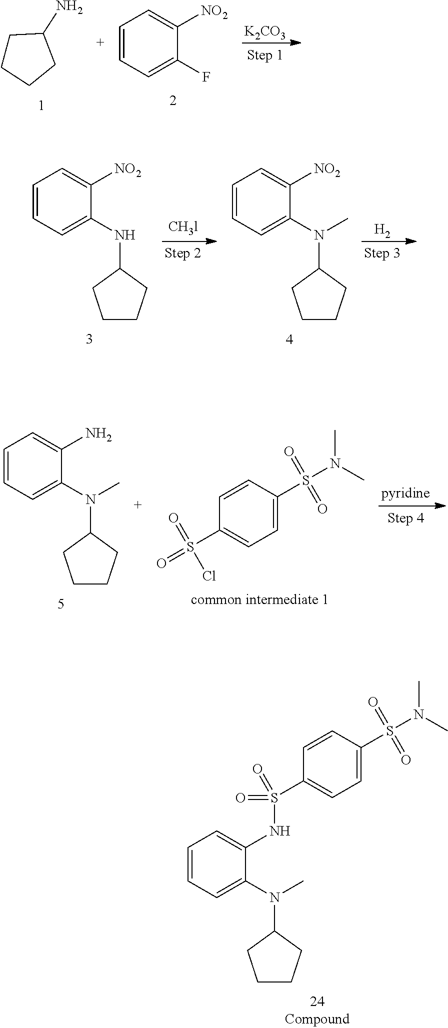

### Step 1

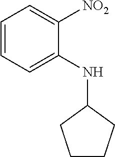 [SMILES: O=[N+]([O-])c1ccccc1NC1CCCC1]

N-cyclopentyl-2-nitroaniline (3) was synthesized from starting materials (1) and (2) according to the procedure of Step 1 in the preparation of Compound 26.

LCMS [M+H]+ calcd for C₁₁H₁₅N₂O₂, 207.11; found, 207.

### Step 2

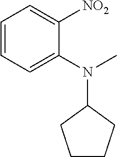 [SMILES: CN(c1ccccc1[N+](=O)[O-])C1CCCC1]

N-cyclopentyl-N-methyl-2-nitroaniline (4) was synthesized from N-cyclopentyl-2-nitroaniline (3) according to the procedure of Step 1 in the preparation of Compound 27.

LCMS [M+H]⁺ calcd for C₁₂H₁₇N₂O₂, 221.12; found, 221.

### Step 3

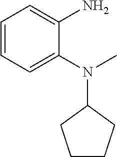

N1-cyclopentyl-N1-methylbenzene-1,2-diamine (5) was synthesized from N-cyclopentyl-N-methyl-2-nitroaniline (4) according to the procedure of Step 2 in the preparation of Compound 27.

LCMS [M+H]⁺ calcd for C₁₂H₁₉N₂, 191.15; found, 191.

### Step 4

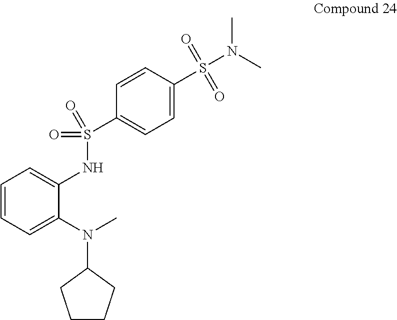 [SMILES: CN(c1ccccc1NS(=O)(=O)c1ccc(S(=O)(=O)N(C)C)cc1)C1CCCC1]

N1-(2-(cyclopentyl(methyl)amino)phenyl)-N4,N4-dimethylbenzene-1,4-disulfonamide (Compound 24) was synthesized from N1-cyclopentyl-N1-methylbenzene-1,2-diamine (5) and common intermediate 1 according to the procedure of Step 3 in the preparation of Compound 27.

¹HNMR (300 MHz, CDCl₃) δ 8.53 (br, 1H), 8.00 (d, J=8.1 Hz, 2H), 7.81 (d, J=8.1 Hz, 2H), 7.63 (d, J=8.1 Hz, 1H), 7.20-7.04 (m, 3H), 3.23-3.13 (m, 1H), 2.69 (s, 6H), 2.27 (s, 3H), 1.68-1.48 (m, 6H), 1.26-1.10 (m, 2H). LCMS [M+H]⁺ calcd for C₂₀H₂₈N₃O₄S₂, 438.14; found, 438.3.

### Example S13. Synthesis of Compound 18

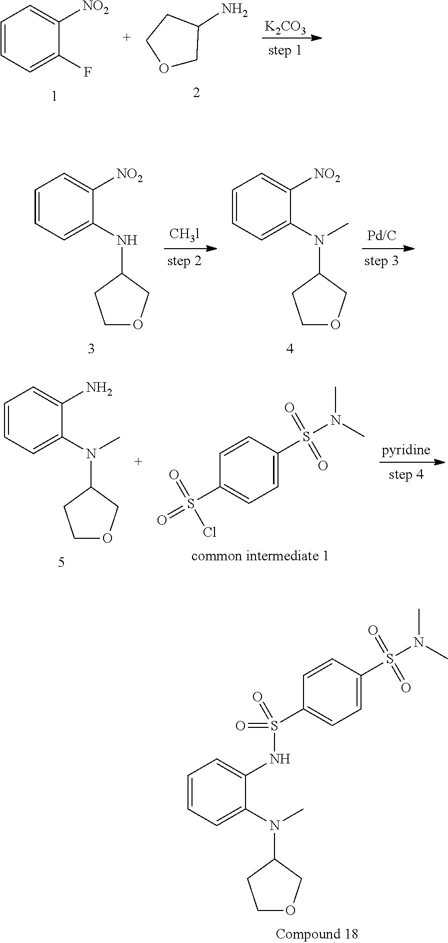

### Step 1

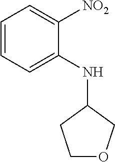 [SMILES: O=[N+]([O-])c1ccccc1NC1CCOC1]

N-(2-nitrophenyl)tetrahydrofuran-3-amine (3) was synthesized from starting materials (1) and (2) according to the procedure of Step 1 in the preparation of Compound 26.

¹HNMR (300 MHz, CDCl₃) δ 8.14-8.18 (m, 2H), 7.45 (t, J=7.5 Hz, 1H), 6.82 (d, J=8.7 Hz, 1H), 6.69 t, J=7.5 Hz, 1H), 4.25 (s, 1H), 4.12-3.89 (m, 3H), 3.79 (m, 1H), 2.35 (m, 1H), 1.98 (m, 1H).

### Step 2

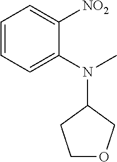 [SMILES: CN(c1ccccc1[N+](=O)[O-])C1CCOC1]

N-methyl-N-(2-nitrophenyl)tetrahydrofuran-3-amine (4) was synthesized from N-(2-nitrophenyl)tetrahydrofuran-3-amine (3) according to the procedure of Step 1 in the preparation of Compound 27.

LCMS [M+H]⁺ calcd for C₁₁H₁₅N₂O₃, 223.10; found, 223.

### Step 3

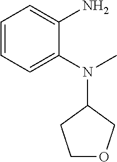

N1-methyl-N1-(tetrahydrofuran-3-yl)benzene-1,2-diamine (5) was synthesized from N-methyl-N-(2-nitrophenyl)tetrahydrofuran-3-amine (4) according to the procedure of Step 2 in the preparation of Compound 27.

LCMS [M+H]⁺ calcd for C₁₁H₁₇N₂O, 193.13; found, 193.

### Step 4

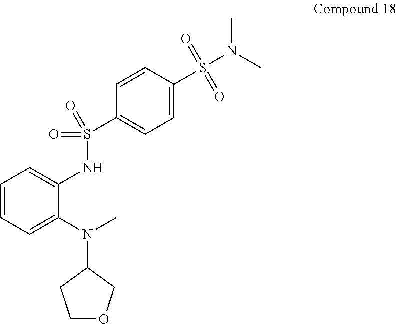 [SMILES: CN(c1ccccc1NS(=O)(=O)c1ccc(S(=O)(=O)N(C)C)cc1)C1CCOC1]

N1,N1-dimethyl-N4-(2-(methyl(tetrahydrofuran-3-yl)amino)phenyl)benzene-1,4-disulfonamide (Compound 18) was synthesized from N1-methyl-N1-(tetrahydrofuran-3-yl)benzene-1,2-diamine (5) and common intermediate 1 according to the procedure of Step 3 in the preparation of Compound 27.

¹HNMR (300 MHz, CDCl₃) δ 8.41 (br, 1H), 8.03 (d, J=6.3 Hz, 2H), 7.83 (d, J=6.3 Hz, 2H), 7.64-7.60 (m, 1H), 7.17-7.12 (m, 2H), 7.08-7.05 (m, 1H), 3.92-3.88 (m, 1H), 3.77-3.72 (m, 1H), 3.61-3.50 (m, 2H), 3.28-3.22 (m, 1H), 2.70 (s, 6H), 2.32 (s, 3H), 1.94-1.88 (m, 1H), 1.65-1.58 (m, 1H). LCMS [M+H]⁺ calcd for C₁₉H₂₆N₃S₅O₂, 440.12; found, 440.3.

### Example S14. Synthesis of Compound 12

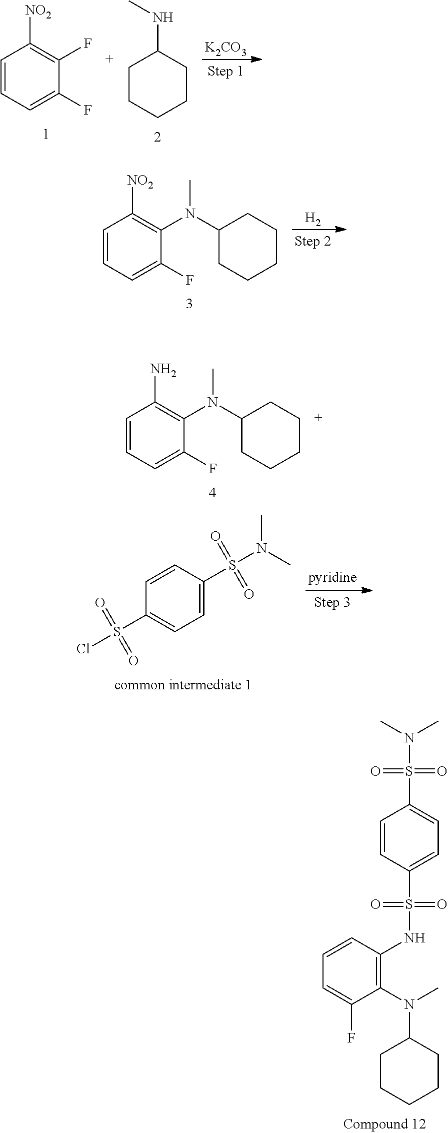

### Step 1

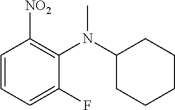 [SMILES: CN(c1c(F)cccc1[N+](=O)[O-])C1CCCCC1]

N-cyclohexyl-2-fluoro-N-methyl-6-nitroaniline (3) was synthesized from starting materials (1) and (2) according to the procedure of Step 1 in the preparation of Compound 26. (No MS response was observed by LC-MS.)

### Step 2

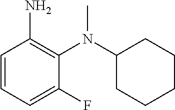 [SMILES: CN(c1c(N)cccc1F)C1CCCCC1]

N1-cyclohexyl-6-fluoro-N1-methylbenzene-1,2-diamine (4) was synthesized from -cyclohexyl-2-fluoro-N-methyl-6-nitroaniline (3) according to the procedure of Step 2 in the preparation of Compound 27.

LCMS [M+H]⁺ calcd for C₁₃H₂₀FN₂, 223; found, 223.

### Step 3

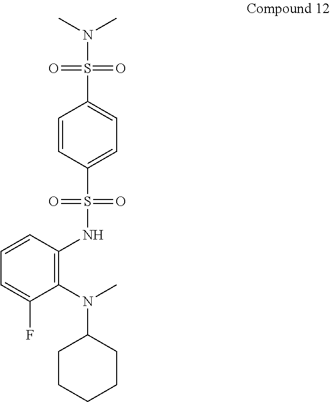

N1-(2-(cyclohexyl(methyl)amino)-3-fluorophenyl)-N4,N4-dimethylbenzene-1,4-disulfonamide (Compound 12) was synthesized from N1-cyclohexyl-6-fluoro-N1-methylbenzene-1,2-diamine (4) and common intermediate 1 according to the procedure of Step 3 in the preparation of Compound 27.

¹HNMR (300 MHz, CDCl₃) δ 8.51 (br, 1H), 8.00 (d, J=8.4 Hz, 2H), 7.84 (d, J=8.4 Hz, 2H), 7.42 (d, J=8.1 Hz, 1H), 7.14-7.07 (m, 1H), 6.74 (t, J=8.7 Hz, 1H), 2.81-2.71 (m, 1H), 2.71 (s, 6H), 2.57 (s, 3H), 2.09-2.00 (m, 1H), 1.78-1.65 (m, 1H), 1.62-1.55 (m, 1H), 1.26-1.06 (m, 6H), 0.64-0.57 (m, 1H). LCMS [M+H]⁺ calcd for C₂₁H₂₉FN₃S₂O₄, 470.15; found, 470.5.

### Example S15. Synthesis of Compound 4

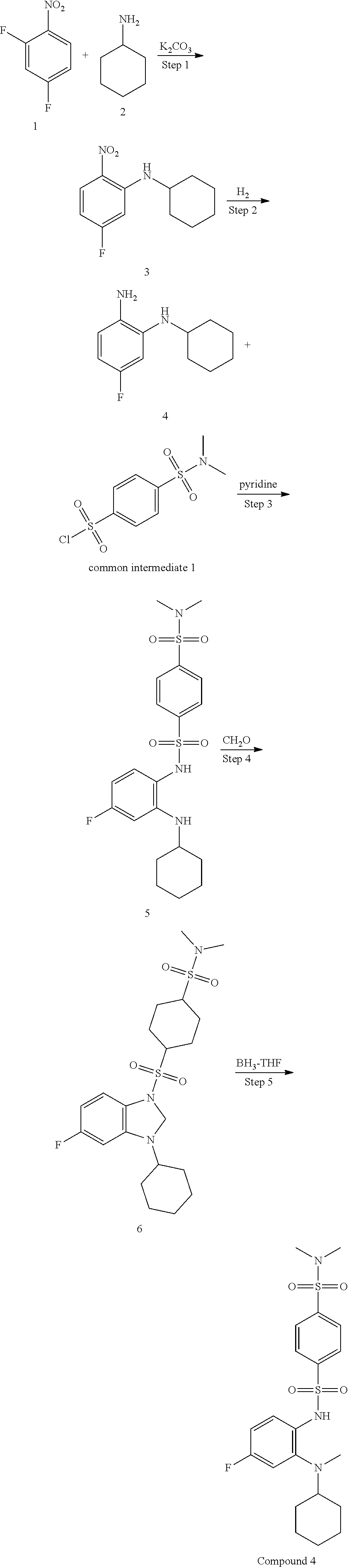

### Step 1

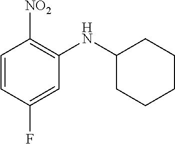 [SMILES: O=[N+]([O-])c1ccc(F)cc1NC1CCCCC1]

N-cyclohexyl-5-fluoro-2-nitroaniline (3) was synthesized from starting materials (1) and (2) according to the procedure of Step 1 in the preparation of Compound 26.

LCMS [M+H]⁺ calcd for C₁₂H₁₆FN₂O₂, 239.11; found, 239.

### Step 2

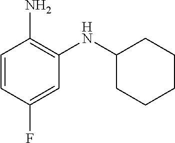 [SMILES: Nc1ccc(F)cc1NC1CCCCC1]

N1-cyclohexyl-5-fluorobenzene-1,2-diamine (4) was synthesized from N-cyclohexyl-5-fluoro-2-nitroaniline (3) according to the procedure of Step 2 in the preparation of Compound 27.

LCMS [M+H]⁺ calcd for C₁₂H₁₈FN₂, 209.14; found, 209.

### Step 3

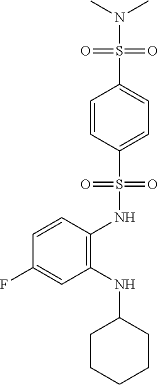

N1-(2-(cyclohexylamino)-4-fluorophenyl)-N4,N4-dimethylbenzene-1,4-disulfonamide (5) was synthesized from N1-cyclohexyl-5-fluorobenzene-1,2-diamine (4) and common intermediate 1 according to the procedure of Step 3 in the preparation of Compound 27.

LCMS [M+H]⁺ calcd for C₂₀H₂₇FN₃O₄S₂, 456.13; found, 456.

### Step 4

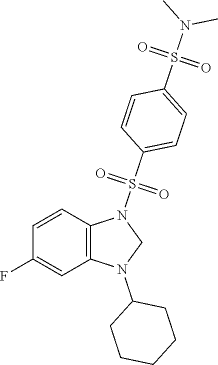 [SMILES: CN(C)S(=O)(=O)c1ccc(S(=O)(=O)N2CN(C3CCCCC3)c3cc(F)ccc32)cc1]

To a solution of N1-(2-(cyclohexylamino)-4-fluorophenyl)-N4,N4-dimethylbenzene-1,4-disulfonamide (5) (260 mg, 0.57 mmol) in MeOH (6 mL) was added formaldehyde (2 mL, 33%). The reaction mixture was stirred at ambient temperature for 2 hr. After the reaction was completed as indicated by TLC, the reaction was quenched with water (20 mL) and extracted with EtOAc (10 mL×2). The combined organic layers were washed with brine, dried over anhydrous Na₂SO₄, filtered, and concentrated to give 240 mg of crude 4-((3-cyclohexyl-5-fluoro-2,3-dihydro-1H-benzo[d]imidazol-1-yl)sulfonyl)-N,N-dimethylbenzenesulfonamide (6).

LCMS [M+H]⁺ calcd for C₂₁H₂₇FN₃O₄S₂, 468.13; found, 468.

### Step 5

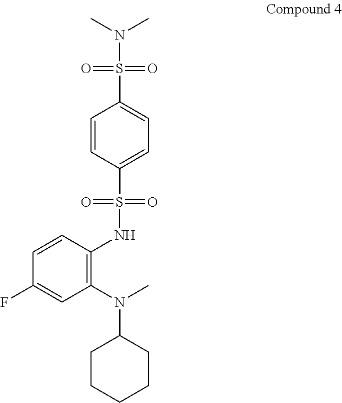 [SMILES: CN(c1cc(F)ccc1NS(=O)(=O)c1ccc(S(=O)(=O)N(C)C)cc1)C1CCCCC1]

Under nitrogen protection, to a solution of crude 4-((3-cyclohexyl-5-fluoro-2,3-dihydro-1H-benzo[d]imidazol-1-yl)sulfonyl)-N,N-dimethylbenzenesulfonamide (6) (240 mg, 0.51 mmol) in THE (6 mL) at 0° C. was added BH₃-THF (1 M, 6 mL) in one portion. The reaction mixture was stirred at rt overnight. After the reaction was completed as indicated by LC-MS, the reaction was quenched with water (15 mL) and extracted with EtOAc (15 mL×2). The combined organic layers were washed with brine, dried over anhydrous Na₂SO₄, filtered, and concentrated under reduced pressure. The resulting oil was purified by silica gel chromatography to give the impure product. The impure product was further purified by prep-HPLC to give 83.1 mg of N1-(2-(cyclohexyl(methyl)amino)-4-fluorophenyl)-N4,N4-dimethylbenzene-1,4-disulfonamide (Compound 4). The yield was 33.6%.

¹HNMR (300 MHz, CDCl₃) δ 8.07 (br, 1H), 7.96 (d, J 8.4 Hz, 2H), 7.83 (d, J 8.4 Hz, 2H), 7.62 (m, 1H), 6.87-6.81 (m, 2H), 2.71 (s, 6H), 2.42-2.32 (m, 1H), 2.27 (s, 3H), 1.68-1.65 (m, 4H), 1.15-0.85 (m, 6H). LCMS [M+H]⁺ calcd for C₂₁H₂₉FN₃S₂O₄, 470.15; found, 470.3.

### Example S16. Synthesis of Compound 3

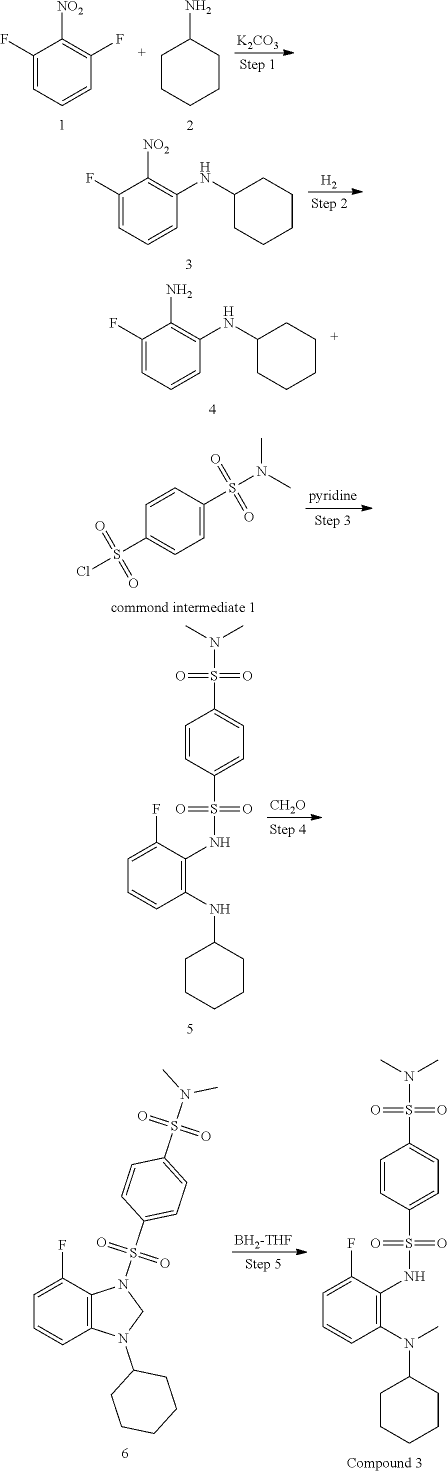

### Step 1

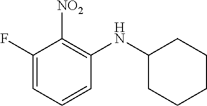 [SMILES: O=[N+]([O-])c1c(F)cccc1NC1CCCCC1]

N-cyclohexyl-3-fluoro-2-nitroaniline (3) was synthesized from starting materials (1) and (2) according to the procedure of Step 1 in the preparation of Compound 26.

LCMS [M+H]⁺ calcd for C₁₂H₁₆FN₂O₂, 239.11; found, 239.

### Step 2

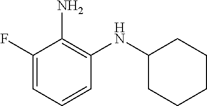 [SMILES: Nc1c(F)cccc1NC1CCCCC1]

N1-cyclohexyl-3-fluorobenzene-1,2-diamine (4) was synthesized from N-cyclohexyl-3-fluoro-2-nitroaniline (3) according to the procedure of Step 2 in the preparation of Compound 27.

LCMS [M+H]⁺ calcd for C₁₂H₁₈FN₂, 209.14; found, 209.

### Step 3

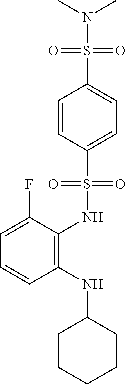 [SMILES: CN(C)S(=O)(=O)c1ccc(S(=O)(=O)Nc2c(F)cccc2NC2CCCCC2)cc1]

N1-(2-(cyclohexylamino)-6-fluorophenyl)-N4,N4-dimethylbenzene-1,4-disulfonamide (5) was synthesized from N1-cyclohexyl-3-fluorobenzene-1,2-diamine (4) and common intermediate 1 according to the procedure of Step 3 in the preparation of Compound 27.

LCMS [M+H]⁺ calcd for C₂₀H₂₇FN₃O₄S₂, 456.13; found, 456.

### Step 4

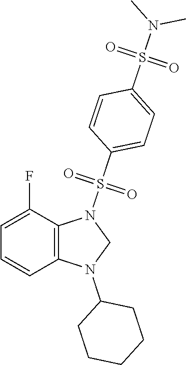 [SMILES: CN(C)S(=O)(=O)c1ccc(S(=O)(=O)N2CN(C3CCCCC3)c3cccc(F)c32)cc1]

4-((3-cyclohexyl-7-fluoro-2,3-dihydro-1H-benzo[d]imidazol-1-yl)sulfonyl)-N,N-dimethylbenzenesulfonamide (6) was synthesized from N1-(2-(cyclohexylamino)-6-fluorophenyl)-N4,N4-dimethylbenzene-1,4-disulfonamide (5) according to the procedure of Step 4 in the preparation of Compound 4.

LCMS [M+H]⁺ calcd for C₂₁H₂₇FN₃O₄S₂, 468.13; found, 468.

### Step 5

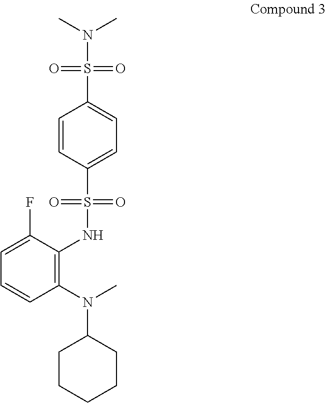 [SMILES: CN(c1cccc(F)c1NS(=O)(=O)c1ccc(S(=O)(=O)N(C)C)cc1)C1CCCCC1]

N1-(2-(cyclohexyl(methyl)amino)-6-fluorophenyl)-N4,N4-dimethylbenzene-1,4-disulfonamide (Compound 3) was synthesized from 4-((3-cyclohexyl-7-fluoro-2,3-dihydro-1H-benzo[d]imidazol-1-yl)sulfonyl)-N,N-dimethylbenzenesulfonamide (6) according to the procedure of Step 5 in the preparation of Compound 4.

¹HNMR (300 MHz, CDCl₃) δ 8.16 (d, J=8.4 Hz, 2H), 7.91 (d, J=8.4 Hz, 2H), 7.09-7.02 (m, 1H), 6.98-6.95 (m, 1H), 6.81 (t, J=9.9 Hz, 1H), 2.84 (m, 1H), 2.76 (s, 6H), 2.59 (s, 3H), 1.84-1.80 (m, 4H), 1.68-1.55 (m, 2H), 1.38-1.25 (m, 4H). LCMS [M+H]⁺ calcd for C₂₁H₂₉FN₃S₂O₄, 470.15; found, 470.3.

### Example S17. Synthesis of Compound 2

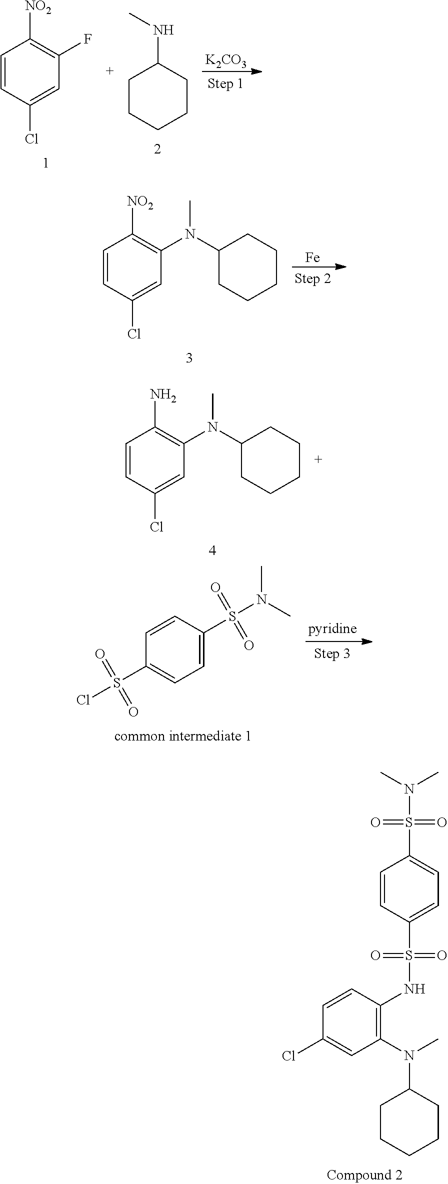

### Step 1

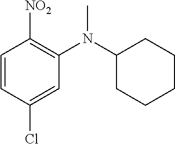 [SMILES: CN(c1cc(Cl)ccc1[N+](=O)[O-])C1CCCCC1]

5-chloro-N-cyclohexyl-N-methyl-2-nitroaniline (3) was synthesized from starting materials (1) and (2) according to the procedure of Step 1 in the preparation of Compound 26.

LCMS [M+H]⁺ calcd for C₁₃H₁₈ClN₂O₂, 269.10; found, 269.

### Step 2

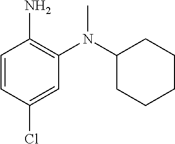 [SMILES: CN(c1cc(Cl)ccc1N)C1CCCCC1]

5-chloro-N1-cyclohexyl-N1-methylbenzene-1,2-diamine (4) was synthesized from 5-chloro-N-cyclohexyl-N-methyl-2-nitroaniline (3) according to the procedure of Step 2 in the preparation of Compound 26.

LCMS [M+H]⁺ calcd for C₁₃H₂₀ClN₂, 239.12; found, 239.

### Step 3

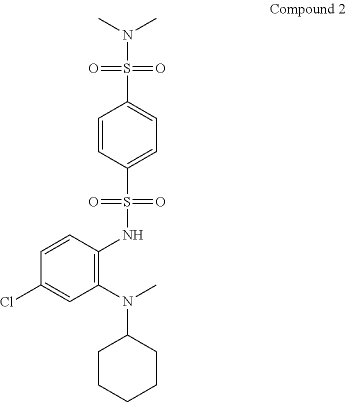

N1-(4-chloro-2-(cyclohexyl(methyl)amino)phenyl)-N4,N4-dimethylbenzene-1,4-disulfonamide (Compound 2) was synthesized from 5-chloro-N1-cyclohexyl-N1-methylbenzene-1,2-diamine (4) and common intermediate 1 according to the procedure of Step 3 in the preparation of Compound 27.

¹HNMR (300 MHz, CDCl₃) δ 8.26 (br s, 1H), 8.20 (d, J=8.1 Hz, 2H), 7.92 (d, J=8.1 Hz, 2H), 7.58 (d, J=8.4 Hz, 1H), 7.18-7.01 (m, 2H), 2.72 (s, 6H), 2.40-2.37 (m, 1H), 2.32 (s, 3H), 1.68-1.66 (m, 4H), 1.11-0.88 (m, 6H). LCMS [M+H]⁺ calcd for C₂₁H₂₉ClN₃S₂O₄, 486.12; found, 486.3.

### Example S18. Synthesis of Compound 1

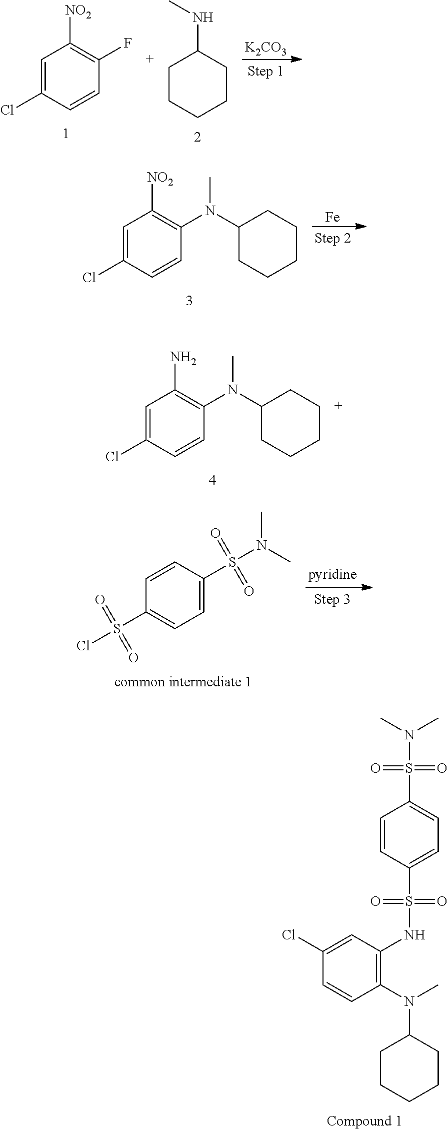

### Step 1

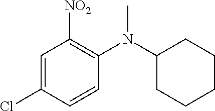 [SMILES: CN(c1ccc(Cl)cc1[N+](=O)[O-])C1CCCCC1]

4-chloro-N-cyclohexyl-N-methyl-2-nitroaniline (3) was synthesized from starting materials (1) and (2) according to the procedure of Step 1 in the preparation of Compound 26.

LCMS [M+H]⁺ calcd for C₁₃H₁₈ClN₂O₂, 269.10; found, 269.

### Step 2

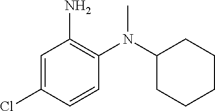 [SMILES: CN(c1ccc(Cl)cc1N)C1CCCCC1]

4-chloro-N1-cyclohexyl-N1-methylbenzene-1,2-diamine (4) was synthesized from 4-chloro-N-cyclohexyl-N-methyl-2-nitroaniline (3) according to the procedure of Step 2 in the preparation of Compound 26.

LCMS [M+H]⁺ calcd for C₁₃H₂₀ClN₂, 239.12; found, 239.

### Step 3

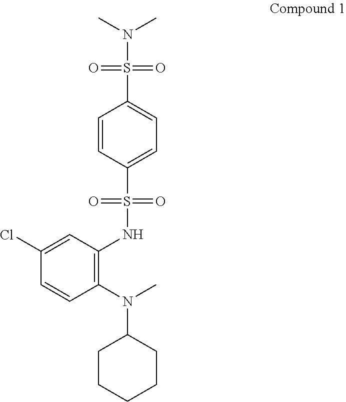

N1-(5-chloro-2-(cyclohexyl(methyl)amino)phenyl)-N4,N4-dimethylbenzene-1,4-disulfonamide (Compound 1) was synthesized from 4-chloro-N1-cyclohexyl-N1-methylbenzene-1,2-diamine (4) and common intermediate 1 according to the procedure of Step 3 in the preparation of Compound 27.

¹HNMR (300 MHz, CDCl₃) δ 8.02 (d, J=8.4 Hz, 2H), 7.86 (d, J=8.4 Hz, 2H), 7.65 (s, 1H), 6.98-7.04 (m, 2H), 2.72 (s, 6H), 2.34-2.41 (m, 1H), 2.32 (s, 3H), 1.53-1.70 (m, 4H), 0.94-1.08 (m, 6H). LCMS [M+H]⁺ calcd for C₂₁H₂₉ClN₃S₂O₄, 486.12; found, 486.4.

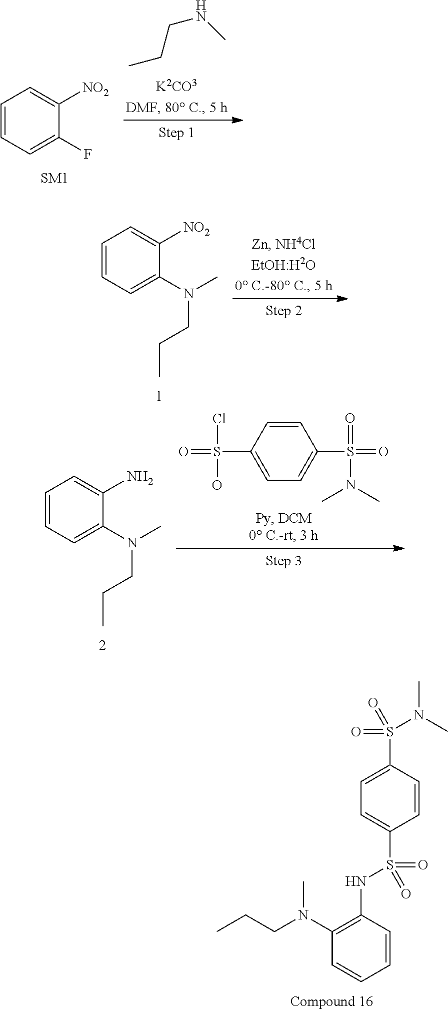

### Example S19. Synthesis of Compound 16

### Step 1

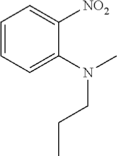

To a stirred solution of 1-fluoro-2-nitrobenzene (SM1) (500 mg, 3.54 mmol) in DMF (5 mL) were added K₂CO₃ (1.2 g, 8.69 mmol) and N-methylpropan-1-amine (310 mg, 4.24 mmol) at room temperature. The reaction mixture was heated at 80° C. and stirred for 5 h. After the reaction was completed as indicated by TLC, the reaction mixture was diluted with water (2 mL) and extracted with EtOAc (2×20 mL). The combined organic layers were washed with brine, dried over anhydrous Na₂SO₄, filtered, and concentrated under reduced pressure to give 460 mg of N-methyl-2-nitro-N-propylaniline (1). The yield was 67%.

^{l}H NMR (400 MHz, CDCl₃) δ 7.72 (d, J=7.8 Hz, 1H), 7.44-7.32 (m, 1H), 7.06 (d, J=8.3 Hz, 1H), 6.82 (t, J=7.6 Hz, 1H), 3.12 (t, J=7.3 Hz, 2H), 2.81 (s, 3H), 1.67-1.59 (m, 2H), 0.89 (t, J=7.3 Hz, 3H); LCMS [M+H]⁺ calcd for C₁₀H₁₅N₂O₂, 195.11; found, 195.3

### Step 2

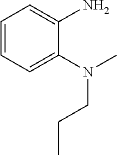

To a stirred solution of N-methyl-2-nitro-N-propylaniline (1) (460 mg, 2.37 mmol) in a mixture of EtOH and H₂O (12 mL, 5:1) were added Zn dust (1.54 g, 23.71 mmol) and NH₄C₁ (1.26 g, 23.71 mmol) at 0° C. The reaction mixture was heated at 80° C. and stirred for 5 h. After the reaction was completed as indicated by TLC, the reaction mixture was filtered through a Celite pad and the filtrate was concentrated under reduced pressure. The crude product was diluted with water (10 mL) and extracted with EtOAc (2×30 mL). The organic layers were washed with brine, dried over anhydrous Na₂SO₄, filtered, and concentrated under reduced pressure to give 420 mg of crude N1-methyl-N1-propylbenzene-1,2-diamine (2). This material was used in the next step without any further purification.

### Step 3

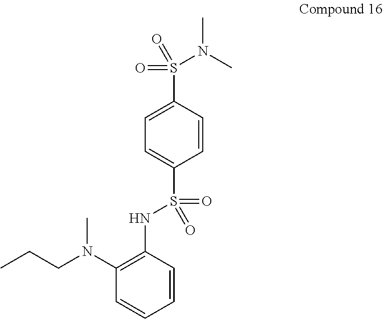 [SMILES: CCCN(C)c1ccccc1NS(=O)(=O)c1ccc(S(=O)(=O)N(C)C)cc1]

To a stirred solution of N1-methyl-N1-propylbenzene-1,2-diamine (2) (350 mg, 2.13 mmol) in CH₂Cl₂ (10 mL) were added pyridine (0.33 mL, 4.08 mmol) and 4-(N,N-dimethylsulfamoyl)benzenesulfonyl chloride (common intermediate 1) (750 mg, 2.64 mmol) at 0° C. The reaction mixture was stirred at room temperature for 3 h. After the reaction was completed as indicated by TLC, the reaction mixture was diluted with water (5 mL) and extracted with CH₂Cl₂ (2×30 mL). The combined organic layers were washed with brine, dried over anhydrous Na₂SO₄, filtered, and concentrated under reduced pressure. The crude product was purified by medium pressure liquid chromatography by eluting with 30-50%0 EtOAc in heptane to give 80 mg of N1,N1-dimethyl-N4-(2-(methyl(propyl)amino)phenyl)benzene-1,4-disulfonamide (Compound 16). The yield was 9.10.

¹H NMR (400 MHz, DMSO-d₆) δ9.22 (br s, 1H), 8.07-8.01 (m, 2H), 7.96-7.89 (m, 2H), 7.23 (dd, J=1.4, 7.9 Hz, 1H), 7.16-7.12 (m, 1H), 7.08 (dt, J=1.5, 7.6 Hz, 1H), 7.04-6.98 (m, 1H), 2.62 (s, 8H), 2.37 (s, 3H), 1.28-1.18 (2H), 0.73 (t, J=7.4 Hz, 3H); LCMS [M+H]⁺ calcd for C₁₈H₂₆N₃O₄S₂, 412.13; found, 412.2.

The following compounds were prepared according to the synthesis of Compound 16

| Structure | Name | Compound No. | Analytical data |
|---|---|---|---|
| 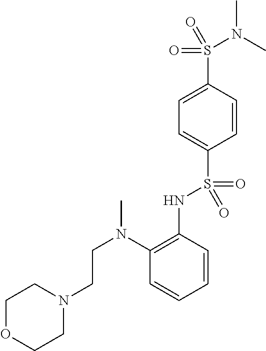 [SMILES: CN(CCN1CCOCC1)c1ccccc1NS(=O)(=O)c1ccc(S(=O)(=O)N(C)C)cc1] | N1,N1-dimethyl- N4-(2-(methyl(2- morpholinoethyl) amino)phenyl) benzene-1,4- disulfonamide | Compound 15 | ¹H NMR (400 MHz, DMSO-d₆) δ 11.79-11.08 (br s, 1H), 7.91- 7.84 (m, 2H), 7.78 (d, J = 8.4 Hz, 2H), 7.50-7.40 (m, 1H), 7.27- 7.13 (m, 3H), 3.84 (t, J = 4.5 Hz, 4H), 2.78-2.70 (m, 2H), 2.59 (s, 6H), 2.48 (br s, 4H), 2.17-2.08 (m, 2H), 1.93 (s, 3H); LCMS = 483.0 [M + H]⁺ HPLC: 99.01% |
| 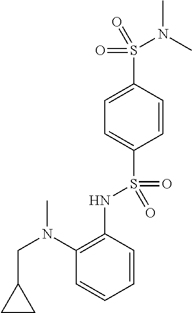 [SMILES: CN(CC1CC1)c1ccccc1NS(=O)(=O)c1ccc(S(=O)(=O)N(C)C)cc1] | N1-(2- ((cyclopropylmethyl) (methyl)amino)phenyl)- N4,N4- dimethylbenzene- 1,4-disulfonamide | Compound 14 | ¹H NMR (400 MHz, DMSO-d₆) δ 9.29 (br s, 1H), 8.05 (d, J = 8.5 Hz, 2H), 7.92 (d, J = 8.5 Hz, 2H), 7.24 (dd, J = 1.6, 7.8 Hz, 1H), 7.18-7.12 (m, 1H), 7.11-6.97 (m, 2H), 2.62 (s, 6H), 2.48 (br s, 2H), 2.45 (s, 3H), 0.77-0.64 (m, 1H), 0.31-0.23 (m, 2H), −0.06- −0.12 (m, 2H); LCMS = 423.7 [M + H]⁺ HPLC: 97.50% |
| 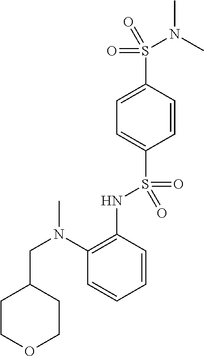 [SMILES: CN(CC1CCOCC1)c1ccccc1NS(=O)(=O)c1ccc(S(=O)(=O)N(C)C)cc1] | N1,N1-dimethyl- N4-(2- (methyl((tetrahydro- 2H-pyran-4- yl)methyl)amino) phenyl)benzene- 1,4-disulfonamide | Compound 11 | ¹H NMR (400 MHz, DMSO-d₆) δ 9.09 (br s, 1H), 8.06 (d, J = 7.8 Hz, 2H), 7.94 (d, J = 7.8 Hz, 2H), 7.22-7.13 (m, 2H), 7.08 (t, J = 7.6 Hz, 1H), 7.03-6.95 (m, 1H), 3.82-3.69 (m, 2H), 3.16 (t, J = 11.5 Hz, 2H), 2.70-2.59 (m, 8H), 2.40 (s, 3H), 1.66-1.55 (m, 1H), 1.50 (d, J = 12.7 Hz, 2H), 1.13-0.97 (m, 2H); LCMS = 468.2 [M + H]⁺ HPLC: 96.64% |
| 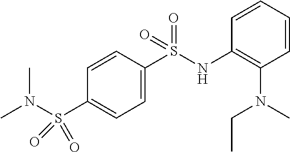 | N1-(2- (ethyl(methyl)amino) phenyl)-N4,N4- dimethylbenzene- 1,4-disulfonamide | Compound 9 | ¹H NMR (400 MHz, DMSO-d₆) δ 9.42-9.20 (m, 1H), 8.09-8.01 (m, 2H), 7.92 (d, J = 8.5 Hz, 2H), 7.25 (dd, J = 1.4, 7.9 Hz, 1H), 7.17-6.97 (m, 3H), 2.70 (q, J = 7.0 Hz, 2H), 2.61 (s, 6H), 2.35 (s, 3H), 0.78 (t, J = 7.2 Hz, 3H); LCMS = 398.3 [M + H]⁺ HPLC: 98.75% |

### Example S20. Synthesis of Compound 13

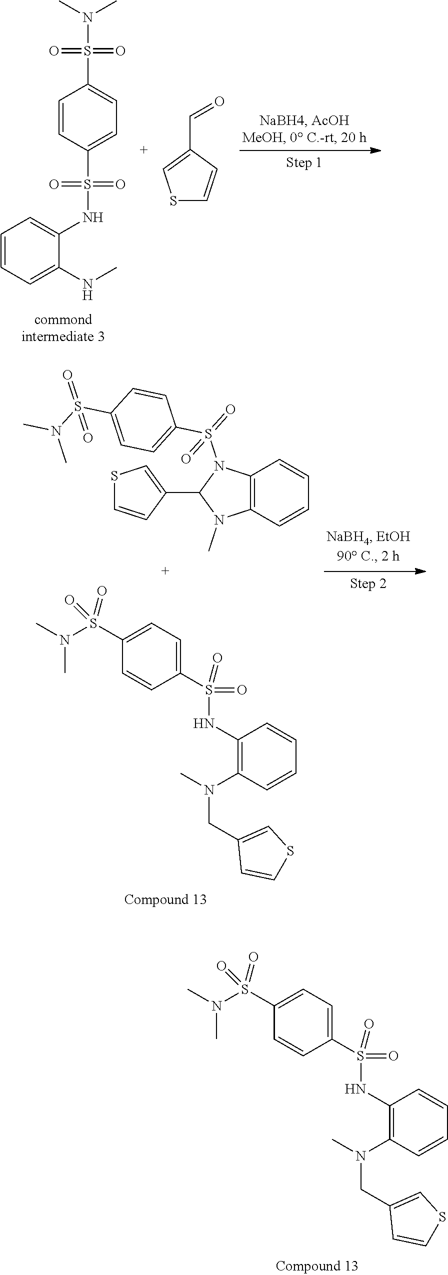

### Step 1

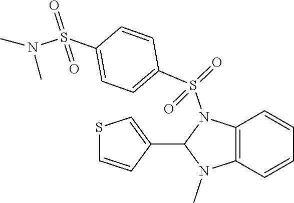 [SMILES: CN1c2ccccc2N(S(=O)(=O)c2ccc(S(=O)(=O)N(C)C)cc2)C1c1ccsc1]

To a solution of N1,N1-dimethyl-N4-(2-(methylamino)phenyl)benzene-1,4-disulfonamide (common intermediate 3) (150 mg, 0.406 mmol) in MeOH (2 mL) were added AcOH (19 mg, 0.32 mmol) and thiophene-3-carbaldehyde (60 mg, 0.528 mmol) at room temperature and stirred for 16 h. The reaction mixture was cooled to 0° C. and NaBH₄ (30 mg, 0.0.79 mmol) was added. The resulting reaction mixture was stirred at room temperature for 4 h. After the reaction was completed as indicated by TLC, the reaction mixture was concentrated and extracted with EtOAc (20 mL×3) and washed with water. The organic layers were dried over anhydrous Na₂SO₄, filtered, and concentrated under reduced pressure to give 170 mg mixture of crude N,N-dimethyl-4-((3-methyl-2-(thiophen-3-yl)-2,3-dihydro-1H-benzo[d]imidazol-1-yl)sulfonyl)benzenesulfonamide (1) and N1,N1-dimethyl-N4-(2-(methyl(thiophen-3-ylmethyl)amino)phenyl)benzene-1,4-disulfonamide (Compound 13). This mixture was used in the next step without any further purification.

### Step 2

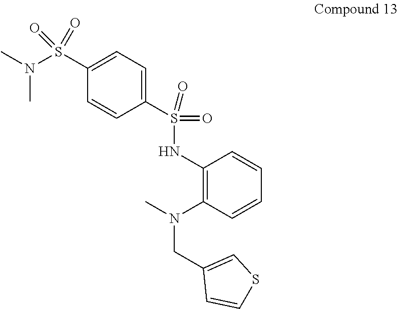 [SMILES: CN(Cc1ccsc1)c1ccccc1NS(=O)(=O)c1ccc(S(=O)(=O)N(C)C)cc1]

To a solution of mixture of crude N,N-dimethyl-4-((3-methyl-2-(thiophen-3-yl)-2,3-dihydro-1H-benzo[d]imidazol-1-yl)sulfonyl)benzenesulfonamide (1) and N1,N1-dimethyl-N4-(2-(methyl(thiophen-3-ylmethyl)amino)phenyl)benzene-1,4-disulfonamide (Compound 13) (170 mg, 0.367 mmol) in EtOH (2 mL) was added NaBH₄ (55 mg, 1.45 mmol) at room temperature. The resulting reaction mixture was heated at 90° C. and stirred for 2 h. After the reaction was completed as indicated by TLC, the reaction mixture was concentrated under reduced pressure. The crude product was purified by medium pressure liquid column chromatography eluting with 20-30% EtOAc in heptane to give 50 mg of N1,N1-dimethyl-N4-(2-(methyl(thiophen-3-ylmethyl)amino)phenyl)benzene-1,4-disulfonamide (Compound 13). The yield was 29%.

¹H NMR (400 MHz, DMSO-d₆) δ 9.49 (br s, 1H), 8.05 (d, J=7.8 Hz, 2H), 7.90 (d, J=7.8 Hz, 2H), 7.41 (br s, 1H), 7.26 (br s, 1H), 7.14 (d, J=7.8 Hz, 1H), 7.08-6.89 (m, 4H), 3.87 (s, 2H), 2.58 (s, 6H), 2.37 (s, 3H); LCMS [M+H]⁺ calcd for C₂₀H₂₄N₃O₄S₃, 466.09; found, 466.8; HPLC: 98.06%

The following compounds were prepared according to the synthesis of Compound 13.

| Structure | Name | Compound No. | Analytical data |
|---|---|---|---|
| 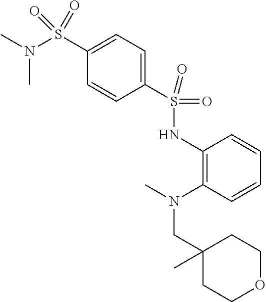 [SMILES: CN(CC1(C)CCOCC1)c1ccccc1NS(=O)(=O)c1ccc(S(=O)(=O)N(C)C)cc1] | N1,N1- dimethyl-N4- (2-(methyl((4- methyltetrahydro- 2H-pyran-4- yl)methyl)amino) phenyl)benzene- 1,4-disulfonamide | Compound 10 | ¹H NMR (400 MHz, DMSO-d₆) δ 9.05 (br s, 1H), 8.09 (br d, J = 7.8 Hz, 2H), 7.94 (br d, J = 8.3 Hz, 2H), 7.31 (br d, J = 7.3 Hz, 1H), 7.24 (br d, J = 7.8 Hz, 1H), 7.09-6.93 (m, 2H), 3.53-3.45 (m, 2H), 3.43-3.35 (m, 2H), 2.89 (s, 2H), 2.62 (s, 6H), 2.47 (br s, 3H), 1.34-1.20 (m, 2H), 1.09-1.01 (m, 2H), 0.81 (s, 3H); LCMS = 482.6 [M + H]⁺ HPLC: 98.51% |
| 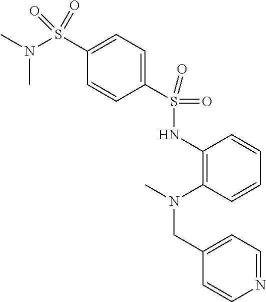 [SMILES: CN(Cc1ccncc1)c1ccccc1NS(=O)(=O)c1ccc(S(=O)(=O)N(C)C)cc1] | N1,N1- dimethyl-N4- (2- (methyl(pyridin- 4-ylmethyl) amino)phenyl) benzene-1,4- disulfonamide | Compound 8 | ¹H NMR (400 MHz, DMSO-d₆) δ 9.62 (s, 1H), 8.45 (d, J = 5.8 Hz, 2H), 8.05 (d, J = 8.5 Hz, 2H), 7.91 (d, J = 8.5 Hz, 2H), 7.29 (d, J = 5.6 Hz, 2H), 7.14- 7.03 (m, 3H), 7.01-6.90 (m, 1H), 3.98 (s, 2H), 2.59 (s, 6H), 2.42 (s, 3H) LCMS = 461.4 [M + H]⁺ HPLC: 97.24% |
| 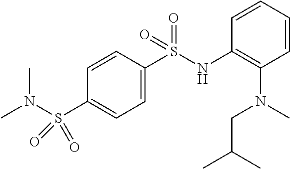 | N1-(2- (isobutyl(methyl) amino)phenyl)- N4,N4- dimethylbenzene- 1,4- disulfonamide | Compound 7 | ¹H NMR (400 MHz, DMSO-d₆) δ 9.00 (br s, 1H), 8.05 (d, J = 8.5 Hz, 2H), 7.93 (d, J = 8.4 Hz, 2H), 7.22 (dd, J = 1.5, 8.0 Hz, 1H), 7.18-7.13 (m, 1H), 7.07 (br t, J = 7.2 Hz, 1H), 7.03-6.97 (m, 1H), 2.62 (s, 6H), 2.53 (d, J = 7.3 Hz, 2H), 2.39 (s, 3H), 1.67- 1.60 (m, 1H), 0.79 (d, J = 6.6 Hz, 6H) LCMS = 426.5 [M + H]⁺ |
| | | | HPLC: 99.57% |
| 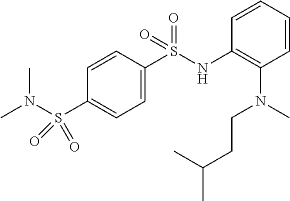 | N1-(2- (isopentyl(methyl) amino)phenyl)- N4,N4- dimethylbenzene- 1,4-disulfonamide | Compound 6 | ¹H NMR (400 MHz, DMSO-d₆) δ 9.27 (br s, 1H), 8.06 (br d, J = 8.3 Hz, 2H), 7.92 (br d, J = 8.3 Hz, 2H), 7.22 (br d, J = 7.8 Hz, 1H), 7.17-6.93 (m, 3H), 2.80- 2.68 (m, 2H), 2.62 (s, 6H), 2.37 (s, 3H), 1.47-1.32 (m, 1H), 1.17- 1.02 (m, 2H), 0.78 (d, J = 6.4 Hz, 6H) LCMS = 440.8 [M + H]⁺ HPLC: 98.51% |
| 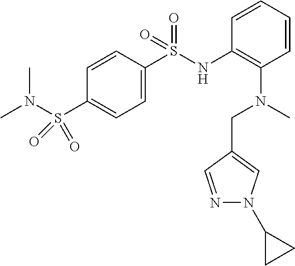 [SMILES: CN(Cc1cnn(C2CC2)c1)c1ccccc1NS(=O)(=O)c1ccc(S(=O)(=O)N(C)C)cc1] | N1-(2-(((1- cyclopropyl- 1H-pyrazol-4- yl)methyl)(methyl) amino)phenyl)- N4,N4- dimethylbenzene- 1,4- disulfonamide | Compound 5 | ¹H NMR (400 MHz, DMSO-d₆) δ 9.43 (br s, 1H), 8.08-8.02 (m, 2H), 7.94-7.87 (m, 2H), 7.51 (s, 1H), 7.17-7.12 (m, 2H), 7.09- 7.02 (m, 1H), 7.01-6.95 (m, 2H), 3.67 (s, 2H), 3.64-3.57 (m, 1H), 2.58 (s, 6H), 2.34 (s, 3H), 0.95-0.87 (m, 4H) LCMS = 490.4 [M + H]⁺ HPLC: 98.61% |

### Example S21. Synthesis of Compound 30

### Step 1

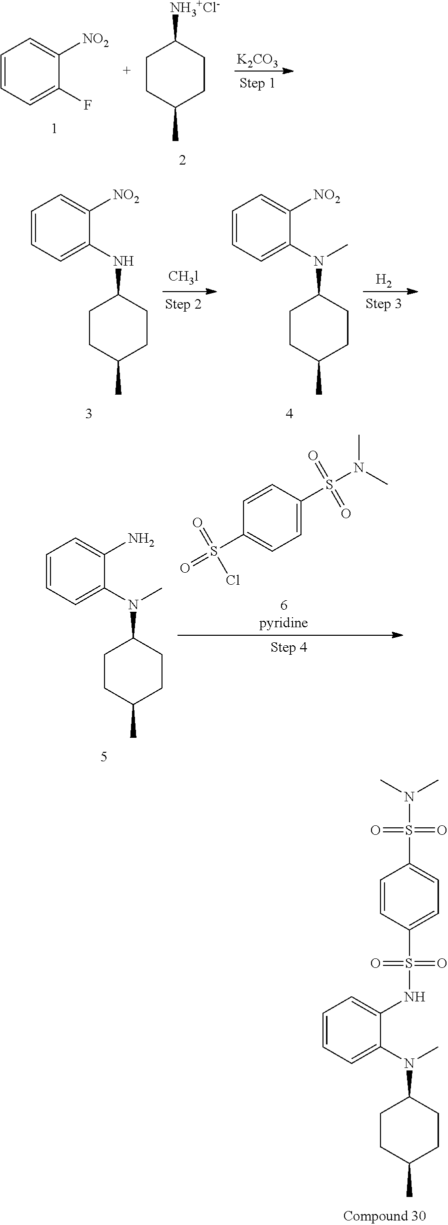

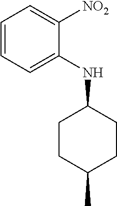 [SMILES: C[C@H]1CC[C@@H](Nc2ccccc2[N+](=O)[O-])CC1]

To a solution of 1-fluoro-2-nitrobenzene (350.0 mg, 2.5 mmol) in DMF (19.0 mL) was added (1s,4s)-4-methylcyclohexanaminium chloride (556.9 mg, 3.7 mmol) and K₂CO₃ (1.0 g, 7.4 mmol). The reaction was stirred at 100° C. for 4 hr. After the reaction was completed as indicated by TLC, the reaction was quenched with saturated aqueous NH₄Cl (60.0 mL) and extracted with EtOAc (60.0 mL×2). The combined organic layer was washed with brine, dried over anhydrous Na₂SO₄, filtered, and concentrated under reduced pressure. The residue was purified by silica gel chromatography (PE:EtOAc=200:1) to give 560.0 mg (2.4 mmol) of the title compound. The yield was 96.4%.

LCMS [M+H]⁺ calcd for C₁₃H₁₉N₂O₂, 235.14; found, 235.10.

### Step 2

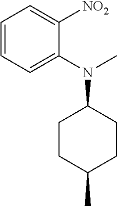 [SMILES: CN(c1ccccc1[N+](=O)[O-])[C@H]1CC[C@@H](C)CC1]

Under nitrogen protection, to a solution of N-((1s,4s)-4-methylcyclohexyl)-2-nitroaniline (560.0 mg, 2.4 mmol) in DMF (32.0 mL) at 0° C. was added NaH (382.4 mg, 9.6 mmol, 60%) portion wise over 2 mins. After the resulting mixture was stirred at 0° C. for 30 mins, iodomethane (1.4 g, 9.6 mmol) was added into the reaction dropwise over 2 mins. The reaction was stirred at rt overnight. After the reaction was completed as indicated by TLC, the reaction was quenched with saturated aqueous NH₄Cl (60.0 mL) and extracted with EtOAc (80.0 mL×2). The combined organic layer was washed with brine, dried over anhydrous Na₂SO₄, filtered, and concentrated under reduced pressure. The residue was purified by silica gel chromatography (PE:EtOAc=200:1) to give 550.0 mg (2.2 mmol) of the title compound. The yield was 92.7%.

LCMS [M+H]⁺ calcd for C₁₄H₂₁N₂O₂, 249.15; found, 249.03.

### Step 3

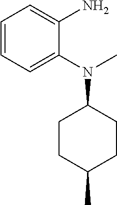 [SMILES: CN(c1ccccc1N)[C@H]1CC[C@@H](C)CC1]

To a solution of N-methyl-N-((1s,4s)-4-methylcyclohexyl)-2-nitroaniline (280.0 mg, 1.1 mmol) in IPA (18 mL) and THF (18 mL) was added Pd/C (90 mg, 10%). The suspension was evacuated and then refilled with hydrogen for three times. The reaction mixture was then stirred under hydrogen atmosphere from a balloon for 3 hr. After the reaction was completed as indicated by TLC, the suspension was filtered through a pad of Celite and the filtered cake was washed with IPA (10 mL). The combined filtrate was concentrated under reduced pressure to give 220.0 mg of the crude title compound. The yield was 89.4%.

LCMS [M+H]⁺ calcd for C₁₄H₂₃N₂, 219.18; found, 219.27.

### Step 4

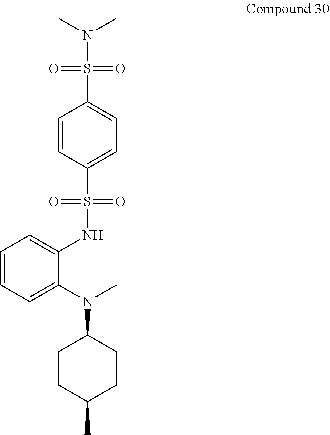 [SMILES: CN(C)S(=O)(=O)c1ccc(S(=O)(=O)Nc2ccccc2N(C)[C@H]2CC[C@@H](C)CC2)cc1]

To a solution of N1-methyl-N1-((1s,4s)-4-methylcyclohexyl)benzene-1,2-diamine (100.0 mg, 0.5 mmol) in THE (5.0 mL) was added 4-(N,N-dimethylsulfamoyl)benzene-1-sulfonyl chloride (169.0 mg, 0.6 mmol) and pyridine (144.9 mg, 1.8 mmol). The reaction mixture was stirred at 60° C. overnight. After the reaction was completed as indicated by TLC, the reaction was quenched with water (20.0 mL) and extracted with EtOAc (25.0 mL×2). The combined organic layer was washed with brine, dried over anhydrous Na₂SO₄, filtered, and concentrated under reduced pressure. The residue was purified by silica gel chromatography to give the impure title compound. The impure product was further purified by preparative HPLC to give 71.6 mg of the title compound. The yield was 33.6%.

¹H-NMR (300 MHz, CDCl₃) δ 8.42 (brs, 1H), 8.02 (d, J=8.4 Hz, 2H), 7.82 (d, J=8.4 Hz, 2H), 7.64 (d, J=7.5 Hz, 1H), 7.19-7.00 (m, 3H), 2.70 (s, 6H), 2.28 (s, 3H), 1.71-1.63 (m, 1H), 1.44-1.21 (m, 8H), 0.90 (d, J=6.9 Hz, 3H). LCMS [M+H]⁺ calcd for C₂₂H₃₂ClN₃S₂O₄, 466; found, 466.29.

The following compounds were prepared according to the synthesis of Compound 30.

| Structure | Name | Compound No. | Analytical data |
|---|---|---|---|
| 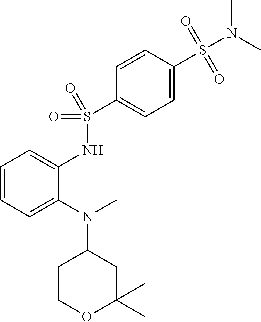 [SMILES: CN(c1ccccc1NS(=O)(=O)c1ccc(S(=O)(=O)N(C)C)cc1)C1CCOC(C)(C)C1] | N1-(2-((2,2- dimethyltetrahydro- 2H-pyran-4- yl)(methyl)amino) phenyl)-N4,N4- dimethylbenzene- 1,4-disulfonamide | Compound 111 | ¹H NMR (300 MHz, CDCl₃) δ 8.42-8.41 (br s, 1H), 8.02 (d, J = 8.1 Hz, 2H), 7.83 (d, J = 8.1 Hz, 2H), 7.58 (d, J = 7.8 Hz, 1H), 7.17-7.02 (m, 3H), 3.75- 3.69 (m, 1H), 3.54 (t, J = 11.4 Hz, 1H), 2.96-2.94 (m, 1H), 2.71 (s, 6H), 2.31 (s, 3H), 1.64- 1.57 (m, 2H), 1.38-1.28 (m, 1H), 1.26-1.21 (m, 1H), 1.17 (s, 3H), 1.23 (s, 3H); LCMS = 481.82 [M + H]⁺ |
| 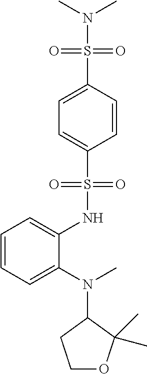 [SMILES: CN(c1ccccc1NS(=O)(=O)c1ccc(S(=O)(=O)N(C)C)cc1)C1CCOC1(C)C] | N1-(2-((2,2- dimethyltetrahydro- furan-3- yl)(methyl)amino) phenyl)-N4,N4- dimethylbenzene- 1,4-disulfonamide | Compound 97 | ¹H-NMR (300 MHz, CDCl₃) δ 8.43 (br, 1H), 8.02 (d, J = 8.4 Hz, 2H), 7.84 (d, J = 8.4 Hz, 2H), 7.61 (d, J = 7.8 Hz, 1H), 7.16-7.04 (m, 3H), 3.71-3.62 (m, 2H), 3.17 (t, J = 8.1 Hz, 1H), 2.71 (s, 6H), 2.31 (s, 3H), 1.85-1.80 (m, 1H), 1.55-1.42 (m, 1H), 1.21 (d, J = 4.5 Hz, 6H); LCMS = 467.89 [M + H]⁺ |
| 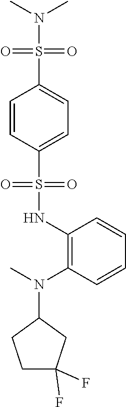 [SMILES: CN(c1ccccc1NS(=O)(=O)c1ccc(S(=O)(=O)N(C)C)cc1)C1CCC(F)(F)C1] | N1-(2-((3,3- difluorocyclopentyl) (methyl)amino) phenyl)-N4,N4- dimethylbenzene- 1,4-disulfonamide | Compound 99 | ¹H NMR (300 MHz, CDCl₃) δ 8.33 (s, 1H), 8.02 (d, J = 6.9 Hz, 2H), 7.84 (d, J = 6.9 Hz, 2H), 7.70-7.56 (m, 1H), 7.24- 7.01 (m, 3H), 3.52-3.33 (m, 1H), 2.71 (s, 6H), 2.47 (s, 3H), 2.24-2.03 (m, 2H), 2.02-1.99 (m, 1H), 1.98-1.67 (m, 2H), 1.48-1.28 (m, 1H); LCMS = 474.3 [M + H]⁺ |
| 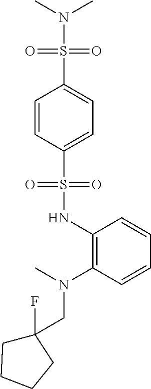 [SMILES: CN(CC1(F)CCCC1)c1ccccc1NS(=O)(=O)c1ccc(S(=O)(=O)N(C)C)cc1] | N1-(2-(((1- fluorocyclopentyl) methyl)(methyl) amino)phenyl)- N4,N4- dimethylbenzene- 1,4-disulfonamide | Compound 93 | ¹H-NMR (300 MHz, CDCl₃) δ 8.57 (br, 1H), 8.03 (d, J = 8.4 Hz, 2H), 7.82 (d, J = 8.4 Hz, 2H), 7.59 (d, J = 7.5 Hz, 1H), 7.17-7.03 (m, 3H), 3.04 (s, 1H), 2.97 (s, 1H), 2.71 (s, 6H), 2.49 (s, 3H), 1.88-1.80 (m, 4H), 1.70-1.56 (m, 2H), 1.55- 1.40 (m, 2H); LCMS = 469.92 [M + H]⁺ |
| 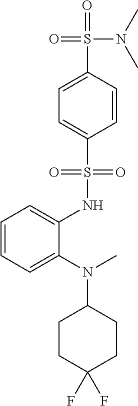 [SMILES: CN(c1ccccc1NS(=O)(=O)c1ccc(S(=O)(=O)N(C)C)cc1)C1CCC(F)(F)CC1] | N1-(2-((4,4- difluorocyclohexyl) (methyl)amino) phenyl)-N4,N4- dimethylbenzene- 1,4-disulfonamide | Compound 41 | ¹H-NMR (300 MHz, CDCl₃) δ 8.30 (br, 1H), 8.01 (d, J = 8.1 Hz, 2H), 7.84 (d, J = 8.1 Hz, 2H), 7.61 (d, J = 7.8 Hz, 1H), 7.17-7.03 (m, 3 H), 2.72 (s, 6H), 2.72-2.70 (m, 1H), 2.37 (s, 3H), 2.10-1.92 (m, 2H), 1.75-1.72 (m, 4H), 1.55-1.42 (m, 2H); LCMS = 488.05 [M + H]⁺ |
| 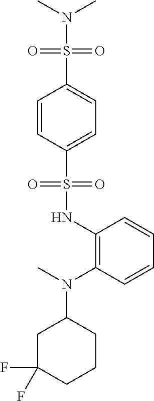 [SMILES: CN(c1ccccc1NS(=O)(=O)c1ccc(S(=O)(=O)N(C)C)cc1)C1CCCC(F)(F)C1] | N1-(2-((3,3- difluorocyclohexyl) (methyl)amino) phenyl)-N4,N4- dimethylbenzene- 1,4-disulfonamide | Compound 104 | ¹H-NMR (300 MHz, CDCl₃) δ 8.29 (br, 1H), 8.01 (d, J = 8.4 Hz, 2H), 7.83 (d, J = 8.4 Hz, 2H), 7.63 (d, J = 7.8 Hz, 1H), 7.18-7.04 (m, 3 H), 2.86-2.78 (m, 1H), 2.71 (s, 6H), 2.37 (s, 3H), 2.19-2.15 (m, 1H), 2.01- 1.95 (m, 1H), 1.80-1.64 (m, 2H), 1.55-1.34 (m, 3H), 1.07- 0.99 (m, 1H); LCMS = 487.91 [M + H]⁺ |
| 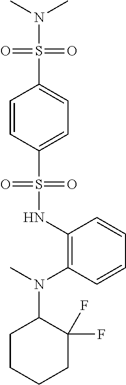 [SMILES: CN(c1ccccc1NS(=O)(=O)c1ccc(S(=O)(=O)N(C)C)cc1)C1CCCCC1(F)F] | N1-(2-((2,2- difluorocyclohexyl) (methyl)amino) phenyl)-N4,N4- dimethylbenzene- 1,4-disulfonamide | Compound 103 | ¹H-NMR (300 MHz, CDCl₃) δ 8.55 (br, 1H), 8.02 (d, J = 8.4 Hz, 2H), 7.81 (d, J = 8.4 Hz, 2H), 7.63 (d, J = 7.8 Hz, 1H), 7.13 (t, J = 7.8(?) Hz, 2H), 7.05-7.00 (m, 1H), 2.71 (s, 6H), 2.71-2.56 (m, 1H), 2.54 (s, 3H), 2.25-2.12 (m, 1H), 1.82-1.73 (m, 3H), 1.52-1.44 (m, 3H), 1.26-1.10 (m, 1H); LCMS = 487.91 [M + H]⁺ |
| 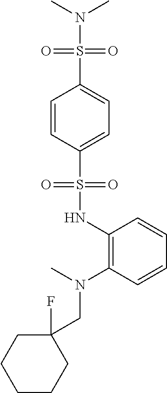 | N1-(2-(((1- fluorocyclohexyl) methyl)(methyl) amino)phenyl)- N4,N4- dimethylbenzene- 1,4-disulfonamide | Compound 96 | ¹H NMR (300 MHz, CDCl₃) δ 8.42 (br s, 1H), 8.02 (d, J = 8.1 Hz, 2H), 7.82 (d, J = 8.1 Hz, 2H), 7.62-7.48 (m, 1H), 7.22- 6.98 (m, 3H), 2.98-2.78 (m, 2H), 2.71 (s, 6H), 2.44 (s, 3H), 1.99-1.73 (m, 2H), 1.73-1.60 (m, 4H), 1.44-1.10 (m, 4H); LCMS = 484.0 [M + H]⁺ |
| 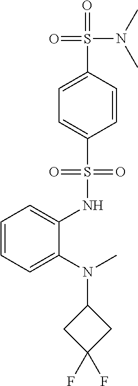 [SMILES: CN(c1ccccc1NS(=O)(=O)c1ccc(S(=O)(=O)N(C)C)cc1)C1CC(F)(F)C1] | N1-(2-((3,3- difluorocyclobutyl) (methyl)amino) phenyl)-N4,N4- dimethylbenzene- 1,4-disulfonamide | Compound 40 | ¹H NMR (300 MHz, CDCl₃) δ 8.21 (br s, 1H), 8.04 (d, J = 8.4 Hz, 2H), 7.84 (d, J = 8.1 Hz, 2H), 7.65 (d, J = 8.4 Hz, 1H), 7.19-7.16 (m, 1H), 7.14-7.04 (m, 2H), 3.48-3.45 (m, 1H), 2.71 (s, 6H), 2.65-2.47 (m, 2H), 2.32 (s, 3H), 2.07-2.01 (m, 2H); LCMS = 459.70 [M + H]⁺ |
| 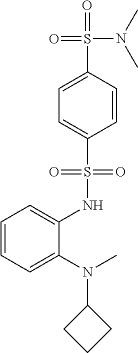 [SMILES: CN(c1ccccc1NS(=O)(=O)c1ccc(S(=O)(=O)N(C)C)cc1)C1CCC1] | N1-(2- (cyclobutyl(methyl) amino)phenyl)- N4,N4- dimethylbenzene- 1,4-disulfonamide | Compound 37 | ¹H-NMR (300 MHz, CD₃OD) δ 8.06 (d, J = 8.4 Hz, 2H), 7.89 (d, J = 8.4 Hz, 2H), 7.61 (d, J = 7.8 Hz, 1H), 7.15-7.02 (m, 3H), 3.54-3.39 (m, 1H), 2.65 (s, 6H), 2.23 (s, 3H), 1.88-1.86 (m, 2H), 1.62-1.49 (m, 4H); LCMS = 423.99 [M + H]⁺ |
| 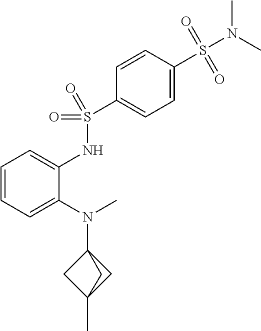 [SMILES: CN(c1ccccc1NS(=O)(=O)c1ccc(S(=O)(=O)N(C)C)cc1)C12CC(C)(C1)C2] | N1,N1-dimethyl-N4- (2-(methyl(3- methylbicyclo[1.1.1] pentan-1- yl)amino)phenyl) benzene-1,4- disulfonamide | Compound 115 | ¹H-NMR (300 MHz, CD₃OD) δ 8.03 (d, J = 8.4 Hz, 2H), 7.96 (d, J = 8.4 Hz, 2H), 7.58 (d, J = 8.1 Hz, 1H), 7.16-7.11 (m, 2H), 7.07-7.02 (m, 1H), 2.66 (s, 6H), 2.36 (s, 3H), 1.50 (s, 6H), 1.14 (s, 3H); LCMS = 449.90 [M + H]⁺ |
| 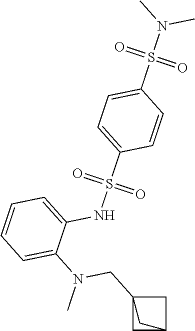 [SMILES: CN(CC12CC(C1)C2)c1ccccc1NS(=O)(=O)c1ccc(S(=O)(=O)N(C)C)cc1] | N1-(2- ((bicyclo[1.1.1] pentan-1- ylmethyl)(methyl) amino)phenyl)- N4,N4- dimethylbenzene- 1,4-disulfonamide | Compound 66 | ¹H NMR (300 MHz, CD₃OD) δ 8.07 (d, J = 8.1 Hz, 2H), 7.89 (d, J = 8.4 Hz, 2H), 7.58 (d, J = 7.5 Hz, 1H), 7.18 (d, J = 7.2 Hz, 1H), 7.13-7.01 (m, 2H), 2.78 (s, 2H), 2.65 (s, 6H), 2.34- 2.31 (m, 4H), 1.55 (s, 6H); LCMS = 449.83 [M + H]⁺ |
| 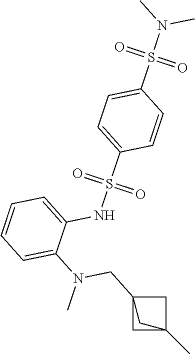 [SMILES: CN(CC12CC(C)(C1)C2)c1ccccc1NS(=O)(=O)c1ccc(S(=O)(=O)N(C)C)cc1] | N1,N1-dimethyl-N4- (2-(methyl((3- methylbicyclo[1.1.1] pentan-1- yl)methyl)amino) phenyl)benzene-1,4- disulfonamide | Compound 65 | ¹H NMR (300 MHz, CDCl₃) δ 8.40 (br s, 1H), 8.20 (d, J = 8.7 Hz, 2H), 7.83 (d, J = 8.4 Hz, 2H), 7.59 (d, J = 7.5 Hz, 1H), 7.12-6.96 (m, 3H), 2.73-2.70 (m, 8H), 2.32 (s, 3H), 1.42 (s, 6H), 1.09 (s, 3H); LCMS = 463.76 [M + H]⁺ |

### Example S22. Synthesis of Compound 39

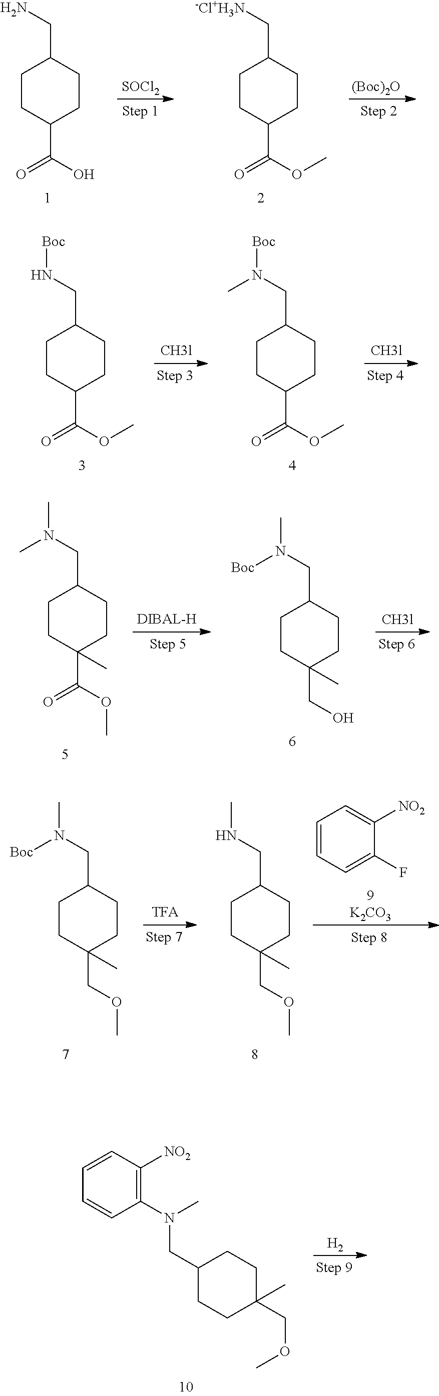

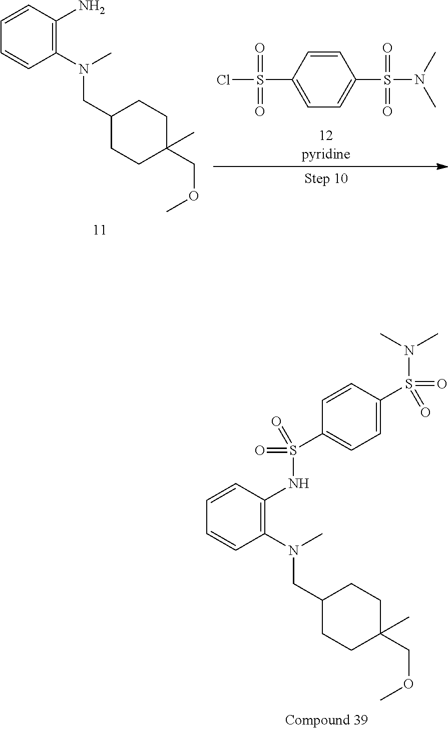 [SMILES: COCC1(C)CCC(CN(C)c2ccccc2N)CC1 ; CN(C)S(=O)(=O)c1ccc(S(=O)(=O)Cl)cc1 ; COCC1(C)CCC(CN(C)c2ccccc2NS(=O)(=O)c2ccc(S(=O)(=O)N(C)C)cc2)CC1]

### Step 1

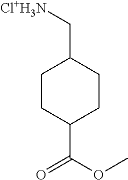

To a solution of 4-(aminomethyl)cyclohexanecarboxylic acid (2.5 g, 15.9 mmol) in MeOH (160.0 mL) was added sulfurous dichloride (7.6 g, 63.6 mmol)) dropwise over 10 mins. The reaction was stirred at rt for 4 hr. After the reaction was completed as indicated by TLC, the reaction was concentrated in vacuum to give 2.7 g (13.0 mmol) of the title compound. The yield was 81.8%.

LCMS [M+H]⁺ calcd for C₉H₁₈NO₂, 172.13; found, 172.25.

### Step 2

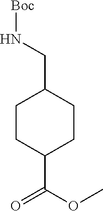 [SMILES: COC(=O)C1CCC(CNC(=O)OC(C)(C)C)CC1]

To a solution of (4-(methoxycarbonyl)cyclohexyl)methanaminium chloride (2.7 g, 13.0 mmol) in a saturated aqueous Na₂CO₃ (75.0 mL) solution and EtOAc (75.0 mL) was added (Boc)₂O (4.3 g, 19.5 mmol,). The reaction was stirred at rt for 6 hr. After the reaction was completed as indicated by TLC, the reaction was quenched with water (60.0 mL) and extracted with EtOAc (60.0 mL×2). The combined organic layer was washed with brine, dried over anhydrous Na₂SO₄, filtered, and concentrated under reduced pressure. The residue was purified by silica gel chromatography (PE:EtOAc=50:1 to 10:1) to give 4.2 g (15.5 mmol) of the title compound. The yield was quantitative.

LCMS [M+Na]⁺ calcd for C₁₄H₂₅NO₄, 271.18; found, 294.27.

### Step 3

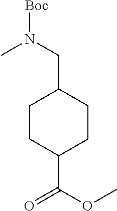

Under nitrogen protection, to a solution of methyl 4-(((tert-butoxycarbonyl)amino)methyl)cyclohexanecarboxylate (4.2 g, 15.5 mmol) in DMF (150.0 mL) at 0° C. was added NaH (1.2 g, 31.0 mmol, 60%). After the resulting mixture was stirred at 0° C. for 30 mins, iodomethane (1.2 g, 31.0 mmol) was added into the reaction. The reaction was stirred at rt overnight. After the reaction was completed as indicated by TLC, the reaction was quenched with water (150.0 mL) and extracted with EtOAc (100.0 mL×2). The combined organic layer was washed with brine, dried over anhydrous Na₂SO₄, filtered, and concentrated under reduced pressure. The residue was purified by silica gel chromatography (PE:EtOAc=40:1 to 30:1) to give 4.7 g (16.5 mmol) of the title compound. The yield was 106.4%.

LCMS [M+Na]⁺ calcd for C₁₅H₂₇NO₄, 285.19; found, 308.20.

### Step 4

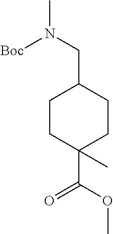 [SMILES: COC(=O)C1(C)CCC(CN(C)C(=O)OC(C)(C)C)CC1]

Under nitrogen protection, to a solution of methyl 4-(((tert-butoxycarbonyl)(methyl)amino)methyl)cyclohexanecarboxylate (3.5 g, 12.3 mmol) in anhydrous THE (100.0 mL) was added LDA (18.4 mL g, 36.8 mmol, 2M) dropwise at −72° C. After the resulting mixture was stirred at −72° C. for 10 mins, iodomethane (2.6 g, 18.4 mmol) was added into the reaction. The reaction was stirred at −72° C. for 3 hr. After the reaction was completed as indicated by TLC, the reaction was quenched with water (100.0 mL) and extracted with EtOAc (80.0 mL×2). The combined organic layer was washed with brine, dried over anhydrous Na₂SO₄, filtered, and concentrated under reduced pressure. The residue was purified by silica gel chromatography (PE:EtOAc=20:1) to give 2.4 g (8.0 mmol) of the title compound. The yield was 65.4%.

LCMS [M+Na]⁺ calcd for C₁₆H₂₉NO₄, 299.21; found, 322.25.

### Step 5

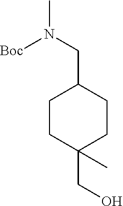 [SMILES: CN(CC1CCC(C)(CO)CC1)C(=O)OC(C)(C)C]

Under nitrogen protection, to a solution of methyl 4-(((tert-butoxycarbonyl)(methyl)amino)methyl)-1-methylcyclohexanecarboxylate (2.4 g, 8.0 mmol) in anhydrous THE (80.0 mL) at 0° C. was added DIBAL-H (3.4 g, 24.0 mmol) dropwise over 5 mins. The reaction was stirred at 0° C. for 2 hr. After the reaction was completed as indicated by TLC, the reaction was quenched with water (100.0 mL) and then the mixture was filtered through a Celite pad and the filtrate was extracted with EtOAc (50.0 mL×2). The combined organic layer was washed with brine, dried over anhydrous Na₂SO₄, filtered, and concentrated under reduced pressure. The residue was purified by silica gel chromatography (PE:EtOAc=8:1) to give 1.3 g (4.8 mmol) of the title compound. The yield was 59.8%.

LCMS [M+Na]⁺ calcd for C₁₅H₂₉NO₃, 271.21; found, 294.27.

### Step 6

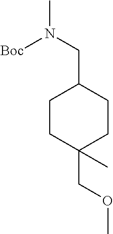 [SMILES: COCC1(C)CCC(CN(C)C(=O)OC(C)(C)C)CC1]

Under nitrogen protection, to a solution of tert-butyl ((4-(hydroxymethyl)-4-methylcyclohexyl)methyl)(methyl)carbamate (1.3 g, 4.8 mmol) in DMF (48.0 mL) at 0° C. was added NaH (383.2 mg, 9.6 mmol, 60%) portion wise over 5 mins. After the resulting mixture was stirred at 0° C. for 20 mins, iodomethane (1.4 g, 9.6 mmol) was added into the reaction dropwise over 2 mins. The reaction was allowed to warm to rt and stir at rt overnight. After the reaction was completed as indicated by TLC, the reaction was quenched with water (400.0 mL) and extracted with EtOAc (150.0 mL×2). The combined organic layer was washed with brine, dried over anhydrous Na₂SO₄, filtered, and concentrated under reduced pressure. The residue was purified by silica gel chromatography (PE:EtOAc=3:1) to give 290.0 mg (1.0 mmol) of the title compound. The yield was 21.2%.

LCMS [M+Na]⁺ calcd for C₁₆H₃₁NO₃, 285.23; found, 308.66.

### Step 7

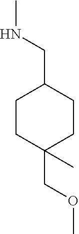

To a solution of tert-butyl ((4-(methoxymethyl)-4-methylcyclohexyl)methyl)(methyl)carbamate (270.0 mg, 1.0 mmol) in DCM (10 mL) was added TFA (3 mL). The reaction was stirred at rt for 3 hrs. After the reaction was completed as indicated by LCMS, the reaction was concentrated under reduced pressure to give 175.0 mg of the crude title compound. The yield was 100%.

LCMS [M+H]⁺ calcd for C₁₁H₂₄NO, 186.18; found, 185.97.

### Step 8

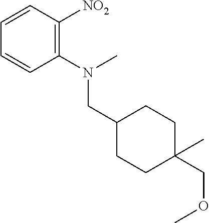 [SMILES: COCC1(C)CCC(CN(C)c2ccccc2[N+](=O)[O-])CC1]

To a solution of 1-(4-(methoxymethyl)-4-methylcyclohexyl)-N-methylmethanamine (175.0 mg, 0.9 mmol) in DMAc (9.0 mL) was added 1-fluoro-2-nitrobenzene (266.5 mg, 1.8 mmol) and K₂CO₃ (522.1 mg, 3.8 mmol). The reaction was stirred at 130° C. for 5 hr. After the reaction was completed as indicated by TLC, the reaction was quenched with water (20.0 mL) and extracted with EtOAc (15.0 mL×2). The combined organic layer was washed with brine, dried over anhydrous Na₂SO₄, filtered, and concentrated under reduced pressure. The residue was purified by silica gel chromatography (PE:EtOAc=100:1 to 50:1) to give 330.0 mg (1.1 mmol) of the title compound. The yield was 114.1%.

LCMS [M+H]⁺ calcd for C₁₇H₂₇N₂O₃, 307.15; found, 307.15.

### Step 9

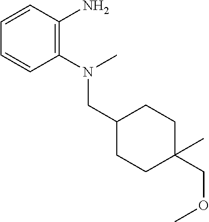 [SMILES: COCC1(C)CCC(CN(C)c2ccccc2N)CC1]

To a solution of N-((4-(methoxymethyl)-4-methylcyclohexyl)methyl)-N-methyl-2-nitroaniline (310.0 mg, 1.0 mmol) in MeOH (10.0 mL) was added Pd/C (30 mg, 10%). The suspension was evacuated and then refilled with hydrogen for three times. The reaction mixture was then stirred under hydrogen (balloon) for 3 hr. After the reaction was completed as indicated by TLC, the suspension was filtered through a pad of Celite and the filtered cake was washed with MeOH (10 mL). The combined filtrate was concentrated under reduced pressure to give 190.0 mg of the crude title compound. The yield was 67.9%.

LCMS [M+H]⁺ calcd for C₁₇H₂₉N₂O, 277.22; found, 277.12.

### Step 10

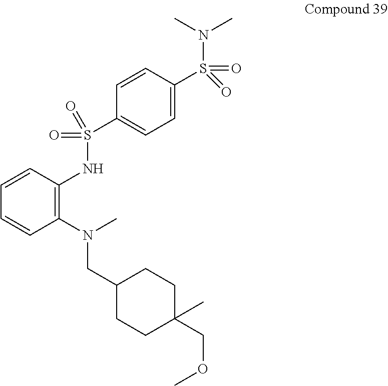 [SMILES: COCC1(C)CCC(CN(C)c2ccccc2NS(=O)(=O)c2ccc(S(=O)(=O)N(C)C)cc2)CC1]

To a solution of N1-((4-(methoxymethyl)-4-methylcyclohexyl)methyl)-N1-methylbenzene-1,2-diamine (177.0 mg, 0.6 mmol) in THE (6.0 mL) was added 4-(N,N-dimethylsulfamoyl)benzene-1-sulfonyl chloride (236.2 mg, 0.8 mmol) and pyridine (202.6 mg, 2.6 mmol). The reaction mixture was stirred at 60° C. overnight. After the reaction was completed as indicated by TLC, the reaction was quenched with water (15.0 mL) and extracted with EtOAc (10.0 mL×2). The combined organic layer was washed with brine, dried over anhydrous Na₂SO₄, filtered, and concentrated under reduced pressure. The residue was purified by silica gel chromatography to give the impure title compound. The impure product was further purified by preparative HPLC to give 33.8 mg of the title compound. The yield was 10.1%.

¹H-NMR (300 MHz, CDCl₃) δ 8.32 (br, 1H), 8.01 (d, J=8.1 Hz, 2H), 7.82 (d, J=8.1 Hz, 2H), 7.56 (d, J=7.8 Hz, 1H), 7.12-7.02 (m, 3H), 3.34 (s, 3H), 3.17 (s, 2H), 2.71 (s, 6H), 2.55 (d, J=6.6 Hz, 2H), 2.31 (s, 3H), 1.65-1.61 (m, 2H), 1.53-1.47 (m, 2H), 1.33-1.17 (m, 2H), 1.03 (d, J=9.3 Hz, 3H), 0.90 (s, 3H). LCMS [M+H]⁺ calcd for C₂₆H₃₈N₃S₂O₅, 524.22; found, 523.89.

The following compound was prepared according to the synthesis of Compound 39.

| Structure | Name | Compound No. | Analytical data |
|---|---|---|---|
| 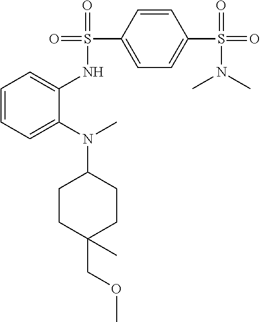 [SMILES: COCC1(C)CCC(N(C)c2ccccc2NS(=O)(=O)c2ccc(S(=O)(=O)N(C)C)cc2)CC1] | N1-(2-((4- (methoxymethyl)-4- methylcyclohexyl) (methyl)amino)phenyl)- N4,N4- dimethylbenzene-1,4- disulfonamide | Compound 42 | ¹H-NMR (300 MHz, CDCl₃) δ 8.57-8.26 (br s, 1H), 8.01 (d, J = 8.4 Hz, 2H), 7.81 (d, J = 8.4 Hz, 2H), 7.61 (d, J = 7.5 Hz, 1H), 7.14-7.09 (m, 2H), 7.06- 7.01 (m, 1H), 3.37 (s, 3H), 3.15 (s, 2H), 2.70 (s, 6H), 2.58-2.47 (m, 1H), 2.28 (s, 3H), 1.62- 1.48 (m, 3H), 1.25-1.17 (m, 3H), 1.00-0.91 (m, 2H), 0.87 (s, 3H). LCMS [M + H]⁺ calcd for C₂₄H₃₆N₃O₅S₂, 510.2; found, 509.82. |

### Example S23. Synthesis of Compound 53

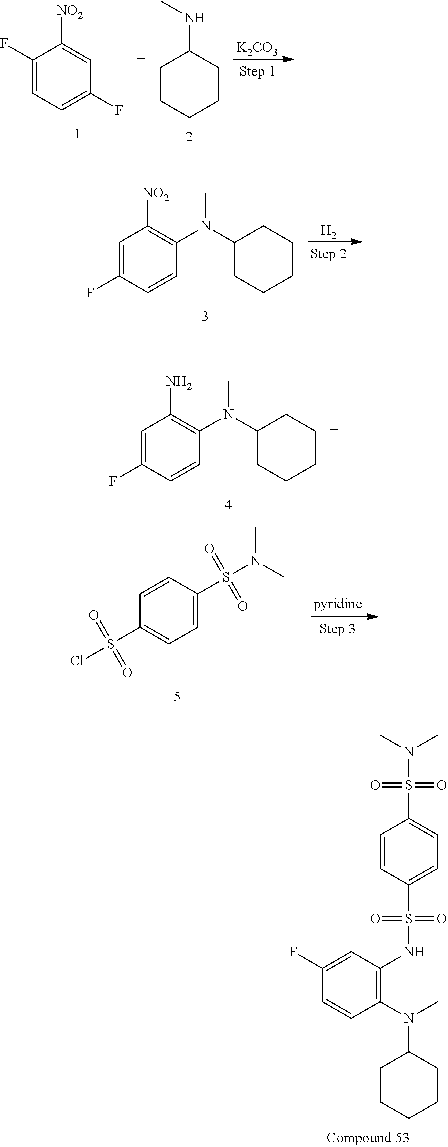

### Step 1

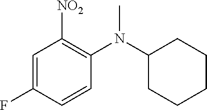 [SMILES: CN(c1ccc(F)cc1[N+](=O)[O-])C1CCCCC1]

To a solution of 1,4-difluoro-2-nitrobenzene (330.0 mg, 2.0 mmol) in DMF (15.0 mL) was added N-methylcyclohexanamine (352.2 mg, 3.1 mmol) and K₂CO₃ (1.2 g, 8.3 mmol). The reaction was stirred at 140° C. for 4 hr. After the reaction was completed as indicated by TLC, the reaction was quenched with water (30.0 mL) and extracted with EtOAc (20.0 mL×2). The combined organic layer was washed with brine, dried over anhydrous Na₂SO₄, filtered, and concentrated under reduced pressure. The residue was purified by silica gel chromatography (PE=100%) to give 500.0 mg (2.0 mmol) of the title compound. The yield was 95.5%.

LCMS [M+H]⁺ calcd for C₁₃H₁₈FN₂O₂, 253.13; found, 253.31.

### Step 2

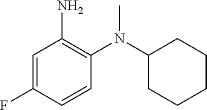 [SMILES: CN(c1ccc(F)cc1N)C1CCCCC1]

To a solution of N-cyclohexyl-4-fluoro-N-methyl-2-nitroaniline (500.0 mg, 2.0 mmol) in MeOH (19.0 mL) was added Pd/C (50 mg, 10%). The suspension was evacuated and then refilled with hydrogen for three times. The reaction mixture was then stirred under hydrogen from a balloon for 3 hr. After the reaction was completed as indicated by TLC, the suspension was filtered through a pad of Celite and the filtered cake was washed with MeOH (10 mL). The combined filtrate was concentrated under reduced pressure to give 400.0 mg of the crude title compound. The yield was 90.8%.

LCMS [M+H]⁺ calcd for C₁₃H₂₀FN₂, 223.15; found, 223.30.

### Step 3

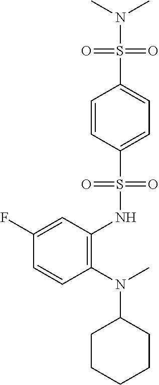 [SMILES: CN(c1ccc(F)cc1NS(=O)(=O)c1ccc(S(=O)(=O)N(C)C)cc1)C1CCCCC1]

To a solution of N1-cyclohexyl-4-fluoro-N1-methylbenzene-1,2-diamine (220.0 mg, 1.0 mmol) in THE (10.0 mL) was added 4-(N,N-dimethylsulfamoyl)benzene-1-sulfonyl chloride (365.1 mg, 1.3 mmol) and pyridine (234.8 mg, 3.0 mmol). The reaction mixture was stirred at 60° C. overnight. After the reaction was completed as indicated by TLC, the reaction was quenched with water (25.0 mL) and extracted with EtOAc (15.0 mL×2). The combined organic layer was washed with brine, dried over anhydrous Na₂SO₄, filtered, and concentrated under reduced pressure. The residue was purified by silica gel chromatography to give the impure title compound. The impure product was further purified by preparative HPLC to give 80.0 mg of the title compound. The yield was 17.2%.

¹H-NMR (300 MHz, CDCl₃) δ 8.01 (d, J=8.1 Hz, 2H), 7.85 (d, J=8.1 Hz, 2H), 7.39 (dd, J=2.7 Hz, 10.2 Hz, 1H), 7.07 (dd, J=6 Hz, 9 Hz, 1H), 6.76-6.70 (m, 1H), 2.72 (s, 6H), 2.48-2.37 (m, 1H), 2.33 (s, 3H), 1.71-1.60 (m, 4H), 1.11-0.88 (m, 6H); LCMS [M+H]⁺ calcd for C₂₁H₂₉FN₃S₂O₄, 470.15; found, 470.47.

The following compound was prepared according to the synthesis of Compound 53.

| | | Compound | |
|---|---|---|---|
| Structure | Name | No. | Analytical data |
| 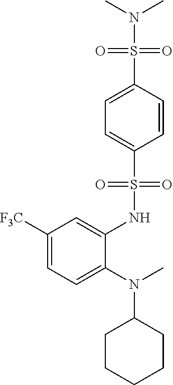 | N1-(2- (cyclohexyl(methyl)amino)- 5-(trifluoromethyl)phenyl)- N4,N4-dimethylbenzene- 1,4-disulfonamide | Compound 110 | ¹H NMR (300 MHz, CDCl₃) δ 8.14 (d, J = 6.9 Hz, 2H), 8.01-7.80 (m, 3H), 7.22-7.13 (m, 1H), 7.39-7.26 (m, 1H), 2.71 (s, 6H), 2.61-2.30 (m, 4H), 1.82-1.50 (m, 5H), 1.19- 0.98 (m, 5H); LCMS = 520.4 [M + H]⁺ |

### Example S24. Synthesis of Compound 52

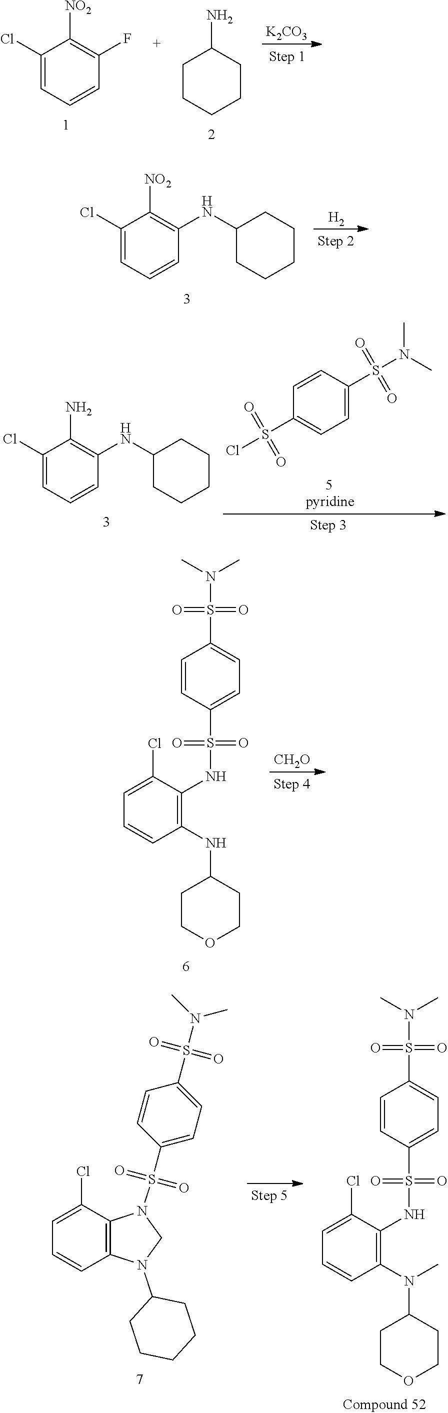

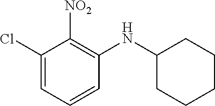 [SMILES: O=[N+]([O-])c1c(Cl)cccc1NC1CCCCC1]

To a solution of 1-chloro-3-fluoro-2-nitrobenzene (300.0 mg, 1.7 mmol) in DMF (15.0 mL) was added cyclohexanamine (254.0 mg, 2.6 mmol) and K₂CO₃ (709.0 mg, 5.1 mmol). The reaction mixture was stirred at 140° C. for 2 hr. After the reaction was completed as indicated by TLC, the reaction was quenched with water (30.0 mL) and extracted with EtOAc (30.0 mL×2). The combined organic layer was washed with water and brine, dried over anhydrous Na₂SO₄, filtered, and concentrated under reduced pressure. The residual oil was purified by silica gel chromatography (PE) to give 415.0 mg of the title compound. The yield was 95.3%.

LCMS [M+H]⁺ calcd for C₁₂H₁₆ClN₂O₂, 255.08; found, 255.30.

### Step 2

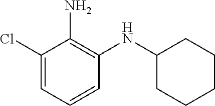 [SMILES: Nc1c(Cl)cccc1NC1CCCCC1]

To a solution of 3-chloro-N-cyclohexyl-2-nitroaniline (390.0 mg, 1.5 mmol) in an aqueous saturated NH₄Cl solution (7.0 mL) and EtOH (7.0 mL) was added Fe powder (257.0 mg, 4.6 mmol) in one portion. The reaction mixture was stirred at 70° C. for 2 hr. After the reaction was completed as indicated by TLC, the suspension was cooled to rt and filtered to remove the undissolved solid. The filtrate was quenched with water (15.0 mL) and extracted with EtOAc (15.0 mL×2). The combined organic layers were washed with water and brine, dried over anhydrous Na₂SO₄, filtered, and concentrated under reduced pressure. The residue was purified by silica gel chromatography (PE:EtOAc=50:1) to give 243.0 mg of the title compound. The yield was 70.6%.

LCMS [M+H]⁺ calcd for C₁₂H₁₈ClN₂, 225.11; found, 225.

### Step 3

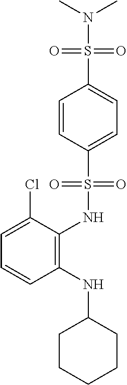 [SMILES: CN(C)S(=O)(=O)c1ccc(S(=O)(=O)Nc2c(Cl)cccc2NC2CCCCC2)cc1]

To a solution of 3-chloro-N1-cyclohexylbenzene-1,2-diamine (220.0 mg, 1.0 mmol) in THE (10 mL) was added the 4-(N,N-dimethylsulfamoyl)benzenesulfonyl chloride (416.0 mg, 1.5 mmol) and pyridine (233.0 mg, 2.9 mmol). The reaction mixture was stirred at 60° C. overnight. After the reaction was completed as indicated by TLC, the reaction was quenched with water (10.0 mL) and extracted with EtOAc (10.0 mL×2). The combined organic layer was washed with brine, dried over anhydrous Na₂SO₄, filtered, and concentrated under reduced pressure. The residue was purified by silica gel chromatography (PE:EtOAc=10:1 to 5:1) to give 150.0 mg of the title compound. The yield was 32.06%.

LCMS [M+H]⁺ calcd for C₂₀H₂₇ClN₃O₄S₂, 472.11; found, 472.45.

### Step 4

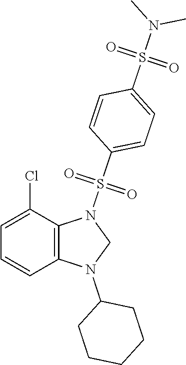 [SMILES: CN(C)S(=O)(=O)c1ccc(S(=O)(=O)N2CN(C3CCCCC3)c3cccc(Cl)c32)cc1]

To a solution of 4-((7-chloro-3-cyclohexyl-2,3-dihydro-1H-benzo[d]imidazol-1-yl)sulfonyl)-N,N-dimethylbenzenesulfonamide (120.0 mg, 0.3 mmol) in THE (2.5 mL) was added formaldehyde (15.3 mg, 0.5 mmol, 37%). The reaction mixture was stirred at ambient temperature for 2 hr. After the reaction was completed as indicated by TLC, the reaction was quenched with water (10.0 mL) and extracted with DCM (10.0 mL×2). The combined organic layer was dried over anhydrous Na₂SO₄, filtered, and concentrated under reduced pressure to give 100 mg of the crude title compound. The crude title compound was used for the next step without purification.

LCMS [M+H]⁺ calcd for C₂₁H₂₇ClN₃O₄S₂, 484.11; found, 483.99.

### Step 5

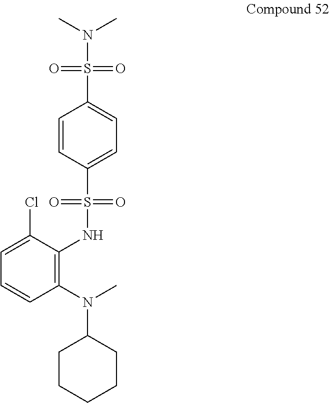 [SMILES: CN(c1cccc(Cl)c1NS(=O)(=O)c1ccc(S(=O)(=O)N(C)C)cc1)C1CCCCC1]

To a solution of 4-((7-chloro-3-cyclohexyl-2,3-dihydro-1H-benzo[d]imidazol-1-yl)sulfonyl)-N,N-dimethylbenzenesulfonamide (100.0 mg, 0.2 mmol) in THE (2.0 mL) was added NaBH₄ (23.0 mg, 0.8 mmol) in four portions over 30 mins. The reaction mixture was stirred at ambient temperature for 2 hr. After the reaction was completed as indicated by TLC, the reaction was quenched with water (10.0 mL) and extracted with EtOAc (10.0 mL×2). The combined organic layer was washed with brine, dried over anhydrous Na₂SO₄, filtered, and concentrated under reduced pressure. The resulting oil was purified by silica gel chromatography to give the impure title compound. The impure title compound was further purified by preparative HPLC to give 22.7 mg of the title compound. The yield was 22.3%.

¹H-NMR (300 MHz, CDCl₃) δ 8.11 (d, J=8.4 Hz, 2H), 7.89 (d, J=8.4 Hz, 2H), 7.08 (s, 3H), 2.86-2.82 (m, 1H), 2.75 (s, 6H), 2.58 (s, 3H), 1.84-1.77 (m, 4H), 1.77-1.64 (m, 1H), 1.36-1.26 (m, 4H), 1.19-1.09 (m, 1H). LCMS [M+H]⁺ calcd for C₂₁H₂₉ClN₃O₄S₂, 486; found, 486.02.

The following compounds were prepared according to the synthesis of Compound 52.

| | | Compound | |
|---|---|---|---|
| Structure | Name | No. | Analytical data |
| 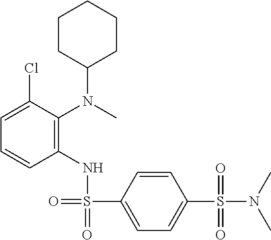 [SMILES: CN(c1c(Cl)cccc1NS(=O)(=O)c1ccc(S(=O)(=O)N(C)C)cc1)C1CCCCC1] | N1-(3-chloro-2- (cyclohexyl(methyl)a- mino)phenyl)-N4,N4- dimethylbenzene-1,4- disulfonamide | Compound 38 | ¹H-NMR (300 MHz, CD3OD) δ 8.08 (d, J = 8.1 Hz, 2H), 7.92 (d, J = 8.1 Hz, 2H), 7.65 (d, J = 7.8 Hz, 1H), 7.18 (t, J = 7.8 Hz, 1H), 7.05 (d, J = 7.8 Hz, 1H), 3.12-3.05 (m, 1H), 2.67 (s, 6H), 2.51 (s, 3H), 2.28-2.24 (m, 1H), 1.81-1.77 (m, 1H), 1.56-1.54 (m, 1H), 1.50-1.42 (m, 1H), 1.34-1.28 (m, 1H), 1.20-1.00 (m, 4H), 0.25-0.20 (m, 1H); LCMS = 486.02 [M + H]+ |
| 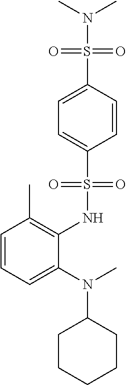 [SMILES: Cc1cccc(N(C)C2CCCCC2)c1NS(=O)(=O)c1ccc(S(=O)(=O)N(C)C)cc1] | N1-(2- (cyclohexyl(methyl)a- mino)-6- methylphenyl)- N4,N4- dimethylbenzene-1,4- disulfonamide | Compound 36 | ¹H-NMR (300 MHz, CD3OD) δ 7.98 (d, J = 8.4 Hz, 2H), 7.91 (d, J = 8.4 Hz, 2H), 7.11 (t, J = 7.8 Hz, 1H), 6.97-6.93 (m, 2H), 2.73 (s, 6H), 2.70-2.65 (m, 1H), 2.28 (s, 3H), 2.24(s, 3H), 1.66- 1.63 (m, 2H), 1.56-1.52 (m, 1H), 1.41-1.37 (m, 2H), 1.21- 0.98 (m, 5H); LCMS = 465.86 [M + H]+ |
| 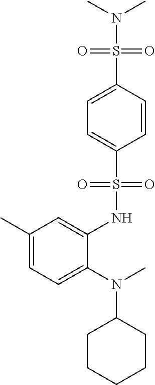 [SMILES: Cc1ccc(N(C)C2CCCCC2)c(NS(=O)(=O)c2ccc(S(=O)(=O)N(C)C)cc2)c1] | N1-(2- (cyclohexyl(methyl)a- mino)-5- methylphenyl)- N4,N4- dimethylbenzene-1,4- disulfonamide | Compound 117 | ¹H-NMR (300 MHz, CD3OD) δ 8.03 (d, J = 8.1 Hz, 2H), 7.89 (d, J = 8.1 Hz, 2H), 7.44 (s, 1H), 7.05 (d, J = 7.8 Hz, 1H), 6.88 (d, J = 7.8 Hz, 1H), 2.67 (s, 6H), 2.54-2.32 (m, 1H), 2.32 (s, 3H), 2.30 (s, 3H), 1.71-1.55 (m, 5H), 1.26-0.98 (m, 5H); LCMS = 465.86 [M + H]+ |
| 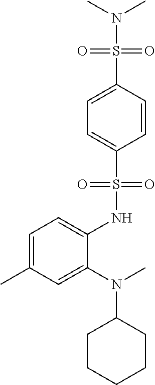 [SMILES: Cc1ccc(NS(=O)(=O)c2ccc(S(=O)(=O)N(C)C)cc2)c(N(C)C2CCCCC2)c1] | N1-(2- (cyclohexyl(methyl)a- mino)-4- methylphenyl)- N4,N4- dimethylbenzene-1,4- disulfonamide | Compound 35 | ¹H NMR (300 MHz, CDCl₃) δ 8.51-8.21 (br s, 1H), 7.97 (d, J = 8.1 Hz, 2H), 7.81 (d, J = 8.1 Hz, 2H), 7.52 (d, J = 7.8 Hz, 1H), 6.91 (d, J = 12.3 Hz, 2H), 2.70 (s, 6H), 2.47-2.39 (m, 1H), 2.26 (s, 6H), 1.65-1.56 (m, 5H), 1.10-0.97 (m, 5H); LCMS = 465.86 [M + H]+ |
| 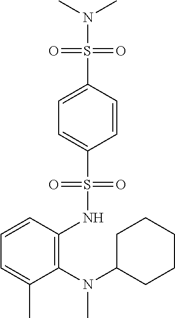 [SMILES: Cc1cccc(NS(=O)(=O)c2ccc(S(=O)(=O)N(C)C)cc2)c1N(C)C1CCCCC1] | N1-(2- (cyclohexyl(methyl)a- mino)-3- methylphenyl)- N4,N4- dimethylbenzene-1,4- disulfonamide | Compound 98 | ¹H NMR (300 MHz, CDCl₃) δ 8.86 (br, 1H ), 7.99 (d, J = 8.4 Hz, 2H), 7.81 (d, J = 8.4 Hz, 2H), 7.49 (d, J = 8.1 Hz, 1H), 7.04 ( t, J = 7.8 Hz, 1 H), 6.78 (d, J = 8.1 Hz, 1H), 2.83-2.79 (m, 1H), 2.71 (s, 6H), 2.45 (s, 3H), 2.30-2.28 (m, 1H), 2.26 (s, 3H), 1.81-1.77 (m, 1H), 1.65- 1.57 (m, 1H), 1.50-1.45 (m, 1H), 1.29-1.25 (m, 1H), 1.21- 1.02 (m, 4H), 0.41-0.33 (m, 1H); LCMS = 466.00 [M + H]+; |
| 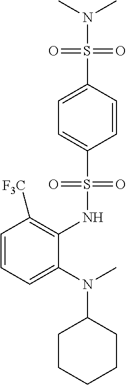 [SMILES: CN(c1cccc(C(F)(F)F)c1NS(=O)(=O)c1ccc(S(=O)(=O)N(C)C)cc1)C1CCCCC1] | N1-(2- (cyclohexyl(methyl)a- mino)-6- (trifluoromethyl)phe- nyl)-N4,N4- dimethylbenzene-1,4- disulfonamide | Compound 92 | ¹H-NMR (300 MHz, CDCl₃) δ 8.02 (d, J = 8.4 Hz, 2H), 7.89 (d, J = 8.4 Hz, 2H), 7.47-7.35 (m, 3H), 2.75 (s, 6H), 2.65-2.58 (m, 1H), 2.42 (s, 3H), 1.76-1.64 (m, 2H), 1.51-1.46 (m, 2H), 1.27-0.91 (m, 6H); LCMS = 519.97 [M + H]+ |
| 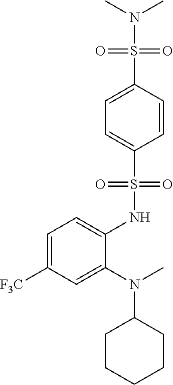 | N1-(2- (cyclohexyl(methyl)a- mino)-4- (trifluoromethyl)phe- nyl)-N4,N4- dimethylbenzene-1,4- disulfonamide | Compound 116 | ¹H-NMR (300 MHz, CDCl₃) δ 8.69-8.21 (br s, 1H), 8.03 (d, J = 8.1 Hz, 2H), 7.86 (d, J = 8.4 Hz, 2H), 7.69 (d, J = 8.1 Hz, 1H), 7.37 (d, J = 9.6 Hz, 2H) 2.72 (s, 6H), 2.48-2.43 (m, 1H), 1.70- 1.67 (m, 4H), 1.14-0.85 (m, 6H); LCMS = 519.90 [M + H]+ |
| 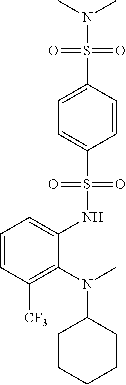 [SMILES: CN(c1c(NS(=O)(=O)c2ccc(S(=O)(=O)N(C)C)cc2)cccc1C(F)(F)F)C1CCCCC1] | N1-(2- (cyclohexyl(methyl) amino)-3- (trifluoromethyl)phe- nyl)-N4,N4- dimethylbenzene- 1,4-disulfonamide | Compound 109 | ¹H-NMR (300 MHZ, CDCl₃) δ 8.84 (s, 1H ), 8.02 (d, J = 8.1 Hz, 2H), 7.85 (d, J = 8.1 Hz, 2H), 7.85 (s, 1H), 7.35-7.30 (m, 2H), 2.85-2.82 ( m, 1 H), 2.71 (s, 6H), 2.47 (s, 3H), 2.38-2.28 (m, 1H), 1.82-1.78 (m, 1H), 1.60-1.57 (m, 1H), 1.49-1.42 (m, 2H), 1.33-1.03 (m, 4H), 0.40-0.33 (m, 1H); LCMS = 519.83 [M + H]+ |
| 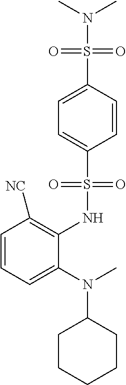 [SMILES: CN(c1cccc(C#N)c1NS(=O)(=O)c1ccc(S(=O)(=O)N(C)C)cc1)C1CCCCC1] | N1-(2-cyano-6- (cyclohexyl(methyl)a- mino)phenyl)-N4,N4- dimethylbenzene-1,4- disulfonamide | Compound 86 | ¹H-NMR (300 MHz, CDCl₃) δ 8.15 (d, J = 8.1 Hz, 2H), 7.91 (d, J = 8.1 Hz, 2H), 7.48-7.35 (m, 2H), 7.25-7.20 (m, 1H), 2.73 (s, 6H), 2.61 (s, 3H), 1.82- 1.73 (m, 4H), 1.72-1.56 (m, 2H), 1.56-1.41 (m, 4H), 1.19- 1.01 (m, 1H); LCMS = 477.3 [M + H]+ |
| 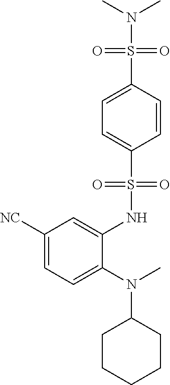 | N1-(5-cyano-2- (cyclohexyl(methyl) amino)phenyl)- N4,N4- dimethylbenzene- 1,4-disulfonamide | Compound 89 | ¹H-NMR (300 MHz, CDCl₃) δ 8.25 (br, 1H), 8.03 (d, J = 8.4 Hz, 2H), 7.90-7.87 (m, 3H), 7.35 (d, J = 8.1 Hz, 1H), 7.18 (d, J = 8.1 Hz, 1 H), 2.74 (s, 6H), 2.55-2.45 (m, 1H), 2.44 (s, 3H), 1.75-1.61 (m, 5H), 1.21-0.93 (m, 5H); LCMS = 476.64 [M + H]+ |
| 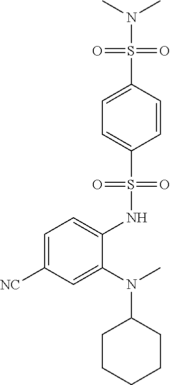 | N1-(4-cyano-2- (cyclohexyl(methyl)a- mino)phenyl)-N4,N4- dimethylbenzene-1,4- disulfonamide | Compound 88 | ¹H-NMR (300 MHz, CDCl₃) δ 8.69-8.21 (br s, 1H), 8.04 (d, J = 8.4 Hz, 2H), 7.88 (d, J = 8.4 Hz, 2H), 7.67 (d, J = 8.4 Hz, 1H), 7.41 (d, J = 8.1 Hz, 2H) 2.73 (s, 6H), 2.45 (s, 4H), 1.71-1.68 (m, 5H), 1.20-1.07 (m, 5H); LCMS = 477.38 [M + H]+ |
| 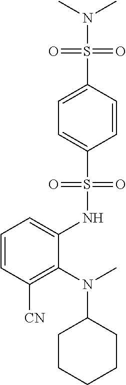 [SMILES: CN(c1c(C#N)cccc1NS(=O)(=O)c1ccc(S(=O)(=O)N(C)C)cc1)C1CCCCC1] | N1-(3-cyano-2- (cyclohexyl(methyl)a- mino)phenyl)-N4,N4- dimethylbenzene-1,4- disulfonamide | Compound 91 | ¹H-NMR (300 MHz, CDCl₃) δ 8.57 (br, 1H), 8.01 (d, J = 8.4 Hz, 2H), 7.87 (m, 3H), 7.34- 7.29 (m, 1H), 7.25-7.20 (m, 1H), 3.13-2.98 (m, 1H), 2.73 (s, 6H), 2.62 (s, 3H), 1.77-1.56 (m, 4H), 1.31-0.76 (m, 6H); LCMS = 477.38 [M + H]+ |
| 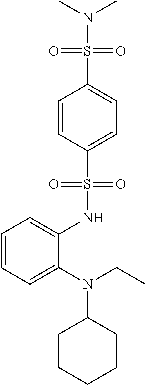 [SMILES: CCN(c1ccccc1NS(=O)(=O)c1ccc(S(=O)(=O)N(C)C)cc1)C1CCCCC1] | N1-(2- (cyclohexyl(ethyl)ami- no)phenyl)-N4,N4- dimethylbenzene-1,4- disulfonamide | Compound 60 | ¹H-NMR (300 MHz, CD₃OD) δ 8.09 (d, J = 8.4 Hz, 2H), 7.89 (d, J = 8.4 Hz, 2H), 7.69 (d, J = 7.5 Hz, 1H), 7.21-7.14 (m, 2H), 7.04 (t, J = 7.5 Hz, 1H), 2.99 (q, J = 6.9 Hz, 2H), 2.66 (s, 6H), 2.58-2.50 (m, 1H), 1.74-1.65 (m, 4H), 1.64-1.55 (m, 1H), 1.12-0.95 (m, 5H), 0.62 (t, J = 6.9 Hz, 3H); LCMS = 465.86 ]M + H]+ |

### Example S25. Synthesis of Compound 87

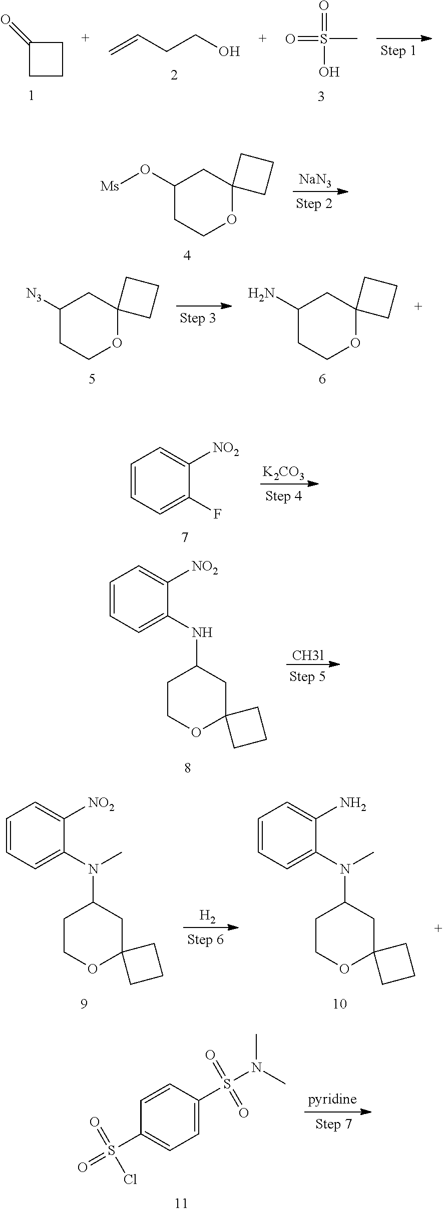

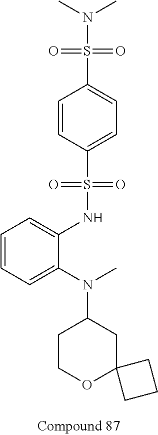 [SMILES: CN(c1ccccc1NS(=O)(=O)c1ccc(S(=O)(=O)N(C)C)cc1)C1CCOC2(CCC2)C1]

### Step 1

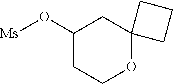 [SMILES: CS(=O)(=O)OC1CCOC2(CCC2)C1]

To a solution of cyclobutanone (2.5 g, 35.7 mmol) and but-3-en-1-ol (2.6 g, 35.7 mmol) in DCM (350.0 mL) was added methanesulfonic acid (6.9 g, 71.3 mmol). The reaction was stirred at rt for 2.5 hr. After the reaction was completed as indicated by TLC, the reaction was quenched with water (300.0 mL) and extracted with DCM (200.0 mL×2). The combined organic layer was washed with brine, dried over anhydrous Na₂SO₄, filtered, and concentrated under reduced pressure. The residue was purified by silica gel chromatography (PE:EtOAc=8:1 to 5:1) to give 6.1 g (27.7 mmol) of the title compound. The yield was 77.6%.

GCMS [M-OMs]calcd for C₈H₁₅O₂, 125.09; found, 125.

### Step 2

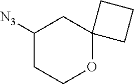 [SMILES: [N-]=[N+]=NC1CCOC2(CCC2)C1]

To a solution of 5-oxaspiro[3.5]nonan-8-yl methanesulfonate (6.0 g, 27.2 mmol) in DMSO (270.0 mL) was added NaN₃ (5.3 g, 81.7 mmol). The reaction was stirred at 80° C. for 4 hr. After the reaction was completed as indicated by TLC, the reaction was quenched with water (500.0 mL) and extracted with EtOAc (200.0 mL×2). The combined organic layer was washed with brine, dried over anhydrous Na₂SO₄, filtered, and concentrated under reduced pressure. The residue was purified by silica gel chromatography (PE:EtOAc=80:1 to 60:1) to give 3.5 g (27.2 mmol) of the title compound. The yield was 76.9%.

Could not be detected by GCMS or LCMS.

### Step 3

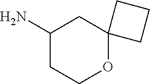

To a solution of 8-azido-5-oxaspiro[3.5]nonane (200.0 mg, 1.2 mmol) in MeOH (12.0 mL) was added Pd/C (20 mg, 10%). The suspension was evacuated and then refilled with hydrogen for three times. The reaction mixture was then stirred under hydrogen (balloon) for 6 hr. After the reaction was completed as indicated by TLC, the suspension was filtered through a pad of Celite and the filtered cake was washed with MeOH (10 mL). The combined filtrate was concentrated under reduced pressure to give 170.0 mg of the crude title compound. The yield was quantitative.

LCMS [M+H]⁺ calcd for C₈H₁₆NO, 142.12; Found, 142.30.

### Step 4

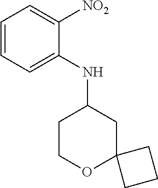 [SMILES: O=[N+]([O-])c1ccccc1NC1CCOC2(CCC2)C1]

This compound was synthesized according to step 1 of the procedure of Synthesis of Compound 30.

LCMS [M+H]⁺ calcd for C₁₄H₁₉N₂O₃, 263.13. found, 263.29.

### Step 5 NO₂

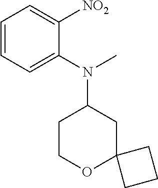 [SMILES: CN(c1ccccc1[N+](=O)[O-])C1CCOC2(CCC2)C1]

This compound was synthesized according to step 2 of the procedure of Synthesis of Compound 30.

LCMS [M+H]⁺ calcd for C₁₅H₂₁N₂O₃, 277.15. found, 276.98.

### Step 6

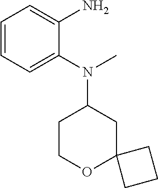 [SMILES: CN(c1ccccc1N)C1CCOC2(CCC2)C1]

This compound was synthesized according to step 3 of the procedure of Synthesis of Compound 30.

LCMS [M+H]⁺ calcd for C₁₅H₂₃N₂O, 247.17. found, 247.02.

### Step 7

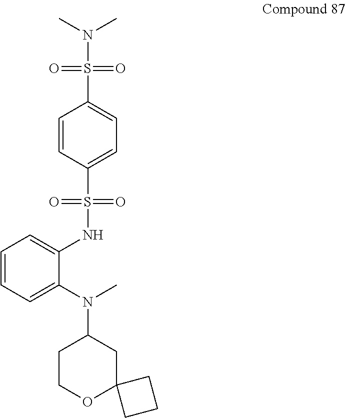 [SMILES: CN(c1ccccc1NS(=O)(=O)c1ccc(S(=O)(=O)N(C)C)cc1)C1CCOC2(CCC2)C1]

This compound was synthesized according to step 4 of the procedure of Synthesis of Compound 30.

¹H-NMR (300 MHz, CDCl₃) δ 8.45 (br, 1H), 8.02 (d, J=8.4 Hz, 2H), 7.83 (d, J=8.4 Hz, 2H), 7.58 (d, J=7.5 Hz, 1H), 7.16-7.06 (m, 3H), 3.75-3.71 (m, 1H), 3.32 (t, J=10.8 Hz, 1H), 2.85-2.73 (m, 1H), 2.71 (s, 6H), 2.35 (s, 3H), 2.12-2.08 (m, 1H), 1.94-1.79 (m, 5H), 1.56-1.45 (m, 2H), 1.38-1.16 (m, 2H). LCMS [M+H]⁺ calcd for C₂₃H₃₂N₃O₅S₂, 494; found, 493.94.

### Example S26. Synthesis of Compound 102

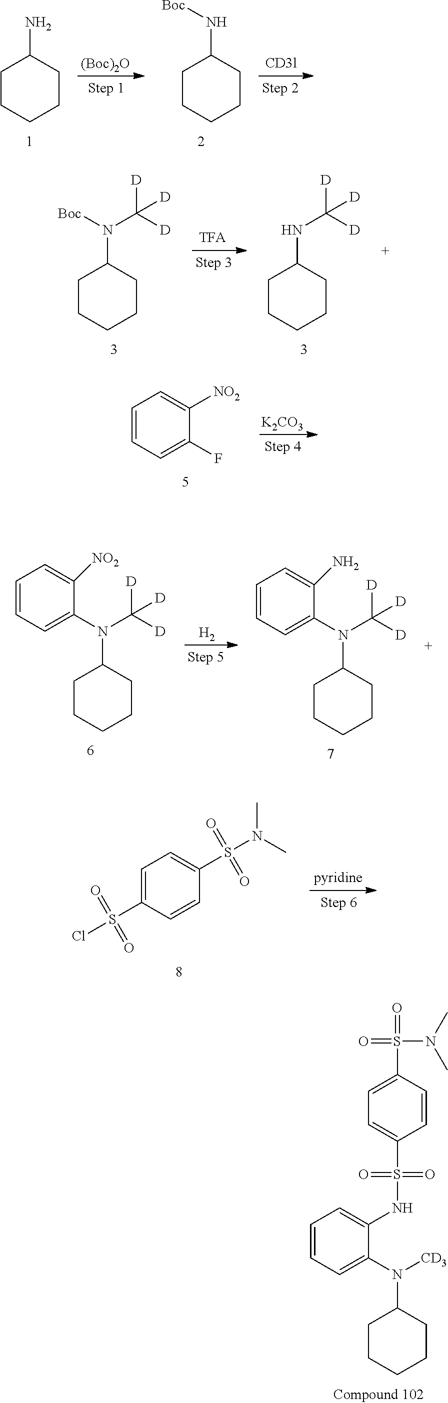

### Step 1

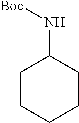 [SMILES: CC(C)(C)OC(=O)NC1CCCCC1]

To a solution of cyclohexanamine (1.0 g, 10.0 mmol) in DCM (100.0 mL) was added DIEA (3.9 g, 30.2 mmol) and (Boc)₂O (3.3 g, 15.1 mmol). The reaction was stirred at rt for 3 hr. After the reaction was completed as indicated by LCMS, the reaction was quenched with water (150.0 mL) and extracted with EtOAc (100.0 mL×2). The combined organic layer was washed with brine, dried over anhydrous Na₂SO₄, filtered, and concentrated under reduced pressure. The residue was purified by silica gel chromatography (PE:EtOAc=50:1) to give 2.0 g (10.0 mmol) of the title compound. The yield was 99.5%.

LCMS [M+H]⁺ calcd for C₁₁H₂₂NO₂, 200.16; found, 200.12.

### Step 2

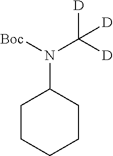 [SMILES: [2H]C([2H])([2H])N(C(=O)OC(C)(C)C)C1CCCCC1]

Under nitrogen protection, to a solution of tert-butyl cyclohexylcarbamate (1.0 g, 5.0 mmol) in DMF (45.0 mL) at 0° C. was added NaH (602.0 mg g, 15.0 mmol, 60%) portion wise over 5 mins. After the resulting mixture was stirred at 0° C. for 30 mins, iodomethane-d₃ (2.2 g, 15.0 mmol) was added into the reaction. The reaction was stirred at rt overnight. After the reaction was completed as indicated by TLC, the reaction was quenched with water (100.0 mL) and extracted with EtOAc (50.0 mL×2). The combined organic layer was washed with brine, dried over anhydrous Na₂SO₄, filtered, and concentrated under reduced pressure. The residue was purified by silica gel chromatography (PE:EtOAc=100:1) to give 1.0 g (4.6 mmol) of the title compound. The yield was 92.1%.

LCMS [M+Na]⁺ calcd for C₁₂H₂D₃NO₂, 216.19; found 239.11.

### Step 3

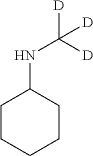 [SMILES: [2H]C([2H])([2H])NC1CCCCC1]

To a solution of tert-butyl cyclohexyl(methyl-d3)carbamate (1.0 g, 4.6 mmol) in DCM (30.0 mL) was added TFA (10.0 mL). The reaction was stirred at rt for 3 hr. After the reaction was completed as indicated by LCMS, the reaction was concentrated under reduced pressure to give 540.0 mg (4.6 mmol) of the title compound. The yield was 100.0%.

LCMS [M+H]⁺ calcd for C₇H₁₃D₃N, 117.14; found, 116.89.

### Step 4

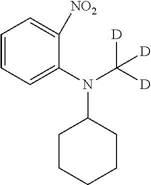 [SMILES: [2H]C([2H])([2H])N(c1ccccc1[N+](=O)[O-])C1CCCCC1]

This compound was synthesized according to step 2 of the procedure of Synthesis of Compound 30.

LCMS [M+H]⁺ calcd for C₁₃H₁₆D₃N₂O₂, 238.16; found, 238.05.

### Step 5

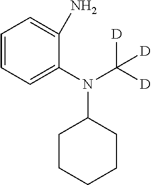 [SMILES: [2H]C([2H])([2H])N(c1ccccc1N)C1CCCCC1]

This compound was synthesized according to step 3 of the procedure of Synthesis of Compound 30.

LCMS [M+H]⁺ calcd for C₁₃H₁₈D₃N₂, 208.18; found, 208.33.

### Step 6

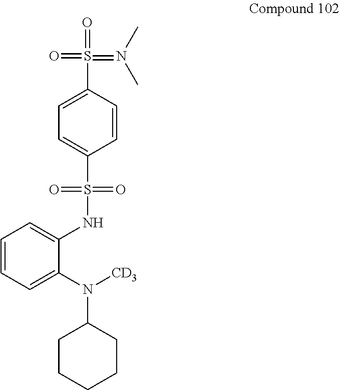

This compound was synthesized according to step 4 of the procedure of Synthesis of Compound 30.

¹H-NMR (300 MHz, CDCl₃) δ 8.56 (br, 1H), 8.00 (d, J=8.1 Hz, 2H), 7.82 (d, J=8.1 Hz, 2H), 7.62 (d, J=7.5 HZ, 1H), 7.14-7.01 (m, 3H), 2.70 (s, 6H), 2.47-2.43 (m, 1H), 1.67-1.56 (m, 5H), 1.07-0.91 (m, 5H); LCMS [M+H]⁺ calcd for C₂₁H₂₇D₃N₃O₄S₂, 455; found, 455.01.

### Example S27. Synthesis of Compound 55

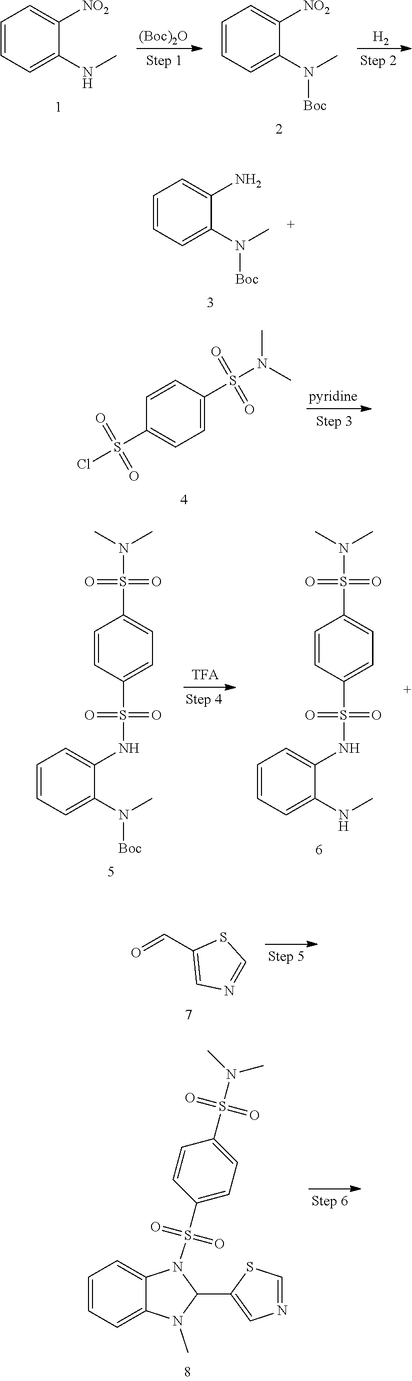

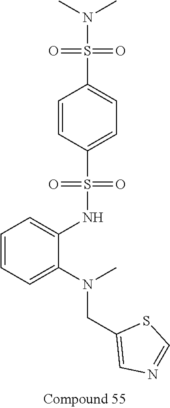 [SMILES: CN(Cc1cncs1)c1ccccc1NS(=O)(=O)c1ccc(S(=O)(=O)N(C)C)cc1]

### Step 1

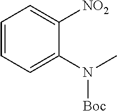 [SMILES: CN(C(=O)OC(C)(C)C)c1ccccc1[N+](=O)[O-]]

To a solution of N-methyl-2-nitroaniline (2.0 g, 13.1 mmol) in THE (20.0 mL) was added DMAP (160.6 mg, 1.3 mmol) and (Boc)₂O (8.6 g, 39.4 mmol). The reaction was stirred at rt overnight. After the reaction was completed as indicated by TLC, the reaction was quenched with water (50.0 mL) and extracted with EtOAc (50.0 mL×2). The combined organic layer was washed with brine, dried over anhydrous Na₂SO₄, filtered, and concentrated under reduced pressure. The residue was purified by silica gel chromatography (PE:EtOAc=200:1 to 30:1) to give 3.1 g (12.3 mmol) of the title compound. The yield was 93.5%.

LCMS [M+Na]⁺ calcd for C₁₂H₁₆N₂O₄, 252.27; found, 275.02.

### Step 2

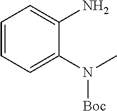 [SMILES: CN(C(=O)OC(C)(C)C)c1ccccc1N]

To a solution of tert-butyl methyl(2-nitrophenyl)carbamate (1.0 g, 4.0 mmol) in MeOH (40.0 mL) was added Pd/C (100 mg, 10%). The suspension was evacuated and then refilled with hydrogen for three times. The reaction mixture was then stirred under hydrogen (balloon) for 3 hr. After the reaction was completed as indicated by TLC, the suspension was filtered through a pad of Celite and the filtered cake was washed with MeOH (20 mL). The combined filtrate was concentrated under reduced pressure. The residue was purified by silica gel chromatography (PE:EtOAc=10:1 to 5:1) to give 830.0 mg (3.7 mmol) of the title compound. The yield was 94.2%.

¹H-NMR (300 MHz, CDCl₃) δ 7.20-6.92 (m, 4H), 6.61 (br, 2H), 3.16 (s, 3H), 1.77-0.88 (br, 9H).

### Step 3

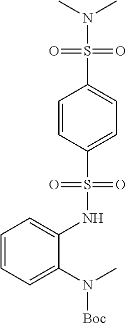 [SMILES: CN(C(=O)OC(C)(C)C)c1ccccc1NS(=O)(=O)c1ccc(S(=O)(=O)N(C)C)cc1]

This compound was synthesized according to step 4 of the procedure of Synthesis of Compound 30.

¹H-NMR (300 MHz, CDCl₃) δ 7.98-7.85 (m, 4H), 7.51-7.42 (m, 1H), 7.30-7.22 (m, 2H), 7.08-7.05 (m, 1H), 2.73 (s, 6H), 2.65 (s, 3H), 1.57-1.33 (m, 9H).

### Step 4

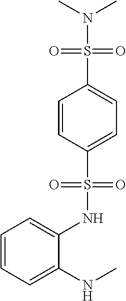 [SMILES: CNc1ccccc1NS(=O)(=O)c1ccc(S(=O)(=O)N(C)C)cc1]

To a solution of tert-butyl (2-((4-(N,N-dimethylsulfamoyl)phenyl)sulfonamido)phenyl)(methyl) carbamate (0.8 g, 1.7 mmol) in DCM (15.0 mL) was added TFA (5.0 mL). The reaction was stirred at rt for 2 hr. After the reaction was completed as indicated by LCMS, the reaction was concentrated under reduced pressure to give 610.0 mg (1.7 mmol) of the title compound. The yield was 96.9%.

LCMS [M+H]⁺ calcd for C₁₅H₂₀N₃O₄S₂, 370.08; found, 369.95.

### Step 5

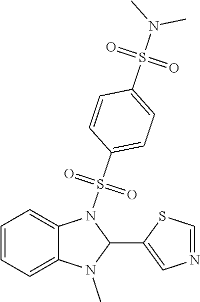 [SMILES: CN1c2ccccc2N(S(=O)(=O)c2ccc(S(=O)(=O)N(C)C)cc2)C1c1cncs1]

To a solution of N1,N1-dimethyl-N4-(2-(methylamino)phenyl)benzene-1,4-disulfonamide (1.0 g, 4.0 mmol) in THE (20.0 mL) was added thiazole-5-carbaldehyde (453.2 mg, 4.0 mmol). The reaction mixture was stirred at 60° C. overnight. After the reaction was completed as indicated by TLC, the reaction was concentrated under reduced pressure. The residue was purified by silica gel chromatography (PE:EtOAc=3:1) to give 700 mg (1.5 mmol) of the title compound. The yield was 75.2%.

¹H-NMR (300 MHz, CDCl₃) δ 8.73 (s, 1H), 7.99 (s, 1H), 7.79 (s, 4H), 7.49 (d, J=7.5 Hz, 1H), 7.06 (t, J=7.5 Hz, 1H), 6.83 (t, J=7.5 Hz, 1H), 6.41 (d, J=7.2 Hz, 2H), 2.70 (s, 6H), 2.50 (s, 3H).

### Step 6

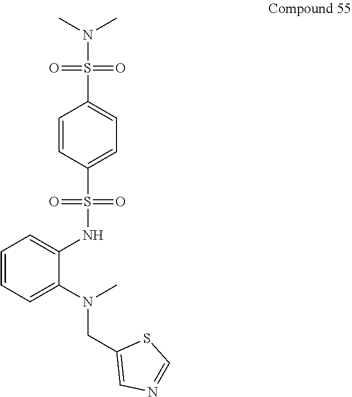 [SMILES: CN(Cc1cncs1)c1ccccc1NS(=O)(=O)c1ccc(S(=O)(=O)N(C)C)cc1]

This compound was synthesized according to step 5 of the procedure of Synthesis of Compound 52.

¹H-NMR (300 MHz, d₆-DMSO) δ 9.53 (s, 1H), 8.91 (s, 1H), 8.05 (d, J=8.4 Hz, 2H), 7.92 (d, J=8.4 Hz, 2H), 7.64 (s, 1H), 7.16 (d, J=7.8 Hz, 1H), 7.07-6.95 (m, 3H), 4.20 (s, 2H), 2.59 (s, 6H), 2.39 (s, 3H). LCMS [M+H]⁺ calcd for C₁₉H₂₃N₄O₄S₃, 467.08; found, 466.77.

### Example S28. Synthesis of Compound 16

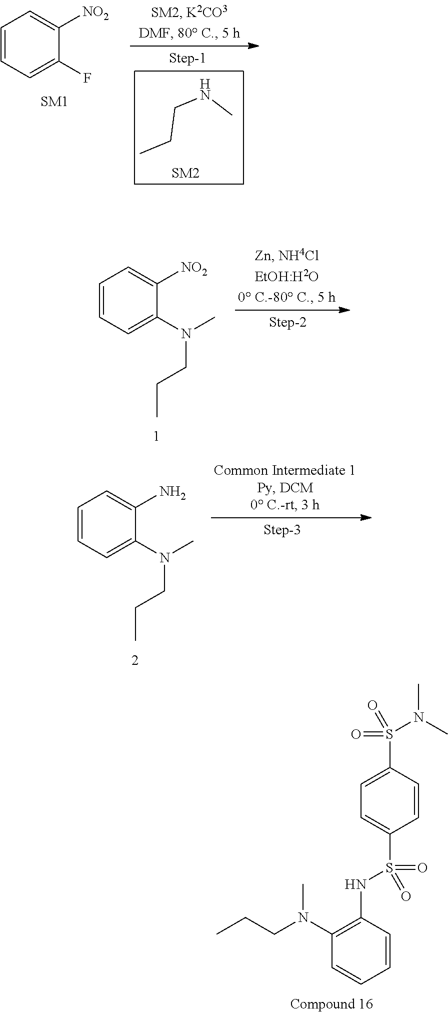

### N-methyl-2-nitro-N-propylaniline (1)

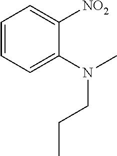

A solution of 1-fluoro-2-nitrobenzene (SM1) (500 mg, 3.54 mmol) in DMF (5 mL) was added to K₂CO₃ (1.2 g, 8.69 mmol) and N-methylpropan-1-amine (SM2) (310 mg, 4.24 mmol) at room temperature. The reaction mixture was heated at 80° C. and stirred for 5 h. After the reaction was completed as indicated by TLC, the reaction mixture was diluted with water (2 mL) and extracted with EtOAc (2×20 mL). The combined organic layers were washed with brine, dried over anhydrous Na₂SO₄, filtered, and concentrated under reduced pressure to give 460 mg of the title compound. The yield was 67%.

¹H NMR (400 MHz, CDCl₃) δ 7.72 (d, J=7.8 Hz, 1H), 7.44-7.32 (m, 1H), 7.06 (d, J=8.3 Hz, 1H), 6.82 (t, J=7.6 Hz, 1H), 3.12 (t, J=7.3 Hz, 2H), 2.81 (s, 3H), 1.67-1.59 (m, 2H), 0.89 (t, J=7.3 Hz, 3H); LCMS [M+H]⁺ calcd for C₁₀H₁₅N₂O₂, 195; found, 195.3

### N1-methyl-N1-propylbenzene-1,2-diamine (2)

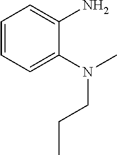

A solution of intermediate 1 (460 mg, 2.37 mmol) in EtOH and H₂O (12 mL, 5:1) was added to Zn dust (1.54 g, 23.71 mmol) and NH₄Cl (1.26 g, 23.71 mmol) at 0° C. The reaction mixture was heated at 80° C. and stirred for 5 h. After the reaction was completed as indicated by TLC, the reaction mixture was filtered through a Celite pad and the filtrate was concentrated under reduced pressure. The crude product was diluted with water (10 mL) and extracted with EtOAc (2×30 mL). The organic layers were washed with brine, dried over anhydrous Na₂SO₄, filtered, and concentrated under reduced pressure to give 420 mg of crude title compound. This material was used in the next step without any further purification.

### N1,N1-dimethyl-N4-(2-(methyl(propyl)amino)phenyl)benzene-1,4-disulfonamide (Compound 16)

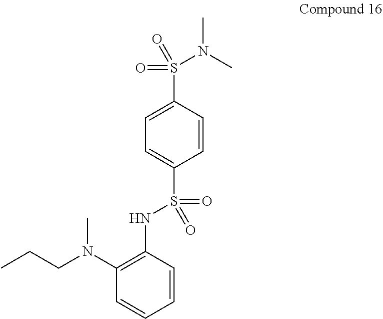 [SMILES: CCCN(C)c1ccccc1NS(=O)(=O)c1ccc(S(=O)(=O)N(C)C)cc1]

A solution of intermediate 2 (350 mg, 2.13 mmol) in CH₂Cl₂ (10 mL) was added to pyridine (0.33 mL, 4.08 mmol) and 4-(N,N-dimethylsulfamoyl)benzenesulfonyl chloride (Common Intermediate 1) (750 mg, 2.64 mmol) at 0° C. The reaction mixture was stirred at room temperature for 3 h. After the reaction was completed as indicated by TLC, the reaction mixture was diluted with water (5 mL) and extracted with CH₂Cl₂ (2×30 mL). The combined organic layers were washed with brine, dried over anhydrous Na₂SO₄, filtered, and concentrated under reduced pressure. The crude product was purified by medium pressure liquid chromatography by eluting with 30-50% EtOAc in heptane to give 80 mg of the title compound. The yield was 9.1%.

¹H NMR (400 MHz, DMSO-d₆) δ 9.22 (br s, 1H), 8.07-8.01 (m, 2H), 7.96-7.89 (m, 2H), 7.23 (dd, J=1.4, 7.9 Hz, 1H), 7.16-7.12 (m, 1H), 7.08 (dt, J=1.5, 7.6 Hz, 1H), 7.04-6.98 (m, 1H), 2.62 (s, 8H), 2.37 (s, 3H), 1.28-1.18 (m, 2H), 0.73 (t, J=7.4 Hz, 3H);

LCMS [M+H]⁺ calcd for C₁₈H₂₆N₃O₄S₂, 412; found, 412.2

The following compounds were prepared according to the synthesis of Compound 16.

| | | Compound | |
|---|---|---|---|
| Structure | Name | No. | Analytical data |
| 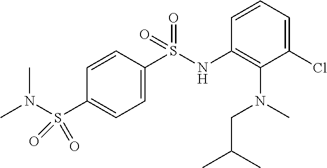 [SMILES: CC(C)CN(C)c1c(Cl)cccc1NS(=O)(=O)c1ccc(S(=O)(=O)N(C)C)cc1] | N1-(3-chloro-2- (isobutyl(methyl)a- mino)phenyl)- N4,N4- dimethylbenzene- 1,4-disulfonamide | Compound 76 | ¹H NMR (400 MHz, DMSO-d₆) δ 9.32 (s, 1H), 8.07 (d, J = 8.3 Hz, 2H), 7.96 (d, J = 8.3 Hz, 2H), 7.22 (d, J = 7.8 Hz, 1H), 7.18-7.06 (m, 2H), 2.74 (br s, 2H), 2.62 (s, 8H), 1.66 - 1.54 (m, 1H), 0.81 (d, J = 6.4 Hz, 7H); LCMS = 460.4 [M + H]⁺ HPLC: 99.58% |
| 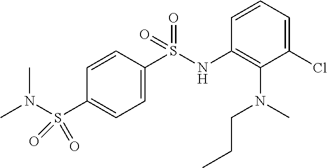 [SMILES: CCCN(C)c1c(Cl)cccc1NS(=O)(=O)c1ccc(S(=O)(=O)N(C)C)cc1] | N1-(3-chloro-2- (methyl(propyl)ami- no)phenyl)- N4,N4- dimethylbenzene- 1,4-disulfonamide | Compound 74 | ¹H NMR (400 MHz, DMSO-d₆) δ 9.49 (s, 1H), 8.06 (d, J = 8.4 Hz, 2H), 7.94 (d, J = 8.3 Hz, 2H), 7.40- 7.31 (m, 1H), 7.20-7.10 (m, 2H), 2.88-2.77 (m, 2H), 2.61 (s, 6H), 2.33 (s, 3H), 1.21-1.07 (m, 2H), LCMS = 446.3 [M + H]⁺ HPLC: 99.38% |
| 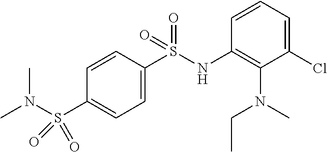 [SMILES: CCN(C)c1c(Cl)cccc1NS(=O)(=O)c1ccc(S(=O)(=O)N(C)C)cc1] | N1-(3-chloro-2- (ethyl(methyl)ami- no)phenyl)- N4,N4- dimethylbenzene- 1,4-disulfonamide | Compound 78 | ¹H NMR (400 MHz, DMSO-d₆) δ 9.58 (s, 1H), 8.06 (d, J = 8.5 Hz, 2H), 7.94 (d, J = 8.5 Hz, 2H), 7.40 (dd, J = 1.5, 7.9 Hz, 1H), 7.21-7.10 (m, 2H), 2.90 (q, J = 7.3 Hz, 2H), 2.60 (s, 6H), 2.45 (s, 3H), 0.70 (t, J = 7.2 Hz, 3H); LCMS = 432.3 [M + H]⁺ HPLC: 99.48% |
| 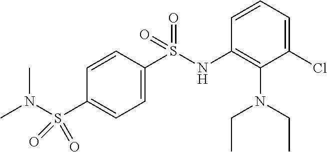 [SMILES: CCN(CC)c1c(Cl)cccc1NS(=O)(=O)c1ccc(S(=O)(=O)N(C)C)cc1] | N1-(3-chloro-2- (diethylamino)phe- nyl)-N4,N4- dimethylbenzene- 1,4-disulfonamide | Compound 77 | ¹H NMR (400 MHz, DMSO-d₆) δ 9.49 (s, 1H), 8.11 (d, J = 8.4 Hz, 2H), 7.94 (d, J = 8.3 Hz, 2H), 7.49 (d, J = 8.1 Hz, 1H), 7.21 (t, J = 8.1 Hz, 1H), 7.11 (d, J = 8.0 Hz, 1H), 2.96 (q, J = 7.0 Hz, 4H), 2.59 (s, 6H), 0.67 (t, J = 7.2 Hz, 6H); LCMS = 446.3 [M + H]⁺ HPLC: 99.34% |
| 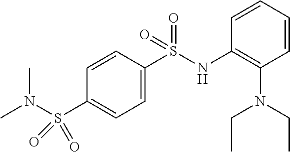 [SMILES: CCN(CC)c1ccccc1NS(=O)(=O)c1ccc(S(=O)(=O)N(C)C)cc1] | N1-(2- (diethylamino)phe- nyl)-N4,N4- dimethylbenzene- 1,4-disulfonamide | Compound 82 | ¹H NMR (400 MHz, DMSO-d₆) δ 9.16 (br s, 1H), 8.07 (d, J = 8.5 Hz, 2H), 7.90 (d, J = 8.5 Hz, 2H), 7.42 (dd, J = 1.8, 7.8 Hz, 2H), 7.21-7.16 (m, 2H), 7.12-7.04 (m, 2H), 2.75 (q, J = 7.1 Hz, 4H), 2.59 (s, 6H), 0.69 (t, J = 7.1 Hz, 6H); LCMS = 412.4 [M + H]⁺ HPLC: 99.57% |

### Example S29. Synthesis of Compound 13

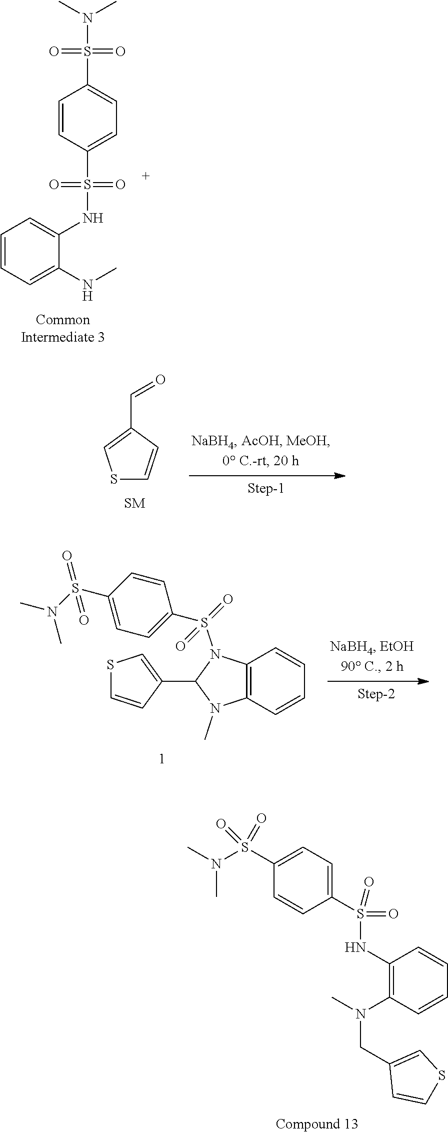 [SMILES: CNc1ccccc1NS(=O)(=O)c1ccc(S(=O)(=O)N(C)C)cc1 ; O=Cc1ccsc1 ; CN1c2ccccc2N(S(=O)(=O)c2ccc(S(=O)(=O)N(C)C)cc2)C1c1ccsc1 ; CN(Cc1ccsc1)c1ccccc1NS(=O)(=O)c1ccc(S(=O)(=O)N(C)C)cc1]

### N,N-dimethyl-4-((3-methyl-2-(thiophen-3-yl)-2,3-dihydro-1H-benzo[diimidazol-1-yl)sulfonyl)benzenesulfonamide (1)

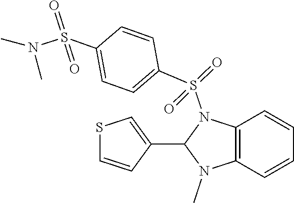 [SMILES: CN1c2ccccc2N(S(=O)(=O)c2ccc(S(=O)(=O)N(C)C)cc2)C1c1ccsc1]

A solution of N1,N1-dimethyl-N4-(2-(methylamino)phenyl)benzene-1,4-disulfonamide (Common Intermediate 3) (150 mg, 0.406 mmol) in MeOH (2 mL) was added to AcOH (19 mg, 0.32 mmol) and thiophene-3-carbaldehyde (SM) (60 mg, 0.528 mmol) at room temperature and stirred for 16 h. The reaction mixture was cooled to 0° C. and NaBH₄ (30 mg, 0.0.79 mmol) was added. The resulting reaction mixture was stirred at room temperature for 4 h. After the reaction was completed as indicated by TLC, the reaction mixture was concentrated and extracted with EtOAc (20 mL×3) and washed with water. The organic layers were dried over anhydrous Na₂SO₄, filtered, and concentrated under reduced pressure to give 170 mg mixture of title compound and intermediate 1. This mixture was used in the next step without any further purification.

### N1,N1-dimethyl-N4-(2-(methyl(thiophen-3-ylmethyl)amino) phenyl)benzene-1,4-disulfonamide (Compound 13)

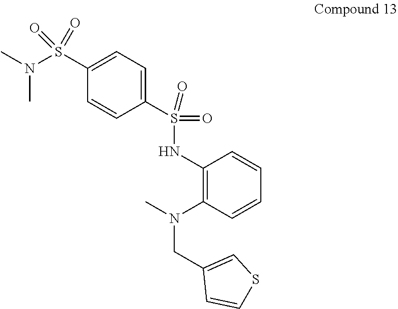 [SMILES: CN(Cc1ccsc1)c1ccccc1NS(=O)(=O)c1ccc(S(=O)(=O)N(C)C)cc1]

A mixture of crude intermediate 1 and Compound 13 (170 mg, 0.367 mmol) in EtOH (2 mL) were added to NaBH₄ (55 mg, 1.45 mmol) at room temperature. The resulting reaction mixture was heated at 90° C. and stirred for 2 h. After the reaction was completed as indicated by TLC, the reaction mixture was concentrated under reduced pressure. The crude product was purified by medium pressure liquid column chromatography eluting with 20-30% EtOAc in heptane to give 50 mg of the title compound. The yield was 29%.

¹H NMR (400 MHz, DMSO-d₆) δ 9.49 (br s, 1H), 8.05 (d, J=7.8 Hz, 2H), 7.90 (d, J=7.8 Hz, 2H), 7.41 (br s, 1H), 7.26 (br s, 1H), 7.14 (d, J=7.8 Hz, 1H), 7.08-6.89 (m, 4H), 3.87 (s, 2H), 2.58 (s, 6H), 2.37 (s, 3H); LCMS [M+H]⁺ calcd for C₂₀H₂₄N₃O₄S₃, 466; found, 466.8; HPLC: 98.06%

The following compounds were prepared according to the synthesis of Compound 13.

| | | Compound | |
|---|---|---|---|
| Structure | Name | No. | Analytical data |
| 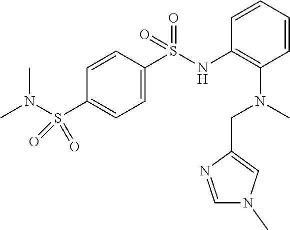 | N1,N1-dimethyl-N4- (2-(methyl((1- methyl-1H-imidazol- 4- yl)methyl)amino)phe- nyl)benzene-1,4- disulfonamide | Compound 59 | ¹H NMR (400 MHz, DMSO- d₆) δ 8.89 (d, J = 8.4 Hz, 2H), 7.80 (d, J = 8.4 Hz, 2H), 7.68 (s, 1H), 7.43 (d, J = 7.6 Hz, 1H), 7.13 (d, J = 7.2 Hz, 1H), 7.07-7.00 (m, 2H), 6.93 (s, 1H), 3.63 (s, 3H), 3.44 (s, 2H), 2.56 (s, 6H), 2.31 (s, 3H); LCMS = 464.0 [M + H]⁺ HPLC: 97.17% |
| 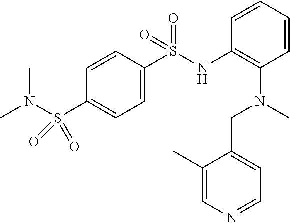 | N1,N1-dimethyl-N4- (2-(methyl((3- methylpyridin-4- yl)methyl)amino)phe- nyl)benzene-1,4- disulfonamide | Compound 58 | ¹H NMR (400 MHz, DMSO- d₆) δ 9.46 (s, 1H), 8.30 (s, 2H), 7.98 (d, J = 8.3 Hz, 2H), 7.87 (d, J = 8.3 Hz, 2H), 7.32 (d, J = 4.4 Hz, 1H), 7.22-7.05 (m, 3H), 7.02-6.92 (m, 1H), 3.94 (s, 2H), 2.59 (s, 6H), 2.47 (s, 3H), 2.15 (s, 3H) LCMS = 475.2 [M + H]⁺ HPLC: 97.06% |
| 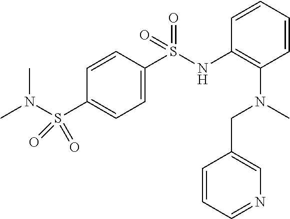 | N1,N1-dimethyl-N4- (2-(methyl(pyridin-3- ylmethyl)amino)phe- nyl)benzene-1,4- disulfonamide | Compound 57 | ¹H NMR (400 MHz, DMSO- d₆) δ 9.63 (s, 1H), 8.52-8.34 (m, 2H), 8.06 (d, J = 8.3 Hz, 2H), 7.92 (d, J = 7.8 Hz, 2H), 7.66 (d, J = 7.8 Hz, 1H), 7.34- 7.22 (m, 1H), 7.16-7.03 (m, 3H), 6.98 (d, J = 6.4 Hz, 1H), 3.97 (s, 2H), 2.58 (s, 6H), 2.38 (s, 3H); LCMS = 461.3 [M + H]⁺ HPLC: 98.32% |
| 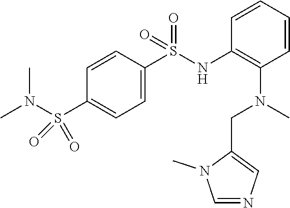 | N1,N1-dimethyl-N4- (2-(methyl((1- methyl-1H-imidazol- 5- yl)methyl)amino)phe- nyl)benzene-1,4- disulfonamide | Compound 56 | ¹H NMR (400 MHz, DMSO- d₆) δ 9.45 (s, 1H), 8.06 (d, J = 8.3 Hz, 2H), 7.93 (d, J = 8.3 Hz, 2H), 7.49 (s, 1H), 7.17- 6.94 (m, 4H), 6.72 (s, 1H), 3.92 (s, 2H), 3.35 (s, 3H), 2.61 (s, 6H), 2.37 (s, 3H) LCMS = 464.5 [M + H]⁺ HPLC: 99.33% |
| 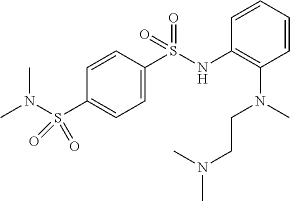 | N1-(2-((2- (dimethylamino)ethyl) (methyl)amino)phe- nyl)-N4,N4- dimethylbenzene-1,4- disulfonamide | Compound 54 | ¹H NMR (400 MHz, DMSO- d₆) δ 7.94-7.87 (m, 2H), 7.83- 7.75 (m, 2H), 7.15 (dd, J = 1.8, 7.7 Hz, 1H), 7.06 (dd, J = 2.0, 7.5 Hz, 1H), 6.87-6.79 (m, 2H), 2.90-2.84 (m, 2H), 2.84-2.78 (m, 2H), 2.66 (s, 6H), 2.60 (s, 6H), 2.47 (s, 3H); LCMS = 441.4 [M + H]⁺ HPLC: 97.93% |
| 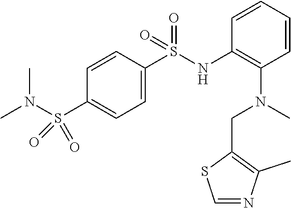 | N1,N1-dimethyl-N4- (2-(methyl((4- methylthiazol-5- yl)methyl)amino)phe- nyl)benzene-1,4- disulfonamide | Compound 51 | ¹H NMR (400 MHz, DMSO- d₆) δ 9.39 (s, 1H), 8.81 (s, 1H), 8.06 (d, J = 8.3 Hz, 2H), 7.93 (d, J = 7.8 Hz, 2H), 7.19 (d, J = 7.3 Hz, 1H), 7.05 (s, 3H), 4.09 (s, 2H), 2.59 (s, 6H), 2.38 (s, 3H), 2.13 (s, 3H) LCMS = 480.9 [M + H]⁺ HPLC: 99.11% |
| 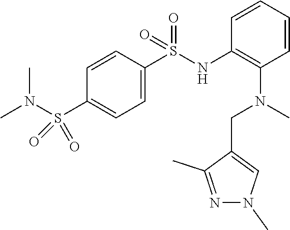 | N1-(2-(((1,3- dimethyl-1H- pyrazol-4- yl)methyl)(methyl)a- mino)phenyl)- N4,N4- dimethylbenzene-1,4- disulfonamide | Compound 50 | ¹H NMR (400 MHz, DMSO- d₆) δ 9.32 (s, 1H), 8.06 (d, J = 7.3 Hz, 2H), 7.91 (d, J = 7.8 Hz, 2H), 7.32 (s, 1H), 7.19 (d, J = 6.8 Hz, 1H), 7.08-6.78 (m, 3H), 3.71-3.57 (m, 5H), 2.59 (s, 6H), 2.31 (s, 3H), 1.73 (s, 3H) LCMS = 477.9 [M + H]⁺ HPLC: 96.28% |
| 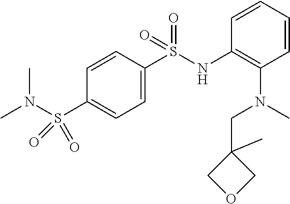 | N1,N1-dimethyl-N4- (2-(methyl((3- methyloxetan-3- yl)methyl)amino)phe- nyl)benzene-1,4- disulfonamide | Compound 49 | ¹H NMR (400 MHz, DMSO- d₆) δ 9.05 (s, 1H), 8.02 (d, J = 8.3 Hz, 2H), 7.93 (d, J = 8.3 Hz, 2H), 7.32-7.17 (m, 2H), 7.14-6.97 (m, 2H), 4.28 (d, J = 5.4 Hz, 2H), 4.11 (d, J = 5.4 Hz, 2H), 2.94 (s, 2H), 2.62 (s, 6H), 2.31 (s, 3H), 1.25 (s, 3H); LCMS = 454.4 [M + H]⁺ HPLC: 96.76% |
| 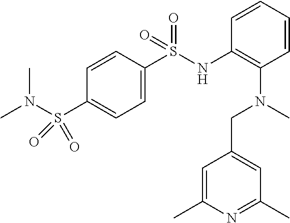 | N1-(2-(((2,6- dimethylpyridin-4- yl)methyl)(methyl)a- mino)phenyl)- N4,N4- dimethylbenzene-1,4- disulfonamide | Compound 48 | ¹H NMR (400 MHz, DMSO- d₆) δ 9.58 (s, 1H), 8.02 (d, J = 8.3 Hz, 2H), 7.89 (d, J = 8.3 Hz, 2H), 7.13-7.03 (m, 3H), 6.99-6.91 (m, 3H), 3.85 (s, 2H), 2.57 (s, 6H), 2.41 (s, 3H), 2.36 (s, 6H); LCMS = 489.4 [M + H]⁺ HPLC: 97.28% |
| 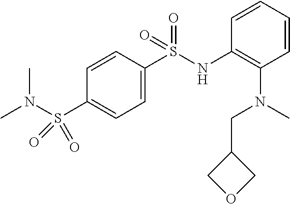 | N1,N1-dimethyl-N4- (2-(methyl(oxetan-3- ylmethyl)amino)phe- nyl)benzene-1,4- disulfonamide | Compound 47 | ¹H NMR (400 MHz, DMSO- d₆) δ 9.34 (d, J = 7.3 Hz, 1H), 8.05 (d, J = 7.3 Hz, 2H), 7.93 (d, J = 7.8 Hz, 2H), 7.28-7.12 (m, 2H), 7.10-6.98 (m, 2H), 4.41 (s, 2H), 4.09 (s, 2H), 2.99 (s, 3H), 2.62 (s, 6H), 2.32 (s, 3H) LCMS = 440.79 [M + H]⁺ HPLC: 99.59% |
| 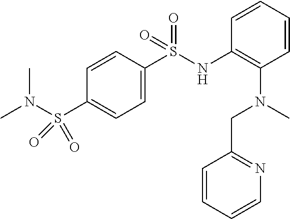 | N1,N1-dimethyl-N4- (2-(methyl(pyridin-2- ylmethyl)amino)phe- nyl)benzene-1,4- disulfonamide | Compound 46 | ¹H NMR (400 MHz, DMSO- d₆) δ 11.72 (s, 1H), 8.69 (d, J = 3.9 Hz, 1H), 7.90-7.74 (m, 5H), 7.49-7.34 (m, 3H), 7.23- 7.15 (m, 1H), 7.11-6.97 (m, 2H), 3.73 (s, 2H), 2.55 (s, 6H), 2.40 (s, 3H) LCMS = 461.75 [M + H]⁺ HPLC: 99.28% |
| 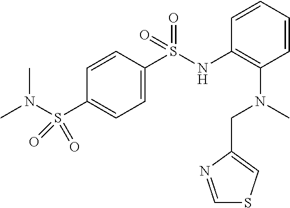 | N1,N1-dimethyl-N4- (2-(methyl(thiazol-4- ylmethyl)amino)phe- nyl)benzene-1,4- disulfonamide | Compound 45 | ¹H NMR (400 MHz, DMSO- d₆) δ 9.96 (s, 1H), 9.16 (s, 1H), 7.92 (d, J = 7.9 Hz, 2H), 7.82 (d, J = 7.9 Hz, 2H), 7.53 (s, 1H), 7.38 (d, J = 7.0 Hz, 1H), 7.16 (d, J = 7.0 Hz, 1H), 7.11- 7.00 (m, 2H), 3.80 (s, 2H), 2.56 (s, 6H), 2.38 (s, 3H) LCMS = 467.3 [M + H]⁺ HPLC: 99.53% |
| 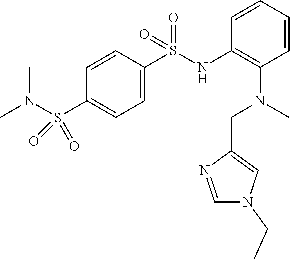 | N1-(2-(((1-ethyl-1H- imidazol-4- yl)methyl)(methyl)a- mino)phenyl)- N4,N4- dimethylbenzene-1,4- disulfonamide | Compound 44 | ¹H NMR (400 MHz, DMSO- d₆) δ 7.93-7.85 (m, 2H), 7.83- 7.73 (m, 3H), 7.43 (d, J = 7.3 Hz, 1H), 7.18-7.10 (m, 1H), 7.09-6.97 (m, 3H), 3.97 (q, J = 7.3 Hz, 2H), 3.44 (s, 2H), 2.56 (s, 6H), 2.32 (s, 3H), 1.34 (t, J = 7.3 Hz, 3H) LCMS = 478.48 [M + H]⁺ HPLC: 99.74% |
| 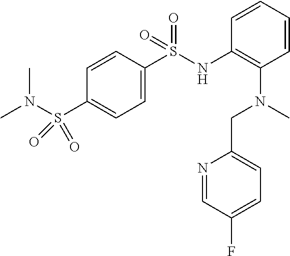 | N1-(2-(((5- fluoropyridin-2- yl)methyl)(methyl)a- mino)phenyl)- N4,N4- dimethylbenzene-1,4- disulfonamide | Compound 43 | ¹H NMR (400 MHz, DMSO- d₆) δ 10.85-10.69 (m, 1H), 8.58 (s, 1H), 7.93-7.86 (m, 2H), 7.84-7.72 (m, 3H), 7.50 (s, 1H), 7.37 (d, J = 6.8 Hz, 1H), 7.15 (s, 1H), 7.03 (d, J = 5.4 Hz, 2H), 3.78 (s, 2H), 2.56 (s, 6H), 2.39 (s, 3H) LCMS = 479.0 [M + H]⁺ HPLC: 97.40% |
| 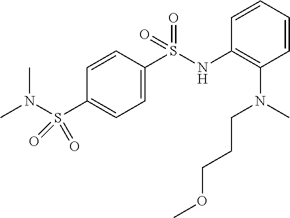 | N1-(2-((3- methoxypropyl)(meth- yl)amino)phenyl)- N4,N4- dimethylbenzene-1,4- disulfonamide | Compound 34 | ¹H NMR (400 MHz, DMSO- d₆) δ 9.23 (s, 1H), 8.05-7.96 (m, 2H), 7.92 (d, J = 7.8 Hz, 2H), 7.29 (d, J = 7.3 Hz, 1H), 7.19-6.95 (m, 3H), 3.30 (s, 2H), 3.24 (s, 3H), 2.68 (d, J = 6.4 Hz, 2H), 2.61 (s, 6H), 2.32 (s, 3H), 1.65-1.39 (m, 2H) LCMS = 442.4 [M + H]⁺ HPLC: 99.08% |
| 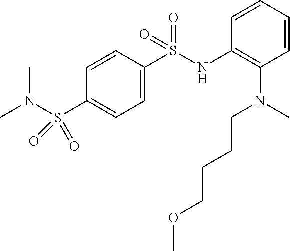 | N1-(2-((4- methoxybutyl)(methyl) amino)phenyl)- N4,N4- dimethylbenzene-1,4- disulfonamide | Compound 33 | ¹H NMR (400 MHz, DMSO- d₆) δ 9.24 (s, 1H), 8.05 (d, J = 8.3 Hz, 2H), 7.92 (d, J = 8.3 Hz, 2H), 7.22 (d, J = 7.3 Hz, 1H), 7.15-6.96 (m, 3H), 3.23 (t, J = 6.1 Hz, 2H), 3.19 (s, 3H), 2.71 (t, J = 7.1 Hz, 2H), 2.62 (s, 6H), 2.35 (s, 3H), 1.42- 1.33 (m, 2H), 1.28 (d, J = 6.4 Hz, 2H) LCMS = 456.4 [M + H]⁺ HPLC: 99.18% |
| 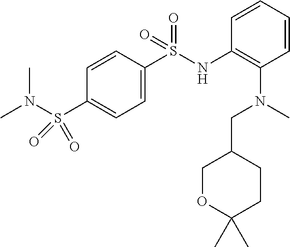 | N1-(2-(((6,6- dimethyltetrahydro- 2H-pyran-3- yl)methyl)(methyl)a- mino)phenyl)- N4,N4- dimethylbenzene-1,4- disulfonamide | Compound 32 | ¹H NMR (400 MHz, DMSO- d₆) δ 9.13 (s, 1H), 8.05 (d, J = 7.9 Hz, 2H), 7.93 (d, J = 7.9 Hz, 2H), 7.26-6.93 (m, 4H), 3.54 (d, J = 9.6 Hz, 1H), 3.18 (t, J = 10.1 Hz, 1H), 2.68 (d, J = 6.1 Hz, 2H), 2.62 (s, 6H), 2.37 (s, 3H), 1.59-1.38 (m, 3H), 1.23 (d, J = 9.2 Hz, 2H), 1.09 (s, 6H) LCMS = 496.6 [M + H]⁺ HPLC: 99.74% |
| 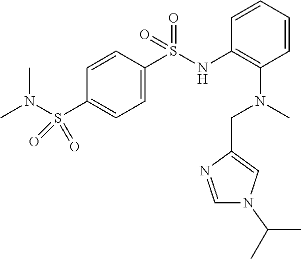 [SMILES: CC(C)n1cnc(CN(C)c2ccccc2NS(=O)(=O)c2ccc(S(=O)(=O)N(C)C)cc2)c1] | N1-(2-(((1-isopropyl- 1H-imidazol-4- yl)methyl)(methyl)a- mino)phenyl)- N4,N4- dimethylbenzene-1,4- disulfonamide | Compound 31 | ¹H NMR (400 MHz, DMSO- d₆) δ 7.91-7.87 (m, 2H), 7.81- 7.79 (m, 2H), 7.79-7.77 (m, 1H), 7.45-7.40 (m, 1H), 7.12 (d, J = 7.9 Hz, 1H), 7.08 (d, J = 1.0 Hz, 1H), 7.05-6.99 (m, 2H), 4.41-4.37 (m, 1H), 3.44 (s, 3H), 2.56 (s, 6H), 2.33 (s, 3H), 1.39 (d, J = 6.6 Hz, 6H); LCMS = 492.2 [M + H]⁺ HPLC: 95.57% |
| 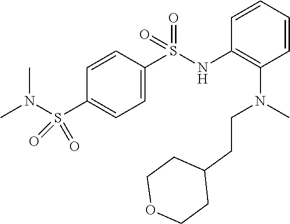 | N1,N1-dimethyl-N4- (2-(methyl(2- (tetrahydro-2H- pyran-4- yl)ethyl)amino)phenyl) benzene-1,4- disulfonamide | Compound 114 | 1H NMR (400 MHz, DMSO- d₆) δ 9.29 (br s, 1H), 8.06 (d, J = 8.4 Hz, 2H), 7.93 (d, J = 8.5 Hz, 2H), 7.21 (dd, J = 1.0, 7.9 Hz, 1H), 7.17-7.13 (m, 1H), 7.08 (t, J = 7.1 Hz, 1H), 7.04- 6.98 (m, 1H), 3.76 (dd, J = 2.9, 10.9 Hz, 2H), 3.21 (t, J = 10.9 Hz, 2H), 2.78-2.69 (m, 2H), 2.62 (s, 6H), 2.37 (s, 3H), 1.43 (d, J = 12.8 Hz, 2H), 1.20- 0.99 (m, 5H); LCMS = 482.2 [M + H]⁺ HPLC: 98.09% |
| 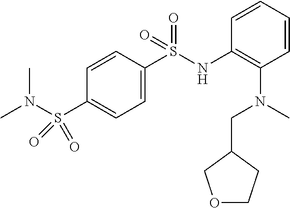 | N1,N1-dimethyl-N4- (2- (methyl((tetrahydrofu- ran-3- yl)methyl)amino)phe- nyl)benzene-1,4- disulfonamide | Compound 112 | 1H NMR (400 MHz, DMSO- d₆) δ 9.25 (br s, 1H), 8.07 (d, J = 8.4 Hz, 2H), 7.93 (d, J = 8.4 Hz, 2H), 7.22-7.17 (m, 2H), 7.08 (t, J = 6.4 Hz, 1H), 7.03- 6.99 (m, 1H), 3.66-3.61 (m, 1H), 3.54-3.48 (m, 2H), 3.25- 3.22 (m, 1H), 2.85-2.79 (m, 1H), 2.75-2.69 (m, 1H), 2.62 (s, 6H), 2.39 (s, 3H), 2.17- 2.04 (m, 1H), 1.79-1.76 (m, 1H), 1.43-1.38 (m, 1H); LCMS = 454.6 [M + H]⁺ |
| | | | HPLC: 96.83% |
| 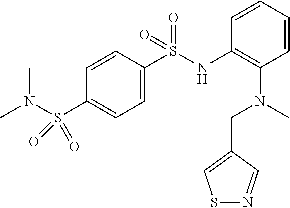 | N1-(2-((isothiazol-4- ylmethyl)(methyl)ami- no)phenyl)-N4,N4- dimethylbenzene-1,4- disulfonamide | Compound 101 | ¹H NMR (400 MHz, DMSO- d₆) δ 9.63 (br s, 1H), 8.77 (s, 1H), 8.38 (s, 1H), 8.06 (d, J = 8.6 Hz, 2H), 7.91 (d, J = 8.4 Hz, 2H), 7.16-7.11 (m, 1H), 7.01-6.95 (m, 3H), 4.03 (s, 2H), 2.59 (s, 6H), 2.38 (s, 3H); LCMS = 467.3 [M + H]+ HPLC: 96.05% |
| 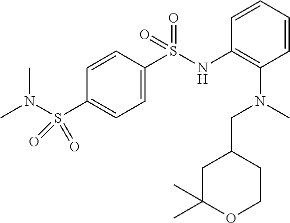 | N1-(2-(((2,2- dimethyltetrahydro- 2H-pyran-4- yl)methyl)(methyl)a- mino)phenyl)- N4,N4- dimethylbenzene-1,4- disulfonamide | Compound 100 | 1H NMR (400 MHz, DMSO- d₆) δ 9.08 (br s, 1H), 8.05 (d, J = 8.3 Hz, 2H), 7.94 (d, J = 8.4 Hz, 2H), 7.17 (d, J = 8.0 Hz, 2H), 7.12-7.05 (t, J = 6.8 Hz, 1H), 7.03-6.95 (t, J = 6.8 Hz, 1H), 3.56-3.53 (m, 1H), 3.46- 3.39 (m, 2H), 2.62 (s, 6H), 2.56 (br s, 2H), 2.42 (s, 3H), 1.50 (d, J = 12.8 Hz, 2H), 1.07 (d, J = 16.5 Hz, 6H); LCMS = 496.0 [M + H]⁺ HPLC: 97.92% |
| 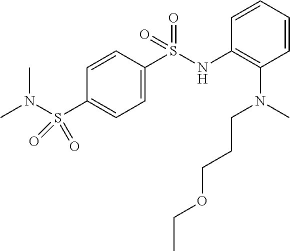 | N1-(2-((3- ethoxypropyl)(methyl) amino)phenyl)- N4,N4- dimethylbenzene-1,4- disulfonamide | Compound 113 | 1H NMR (400 MHZ, DMSO- d₆) δ 9.24 (br s, 1H), 7.98 (d, J = 7.9 Hz, 2H), 7.86 (d, J = 7.9 Hz, 2H), 7.27 (d, J = 9.2 Hz, 2H), 7.15 - 6.98 (m, 2H), 3.41- 3.36 (m, 2H), 3.21 (s, 2H), 2.72-2.61 (m, 2H), 2.57 (s, 6H), 2.26 (br s, 3H), 1.49- 1.38 (m, 2H), 1.06 (t, J = 6.8 Hz, 3H); LCMS = 456.6 [M + H]⁺ HPLC: 97.94% |
| 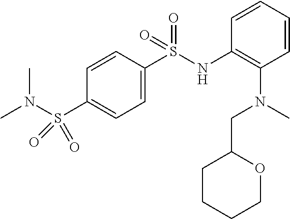 | N1,N1-dimethyl-N4- (2- (methyl((tetrahydro- 2H-pyran-2- yl)methyl)amino)phe- nyl)benzene-1,4- disulfonamide | Compound 108 | 1H NMR (400 MHz, DMSO- d₆) δ 9.76 (br s, 1H), 7.98- 7.88 (m, 4H), 7.45 (d, J = 4.4 Hz, 1H), 7.13 (br s, 1H), 7.05 (br s, 2H), 4.16 (d, J = 11.0 Hz, 1H), 3.62-3.48 (m, 2H), 2.60 (s, 7H), 2.39 (s, 3H), 2.35- 2.24 (m, 1H), 1.80 (d, J = 11.0 Hz, 1H), 1.57 (br s, 4H), 1.42 (d, J = 13.2 Hz, 1H); LCMS = 468.0 [M + H]⁺ HPLC: 99.91% |
| 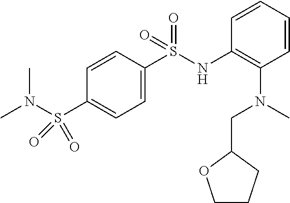 | N1,N1-dimethyl-N4- (2- (methyl((tetrahydrofu- ran-2- yl)methyl)amino)phe- nyl)benzene-1,4- disulfonamide | Compound 107 | 1H NMR (400 MHz, DMSO- d₆) δ 8.40 (s, 1H), 7.96-7.91 (m, 2H), 7.88-7.82 (m, 2H), 7.34-7.29 (m, 1H), 7.07-7.01 (m, 1H), 6.97-6.89 (m, 2H), 4.03-3.95 (m, 1H), 3.93-3.86 (m, 1H), 3.76-3.69 (m, 1H), 2.91-2.88 (m, 1H), 2.60 (s, 6H), 2.47 (s, 4H), 1.84-1.82 (m, 3H), 1.36-1.33 (m, 1H); LCMS = 454.1 [M + H]⁺ HPLC: 99.79% |
| 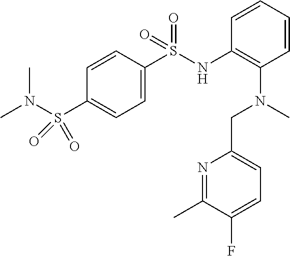 | N1-(2-(((5-fluoro-6- methylpyridin-2- yl)methyl)(methyl)a- mino)phenyl)- N4,N4- dimethylbenzene-1,4- disulfonamide | Compound 85 | 1H NMR (400 MHz, DMSO- d₆) δ 10.49 (s, 1H), 7.89-7.78 (m, 4H), 7.66 (t, J = 9.0 Hz, 1H), 7.52-7.45 (m, 1H), 7.29- 7.22 (m, 1H), 7.21-7.15 (m, 1H), 7.12-7.03 (m, 2H), 3.57 (s, 2H), 2.67 (br s, 3H), 2.64- 2.60 (m, 6H), 2.34 (s, 3H); LCMS = 493.5 [M + H]⁺ HPLC: 95.85% |
| 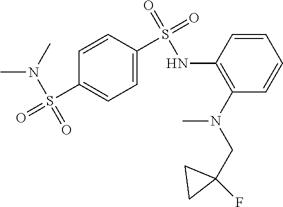 [SMILES: CN(CC1(F)CC1)c1ccccc1NS(=O)(=O)c1ccc(S(=O)(=O)N(C)C)cc1] | N1-(2-(((1- fluorocyclopropyl)meth- yl)(methyl)amino) phenyl)-N4,N4- dimethylbenzene-1,4- disulfonamide | Compound 95 | 1H NMR (400 MHz, DMSO- d₆) δ 9.13-9.04 (m, 1H), 8.06- 7.99 (m, 2H), 7.96-7.90 (m, 2H), 7.27-7.18 (m, 2H), 7.15- 7.01 (m, 2H), 3.12-3.01 (m, 2H), 2.61 (s, 6H), 2.56 (s, 3H), 0.92-0.79 (m, 2H), 0.49-0.37 (m, 2H); LCMS = 442.5 [M + H]⁺ HPLC: 99.89% |
| 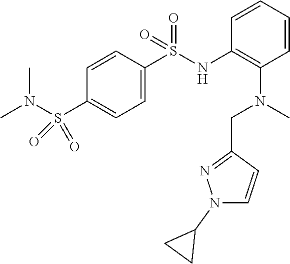 | N1-(2-(((1- cyclopropyl-1H- pyrazol-3- yl)methyl)(methyl)a- mino)phenyl)- N4,N4- dimethylbenzene-1,4- disulfonamide | Compound 94 | 1H NMR (400 MHz, DMSO- d₆) δ 9.48 (br s, 1H), 7.97 (d, J = 8.4 Hz, 2H), 7.86 (d, J = 8.4 Hz, 2H), 7.64 (s, 1H), 7.27 (d, J = 7.2 Hz, 1H), 7.09-6.97 (m, 3H), 5.97 (s, 1H), 3.65 (s, 3H), 2.58 (s, 6H), 2.33 (s, 3H), 1.04-1.03 (m, 2H), 0.93-0.92 (m, 2H); LCMS = 490.0 [M + H]⁺ HPLC: 99.22% |
| 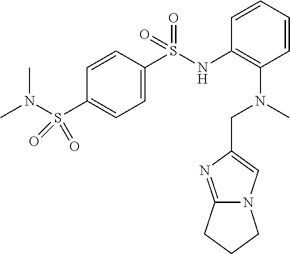 | N1-(2-(((6,7-dihydro- 5H-pyrrolo[1,2- a]imidazol-2- yl)methyl)(methyl)a- mino)phenyl)- N4,N4- dimethylbenzene-1,4- disulfonamide | Compound 83 | 1H NMR (400 MHz, CDCl₃) δ 7.96 (d, J = 8.4 Hz, 2H), 7.72 (d, J = 8.5 Hz, 2H), 7.65-7.61 (m, 1H), 7.11-7.00 (m, 3H), 6.62 (s, 1H), 4.00-3.93 (m, 2H), 3.54 (s, 2H), 2.94 (t, J = 7.2 Hz, 2H), 2.71 (s, 6H), 2.67- 2.60 (m, 2H), 2.43 (s, 3H), 1- exchangeable proton was not observed in 1H NMR; LCMS = 490.2 [M + H]⁺ HPLC: 97.06% |
| 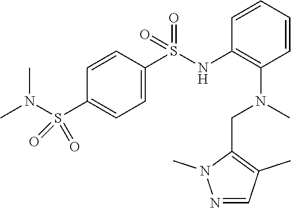 | N1-(2-(((1,4- dimethyl-1H- pyrazol-5- yl)methyl)(methyl)a- mino)phenyl)- N4,N4- dimethylbenzene-1,4- disulfonamide | Compound 80 | ¹H NMR (400 MHz, DMSO- d₆) δ 9.30 (br s, 1H), 8.07 (d, J = 6.8 Hz, 3H), 7.94 (d, J = 6.8 Hz, 3H), 7.21-6.99 (m, 7H), 3.94 (br s, 3H), 3.55 (s, 4H), 2.34 (br s, 3H), 1.80 (s, 3H); LCMS = 478.4 [M + H]⁺ HPLC: 98.35% |
| 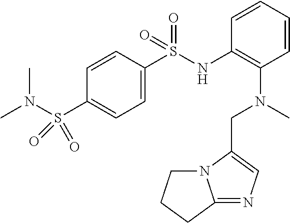 | N1-(2-(((6,7-dihydro- 5H-pyrrolo[1,2- a]imidazol-3- yl)methyl)(methyl)a- mino)phenyl)- N4,N4- dimethylbenzene-1,4- disulfonamide | Compound 79 | 1H NMR (400 MHz, DMSO- d₆) δ 8.18 (s, 1H), 8.07 (d, J = 8.5 Hz, 2H), 7.93 (d, J = 8.5 Hz, 2H), 7.14 (dd, J = 1.7, 7.7 Hz, 1H), 7.05-6.93 (m, 3H), 6.65 (s, 1H), 3.83 (s, 2H), 3.36 (t, J = 7.2 Hz, 2H), 2.63-2.61 (m, 2H), 2.60 (s, 6H), 2.37 (s, 3H), 2.35-2.30 (m, 2H); LCMS = 490.2 [M + H]⁺ HPLC: 99.05% |
| 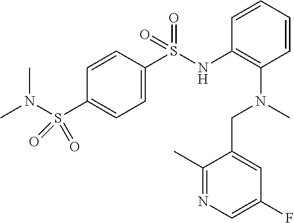 [SMILES: Cc1ncc(F)cc1CN(C)c1ccccc1NS(=O)(=O)c1ccc(S(=O)(=O)N(C)C)cc1] | N1-(2-(((5-fluoro-2- methylpyridin-3- yl)methyl)(methyl)a- mino)phenyl)- N4,N4- dimethylbenzene-1,4- disulfonamide | Compound 75 | 1H NMR (400 MHz, DMSO- d₆) δ 9.59 (s, 1H), 8.29-8.26 (m, 1H), 7.98 (d, J = 8.3 Hz, 2H), 7.86 (d, J = 8.0 Hz, 2H), 7.07 (d, J = 1.4 Hz, 1H), 7.21- 7.05 (m, 3H), 7.05 (d, J = 1.4 Hz, 1H), 3.96 (s, 2H), 2.58 (s, 6H), 2.47 (s, 3H), 2.34-2.31 (m, 3H); LCMS = 493.4 [M + H]⁺ HPLC: 99.62% |

### Example S30. Scheme for Synthesis of Compound 105

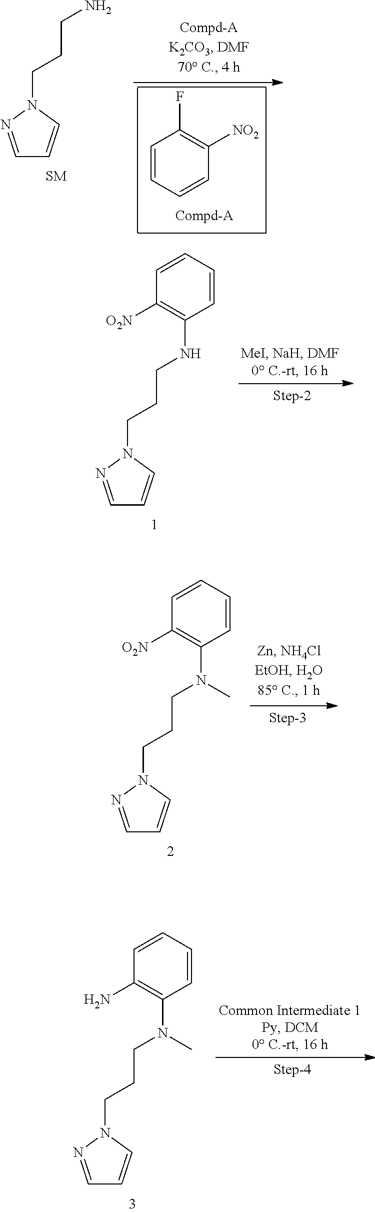 [SMILES: NCCCn1cccn1 ; O=[N+]([O-])c1ccccc1F ; O=[N+]([O-])c1ccccc1NCCCn1cccn1 ; CN(CCCn1cccn1)c1ccccc1[N+](=O)[O-] ; CN(CCCn1cccn1)c1ccccc1N]

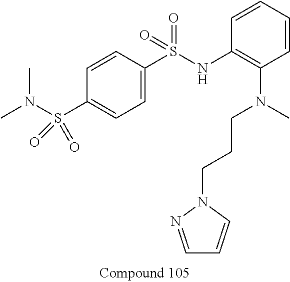

### N-(3-(1H-pyrazol-1-yl)propyl)-2-nitroaniline (1)

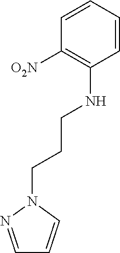 [SMILES: O=[N+]([O-])c1ccccc1NCCCn1cccn1]

A solution of 3-(1H-pyrazol-1-yl)propan-1-amine (SM) (200 mg, 1.6 mmol) in DMF (5 mL) was added to K₂CO₃ (441 mg, 3.20 mmol) and 1-fluoro-2-nitrobenzene (compd-A) (270 mg, 1.92 mmol) at 0° C. and stirred for 15 min. The reaction mixture was heated at 70° C. and stirred for 4 h. After the reaction was completed as indicated by TLC, the reaction mixture was diluted with water (20 mL) and extracted with EtOAc (2×25 mL). The combined organic layers were dried over anhydrous Na₂SO₄, filtered, and concentrated under reduced pressure to give 400 mg of the title compound. The crude was used as such in the next step without further purification and analysis.

### Synthesis of N-(3-(1H-pyrazol-1-yl)propyl)-N-methyl-2-nitroaniline (2)

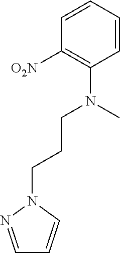 [SMILES: CN(CCCn1cccn1)c1ccccc1[N+](=O)[O-]]

A solution of intermediate 1 (500 mg, 2.03 mmol) in DMF (5 mL) was added to NaH (250 mg, 6.09 mmol) and CH₃I (577 mg, 4.06 mmol) at 0° C. The reaction mixture was allowed to reach room temperature and stirred for 16 h. After the reaction was completed as indicated by TLC, the reaction mixture was diluted with cold water (10 mL) and extracted with EtOAc (2×25 mL). The combined organic layers were dried over anhydrous Na₂SO₄, filtered, and concentrated under reduced pressure to give 528 mg of the title compound. The crude was used as such in the next step without further purification. LCMS [M+H]⁺ calcd for C₁₃H₁₆N₄O₂, 260.13; found, 261.35.

### N1-(3-(1H-pyrazol-1-yl)propyl)-N1-methylbenzene-1,2-diamine (3)

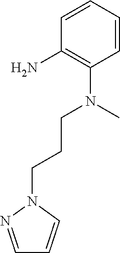 [SMILES: CN(CCCn1cccn1)c1ccccc1N]

A solution of intermediate 2 (528 mg, 2.03 mmol) in a mixture of solvents ethanol and water (10 mL, 4:1) was added to Zn dust (1.32 g, 20.30 mmol) and NH₄Cl (1.07 g, 20.30 mmol) at room temperature. The reaction mixture was heated to 85° C. and stirred for 1 h. After the reaction was completed as indicated by TLC, the reaction mixture was diluted with water (20 mL) and extracted with DCM (2×25 mL). The combined organic layers were dried over anhydrous Na₂SO₄, filtered, and concentrated under reduced pressure to give 450 mg of the title compound. The crude was used as such in the next step without further purification. LCMS [M+H]⁺ calcd for C₁₃H₁₈N₄, 230.15; found, 231.4

### N1-(2-((3-(1H-pyrazol-1-yl)propyl)(methyl)amino)phenyl)-N4,N4-dimethylbenzene-1,4-disulfonamide (Compound 105)

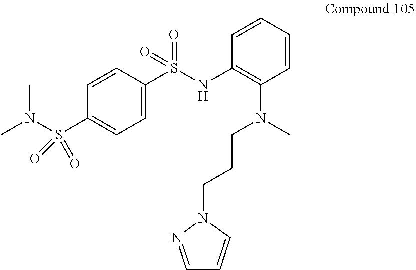 [SMILES: CN(CCCn1cccn1)c1ccccc1NS(=O)(=O)c1ccc(S(=O)(=O)N(C)C)cc1]

A solution of compound 3 (450 mg, 1.95 mmol) in DCM (5 mL) was added to 4-(N,N-dimethylsulfamoyl)benzenesulfonyl chloride Common Intermediate 1 (664 mg, 2.34 mmol) and pyridine (309 mg, 3.91 mmol) at 0° C. The reaction mixture was allowed to reach room temperature and stirred for 16 h. After the reaction was completed as indicated by TLC, the reaction mixture was diluted with water (25 mL) and extracted with DCM (2×25 mL). The combined organic layers were dried over anhydrous Na₂SO₄, filtered, and concentrated under reduced pressure. The crude product was to give 450 mg of the title compound. The crude was purified by medium pressure liquid chromatography by eluting with 5000 EtOAc in heptane to give 60 mg of the title compound. The yield was 6D.

¹HNMR (400 2-(z, DMSO-d₆) δ=8.06-8.04 (m, 1H), 8.04-8.02 (m, 1H), 7.93 (s, 1H), 7.91 (s, 1H), 7.44 (d, J=1.3 Hz, 1H), 7.23 (d, J=1.6 Hz, 1H), 7.22 (d, J=1.5 Hz, 1H), 7.12-6.98 (m, 4H), 6.22 (t, J=2.0 Hz, 1H), 4.10 (t, J=6.8 Hz, 2H), 2.68 (t, J=7.0 Hz, 2H), 2.61 (s, 6H), 2.24 (s, 3H), 1.80 (t, J=6.9 Hz, 2H); LCMS [M+H calcd for C₂₁H₂₇N₅O₄S₂, 477.15; found, 478.39; HPLC: 99.4998

The following compounds were prepared according to the synthesis of Compound 105.

| | | Compound | |
|---|---|---|---|
| Structure | Name | No. | Analytical data |
| 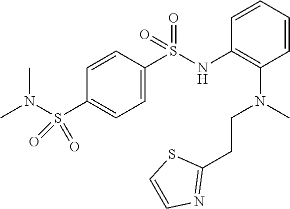 | N1,N1-dimethyl-N4- (2-(methyl(2- (thiazol-2- yl)ethyl)amino)phenyl) benzene-1,4- disulfonamide | Compound 90 | 1H NMR (400 MHz, DMSO- d₆) δ 10.19 (s, 1H), 7.90-7.81 (m, 5H), 7.66 (d, J = 3.3 Hz, 1H), 7.41-7.37 (m, 1H), 7.23 (dd, J = 3.5, 5.9 Hz, 1H), 7.12- 7.08 (m, 2H), 3.17-3.12 (m, 2H), 3.03-2.97 (m, 2H), 2.58 (s, 6H), 2.28 (s, 3H); LCMS = 481.8 [M + H]⁺ HPLC: 95.80% |
| 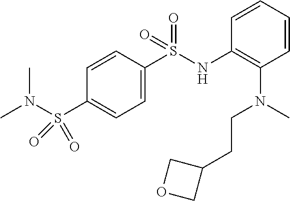 | N1,N1-dimethyl-N4- (2-(methyl(2- (oxetan-3- yl)ethyl)amino)phenyl) benzene-1,4- disulfonamide | Compound 62 | 1H NMR (400 MHz, CD₃OD) δ = 7.79-7.73 (m, 4H), 7.26- 7.24 (m, 1H), 6.98-6.94 (m, 1H), 6.80-6.74 (m, 2H), 3.69- 3.59 (m, 2H), 2.89-2.84 (m, 1H), 2.61 (d, J = 2.1 Hz, 7H), 2.58 (s, 3H), 2.54-2.50 (m, 3H), 2.43 (s, 1H), exchangeable proton was not observed; LCMS = 452.0 [M − H]⁺ HPLC: 98.09% |
| 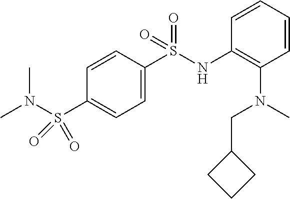 | N1-(2- ((cyclobutylmethyl)(meth- yl)amino)phenyl)- N4,N4- dimethylbenzene-1,4- disulfonamide | Compound 106 | 1H NMR (400 MHz, DMSO- d₆) δ 8.06-7.98 (m, 1H), 7.94- 7.85 (m, 5H), 7.26-7.17 (m, 1H), 7.07 (br s, 2H), 3.88 (s, 6H), 2.74 (d, J = 2.4 Hz, 1H), 2.58 (s, 3H), 2.41 (br s, 2H), 1.70-1.55 (m, 2H), 1.45-1.32 (m, 4H); LCMS = 438.0 [M + H]⁺ HPLC: 99.66% |
| 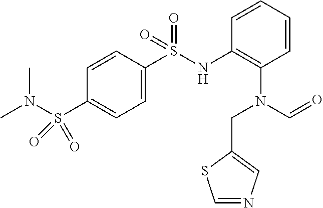 [SMILES: CN(C)S(=O)(=O)c1ccc(S(=O)(=O)Nc2ccccc2N(C=O)Cc2cncs2)cc1] | N-(2-((4-(N,N- dimethylsulfamoyl)phe- nyl)sulfonamido)phe- nyl)-N-(thiazol-5- ylmethyl)formamide | Compound 61 | 1H NMR (400 MHz, DMSO- d₆) δ 8.89 (d, J = 0.6 Hz, 1H), 8.01 (d, J = 0.7 Hz, 1H), 8.00- 7.98 (m, 3H), 7.96-7.95 (m, 2H), 7.94-7.93 (m, 1H), 7.55 (d, J = 0.7 Hz, 1H), 7.28 (d, J = 1.5 Hz, 1H), 7.12 (d, J = 1.3 Hz, 1H), 7.10 (d, J = 1.3 Hz, 1H), 6.88 (d, J = 1.5 Hz, 1H), 6.86 (d, J = 1.5 Hz, 1H), 2.72 (s, 6H) LCMS = 481.0 [M + H]⁺ HPLC: 98.30% |

### Example S31. Synthesis of Compound 73

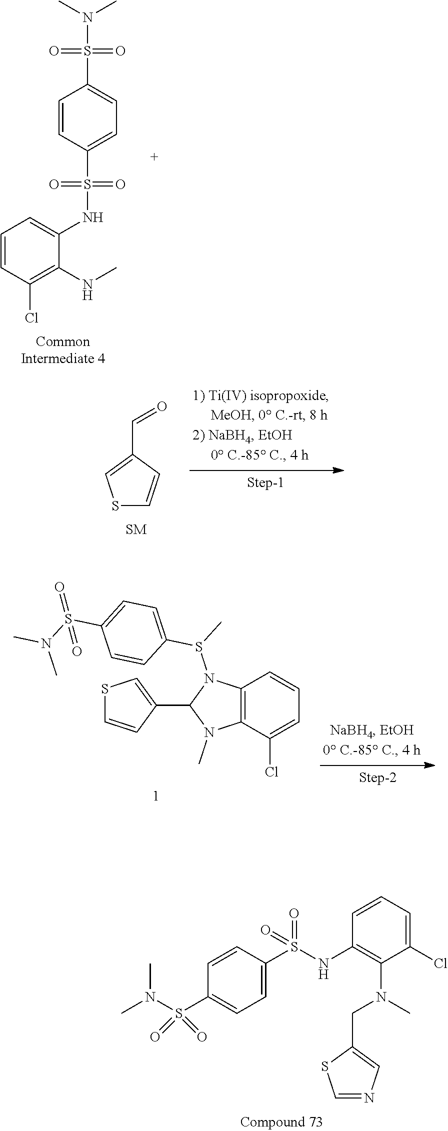

### 4-((4-Chloro-3-methyl-2-(thiophen-3-yl)-2,3-dihydro-1H-benzo[d]imidazol-1-yl)sulfonyl)-N,N-dimethylbenzenesulfonamide (1)

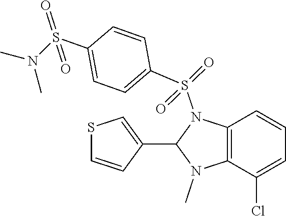 [SMILES: CN1c2c(Cl)cccc2N(S(=O)(=O)c2ccc(S(=O)(=O)N(C)C)cc2)C1c1ccsc1]

A solution of thiophene-3-carbaldehyde (SM) (45 mg, 0.40 mmol) in MeOH (5 mL) was added to titanium isopropoxide (113 mg, 0.40 mmol) at 0° C. and stirred for 5 min. Then to the reaction mixture was added N1-(3-chloro-2-(methylamino)phenyl)-N4,N4-dimethylbenzene-1,4-disulfonamide (Common Intermediate 4) (130 mg, 0.267 mmol) at room temperature and stirred for 8 h. The reaction mixture was cooled to 0° C. and NaBH₄ (19 mg, 0.53 mmol) was added. The resulting reaction mixture was stirred at room temperature for 2 h. After the reaction was completed as indicated by TLC, the reaction mixture was concentrated and extracted with EtOAc and water. The organic layers were dried over anhydrous Na₂SO₄, filtered, and concentrated under reduced pressure to give 170 mg mixture of crude title compound and Compound 73. This mixture was used in the next step without any further purification.

### N1-(3-chloro-2-(methyl(thiazol-5-ylmethyl)amino)phenyl)-N4,N4-dimethylbenzene-1,4-disulfonamide (Compound 73)

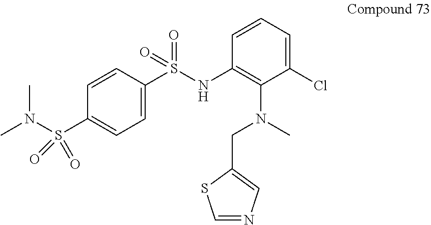 [SMILES: CN(Cc1cncs1)c1c(Cl)cccc1NS(=O)(=O)c1ccc(S(=O)(=O)N(C)C)cc1]

A mixture of crude intermediate 1 and Compound 73 (100 mg, 0.20 mmol) in EtOH (4 mL) was added to NaBH₄ (29 mg, 0.80 mmol) at 0° C. The reaction mixture was heated at 85° C. and stirred for 4 h. After the reaction was completed as indicated by TLC, the reaction mixture was washed with ethyl acetate and water. The combined organic layers were dried over anhydrous Na₂SO₄, filtered, and concentrated under reduced pressure. The crude product was purified by medium pressure liquid chromatography by eluting with 30-50% EtOAc in hexane to give 30 mg of the title compound. The yield was 29%.

¹H NMR (400 MHz, DMSO-d₆) δ 9.60 (s, 1H), 8.93 (s, 1H), 8.05-8.00 (m, 2H), 7.93 (d, J=8.5 Hz, 2H), 7.73 (s, 1H), 7.18-7.09 (m, 3H), 4.38 (s, 2H), 2.60 (s, 6H), 2.52 (s, 3H); LCMS [M+H]⁺ calcd for C₁₉H₂₂N₄O₄S₃, 501; found, 501.0; HPLC: 99.75%

The following compounds were prepared according to the synthesis of Compound 73.

| | | Compound | |
|---|---|---|---|
| Structure | Name | No. | Analytical data |
| 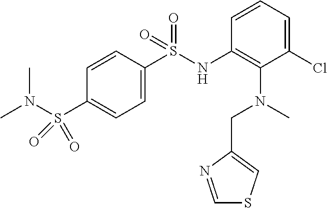 [SMILES: CN(Cc1cscn1)c1c(Cl)cccc1NS(=O)(=O)c1ccc(S(=O)(=O)N(C)C)cc1] | N1-(3-chloro-2- (methyl(thiazol-4- ylmethyl)amino)phe- nyl)-N4,N4- dimethylbenzene-1,4- disulfonamide | Compound 64 | ¹H NMR (400 MHz, DMSO- d₆) δ 9.23 (s, 1H), 7.94 (d, J = 8.6 Hz, 3H), 7.83 (d, J = 8.5 Hz, 3H), 7.63 (d, J = 2.0 Hz, 1H), 7.39 (d, J = 7.6 Hz, 1H), 2.68-2.65 (m, 1H), 2.61 (s, 6H), 2.33 (s, 4H); LCMS = 500.8 [M + H]⁺ HPLC: 99:55% |
| 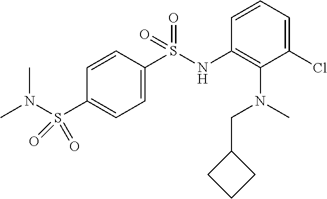 [SMILES: CN(CC1CCC1)c1c(Cl)cccc1NS(=O)(=O)c1ccc(S(=O)(=O)N(C)C)cc1] | N1-(3-chloro-2- ((cyclobutylmethyl)(meth- yl)amino)phenyl)- N4,N4- dimethylbenzene-1,4- disulfonamide | Compound 69 | ¹H NMR (400 MHz, DMSO- d₆) δ 9.38 (s, 1H), 8.09 (d, J = 8.3 Hz, 2H), 7.95 (d, J = 8.3 Hz, 2H), 7.37 (d, J = 7.8 Hz, 1H), 7.19-7.07 (m, 2H), 2.93 (d, J = 6.8 Hz, 2H), 2.67 (br s, 3H), 2.61 (s, 6H), 2.33 (br s, 1H), 1.70-1.60 (m, 6H); LCMS = 472.4 [M + H]⁺ HPLC: 99:57% |
| 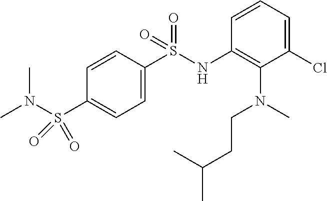 [SMILES: CC(C)CCN(C)c1c(Cl)cccc1NS(=O)(=O)c1ccc(S(=O)(=O)N(C)C)cc1] | N1-(3-chloro-2- (isopentyl(methyl)ami- no)phenyl)-N4,N4- dimethylbenzene-1,4- disulfonamide | Compound 67 | ¹H NMR (400 MHz, DMSO- d₆) δ 9.53 (s, 1H), 8.08 (d, J = 8.6 Hz, 2H), 7.94 (d, J = 8.5 Hz, 2H), 7.37 (dd, J = 1.9, 7.6 Hz, 1H), 7.18-7.10 (m, 2H), 2.94 (t, J = 8.0 Hz, 2H), 2.61 (s, 6H), 2.45 (s, 4H), 1.46- 1.36 (m, 1H), 0.76 (d, J = 6.6 Hz, 6H); LCMS = 474.4 [M + H]⁺ HPLC: 98.38% |

### Example S32. Synthesis of Compound 72

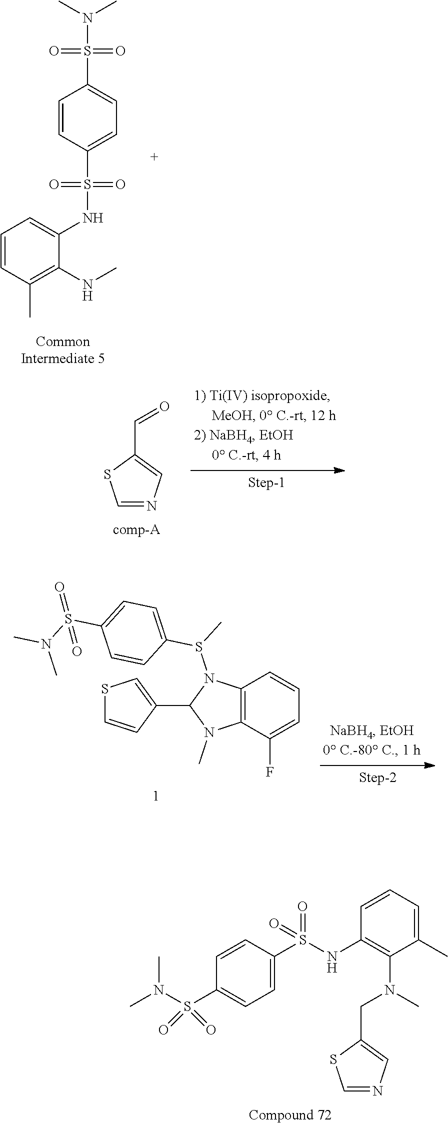

### 4-((3,4-Dimethyl-2-(thiophen-3-yl)-2,3-dihydro-1H-benzo[d]imidazol-1-yl)sulfonyl)-N,N-dimethylbenzenesulfonamide (1)

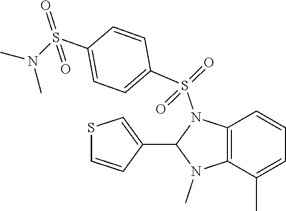 [SMILES: Cc1cccc2c1N(C)C(c1ccsc1)N2S(=O)(=O)c1ccc(S(=O)(=O)N(C)C)cc1]

A solution of Common Intermediate 5 (0.14 g, 0.36 mmol) in MeOH (1.4 mL) was added to titanium isopropoxide (0.15 mL, 0.55 mmol) at 0° C. and stirred for 10 min. Then to the reaction mixture was added thiazole-5-carbaldehyde Comp-A (0.05 g, 0.44 mmol). The reaction mixture was allowed to reach room temperature and stirred for 12 h. The reaction mixture was cooled to 0° C. and NaBH₄ (0.02 g, 0.55 mmol) was added. The resulting reaction mixture was stirred at room temperature for 4 h. After the reaction was completed as indicated by TLC, the reaction mixture was filtered through celite bed, and filtered, and the filtrate was concentrated and extracted with DCM and water. The organic layers were dried over anhydrous Na₂SO₄, filtered, and concentrated under reduced pressure to give 0.15 g of 1 and Compound 72 as a mixture. This mixture was used in the next step without any further purification.

LCMS [M−H]+ calcd for C₂₁H₂₂N₃O₄S₃, 477.09; found, 479.3

### N1,N1-Dimethyl-N4-(3-methyl-2-(methyl(thiazol-5-ylmethyl)amino)phenyl)benzene-1,4-disulfonamide (Compound 72)

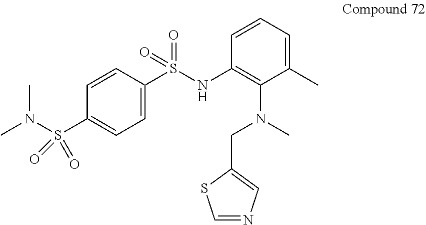

A mixture of crude intermediate 1 (0.15 g, 0.31 mmol) in EtOH (1.5 mL) was added to NaBH₄ (0.059 g, 1.56 mmol) at 0° C. The reaction mixture was heated at 80° C. and stirred for 1 h. After the reaction was completed as indicated by TLC, the reaction mixture was evaporated under vacuum then extracted with ethyl acetate and water. The combined organic layers were washed with brine, dried over anhydrous Na₂SO₄, filtered, and concentrated under reduced pressure. The crude product was purified by medium pressure liquid chromatography by eluting with 80-90% EtOAc in heptane to give 0.02 g of the title compound. The yield was 17%.

1H NMR (400 MHz, DMSO-d₆) δ=10.67-10.61 (m, 1H), 9.27 (d, J=1.8 Hz, 1H), 7.95-7.90 (m, 2H), 7.86-7.80 (m, 2H), 7.60 (s, 1H), 7.33-7.27 (m, 1H), 7.07 (t, J=7.8 Hz, 1H), 6.94-6.87 (m, 1H), 2.69-2.65 (m, 2H), 2.56 (s, 6H), 2.23 (s, 6H); LCMS [M+H]⁺ calcd for C₂₀H₂₅N₄O₄S₃, 481; found, 481.4; HPLC: 98.82%

The following compounds were prepared according to the synthesis of Compound 72.

| | | Compound | |
|---|---|---|---|
| Structure | Name | No. | Analytical data |
| 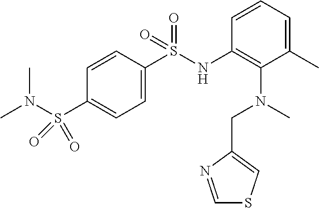 | N1,N1-dimethyl-N4- (3-methyl-2- (methyl(thiazol-4- ylmethyl)amino)phenyl) benzene-1,4- disulfonamide | Compound 71 | 1H NMR (400 MHz, DMSO- d₆) δ 8.99 (s, 1H), 8.01 (d, J = 8.4 Hz, 3H), 7.95-7.89 (m, 2H), 7.80-7.73 (m, 1H), 6.99- 6.88 (m, 3H), 4.31 (s, 2H), 2.59 (s, 6H), 2.35-2.30 (m, 3H), 2.22 (s, 3H); LCMS = 481.4 [M + H]⁺ HPLC: 99.39% |

### Example S33. Synthesis of Compound 70

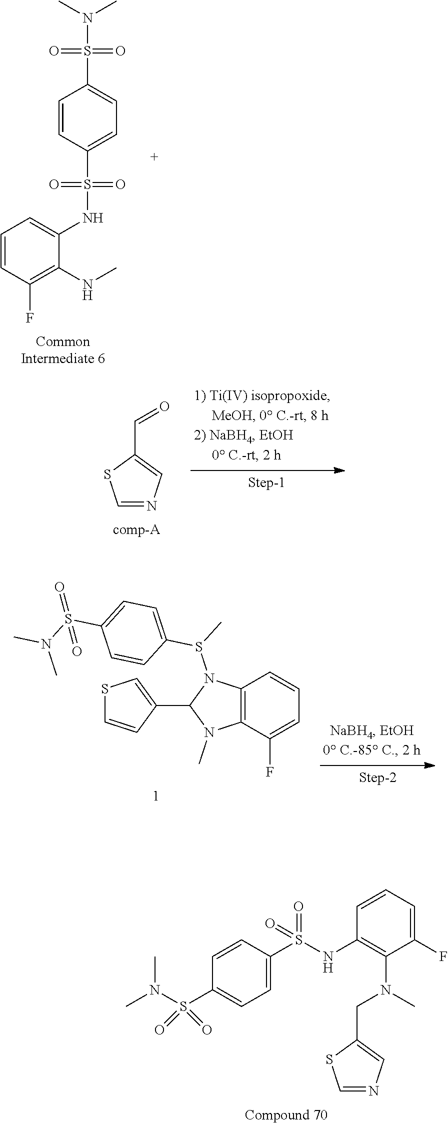

### 4-((4-Fluoro-3-methyl-2-(thiophen-3-yl)-2,3-dihydro-1H-benzo[d]imidazol-1-yl)sulfonyl)-N,N-dimethylbenzenesulfonamide (1)

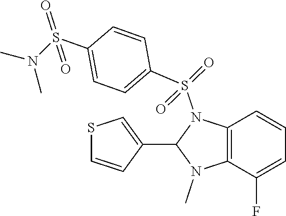 [SMILES: CN1c2c(F)cccc2N(S(=O)(=O)c2ccc(S(=O)(=O)N(C)C)cc2)C1c1ccsc1]

A solution of thiazole-5-carbaldehyde (comp A) (48 mg, 0.42 mmol) in MeOH (4 mL) was added to titanium isopropoxide (121 mg, 0.42 mmol) at 0° C. and stirred for 5 min. Then to the reaction mixture was added N1-(3-fluoro-2-(methylamino)phenyl)-N4,N4-dimethylbenzene-1,4-disulfonamide (Common Intermediate 6) (110 mg, 0.28 mmol) at room temperature and stirred for 8 h. The reaction mixture was cooled to 0° C. and NaBH₄ (21 mg, 0.57 mmol) was added. The resulting reaction mixture was stirred at room temperature for 2 h. After the reaction was completed as indicated by TLC, the reaction mixture was concentrated and extracted with EtOAc and water. The organic layers were dried over anhydrous Na₂SO₄, filtered, and concentrated under reduced pressure to give 100 mg mixture of crude title intermediate 1 and Compound 70. This mixture was used in the next step without any further purification.

### N1-(3-Fluoro-2-(methyl(thiazol-5-ylmethyl)amino)phenyl)-N4,N4-dimethylbenzene-1,4-disulfonamide (Compound 70)

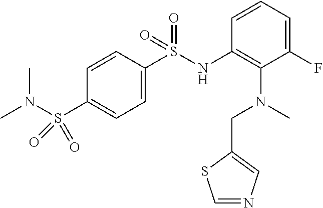 [SMILES: CN(Cc1cncs1)c1c(F)cccc1NS(=O)(=O)c1ccc(S(=O)(=O)N(C)C)cc1]

A mixture of crude 1 and Compound 70 (100 mg, 0.20 mmol) in EtOH (5 mL) was added to NaBH₄ (30 mg, 0.83 mmol) at 0° C. The reaction mixture was heated at 85° C. and stirred for 2 h. After the reaction was completed as indicated by TLC, the reaction mixture was washed with ethyl acetate and water. The combined organic layers were dried over anhydrous Na₂SO₄, filtered, and concentrated under reduced pressure. The crude product was purified by medium pressure liquid chromatography by eluting with 30-50% EtOAc in hexane to give 30 mg of the title compound. The yield was 30%.

¹H NMR (400 MHz, DMSO-d₆) δ=9.63-9.51 (m, 1H), 8.93 (s, 1H), 8.05 (d, J=8.2 Hz, 2H), 7.92 (d, J=7.9 Hz, 2H), 7.67 (s, 1H), 7.12 (s, 2H), 6.98-6.87 (m, 1H), 4.17 (s, 2H), 2.67 (s, 6H), 2.44 (s, 3H); LCMS [M+H]⁺ calcd for C₁₉H₂₂FN₄O₄S₃, 485; found, 485.4; HPLC: 99.80%

The following compounds were prepared according to the synthesis of Compound 70.

| | | Compound | |
|---|---|---|---|
| Structure | Name | No. | Analytical data |
| 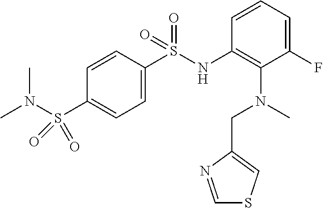 [SMILES: CN(Cc1cscn1)c1c(F)cccc1NS(=O)(=O)c1ccc(S(=O)(=O)N(C)C)cc1] | N1-(3-fluoro-2- (methyl(thiazol-4- ylmethyl)amino)phenyl)- N4,N4- dimethylbenzene-1,4- disulfonamide | Compound 68 | 1H NMR (400 MHz, DMSO- d₆) δ 10.16 (s, 1H), 9.19 (d, J = 1.0 Hz, 1H), 7.95 (d, J = 8.3 Hz, 2H), 7.85 (d, J = 8.3 Hz, 2H), 7.62-7.56 (m, 1H), 7.31- 7.24 (m, 1H), 7.23-7.14 (m, 1H), 7.02-6.92 (m, 1H), 3.92 (s, 2H), 2.56 (s, 6H), 2.35 (s, 3H); LCMS = 485.3 [M + H]⁺ HPLC: 99.70% |

### Example S34. Synthesis of Compound 84

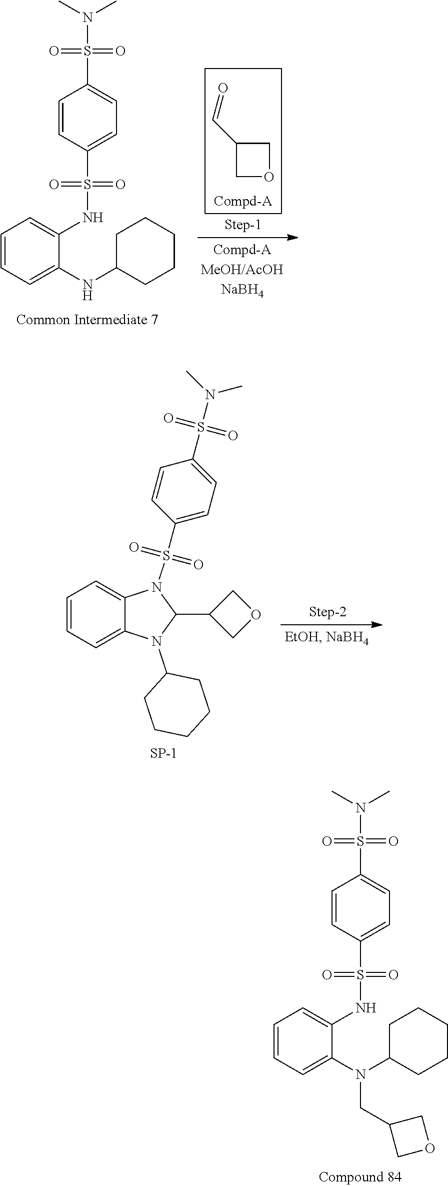

### 4-((3-Cyclohexyl-2-(oxetan-3-yl)-2,3-dihydro-1H-benzo[d]imidazol-1-yl)sulfonyl)-N,N-dimethylbenzenesulfonamide (SP-1)

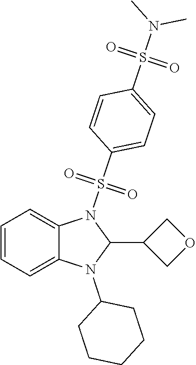 [SMILES: CN(C)S(=O)(=O)c1ccc(S(=O)(=O)N2c3ccccc3N(C3CCCCC3)C2C2COC2)cc1]

A solution of Common Intermediate 7 (200 mg, 0.46 mmol) in methanol (4 mL) was added to oxetane-3-carbaldehyde Compd-A (47 mg, 0.55 mmol) and acetic acid (1 mL) at 0° C. The reaction mixture was allowed to warm to room temperature and stirred for 16 h. The reaction mixture was cooled to 0° C. was added NaBH₄ (26 mg, 0.68 mmol) portion wise. The resulting reaction mixture was allowed to warm to room temperature and stirred for 4 h. After the reaction was completed as indicated by TLC, the reaction mixture was diluted with water and extracted with DCM (2×25 mL). The organic layers were dried over anhydrous Na₂SO₄, filtered, and concentrated under reduced pressure to give 200 mg of title compound. The yield was 86%.

LCMS [M+H]+ calcd for C₂₄H₃₂N₃O₅S₂, 506; found, 506.2.

### N1-(2-(Cyclohexyl(oxetan-3-ylmethyl)amino)phenyl)-N4,N4-dimethylbenzene-1,4-disulfonamide (Compound 84)

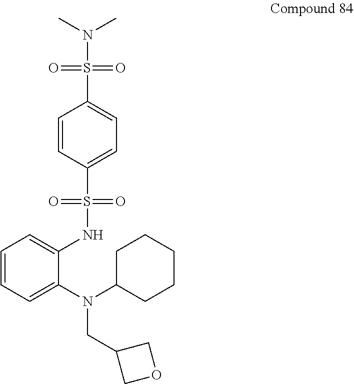 [SMILES: CN(C)S(=O)(=O)c1ccc(S(=O)(=O)Nc2ccccc2N(CC2COC2)C2CCCCC2)cc1]

A solution of SP-1 (200 mg, 0.39 mmol) in ethanol (4 mL) was added to NaBH₄ (60 mg, 1.58 mmol) at 0° C. The reaction mixture was heated to 80° C. and stirred for 3 h. After the reaction was completed as indicated by TLC, the reaction mixture was diluted with water and extracted with DCM (2×25 mL). The organic layers were dried over anhydrous Na₂SO₄, filtered, and concentrated under reduced pressure. The crude product was purified by medium pressure column chromatography, eluted with 30% ethyl acetate in heptane to give 15 mg of title compound. The yield was 7.5%.

¹H NMR (400 MHz, DMSO-d₆) δ 8.79 (br s, 1H), 8.12 (d, J=8.5 Hz, 3H), 7.96 (d, J=8.5 Hz, 1H), 7.41 (d, J=1.5 Hz, 1H), 7.39 (d, J=1.4 Hz, 1H), 7.11 (dt, J=1.4, 7.7 Hz, 1H), 7.07-7.02 (m, 1H), 4.19 (dd, J=5.9, 7.8 Hz, 2H), 3.21-3.17 (m, 3H), 2.68-2.65 (m, 1H), 2.62 (s, 6H), 2.34-2.31 (m, 1H), 1.71-1.58 (m, 5H), 1.06-0.96 (m, 6H); LCMS [M+H]⁺ calcd for C₂₄H₃₃N₃O₅S₂, 507.19; found, 508.2. HPLC: 95.04%.

The following compounds were prepared according to the synthesis of Compound 84.

| | | Compound | |
|---|---|---|---|
| Structure | Name | No. | Analytical data |
| 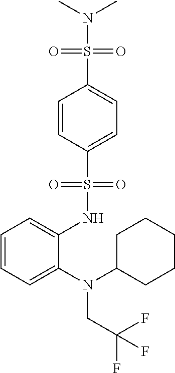 [SMILES: CN(C)S(=O)(=O)c1ccc(S(=O)(=O)Nc2ccccc2N(CC(F)(F)F)C2CCCCC2)cc1] | N1-(2- (cyclohexyl(2,2,2- trifluoroethyl)amino) phenyl)-N4,N4- dimethylbenzene-1,4- disulfonamide | Compound 81 | 1H NMR (400 MHz, DMSO- d₆) δ 8.44 (s, 1H), 8.10-8.06 (m, 2H), 7.97-7.93 (m, 2H), 7.46 (dd, J = 1.4, 8.1 Hz, 1H), 7.38 (dd, J = 1.4, 7.9 Hz, 1H), 7.17 (dt, J = 1.4, 7.8 Hz, 1H), 7.10 (dd, J = 1.5, 7.8 Hz, 1H), 3.81-3.76 (m, 3H), 2.61 (s, 6H), 1.73-1.60 (m, 4H), 1.47 (br d, J = 3.3 Hz, 1H), 1.12 (q, J = 11.0 Hz, 2H), 1.03-0.95 (m, 3H); LCMS = 520.0 [M + H]⁺ HPLC: 98.49% |
| 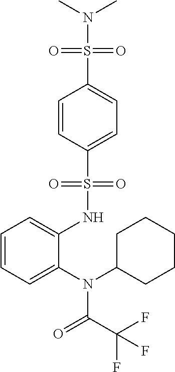 [SMILES: CN(C)S(=O)(=O)c1ccc(S(=O)(=O)Nc2ccccc2N(C(=O)C(F)(F)F)C2CCCCC2)cc1] | N-cyclohexyl-N-(2- ((4-(N,N- dimethylsulfamoyl)phe- nyl)sulfonamido)phe- nyl)-2,2,2- trifluoroacetadmie | Compound 63 | 1H NMR (400 MHz, DMSO- d₆) δ 10.77 (s, 1H), 8.17-8.12 (m, 2H), 7.99 (d, J = 8.6 Hz, 2h), 7.42-7.35 (m, 2H), 7.18- 7.14 (m, 1H), 7.12-7.07 (m, 1H), 2.64 (s, 6H), 2.34-2.31 (m, 1H), 1.96-1.81 (m, 2H), 1.74-1.67 (m, 1H), 1.62-1.54 (m, 1H), 1.54-1.47 (m, 1H), 1.31-1.17 (m, 4H), 0.89-0.76 (m, 2H); LCMS = 532.2 [M − H]⁺ HPLC: 98.70% |

### BIOLOGICAL EXAMPLES

### Example B1. Calcium Flux in HEK293 Overexpressing TRPML1 Plasma Membrane Variant—Calcium 6 Dye

### Example B1.1 Materials

Cells: HEK293/TRPML1-PM clone A5, clonal cell line containing mutant TRPML1 (both N- and C-terminal dileucine targeting motifs deleted) that localizes to the plasma membrane

Culture media: EMEM (ATCC cat. #30-2003, 10% FBS (Hyclone defined cat. #SH30070.03), 1% pen/strep (Gibco cat. #15140-122), 200 mg/ml G418 (Gibco cat. #10131-035)

0.05% Trypsin: Gibco cat. #25300-054

Plates: Perkin Elmer Viewplate cat. #6007460, black optically clear, tissue culture treated 384-well plate

Calcium dye: Molecular Devices FLIPR Calcium 6 Assay Kit, cat. #R8190 or Fluo-4 Direct from Thermo Fisher, cat. #F10471

Tips: Molecular Devices 384-well FLIPR Tetra black tips, cat. #9000-0764 Example B1.2 Method

HEK293/TRPML1-PM clone A5 cells were trypsinized from flasks, counted, then diluted in media to 15,000 cells per 25 μL, and seeded into a black, 384-well viewplate, and cultured overnight.

One vial of calcium 6 dye was diluted with 10 mL of assay buffer (HBSS+20 mM HEPES pH 7.4) and 25 μL of the dilution was added per well to the cells.

After incubating for 2 hours at 37° C., the plate was moved to the FLIPR in the read position.

The compounds were diluted in EMEM with 0.125% FBS to 5× concentration and loaded in a 384-well plate according to the desired plate map for the assay, including DMSO alone and carbachol controls, then the plate was moved to the FLIPR in the source 2 position.

A box of tips was placed in the source 1 position within the FLIPR to complete the assay set up.

Once the run was started, an initial baseline fluorescent measurement was performed for 10 seconds (470-495 nm excitation and 515-575 nm emission) prior to adding the compounds.

12.5 μL of 5× test compound, DMSO alone, or carbachol were added via the FLIPR and real-time fluorescence measurements were performed for an additional 170 seconds with 1 read per second.

The data was normalized to baseline by calculating maximum fluorescence minus minimum fluorescence and plotted as response (fold) over the baseline versus log (inhibitor concentration). The EC₅₀ was determined using a 4-parameter logistic curve fit in Graphpad Prism 9.

### Example B2. TFEB Translocation Assay

### Example B2.1 Reagents

Rabbit TFEB antibody, Cell Signaling Technology #4240S

PFA 16% MeOH-free, Election Microscopy Sciences #50-980-487

HCS CellMask Deep Red, ThermoFisher Scientific #H32721

Hoechst 33342, AbCam #ab228551

Plates: Perkin Elmer Phenoplate #605730 black optically clear, tissue culture treated 384-well plate

### Example B2.2 Method, Day 1: Cells Seeded in 384WP

Cells were seeded in the afternoon. The outer one row of the plate was not used to avoid outer well edge effects.

HeLa cells were detached using trypsin, and the cells were counted using a cell counter.

4000 HeLa cells were seeded per well in 50 mL growth media (DMEM with 10% FBS, 1 mM sodium pyruvate and Pen-Strep).

### Example B2.3 Method, Day 2: Assay with TRPML1 Compounds+Staining

Compounds were added to cells using a Tecan D300e dispenser.

Cells were incubated for 90 mins at 37° C. with the compounds (compound range: 80 nM-10 mM (2× step dilution—8 dilution steps, 3 replicates per condition)

The cells were fixed by adding 16.5 μL of 16% PFA (4% final) and incubating for 15 min at room temperature.

The cells were washed with 3× in PBS-TX (PBS+0.1% Triton X-100)−1×100 μL followed by 2×50 μL.

The cells were blocked and permeabilized in 30 μL of blocking buffer (PBS-TX+5% goat serum) for 1 h.

The cells were incubated with TFEB antibody (1:200, CST 4240S) in 30 μL blocking buffer overnight at 4° C. The plates were kept damp to avoid evaporation.

### Example B2.4 Method, Day 3: Staining+Imaging

The cells were washed 3× in PBS-TX—3×100 μL with a Biotek EL406 washer.

The cells were incubated with Goat anti-Rb Alexa-Fluor 488 (1:2000), cell mask (1:10,000) and Hoechst (1:10,000) in 30 μL blocking buffer for 1 h at room temperature. The solution was dispensed using a 5 μL peristaltic cassette (EL406).

The cells were washed 3× in PBS-TX—3×100 μL with a EL406 washer, then 50 mL PBS was dispensed using a 5 μL peristaltic cassette (EL406).

The cells were imaged on an Operetta CLS High-content imaging system using a 20× water immersion objective (NA 1.0) the same day.

Image analysis was performed using Harmony V4.9 (Perkin Elmer). Briefly, nuclei were segmented from background-corrected images using Hoechst intensity, and cell body segmented using HCS CellMask DeepRed. A sliding parabola filter was applied to the AlexaFluor 488 channel (TFEB), before sum pixel intensity in the nuclear and cytoplasmic regions was calculated. A ratio of nuclear to cytoplasmic TFEB was used to define TFEB translocation from the cytosolic to nuclear compartment. The EC50 values derived by fitting the data to a sigmoidal dose response curve using Prism 9 (Graphpad).

The data from the assays described in Example B1 and Example B2 are summarized in Table 2. Compounds with EC₅₀ values at or below 10 M are considered TRPML1 activators.

TABLE 2

| Compound | TRPML1 calcium flux (FLIPR) | TFEB translocation |
|---|---|---|
| No. | dose response: EC50 (μM) | assay: EC50 (μM) |
| 1 | 1.24 | — |
| 2 | 3 | — |
| 3 | 4.76 | — |
| 4 | 2.21 | — |
| 5 | 2.46 | — |
| 6 | 0.29 | 4.86 |
| 7 | 0.51 | — |
| 8 | 3.69 | — |
| 9 | 3.46 | — |
| 10 | 0.87 | 2.34 |
| 11 | 2.1 | — |
| 12 | 0.72 | <0.08 |
| 13 | 0.67 | 1.35 |
| 14 | 1.56 | 2.03 |
| 15 | >10.0 | — |
| 16 | 2.37 | — |
| 17 | 0.19 | 0.45 |
| 18 | 1.14 | >9.9 |
| 19 | 2.03 | — |
| 20 | 0.26 | 0.27 |
| 21 | >10.0 | — |
| 22 | >10.0 | — |
| 23 | 0.41 | 0.20 |
| 24 | 0.28 | 1.87 |
| 25 | 0.42 | 0.54 |
| 26 | 0.30 | 0.83 |
| 27 | >10.0 | — |
| 28 | 0.35 | 1.1 |
| 29 | 0.51 | 2.27 |
| 30 | 0.36 | 1.17 |
| 31 | 8.98 | — |
| 32 | 0.81 | — |
| 33 | 2.74 | — |
| 34 | 3.2 | — |
| 35 | 0.33 | — |
| 36 | 2.18 | — |
| 37 | 0.75 | — |
| 38 | 0.24 | — |
| 39 | 0.42 | — |
| 40 | 0.41 | — |
| 41 | 0.27 | — |
| 42 | 0.82 | — |
| 43 | 0.88 | — |
| 44 | >10.0 | — |
| 45 | 2.06 | — |
| 46 | 2.56 | — |
| 47 | >10.0 | — |
| 48 | 7.45 | — |
| 49 | 1.14 | 7.97 |
| 50 | 6.55 | — |
| 51 | 3.05 | — |
| 52 | 5.78 | — |
| 53 | 1.06 | — |
| 54 | >10 | — |
| 55 | 0.25 | 0.87 |
| 56 | >10.0 | — |
| 57 | 4.61 | — |
| 58 | 6.88 | — |
| 59 | 2.24 | — |
| 60 | 0.0867 | — |
| 61 | >30 | — |
| 62 | >30 | — |
| 63 | 11 | — |
| 64 | 0.453 | 1.39 |
| 65 | 0.319 | 0.396 |
| 66 | 0.236 | 0.927 |
| 67 | 0.42 | 0.353 |
| 68 | 1.03 | 2.23 |
| 69 | 0.421 | 0.339 |
| 70 | 1.34 | 6.96 |
| 71 | 0.818 | 4.04 |
| 72 | 0.572 | 1.76 |
| 73 | 0.996 | 4.85 |
| 74 | 0.242 | 0.676 |
| 75 | 2.14 | — |
| 76 | 0.521 | 0.33 |
| 77 | 0.472 | 0.705 |
| 78 | 0.868 | 1.46 |
| 79 | 28.5 | — |
| 80 | 5.94 | — |
| 81 | 0.461 | 0.323 |
| 82 | 3.76 | — |
| 83 | >30 | — |
| 84 | 0.567 | 0.664 |
| 85 | 0.447 | 1 |
| 86 | >30 | — |
| 87 | 0.823 | 0.674 |
| 88 | 2.14 | — |
| 89 | 0.681 | 2.4 |
| 90 | 1.4 | — |
| 91 | 0.0772 | 0.199 |
| 92 | 7.44 | — |
| 93 | 0.857 | 0.649 |
| 94 | 2.53 | — |
| 95 | 0.274 | 1.27, 0.915 |
| 96 | 0.247 | 0.14 |
| 97 | 0.347 | 0.516 |
| 98 | 0.0466 | 0.117 |
| 99 | 0.433 | 0.573 |
| 100 | 1.16 | — |
| 101 | 3.02 | — |
| 102 | 0.412 | 0.388 |
| 103 | 1.97 | — |
| 104 | 0.715 | 0.573 |
| 105 | 4.36 | — |
| 106 | 0.371 | 0.464 |
| 107 | 1.39 | — |
| 108 | 0.79 | 0.431 |
| 109 | 0.28 | — |
| 110 | 0.256 | 0.246 |
| 111 | 0.812 | 3.29 |
| 112 | 3.72 | — |
| 113 | >10.0 | — |
| 114 | 4.02 | — |
| 115 | 1.1 | 0.758 |
| 116 | 1.03 | — |
| 117 | 0.353 | 0.0837 |
| 118 | 0.276 | 0.529 |

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula (I): 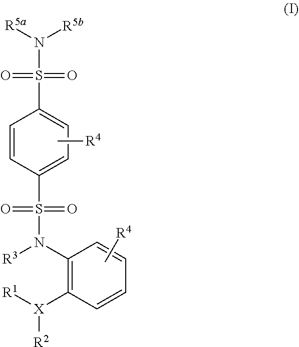
or a pharmaceutically acceptable salt thereof, wherein
X is N or H;
R¹ is chosen from H and optionally substituted alkyl;
R² is chosen from optionally substituted alkyl, optionally substituted cycloalkyl, aryl, and optionally substituted heterocyclyl;
or, when X is H, then R¹ and R² are absent;
R³ is H, —CH₃, or optionally substituted C₂-C₆ alkyl;
or R¹ and R³ together with the atoms attached thereto form a 5- to 7-membered ring;
each R⁴ is independently H, halo, cyano, or optionally substituted alkyl; and
R^{5a} and R^{5b} are each independently H or optionally substituted alkyl, with the proviso that R² is not pyrrolidine.

2. The compound of claim 1, or a pharmaceutically acceptable salt thereof, wherein X is N.

3. The compound of claim 1, or a pharmaceutically acceptable salt thereof, wherein X is H.

4. The compound of claims 1 or 2, or a pharmaceutically acceptable salt thereof, wherein R¹ is H.

5. The compound of claims 1 or 2, or a pharmaceutically acceptable salt thereof, wherein R¹ is C₁-C₆ alkyl optionally substituted with halo, oxo, or 3-6 membered heterocyclyl that contains at least O.

6. The compound of claim 5, or a pharmaceutically acceptable salt thereof, wherein R¹ is C₁-C₃ alkyl optionally substituted with halo, oxo, or 4-6 membered heterocyclyl that contains only O.

7. The compound of claim 6, or a pharmaceutically acceptable salt thereof, wherein R¹ is

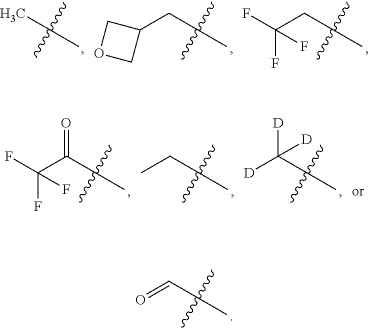

8. The compound of any one of claims 1, 2 or 4-7, or a pharmaceutically acceptable salt thereof, wherein R² is optionally substituted C₁-C₆ alkyl.

9. The compound of claim 8, or a pharmaceutically acceptable salt thereof, wherein R² is optionally substituted C₁-C₅ alkyl.

10. The compound of claim 9, or a pharmaceutically acceptable salt thereof, wherein R² is

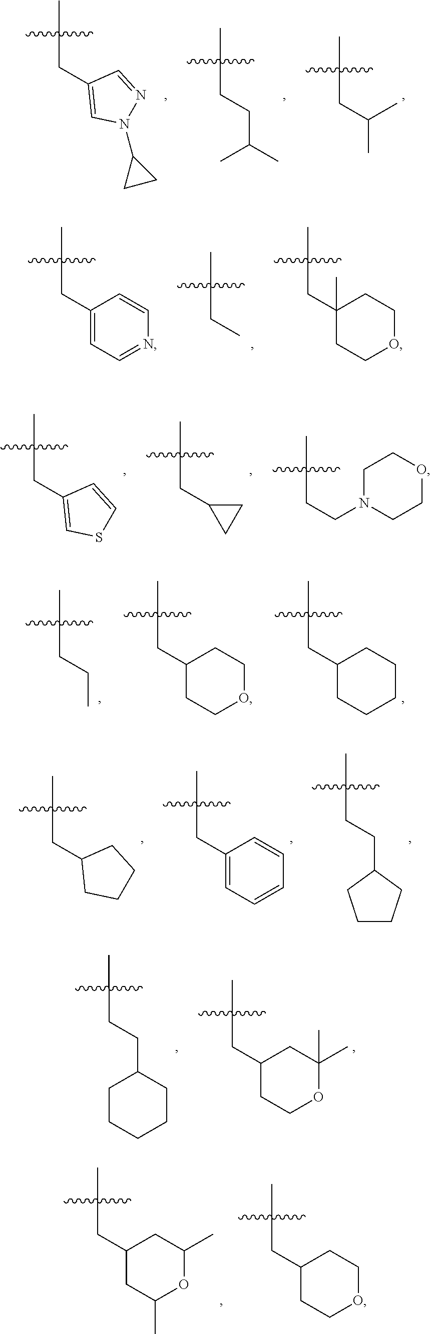

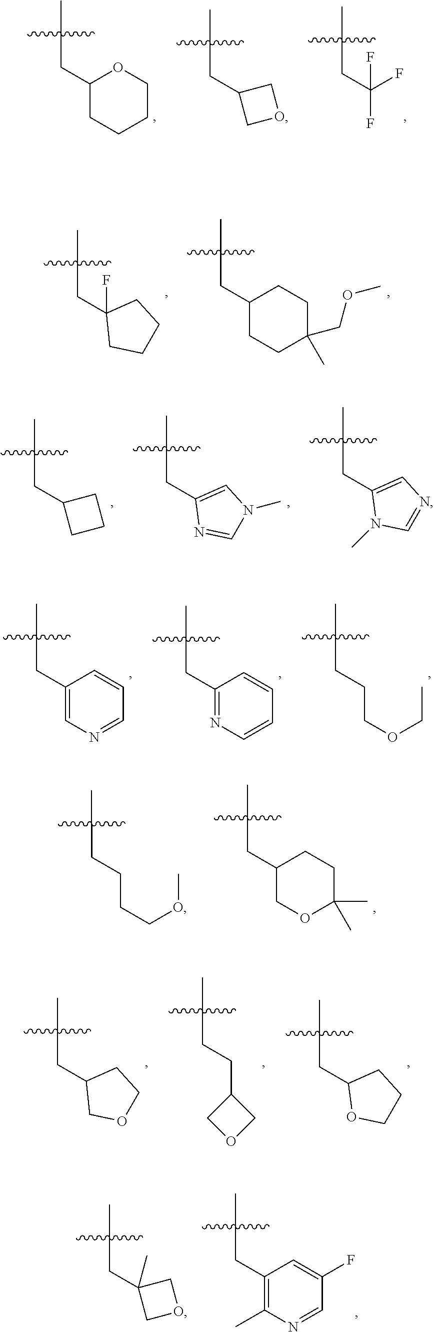

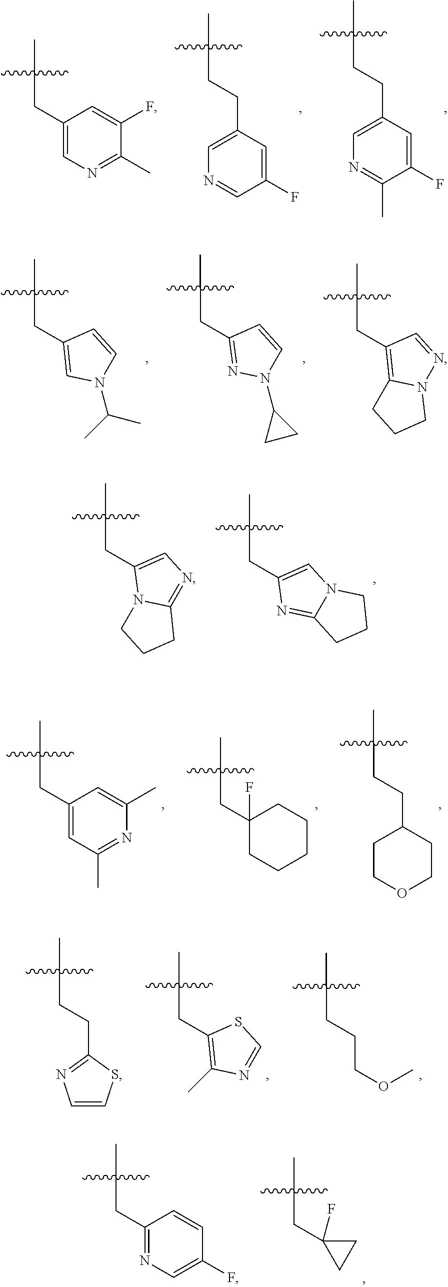

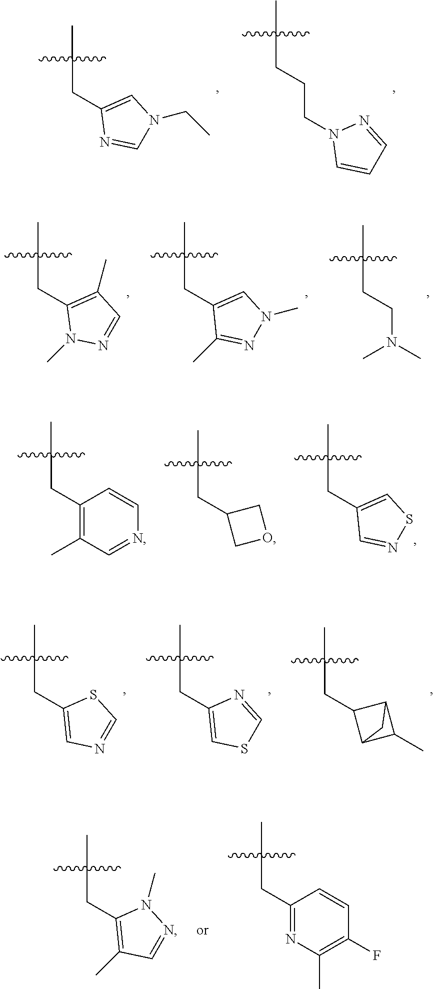

11. The compound of any one of claims 1, 2, or 4-7, or a pharmaceutically acceptable salt thereof, wherein R² is optionally substituted C₃-C₆ cycloalkyl.

12. The compound of claim 11, or a pharmaceutically acceptable salt thereof, wherein R² is optionally substituted C₄-C₆ cycloalkyl.

13. The compound of claim 12, or a pharmaceutically acceptable salt thereof, wherein R² is

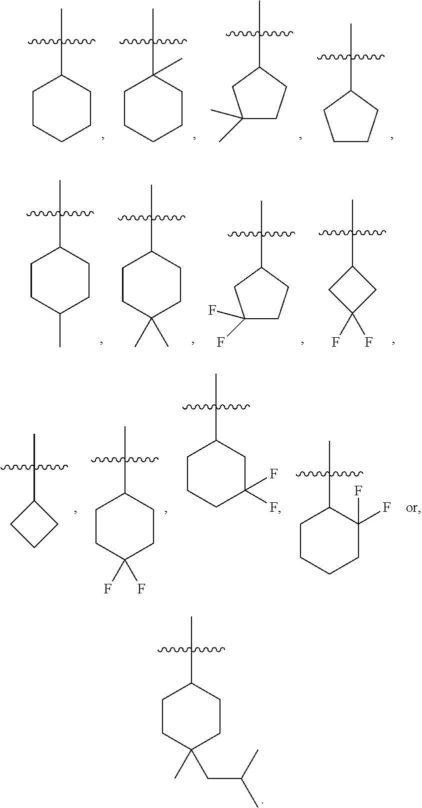

14. The compound of any one of claims 1, 2, or 4-7, or a pharmaceutically acceptable salt thereof, wherein R² is 6-membered aryl.

15. The compound of claim 14, or a pharmaceutically acceptable salt thereof, wherein R² is phenyl.

16. The compound of any one of claims 1, 2, or 4-7, or a pharmaceutically acceptable salt thereof, wherein R² is 6-membered heterocyclyl that contains only N optionally substituted with C₁-C₆ alkyl, —C(O)O(C₁-C₆ alkyl), —C(O)(C₁-C₆ alkyl), or —C(O)(C₁-C₆ cycloalkyl).

17. The compound of claim 16, or a pharmaceutically acceptable salt thereof, wherein R² is

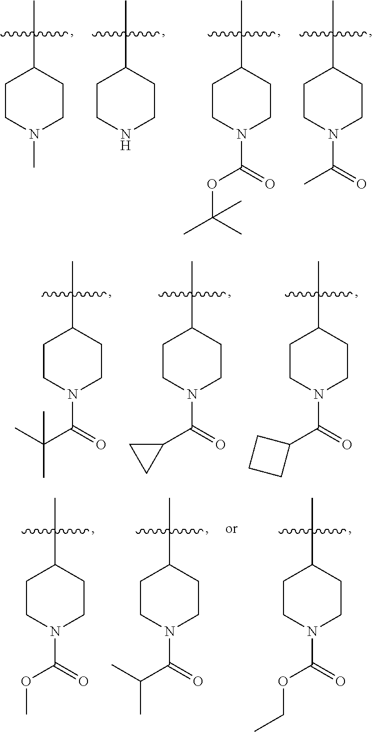

18. The compound of any one of claims 1, 2, or 4-7, or a pharmaceutically acceptable salt thereof, wherein R² is 3-10 membered heterocyclyl that contains only O optionally substituted with —CH₃.

19. The compound of claim 18, or a pharmaceutically acceptable salt thereof, wherein R² is

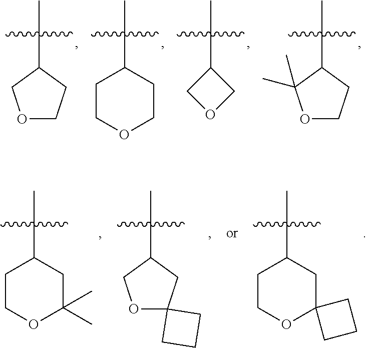

20. The compound of any one of claims 1-19, or a pharmaceutically acceptable salt thereof, wherein R³ is H.

21. The compound of any one of claims 1-19, or a pharmaceutically acceptable salt thereof, wherein R³ is —CH₃.

22. The compound of any one of claims 1-19, or a pharmaceutically acceptable salt thereof, wherein R³ is optionally substituted —CH₂CH₃.

23. The compound of any one of claims 1, 2, or 8-19, or a pharmaceutically acceptable salt thereof, wherein R¹ and R³ together with the atoms attached thereto form a 5-membered ring.

24. The compound of any one of claims 1-23, or a pharmaceutically acceptable salt thereof, wherein R⁴ is H.

25. The compound of any one of claims 1-23, or a pharmaceutically acceptable salt thereof, wherein R⁴ is F or Cl.

26. The compound of any one of claims 1-23, or a pharmaceutically acceptable salt thereof, wherein R⁴ is C₁-C₃ alkyl optionally substituted with halo.

27. The compound of claim 26, or a pharmaceutically acceptable salt thereof, wherein R⁴ is —CH₃ optionally substituted with one or more F.

28. The compound of any one of claims 1-23, or a pharmaceutically acceptable salt thereof, wherein R⁴ is cyano.

29. The compound of any one of claims 1-28, or a pharmaceutically acceptable salt thereof, wherein R^{5a} and R^{5b} are each independently H or C₁-C₃ alkyl.

30. The compound of claim 29, or a pharmaceutically acceptable salt thereof, wherein R^{5a} and R^{5b} are —CH₃.

31. The compound of any one of claims 1, 2, 4-20, 24-28, or 29-30, or a pharmaceutically acceptable salt thereof, wherein the compound is Formula (II):

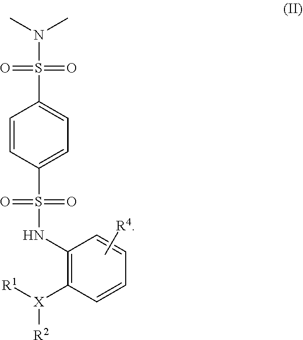

32. The compound of any one of claims 1, 2, 4-19, 21, 24, or 29-30, or a pharmaceutically acceptable salt thereof, wherein the compound is Formula (III):

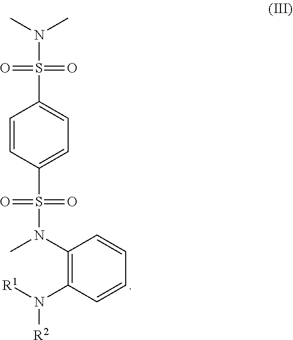

33. The compound of any one of claims 1, 2, 5-10, 20, 24-28, or 29-30, or a pharmaceutically acceptable salt thereof, wherein the compound is Formula (IV):

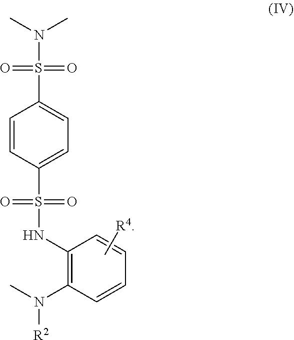

34. The compound of any one of claims 1, 2, 5-10, 21, 24, or 29-30, or a pharmaceutically acceptable salt thereof, wherein the compound is Formula (V):

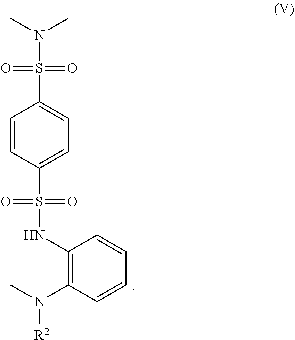

35. A compound selected from the compounds of Table 1 or a pharmaceutically acceptable salt thereof.

36. A compound and/or a pharmaceutically acceptable salt of any one of claims 1-35, wherein one or more hydrogen atoms attached to carbon atoms of the compound are replaced by deuterium atoms.

37. A pharmaceutical composition comprising the compound of any one of claims 1-36, or a pharmaceutically acceptable salt thereof, and a pharmaceutically acceptable excipient.

38. A method of modulating Mucolipin TRP channel subfamily 1 (TRPML1) comprising contacting TRPML1 with an effective amount of the compound of any one of claims 1-36, or a pharmaceutically acceptable salt thereof, or the pharmaceutical composition of claim 37.

39. A method of treating a disease associated with TRPML1 comprising administering to the subject an effective amount of the compound of any one of claims 1-36, or a pharmaceutically acceptable salt thereof, or the pharmaceutical composition of claim 37.

40. The method of claim 39, wherein the disease is a neurodegenerative disease, lysosomal storage disease, metabolic disease, cardiovascular disease, inflammatory disorder, immunological disorder, cancer, or aging.

41. The method of claim 39, wherein the disease is a neurodegenerative disease.

42. The method of claim 41, wherein the neurodegenerative disease is selected from Alzheimer's disease, Parkinson's disease, amyotrophic lateral sclerosis (ALS), HIV-associated dementia, Charcot-Marie-Tooth disease and Huntington's disease.
